(19) United States
(12) Patent Application Publication
(10) Pub. No.: US 2012/0321647 A1
(43) Pub. Date: Dec. 20, 2012

(54) STRUCTURED RNA MOTIFS AND COMPOUNDS AND METHODS FOR THEIR USE

(75) Inventors: Ronald R. Breaker, Guilford, CT (US); Zasha Weinberg, New Haven, CT (US)

(73) Assignee: YALE UNIVERSITY

(21) Appl. No.: 13/521,685

(22) PCT Filed: Jan. 12, 2011
(86) PCT No.: PCT/US11/20933

§ 371 Date: Jul. 11, 2012

Related U.S. Application Data
(60) Provisional application No. 61/335,852, filed on Jan. 12, 2010.

(51) Int. Cl. A61K 31/7076; C07H 21/02; A61K 31/197; C12N 1/20; A61K 39/40; A61P 31/04; C12N 15/63; C12Q 1/68

(52) U.S. Cl. 424/178.1; 435/320.1; 536/24.1; 435/6.1; 435/252.1; 514/46; 514/563

(57) ABSTRACT

Disclosed are compositions and methods involing riboswitches and RNA motifs. For example, disclosed are compositions and methods involving glutamine-responsive riboswitches, S-adenosylmethionine-repsonsive riboswitches, S-adenosylhomocysteine-repsonsive riboswitches, glutamine riboswitches, SAM/SAH riboswitches, glnA riboswitches, Downstream-peptide riboswitches, crcB riboswitches, pfl riboswitches, yjdF riboswitches, manA riboswitches, wcaG riboswitches, epsC riboswitches, ykkC-III riboswitches, psaA riboswitches, psbA riboswitches, PhotoRC-I riboswitches, PhotoRC-II riboswitches, and psbNH riboswitches.

### CROSS-REFERENCE TO RELATED APPLICATIONS

This application claims benefit of U.S. Provisional Application No. 61/335,852, filed Jan. 12, 2010. U.S. Provisional Application No. 61/335,852, filed Jan. 12, 2010, is hereby incorporated herein by reference in its entirety.

### STATEMENT REGARDING FEDERALLY SPONSORED RESEARCH

This invention was made with government support under Grant Nos. GMO2278 and RR19895-02 awarded by the National Institutes of Health (NIH). The government has certain rights in the invention.

### REFERENCE TO SEQUENCE LISTING

The Sequence Listing submitted Jan. 12, 2011 as a text file named “YU_{—}16_{—}9001_AMD_AFD_Sequence_Listing.txt,” created on Jan. 12, 2011, and having a size of 78,746 bytes is hereby incorporated by reference pursuant to 37 C.F.R. §1.52(e)(5).

### FIELD OF THE INVENTION

The disclosed invention is generally in the field of gene expression and specifically in the area of regulation of gene expression.

### BACKGROUND OF THE INVENTION

Precision genetic control is an essential feature of living systems, as cells must respond to a multitude of biochemical signals and environmental cues by varying genetic expression patterns. Most known mechanisms of genetic control involve the use of protein factors that sense chemical or physical stimuli and then modulate gene expression by selectively interacting with the relevant DNA or messenger RNA sequence. Proteins can adopt complex shapes and carry out a variety of functions that permit living systems to sense accurately their chemical and physical environments. Protein factors that respond to metabolites typically act by binding DNA to modulate transcription initiation (e.g. the lac repressor protein; Matthews, K. S., and Nichols, J. C., 1998, Prog. Nucleic Acids Res. Mol. Biol. 58, 127-164) or by binding RNA to control either transcription termination (e.g. the PyrR protein; Switzer, R. L., et al., 1999, Prog. Nucleic Acids Res. Mol. Biol. 62, 329-367) or translation (e.g. the TRAP protein; Babitzke, P., and Gollnick, P., 2001, J. Bacteriol. 183, 5795-5802). Protein factors respond to environmental stimuli by various mechanisms such as allosteric modulation or post-translational modification, and are adept at exploiting these mechanisms to serve as highly responsive genetic switches (e.g. see Ptashne, M., and Gann, A. (2002). Genes and Signals. Cold Spring Harbor Laboratory Press, Cold Spring Harbor, N.Y.).

In addition to the widespread participation of protein factors in genetic control, it is also known that RNA can take an active role in genetic regulation. Recent studies have begun to reveal the substantial role that small non-coding RNAs play in selectively targeting mRNAs for destruction, which results in down-regulation of gene expression (e.g. see Hannon, G. J. 2002, Nature 418, 244-251 and references therein). This process of RNA interference takes advantage of the ability of short RNAs to recognize the intended mRNA target selectively via Watson-Crick base complementation, after which the bound mRNAs are destroyed by the action of proteins. RNAs are ideal agents for molecular recognition in this system because it is far easier to generate new target-specific RNA factors through evolutionary processes than it would be to generate protein factors with novel but highly specific RNA binding sites.

Although proteins fulfill most requirements that biology has for enzyme, receptor and structural functions, RNA also can serve in these capacities. For example, RNA has sufficient structural plasticity to form numerous ribozyme domains (Cech & Golden, Building a catalytic active site using only RNA. In: The RNA World R. F. Gesteland, T. R. Cech, J. F. Atkins, eds., pp. 321-350 (1998); Breaker, In vitro selection of catalytic polynucleotides. Chem. Rev. 97, 371-390 (1997)) and receptor domains (Osborne & Ellington, Nucleic acid selection and the challenge of combinatorial chemistry. Chem. Rev. 97, 349-370 (1997); Hermann & Patel, Adaptive recognition by nucleic acid aptamers. Science 287, 820-825 (2000)) that exhibit considerable enzymatic power and precise molecular recognition. Furthermore, these activities can be combined to create allosteric ribozymes (Soukup & Breaker, Engineering precision RNA molecular switches. Proc. Natl. Acad. Sci. USA 96, 3584-3589 (1999); Seetharaman et al., Immobilized riboswitches for the analysis of complex chemical and biological mixtures. Nature Biotechnol. 19, 336-341 (2001)) that are selectively modulated by effector molecules.

Bacterial riboswitch RNAs are genetic control elements that are located primarily within the 5′-untranslated region (5′-UTR) of the main coding region of a particular mRNA. Structural probing studies (discussed further below) reveal that riboswitch elements are generally composed of two domains: a natural aptamer (T. Hermann, D. J. Patel, Science 2000, 287, 820; L. Gold, et al., Annual Review of Biochemistry 1995, 64, 763) that serves as the ligand-binding domain, and an ‘expression platform’ that interfaces with RNA elements that are involved in gene expression (e.g. Shine-Dalgarno (SD) elements; transcription terminator stems).

### BRIEF SUMMARY OF THE INVENTION

Disclosed are compositions and methods involing riboswitches and RNA motis. For example, disclosed are regulatable gene expression constructs comprising, for example, a nucleic acid molecule encoding an RNA comprising a riboswitch operably linked to a coding region, wherein the riboswitch regulates expression of the RNA, wherein the riboswitch and coding region are heterologous. The riboswitch can be, for example, a glutamine-responsive riboswitch, an S-adenosylmethionine-repsonsive riboswitch, an S-adenosylhomocysteine-repsonsive riboswitch, a glutamine riboswitch, a SAM/SAH riboswitch, a glnA riboswitch, a Downstream-peptide riboswitch, a crcB riboswitch, a pfl riboswitch, a yjdF riboswitch, a manA riboswitch, a wcaG riboswitch, an epsC riboswitch, a ykkC-III riboswitch, a psaA riboswitch, a psbA riboswitch, a PhotoRC-I riboswitch, a PhotoRC-II riboswitch, or a psbNH riboswitch. Also disclosed are, for example, riboswitches, wherein the riboswitch is a non-natural derivative of a naturally-occurring riboswitch. The naturally-occurring riboswitch can be, for example, a glutamine-responsive riboswitch, an S-adenosylmethionine-repsonsive riboswitch, an S-adenosylhomocysteine-repsonsive riboswitch, a glutamine riboswitch, a SAM/SAH riboswitch, a glnA riboswitch, a Downstream-peptide riboswitch, a crcB riboswitch, apt/riboswitch, a yjdF riboswitch, a manA riboswitch, a wcaG riboswitch, an epsC riboswitch, a ykkC-III riboswitch, a psaA riboswitch, a psbA riboswitch, a PhotoRC-I riboswitch, a PhotoRC-II riboswitch, or a psbNH riboswitch.

Also disclosed are, for example, methods of detecting a compound of interest, the method comprising, for example, bringing into contact a sample and a riboswitch, wherein the riboswitch is activated by the compound of interest, wherein the riboswitch produces a signal when activated by the compound of interest, wherein the riboswitch produces a signal when the sample contains the compound of interest. The riboswitch can be, for example, a glutamine-responsive riboswitch, an S-adenosylmethionine-repsonsive riboswitch, an S-adenosylhomocysteine-repsonsive riboswitch, a glutamine riboswitch, a SAM/SAH riboswitch, a glnA riboswitch, a Downstream-peptide riboswitch, a crcB riboswitch, a pfl riboswitch, a yjdF riboswitch, a manA riboswitch, a wcaG riboswitch, an epsC riboswitch, a ykkC-III riboswitch, a psaA riboswitch, a psbA riboswitch, a PhotoRC-I riboswitch, a PhotoRC-II riboswitch, or a psbNH riboswitch.

Also disclosed are methods of identifying compounds that interact with, modulates, inhibits, blocks, deactivates, and/or activates a riboswitch, such as a glutamine riboswitch. For example, disclosed are, for example, methods comprising, for example, (a) testing a compound for altering gene expression of a gene encoding an RNA comprising a riboswitch, wherein the alteration is via the riboswitch, and (b) altering gene expression by bringing into contact a cell and a compound that altered gene expression in step (a), wherein the cell comprises a gene encoding an RNA comprising a riboswitch, wherein the compound inhibits expression of the gene by binding to the riboswitch. The riboswitch can be, for example, a glutamine-responsive riboswitch, an S-adenosylmethionine-repsonsive riboswitch, an S-adenosylhomocysteine-repsonsive riboswitch, a glutamine riboswitch, a SAM/SAH riboswitch, a glnA riboswitch, a Downstream-peptide riboswitch, a crcB riboswitch, a pfl riboswitch, a yjdF riboswitch, a manA riboswitch, a wcaG riboswitch, an epsC riboswitch, a ykkC-III riboswitch, a psaA riboswitch, a psbA riboswitch, a PhotoRC-I riboswitch, a PhotoRC-II riboswitch, or a psbNH riboswitch.

Also disclosed are methods of identifying riboswitches, the method comprising, for example, assessing in-line spontaneous cleavage of an RNA molecule in the presence and absence of a compound, wherein the RNA molecule is encoded by a gene regulated by the compound, wherein a change in the pattern of in-line spontaneous cleavage of the RNA molecule indicates a riboswitch. The RNA can comprise a glutamine-responsive riboswitch or a derivative of a glutamine-responsive riboswitch and the compound can be glutamine. The RNA can comprise an S-adenosylhomocysteine-repsonsive riboswitch or a derivative of an S-adenosylhomocysteine-repsonsive riboswitch and the compound can be S-adenosylhomocysteine. The RNA can comprise an S-adenosylmethionine-repsonsive riboswitch or a derivative of an S-adenosylmethionine-repsonsive riboswitch and the compound can be S-adenosylmethionine.

Also disclosed are methods of altering gene expression, the method comprising, for example, bringing into contact a compound and a cell, wherein the cell comprises a gene encoding an RNA comprising, for example, a glutamine-responsive riboswitch, an S-adenosylmethionine-repsonsive riboswitch, an S-adenosylhomocysteine-repsonsive riboswitch, a glutamine riboswitch, a SAM/SAH riboswitch, a glnA riboswitch, a Downstream-peptide riboswitch, a crcB riboswitch, a pfl riboswitch, a yjdF riboswitch, a manA riboswitch, a wcaG riboswitch, an epsC riboswitch, a ykkC-III riboswitch, a psaA riboswitch, a psbA riboswitch, a PhotoRC-I riboswitch, a PhotoRC-II riboswitch, or a psbNH riboswitch.

The riboswitch can comprise an aptamer domain and an expression platform domain, wherein the aptamer domain and the expression platform domain are heterologous. The riboswitch can comprise two or more aptamer domains and an expression platform domain, wherein at least one of the aptamer domains and the expression platform domain are heterologous. In some forms, at least two of the aptamer domains exhibit cooperative binding. The riboswitch can comprise, for example, a glnA motif, a Downstream-peptide motif, a SAM/SAH motif, a crcB motif, a pfl motif, a yjdF motif, a manA motif, a wcaG motif, a epsC motif, a ykkC-III motif, a psaA motif, a psbA motif, a PhotoRC-I motif, a PhotoRC-II motif, or a psbNH motif.

The riboswitch can be activated by a trigger molecule, wherein the riboswitch produces a signal when activated by the trigger molecule. The riboswitch can have, for example, one of the consensus structures of FIG. 1, FIG. 2, FIG. 3, FIG. 4, or FIG. 5.

In some forms, the riboswitch can comprise an aptamer domain and an expression platform domain wherein the aptamer domain is derived from a naturally-occurring glutamine-responsive riboswitch, S-adenosylmethionine-repsonsive riboswitch, S-adenosylhomocysteine-repsonsive riboswitch, glutamine riboswitch, SAM/SAH riboswitch, glnA riboswitch, Downstream-peptide riboswitch, crcB riboswitch, pfl riboswitch, yjdF riboswitch, manA riboswitch, wcaG riboswitch, epsC riboswitch, ykkC-III riboswitch, psaA riboswitch, psbA riboswitch, PhotoRC-I riboswitch, PhotoRC-II riboswitch, or psbNH riboswitch. In some forms, the aptamer domain can be the aptamer domain of a naturally-occurring glutamine-responsive riboswitch, S-adenosylmethionine-repsonsive riboswitch, S-adenosylhomocysteine-repsonsive riboswitch, glutamine riboswitch, SAM/SAH riboswitch, glnA riboswitch, Downstream-peptide riboswitch, crcB riboswitch, pfl riboswitch, yjdF riboswitch, manA riboswitch, wcaG riboswitch, epsC riboswitch, ykkC-III riboswitch, psaA riboswitch, psbA riboswitch, PhotoRC-I riboswitch, PhotoRC-II riboswitch, or psbNH riboswitch.

The aptamer domain can have the consensus structure of an aptamer domain of the naturally-occurring riboswitch. In some forms, the aptamer domain can consist of only base pair conservative changes of the naturally-occurring riboswitch.

In some forms, the riboswitch changes conformation when activated by the compound of interest, wherein the change in conformation produces a signal via a conformation dependent label. In some forms, the riboswitch changes conformation when activated by the compound of interest, wherein the change in conformation causes a change in expression of an RNA linked to the riboswitch, wherein the change in expression produces a signal. In some forms, the signal is produced by a reporter protein expressed from the RNA linked to the riboswitch.

In some forms, the cell can be identified as being in need of altered gene expression. The cell can be a bacterial cell. The compound can kill or inhibit the growth of the bacterial cell. The compound and the cell can be brought into contact by administering the compound to a subject. The cell can be a bacterial cell in the subject, wherein the compound kills or inhibits the growth of the bacterial cell. The subject can have a bacterial infection. The compound can be administered in combination with another antimicrobial compound. The compound can inhibit bacterial growth in a biofilm.

Additional advantages of the disclosed method and compositions will be set forth in part in the description which follows, and in part will be understood from the description, or can be learned by practice of the disclosed method and compositions. The advantages of the disclosed method and compositions will be realized and attained by means of the elements and combinations particularly pointed out in the appended claims. It is to be understood that both the foregoing general description and the following detailed description are exemplary and explanatory only and are not restrictive of the invention as claimed.

### BRIEF DESCRIPTION OF THE DRAWINGS

The accompanying drawings, which are incorporated in and constitute a part of this specification, illustrate several embodiments of the disclosed method and compositions and together with the description, serve to explain the principles of the disclosed method and compositions.

FIGS. 1A, 1B, 1C, and 1D show the SAM/SAH riboswitches. (A) SAM/SAH motif consensus diagram. Additional base pairing interactions are discussed in Example 3. Solid filled circles indicate a nucleotide is present at that position in 97% of the motifs; cross hatched circles indicate a nucleotide is present at that position in 90% of the motifs; parallel line circles indicate a nucleotide is present at that position in 75% of the motifs; and open circles indicate a nucleotide is present at that position in 50% of the motifs. An uppercase nucleotide letter inside an open circle indicates that 97% of the nucleotides at that position have sequence identity with the nucleotide indicated by the letter; an uppercase nucleotide letter not inside a circle indicates that 90% of the nucleotides at that position have sequence identity with the nucleotide indicated by the letter; and a lowercase nucleotide letter indicates that 75% of the nucleotides at that position have sequence identity with the nucleotide indicated by the letter. A dash (—) between paired nucleotide indicates base pairing; triple dots (• • •) between paired nucleotides indicates that covarying mutations were observed; double dots (• •) between paired nucleotides indicates that compatible mutations were observed; and a single dot (•) between paired nucleotides indicates that no mutations were observed. (B) Sequence and proposed secondary structure of SK209-52 RNA. In-line probing annotations are derived from the data in C. Asterisks identify G residues added to improve in vitro transcription yield. Enclosed positions indicate areas of the RNA where internucleotide linkages undergo reduced (uppercase letter in diamond), constant (uppercase letter in circle), or increased (lowercase letter in circle) scission as ligand concentrations are increased when subjected to in-line probing. Lowercase letter in diamond means no data. (C) In-line probing gel with lanes loaded with 5′ ³²P-labeled RNAs subjected to no reaction (NR), partial digestion with RNase T1 (T1), partial digest under alkaline pH (⁻OH), in-line probing reaction without added compound (—), or in-line probing reactions with various concentrations of SAM. Selected bands in the RNase T1 partial digest lane (products of cleavage 3′ of G residues) are numbered according to the nucleotide positions in B. Uncleaved precursor (Pre) and two internucleotide linkages whose cleavage rates are strongly affected by SAM (3′ of nucleotides 42 and 45) are marked. (D) Plot of the normalized fraction of RNAs whose cleavage sites (linkage 23 not shown in C) have undergone modulation versus the concentration of SAM present during the in-line probing reaction. The curve represents an ideal one-to-one binding interaction with a K_{D} of 8.6 μM.

FIGS. 2A, 2B, 2C, 2D, 2E, 2F, and 2G show motifs of the crcB, yjdF, wcaG, manA, pfl, epsC and ykkC-III riboswitches Annotations are as described in FIG. 1A and its description. Question marks signify base-paired regions (“P4?” in yjdF, “P2?” in pfl, and “pseudoknot?” in manA) with weaker covariation or structural conservation. The pseudoknot in the epsC motif was predicted by others. T Annotations are as described in FIG. 1A and its description.

FIG. 3 shows motifs of the glnA and Downstream-peptide riboswitches. Annotations are as described in FIG. 1A and its description. Purple lines and numbers indicate conserved sequences or structures common to the two motifs. Annotations are as described in FIG. 1A and its description.

FIGS. 4A, 4B, 4C, and 4D show cyanobacterial motifs related to photosynthesis. Annotations are as described in FIG. 1A and its description.

FIGS. 5A, 5B, 5C, 5D, 5E, 5F, 5G, 5H, 5I, 5J, 5K, and 5M show examples of other RNA motifs. Annotations are as described in FIG. 1A and its description. The Bacteroidales-1 motif has more conserved nucleotides than depicted (see FIG. 11).

FIGS. 6A and 6B show binding characteristics of SAM/SAH riboswitches. (A) The dissociation constant (K_{D}) was calculated for SK209-52 RNA when binding compounds related to SAM. The structures of SAM and SAH are marked. The horizontal dashed line indicates the K_{D} for SAM. (B) Two previously established SAM-binding RNAs (156 metA, 62 metY) and SK209-52 RNA were subjected to equilibrium dialysis experiments. An SK209-52 RNA mutant, termed “A48U”, was also tested. The SAM209-52 structure is shown at left, with the A48U mutation. At right is shown the ratios of the counts/minute in chamber B (containing the RNA) divided by that in chamber A (containing SAM with a ³H-labeled methyl group). Error bars reflect three independent experiments. The horizontal dashed line indicates a ratio of 1, which is the expected value when no RNA is used, or when an RNA that does not bind SAM is used.

FIGS. 7A, 7B, 7C, and 7D show genes regulated by pfl RNAs in the context of purine and one-carbon metabolism. This diagram is adapted from a previously published diagram regarding purine metabolism (Ravcheev et al. Purine regulon of gamma-proteobacteria: a detailed description. Russian Journal of Genetics 2002, 38:1015-1025).

The apparent regulation by a pfl RNA of an rpiB gene was observed in more recent homology searches (unpublished data). Genes known to be regulated by purR in Gram-positive or Gram-negative bacteria were collected from previous reports (Ravcheev et al. Purine regulon of gamma-proteobacteria: a detailed description. Russian Journal of Genetics 2002, 38:1015-1025; Weng et al. Identification of the Bacillus subtilis pur operon repressor. Proc Natl Acad Sci USA 1995, 92:7455-7459; Johansen et al. Definition of a second Bacillus subtilis pur regulon comprising the pur and xpt-pbuX operons plus pbuG, nupG (yxjA), and pbuE (ydhL). J Bacteriol 2003, 185:5200-5209). The compound formyltetrahydrofolate is repeated three times in the diagram, as indicated by the label “same compound”.

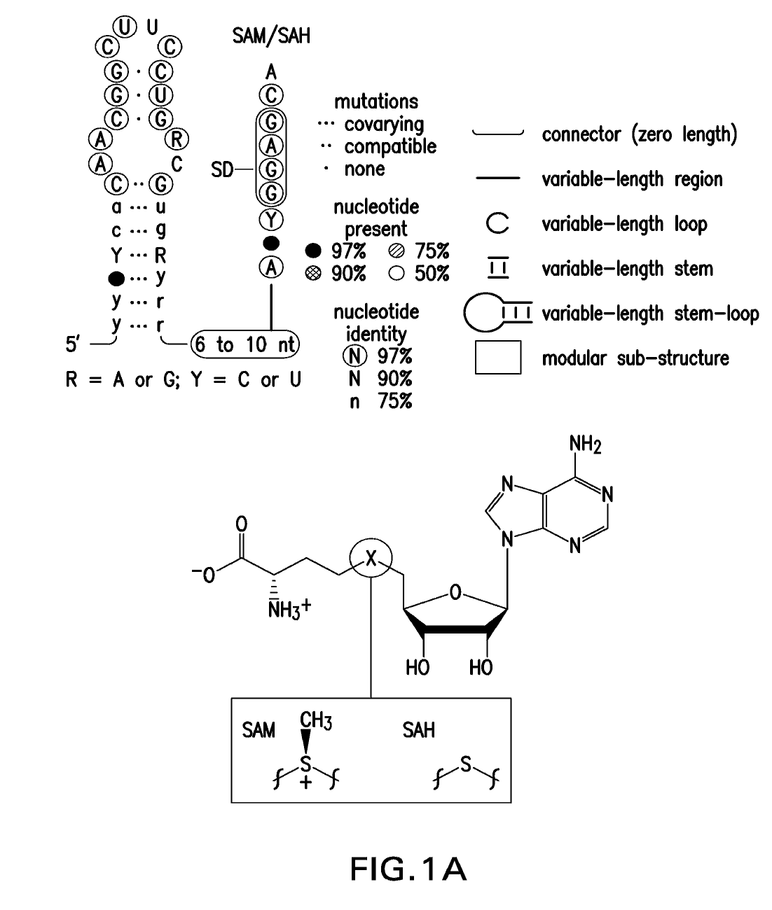

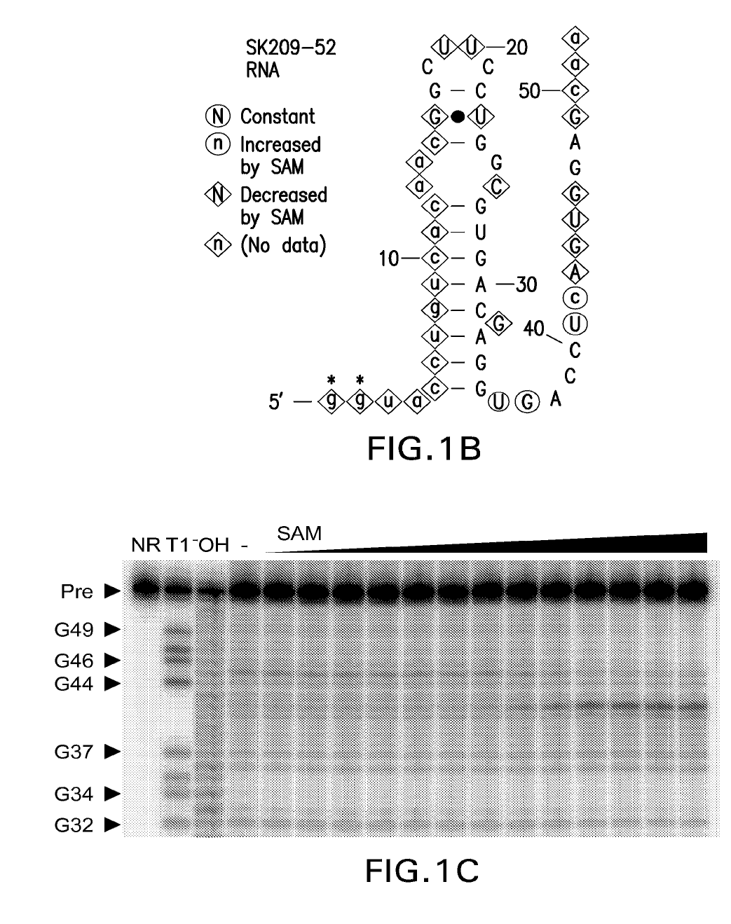

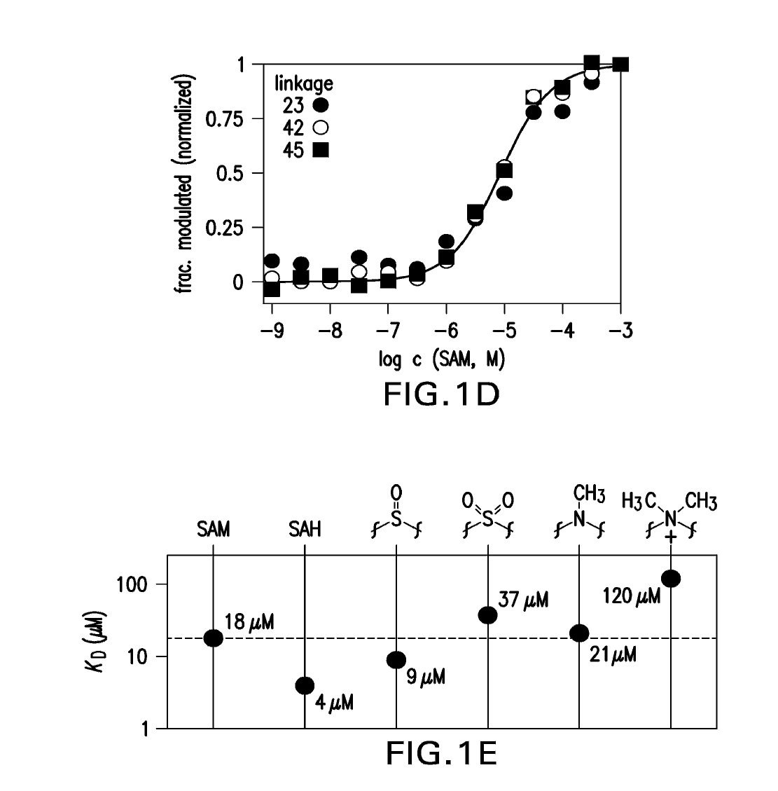

FIGS. 8A and 8B show a comparison of ykkC-III and mini-ykkC motifs. Conserved sequences with the consensus ACGA present in the ykkC-III or mini-ykkC motifs are shaded blue. The ykkC-III motif contains AC and GA sequences that can together comprise an ACGA sequence. The mini-ykkC motif drawing is derived from a previous depiction (Weinberg et al., 2007). Annotations are as described in FIG. 1A and its description.

FIGS. 9A, 9B, 9C, 9D, and 9E show 6S-Flavo, aceE, Acido-1, Acido-Lenti-1, and Actino-pnp motifs. Annotations are as described in FIG. 1A and its description. FIGS. 10A, 10B, 10C, 10D and 10E show AdoCbl-variant, asd, atoC, Bacillaceae-1, and Bacillus-plasmid motifs. Annotations are as described in FIG. 1A and its description.

FIGS. 11A, 11B, 11C, 11D, and 11E show Bacteroidales-1, Bacteroides-1, Bacteroides-2, c4 antisense RNA, and c4 antisense RNA target a1b1 motifs. Annotations are as described in FIG. 1A and its description.

FIGS. 12A, 12B, 12C, 12D, and 12E show Chlorobi-1, Chlorobi-RRM, Chloroflexi-1, Clostridiales-1, and Collinsella-1 motifs. Annotations are as described in FIG. 1A and its description.

FIGS. 13A, 13B, 13C, 13D, and 13E show crcB, Cyano-1, Cyano-2, Desulfotalea-1, and Downstream-peptide motifs. Annotations are as described in FIG. 1A and its description.

FIGS. 14A, 14B, 14C, and 14D show Dictyoglomi-1, epsC, fixA, and Flavo-1 motifs. Annotations are as described in FIG. 1A and its description.

FIGS. 15A, 15B, 15C, 15D, 15E, and 15F show flpD, flg-Rhizobiales, gabT, Gamma-cis-1, glnA, and GUCCY-hairpin motifs. Annotations are as described in FIG. 1A and its description.

FIGS. 16A, 16B, 16C, 16D, 16E, and 16F show Gut-1, gyrA, hopC, icd, JUMPstart, and L17 downstream element motifs. Annotations are as described in FIG. 1A and its description.

FIGS. 17A, 17B, 17C, 17D, 17E, and 17F show iactis-plasmid, Lacto-int, Lacto-rpoB, Lacto-usp, leu/phe leader, and Lnt motifs. Annotations are as described in FIG. 1A and its description.

FIGS. 18A, 18B, 18C, 18D, and 18E show manA, Methylobacterium-1, metK-Rhodobacter, Moco-II, and msiK motifs. Annotations are as described in FIG. 1A and its description.

FIGS. 19A, 19B, 19C, and 19D show Ocean-V, Ocean-VI, pan, and Pedo-repair motifs. Annotations are as described in FIG. 1A and its description.

FIGS. 20A, 20B, 20C, 20D, and 20E show pfl, psaA, pheA, PhotoRC-I, and PhotoRC-II motifs. Annotations are as described in FIG. 1A and its description.

FIGS. 21A, 21B, and 21C show Polynucleobacter-1, potC, and Pseudomon-1 motifs. Annotations are as described in FIG. 1A and its description.

FIGS. 22A, 22B, 22C, 22D, 22E, and 22F show psbNH, Pseudomon-2, Pseudomon-groES, Pseudomon-Rho, Pyrobac-1, and Pyrobac-HINT 1 motifs. Annotations are as described in FIG. 1A and its description.

FIGS. 23A, 23B, 23C, 23D, and 23E show radC, Rhizobiales-1, Rhizobiales-2, Rhodopirellula-1, and rmf motifs. Annotations are as described in FIG. 1A and its description.

FIGS. 24A, 24B, 24C, 24D, and 24E show rne-H, SAM-Chlorobi, and SAM-1-nil motifs. Annotations are as described in FIG. 1A and its description.

FIGS. 25A, 25B, 25C, 25D, 25E, and 25F show SAM/SAH, sanguinis-hairpin, sbcD, ScRE, Soil-1, and Solibacter-1 motifs. Annotations are as described in FIG. 1A and its description.

FIGS. 26A, 26B, and 26C show STAXI, sucA-II, and sucC motifs. Annotations are as described in FIG. 1A and its description.

FIGS. 27A, 27B, 27C, and 27D show Termite-fig, Termite-leu, traJ-II, and TwoAYGGAY motifs. Annotations are as described in FIG. 1A and its description.

FIGS. 28A, 28B, 28C, and 28D show wcaG, Whalefall-1, yjdF, and ykkC-III motifs. Annotations are as described in FIG. 1A and its description.

FIGS. 29A and 29B show a consensus sequence and secondary structure models for two riboswitch aptamcr families. (A) The glnA motif is a 3-stem junction (stems arc named P1, P2 and P3) that carries an E-loop and a possible single long-distance base pair (dashed line). (B) The Downstream-peptide motif is formed by three extended base-paired substructures wherein P1 and P2 are nearly identical to those of the glnA motif. The motif lacks P3 and E-loop features, but nucleotides in this region form a pseudoknot. Like glnA RNAs, the Downstream-peptide motif can potentially form a single long-range base pair. The two motifs also carry identical nucleotides at the base of P1 and in the junction. These models are derived using methods and data reported previously (Weinberg et al., Genome Biol 2010; 11:R31).

FIGS. 30A, 30B, and 30C show the 67 glnA RNA binds L-glutamine. (A) Sequence and secondary structural model for the 67 glnA RNA from S. elongates. Enclosed positions indicate areas of the RNA where internucleotide linkages undergo reduced (uppercase letter in diamond) or constant (uppercase letter in circle) scission as ligand concentrations are increased when subjected to in-line probing (data from B).

Nucleotides depicted in lowercase identify guanosine residues added to the construct to facilitate efficient in vitro transcription. Asterisks indicate the boundaries of the annotations for in-line probing results that could be clearly resolved by PAGE. (B) In-line probing analysis of 5′ ³²P-labeled 67 glnA RNA. Precursor RNAs (Pre) were loaded onto gel lanes after treatment as follows: NR, no reaction; T1, partial digest with RNase T1 (cleaves after G residues); ⁻OH, partial alkaline-mediated degradation; -, RNA subjected to in-line probing conditions without the addition of L-glutamine; and [L-glutamine], RNAs incubated under in-line probing conditions in the presence of various concentrations of L-glutamine ranging from 1 μM to 10 mM. Vertical lines designate areas where band intensities decrease as the RNA is exposed to higher concentrations of ligand. Band intensities of numbered regions were quantified and used to assess the extent of ligand binding. (C) Plot of the normalized fraction of band modulation (interpreted as fraction of RNAs bound to ligand) versus the logarithm of the concentration of ligand. Regions are as depicted in B. The line represents the curve expected for a 1-to-1 RNA-ligand interaction with a K_{D} of 575 μM.

FIGS. 31A and 31B show tandem glutamine aptamers. (A) Distribution of glutamine aptamers among single, double and triple arrangements. Aptamers were grouped together if the amount of intervening sequence was less than 100 nucleotides. (B) Consensus sequence and structure of the most common tandem arrangement of glutamine aptamers. Annotations are as described in FIG. 1A and its description.

FIGS. 32A, 32B, and 32C show that the 83 DP RNA is an aptamer for glutamine. (A) Sequence and secondary structure of the 83 DP RNA. Lowercase letters in a circle indicate internucleotide linkages that undergo greater scission when the RNA is exposed to ligand. Other annotations are as described for FIG. 30A. (B) In-line probing analysis of 5′ ³²P-labeled 83 DP RNA with various concentrations of L-glutamine ranging from 10 μM to 10 mM. Annotations are as described for FIG. 30B, with the exception that arrows indicate specific bands which were used to make the K_{D} plot in C. (C) Plot representing ligand binding as described for FIG. 30C. The line represents the curve expected for a 1-to-1 interaction using a K_{D} value 5 mM.

### DETAILED DESCRIPTION OF THE INVENTION

The disclosed methods, compounds, and compositions can be understood more readily by reference to the following detailed description of particular embodiments and the Examples included therein and to the Figures and their previous and following description.

Messenger RNAs are typically thought of as passive carriers of genetic information that are acted upon by protein- or small RNA-regulatory factors and by ribosomes during the process of translation. It was discovered that certain mRNAs carry natural aptamer domains and that binding of specific metabolites directly to these RNA domains leads to modulation of gene expression. Natural riboswitches exhibit two surprising functions that are not typically associated with natural RNAs. First, the mRNA element can adopt distinct structural states wherein one structure serves as a precise binding pocket for its target metabolite. Second, the metabolite-induced allosteric interconversion between structural states causes a change in the level of gene expression by one of several distinct mechanisms. Riboswitches typically can be dissected into two separate domains: one that selectively binds the target (aptamer domain) and another that influences genetic control (expression platform). It is the dynamic interplay between these two domains that results in metabolite-dependent allosteric control of gene expression.

Distinct classes of riboswitches have been identified and are shown to selectively recognize activating compounds (referred to herein as trigger molecules). For example, coenzyme B₁₂, glycine, thiamine pyrophosphate (TPP), and flavin mononucleotide (FMN) activate riboswitches present in genes encoding key enzymes in metabolic or transport pathways of these compounds. The aptamer domain of each riboswitch class conforms to a highly conserved consensus sequence and structure. Thus, sequence homology searches can be used to identify related riboswitch domains. Riboswitch domains have been discovered in various organisms from bacteria, archaca, and cukarya.

Riboswitches are genetic regulatory elements composed solely of RNA that bind metabolites and control gene expression commonly without the involvement of protein factors (Breaker R R. Riboswitches: from ancient gene-control systems to modern drug targets. Future Microbiol 2009; 4:771-773). Most simple riboswitches are composed of an aptamer domain and an expression platform, where the aptamer functions as a receptor for a specific metabolite and the expression platform modulates the expression of one or more genes in a ligand-dependent fashion (Barrick et al. The distributions, mechanisms, and structures of metabolite-binding riboswitches. Genome Bio12007; 8:R239; Dambach et al. Expanding roles for metabolite-sensing regulatory RNAs. Curr Opin Microbiol 2009; 12:161-169). Riboswitches are usually found in the 5′ untranslated regions (UTRs) of bacterial mRNAs and often control gene expression in cis either at the level of transcription or translation, although other regulatory mechanisms are also known (Roth et al. The structural and functional diversity of metabolite-binding riboswitches Annu Rev Biochem 2009; 78:305-334). In most cases, metabolite binding triggers a structural rearrangement that affects the formation of either a terminator stem or a base-paired element that occludes the ribosome binding site. In addition, there is a known example of a trans-acting riboswitch (Loh et al. A trans-acting riboswitch controls expression of the virulence regulator PrfA in listeria monocytogenes. Cell 2009; 139:770-779) as well as eukaryotic riboswitches (Wachter A. Riboswitch-mediated control of gene expression in eukaryotes. RNA Biol 2010; 7:67-76) that modulate expression by controlling alternative mRNA spicing in algae (Croft et al. Thiamine biosynthesis in algae is regulated by riboswitches. Proc Natl Acad Sci USA 2007; 104:20770-20775), plants (Wachter et al. Riboswitch control of gene expression in plants by splicing and alternative 3′ end processing of mRNAs. Plant Cell 2007; 19:3437-3450), and fungi (Cheah et al. Control of alternative RNA splicing and gene expression by eukaryotic riboswitches. Nature 2007; 447:497-500).

Comparative sequence analysis methods have been developed for novel riboswitch class discovery (Rodionov et al. Regulation of lysine biosynthesis and transport genes in bacteria: yet another RNA riboswitch? Nucleic Acids Res 2003; 31:6748-6757; Barrick et al. New RNA motifs suggest an expanded scope for riboswitches in bacterial genetic control. Proc Natl Acad Sci USA 2004; 101:6421-6426; Weinberg et al., 2007). These techniques involve computational searches through genomic and metagenomic databases for sequences that are conserved both in their primary and secondary structures (Yao et al. A computational pipeline for high-throughput discovery of cis-regulatory noncoding RNA in prokaryotes. PLoS Comput Biol 2007; 3:e126; Tseng et al. Finding non-coding RNAs through genome-scale clustering. J Bioinform Comput Bio12009; 7:373-388). Through one of these searches, the glnA motif and the Downstream-peptide motif (FIG. 29) were discovered in cyanobacteria and marine metagenomic sequences (Weinberg et al., Genome Biol 2010; 11:R31).

Structured noncoding RNAs perform many functions that are essential for protein synthesis, RNA processing, and gene regulation. Structured RNAs can be detected by comparative genomics, in which homologous sequences are identified and inspected for mutations that conserve RNA secondary structure. By applying a comparative genomics-based approach to genome and metagenome sequences from bacteria and archaea, 104 structured RNA motifs were identified. Three metabolite-binding RNA motifs were validated, including one that binds the coenzyme S-adenosylmethionine, and a further nine metabolite-binding RNA motifs were identified. New-found cis-regulatory RNA motifs are implicated in photosynthesis or nitrogen regulation in cyanobacteria, purine and one-carbon metabolism, stomach infection by Helicobacter, and many other physiological processes. A riboswitch termed crcB is represented in both bacteria and archaea. Another RNA motif controls gene expression from 3′ untranslated regions (UTRs) of mRNAs, which is unusual for bacteria. Many noncoding RNAs that act in trans are also revealed, and several of the noncoding RNA motifs are found mostly or exclusively in metagenome DNA sequences. This work greatly expands the variety of highly-structured noncoding RNAs known to exist in bacteria and archaea.

### A. General Organization of Riboswitch RNAs

Bacterial riboswitch RNAs are genetic control elements that are located primarily within the 5′-untranslated region (5′-UTR) of the main coding region of a particular mRNA. Structural probing studies (discussed further below) reveal that riboswitch elements are generally composed of two domains: a natural aptamer (T. Hermann, D. J. Patel, Science 2000, 287, 820; L. Gold, et al., Annual Review of Biochemistry 1995, 64, 763) that serves as the ligand-binding domain, and an ‘expression platform’ that interfaces with RNA elements that are involved in gene expression (e.g. Shine-Dalgarno (SD) elements; transcription terminator stems). These conclusions are drawn from the observation that aptamer domains synthesized in vitro bind the appropriate ligand in the absence of the expression platform (see Examples 2, 3 and 6 of U.S. Application Publication No. 2005-0053951). Moreover, structural probing investigations indicate that the aptamer domain of most riboswitches adopts a particular secondary- and tertiary-structure fold when examined independently, that is essentially identical to the aptamer structure when examined in the context of the entire 5′ leader RNA. This indicates that, in many cases, the aptamer domain is a modular unit that folds independently of the expression platform (see Examples 2, 3 and 6 of U.S. Application Publication No. 2005-0053951).

Ultimately, the ligand-bound or unbound status of the aptamer domain is interpreted through the expression platform, which is responsible for exerting an influence upon gene expression. The view of a riboswitch as a modular element is further supported by the fact that aptamer domains are highly conserved amongst various organisms (and even between kingdoms as is observed for the TPP riboswitch), (N. Sudarsan, et al., RNA 2003, 9, 644) whereas the expression platform varies in sequence, structure, and in the mechanism by which expression of the appended open reading frame is controlled. For example, ligand binding to the TPP riboswitch of the tenA mRNA of B. subtilis causes transcription termination (A. S. Mironov, et al., Cell 2002, 111, 747). This expression platform is distinct in sequence and structure compared to the expression platform of the TPP riboswitch in the thiM mRNA from E. coli, wherein TPP binding causes inhibition of translation by a SD blocking mechanism (see Example 2 of U.S. Application Publication No. 2005-0053951). The TPP aptamer domain is easily recognizable and of near identical functional character between these two transcriptional units, but the genetic control mechanisms and the expression platforms that carry them out are very different.

Aptamer domains for riboswitch RNAs typically range from ˜70 to 170 nt in length (FIG. 11 of U.S. Application Publication No. 2005-0053951). This observation was somewhat unexpected given that in vitro evolution experiments identified a wide variety of small molecule-binding aptamers, which are considerably shorter in length and structural intricacy (T. Hermann, D. J. Patel, Science 2000, 287, 820; L. Gold, et al., Annual Review of Biochemistry 1995, 64, 763; M. Famulok, Current Opinion in Structural Biology 1999, 9, 324). Although the reasons for the substantial increase in complexity and information content of the natural aptamer sequences relative to artificial aptamers remains to be proven, this complexity is believed required to form RNA receptors that function with high affinity and selectivity. Apparent K_{D} values for the ligand-riboswitch complexes range from low nanomolar to low micromolar. It is also worth noting that some aptamer domains, when isolated from the appended expression platform, exhibit improved affinity for the target ligand over that of the intact riboswitch. (−10 to 100-fold) (see Example 2 of U.S. Application Publication No. 2005-0053951). Presumably, there is an energetic cost in sampling the multiple distinct RNA conformations required by a fully intact riboswitch RNA, which is reflected by a loss in ligand affinity. Since the aptamer domain must serve as a molecular switch, this might also add to the functional demands on natural aptamers that might help rationalize their more sophisticated structures.

### B. Riboswitch Regulation of Transcription Termination in Bacteria

Bacteria primarily make use of two methods for termination of transcription. Certain genes incorporate a termination signal that is dependent upon the Rho protein, (J. P. Richardson, Biochimica et Biophysica Acta 2002, 1577, 251). while others make use of Rho-independent terminators (intrinsic terminators) to destabilize the transcription elongation complex (I. Gusarov, E. Nudler, Molecular Cell 1999, 3, 495; E. Nudler, M. E. Gottesman, Genes to Cells 2002, 7, 755). The latter RNA elements are composed of a GC-rich stem-loop followed by a stretch of 6-9 uridyl residues. Intrinsic terminators are widespread throughout bacterial genomes (F. Lillo, et al., 2002, 18, 971), and are typically located at the 3′-termini of genes or operons. Interestingly, an increasing number of examples are being observed for intrinsic terminators located within 5′-UTRs.

Amongst the wide variety of genetic regulatory strategies employed by bacteria there is a growing class of examples wherein RNA polymerase responds to a termination signal within the 5′-UTR in a regulated fashion (T. M. Henkin, Current Opinion in Microbiology 2000, 3, 149). During certain conditions the RNA polymerase complex is directed by external signals either to perceive or to ignore the termination signal. Although transcription initiation might occur without regulation, control over mRNA synthesis (and of gene expression) is ultimately dictated by regulation of the intrinsic terminator. Presumably, one of at least two mutually exclusive mRNA conformations results in the formation or disruption of the RNA structure that signals transcription termination. A trans-acting factor, which in some instances is a RNA (F. J. Grundy, et al., Proceedings of the National Academy of Sciences of the United States of America 2002, 99, 11121; T. M. Henkin, C. Yanofsky, Bioessays 2002, 24, 700) and in others is a protein (J. Stulke, Archives of Microbiology 2002, 177, 433), is generally required for receiving a particular intracellular signal and subsequently stabilizing one of the RNA conformations. Riboswitches offer a direct link between RNA structure modulation and the metabolite signals that are interpreted by the genetic control machinery.

Riboswitches must be capable of discriminating against compounds related to their natural ligands to prevent undesirable regulation of metabolic genes. However, it is possible to generate analogs that trigger riboswitch function and inhibit bacterial growth, as has been demonstrated for riboswitches that normally respond to lysine (Sudarsan 2003) and thiamine pyrophosphate (Sudarsan 2006).

Disclosed are compositions and methods involing riboswitches and RNA motis. For example, disclosed are regulatable gene expression constructs comprising, for example, a nucleic acid molecule encoding an RNA comprising a riboswitch operably linked to a coding region, wherein the riboswitch regulates expression of the RNA, wherein the riboswitch and coding region are heterologous. The riboswitch can be, for example, a glutamine-responsive riboswitch, an S-adenosylmethionine-repsonsive riboswitch, an S-adenosylhomocysteine-repsonsive riboswitch, a glutamine riboswitch, a SAM/SAH riboswitch, a glnA riboswitch, a Downstream-peptide riboswitch, a crcB riboswitch, a pfl riboswitch, a yjdF riboswitch, a manA riboswitch, a wcaG riboswitch, an epsC riboswitch, a ykkC-III riboswitch, a psaA riboswitch, a psbA riboswitch, a PhotoRC-I riboswitch, a PhotoRC-II riboswitch, or a psbNH riboswitch.

Also disclosed are, for example, riboswitches, wherein the riboswitch is a non-natural derivative of a naturally-occurring riboswitch. The naturally-occurring riboswitch can be, for example, a glutamine-responsive riboswitch, an S-adenosylmethionine-repsonsive riboswitch, an S-adenosylhomocysteine-repsonsive riboswitch, a glutamine riboswitch, a SAM/SAH riboswitch, a glnA riboswitch, a Downstream-peptide riboswitch, a crcB riboswitch, a pfl riboswitch, a yjdF riboswitch, a manA riboswitch, a wcaG riboswitch, an epsC riboswitch, a ykkC-III riboswitch, a psaA riboswitch, a psbA riboswitch, a PhotoRC-I riboswitch, a PhotoRC-II riboswitch, or a psbNH riboswitch.

Also disclosed are, for example, methods of detecting a compound of interest, the method comprising, for example, bringing into contact a sample and a riboswitch, wherein the riboswitch is activated by the compound of interest, wherein the riboswitch produces a signal when activated by the compound of interest, wherein the riboswitch produces a signal when the sample contains the compound of interest. The riboswitch can be, for example, a glutamine-responsive riboswitch, an S-adenosylmethionine-repsonsive riboswitch, an S-adenosylhomocysteine-repsonsive riboswitch, a glutamine riboswitch, a SAM/SAH riboswitch, a glnA riboswitch, a Downstream-peptide riboswitch, a crcB riboswitch, a pfl riboswitch, a yjdF riboswitch, a manA riboswitch, a wcaG riboswitch, an epsC riboswitch, a ykkC-III riboswitch, a psaA riboswitch, a psbA riboswitch, a PhotoRC-I riboswitch, a PhotoRC-II riboswitch, or a psbNH riboswitch.

Also disclosed are methods of identifying compounds that interact with, modulates, inhibits, blocks, deactivates, and/or activates a riboswitch, such as a glutamine riboswitch. For example, disclosed are, for example, methods comprising, for example, (a) testing a compound for altering gene expression of a gene encoding an RNA comprising a riboswitch, wherein the alteration is via the riboswitch, and (b) altering gene expression by bringing into contact a cell and a compound that altered gene expression in step (a), wherein the cell comprises a gene encoding an RNA comprising a riboswitch, wherein the compound inhibits expression of the gene by binding to the riboswitch. The riboswitch can be, for example, a glutamine-responsive riboswitch, an S-adenosylmethionine-repsonsive riboswitch, an S-adenosylhomocysteine-repsonsive riboswitch, a glutamine riboswitch, a SAM/SAH riboswitch, a glnA riboswitch, a Downstream-peptide riboswitch, a crcB riboswitch, apfl riboswitch, a yjdF riboswitch, a manA riboswitch, a wcaG riboswitch, an epsC riboswitch, a ykkC-III riboswitch, a psaA riboswitch, a psbA riboswitch, a PhotoRC-I riboswitch, a PhotoRC-II riboswitch, or a psbNH riboswitch.

Also disclosed are methods of identifying riboswitches, the method comprising, for example, assessing in-line spontaneous cleavage of an RNA molecule in the presence and absence of a compound, wherein the RNA molecule is encoded by a gene regulated by the compound, wherein a change in the pattern of in-line spontaneous cleavage of the RNA molecule indicates a riboswitch. The RNA can comprise a glutamine-responsive riboswitch or a derivative of a glutamine-responsive riboswitch and the compound can be glutamine. The RNA can comprise an S-adenosylhomocysteine-repsonsive riboswitch or a derivative of an S-adenosylhomocysteine-repsonsive riboswitch and the compound can be S-adenosylhomocysteine. The RNA can comprise an S-adenosylmethionine-repsonsive riboswitch or a derivative of an S-adenosylmethionine-repsonsive riboswitch and the compound can be S-adenosylmethionine.

Also disclosed are methods of altering gene expression, the method comprising, for example, bringing into contact a compound and a cell, wherein the cell comprises a gene encoding an RNA comprising, for example, a glutamine-responsive riboswitch, an S-adenosylmethionine-repsonsive riboswitch, an S-adenosylhomocysteine-repsonsive riboswitch, a glutamine riboswitch, a SAM/SAH riboswitch, a glnA riboswitch, a Downstream-peptide riboswitch, a crcB riboswitch, a pfl riboswitch, a yjdF riboswitch, a manA riboswitch, a wcaG riboswitch, an epsC riboswitch, a ykkC-III riboswitch, a psaA riboswitch, a psbA riboswitch, a PhotoRC-I riboswitch, a PhotoRC-II riboswitch, or a psbNH riboswitch.

The riboswitch can comprise an aptamer domain and an expression platform domain, wherein the aptamer domain and the expression platform domain are heterologous. The riboswitch can comprise two or more aptamer domains and an expression platform domain, wherein at least one of the aptamer domains and the expression platform domain are heterologous. In some forms, at least two of the aptamer domains exhibit cooperative binding. The riboswitch can comprise, for example, a glnA motif, a Downstream-peptide motif, a SAM/SAH motif, a crcB motif, a pfl motif, a yjdF motif, a manA motif, a wcaG motif, a epsC motif, a ykkC-III motif, a psaA motif, a psbA motif, a PhotoRC-I motif, a PhotoRC-II motif, or a psbNH motif.

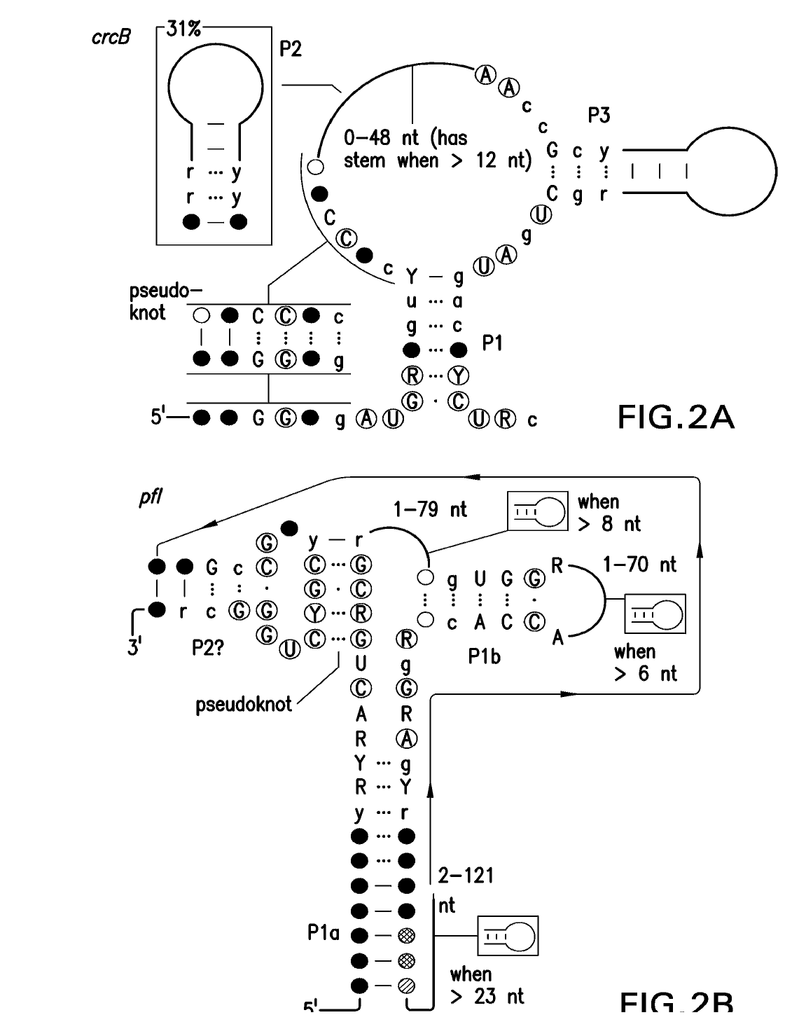

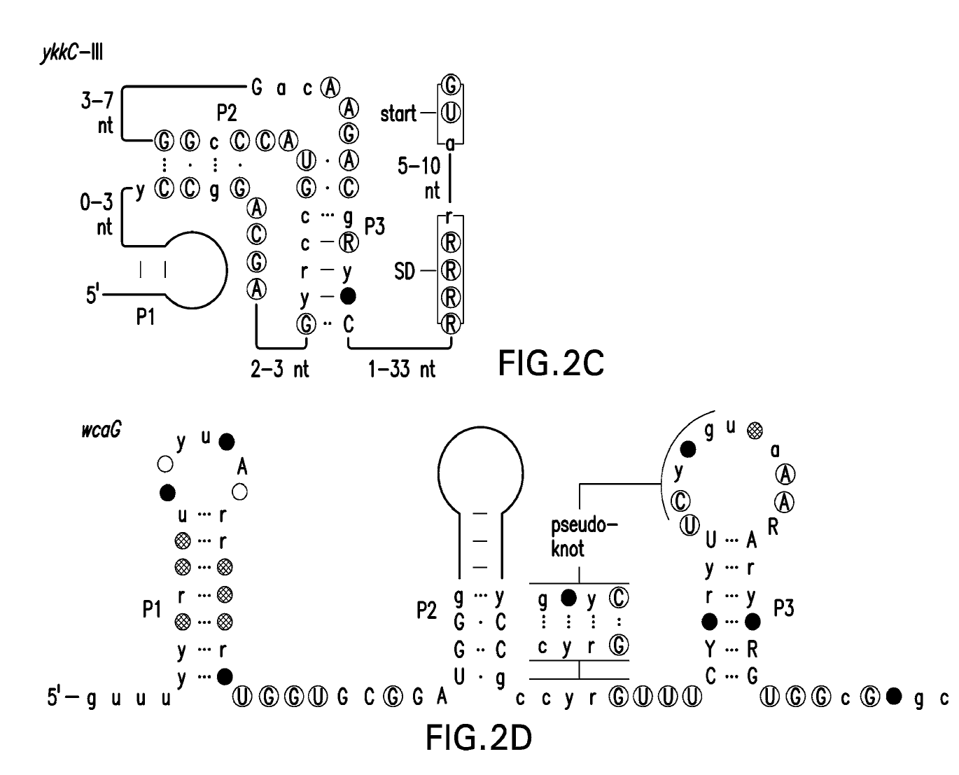

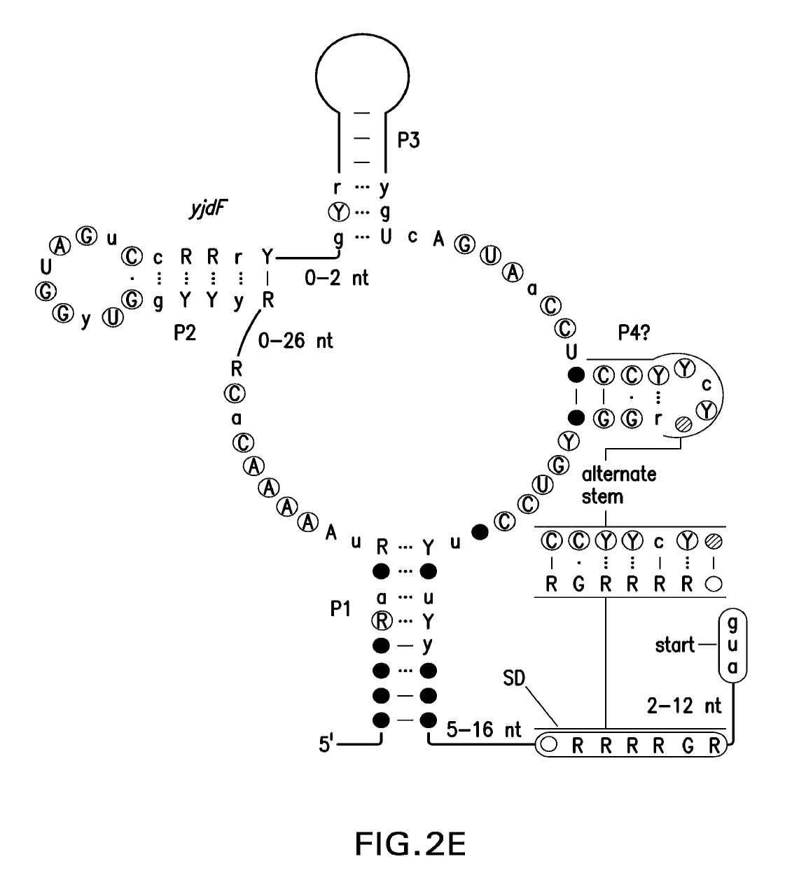

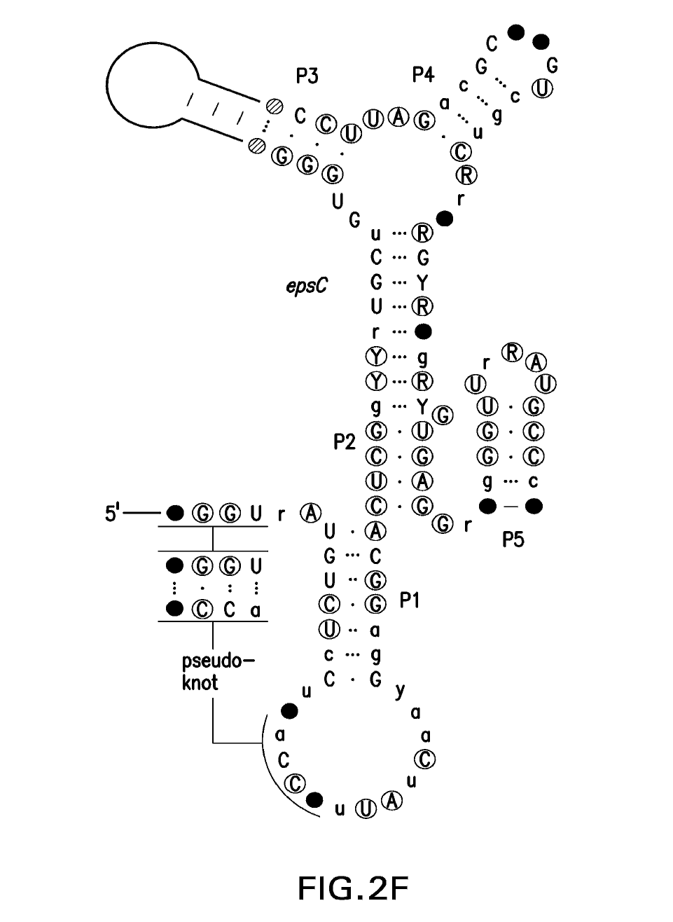

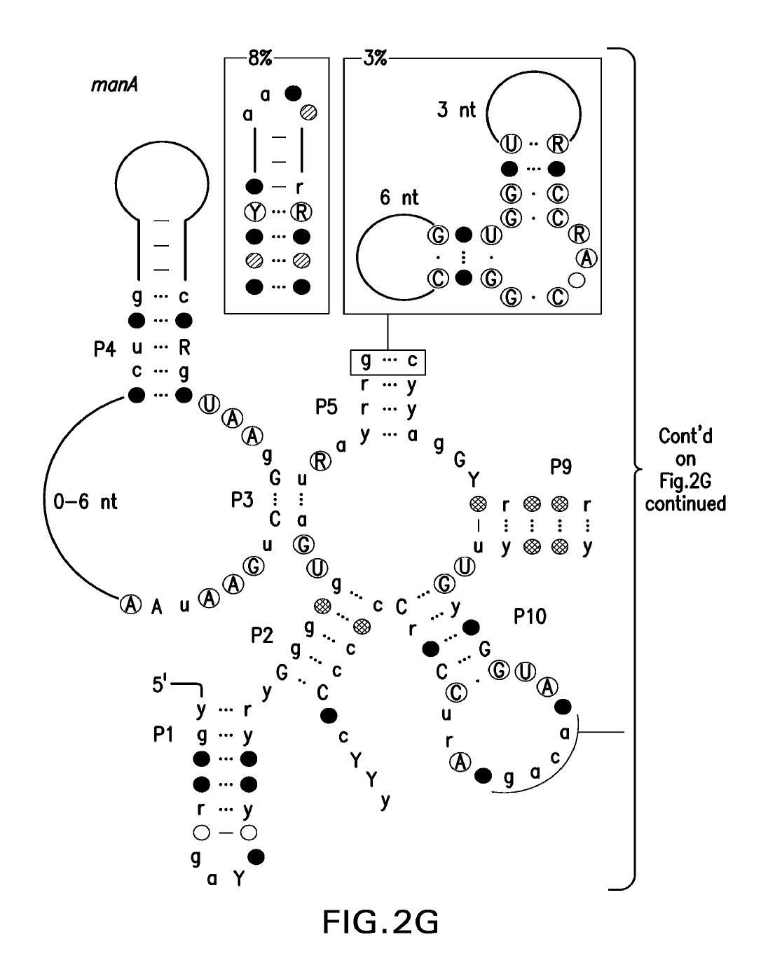

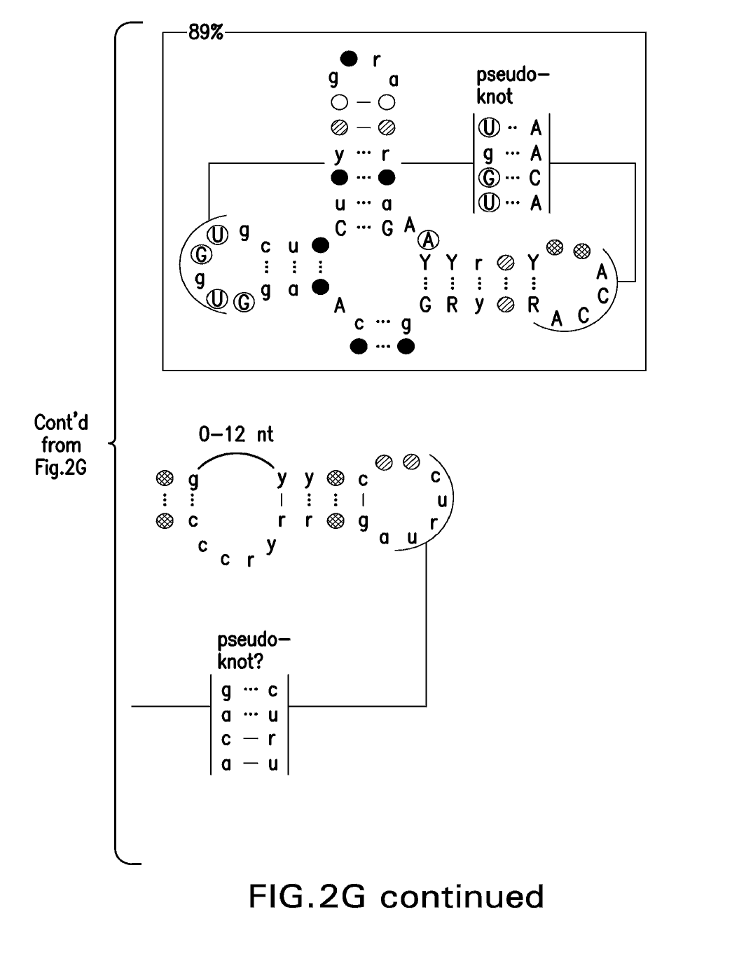

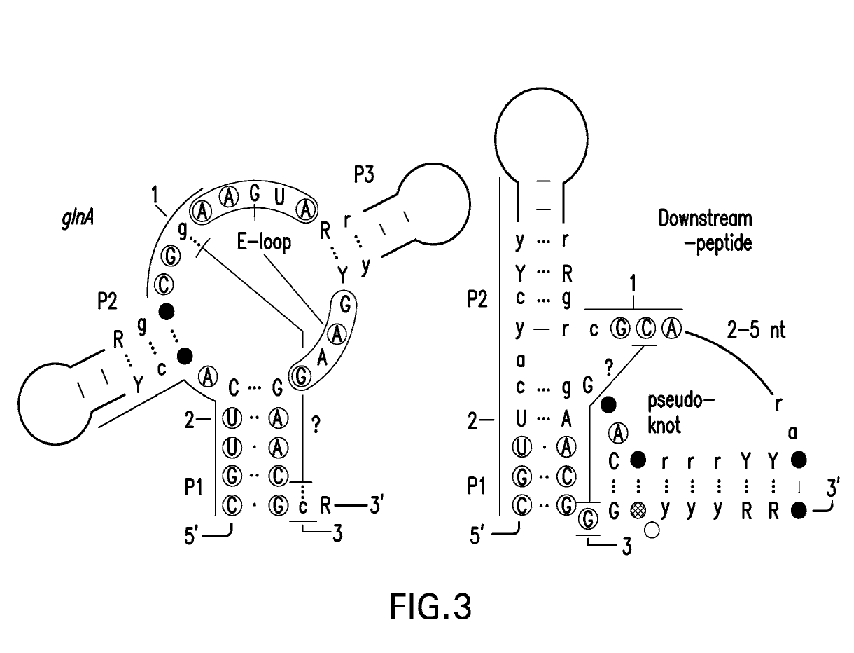

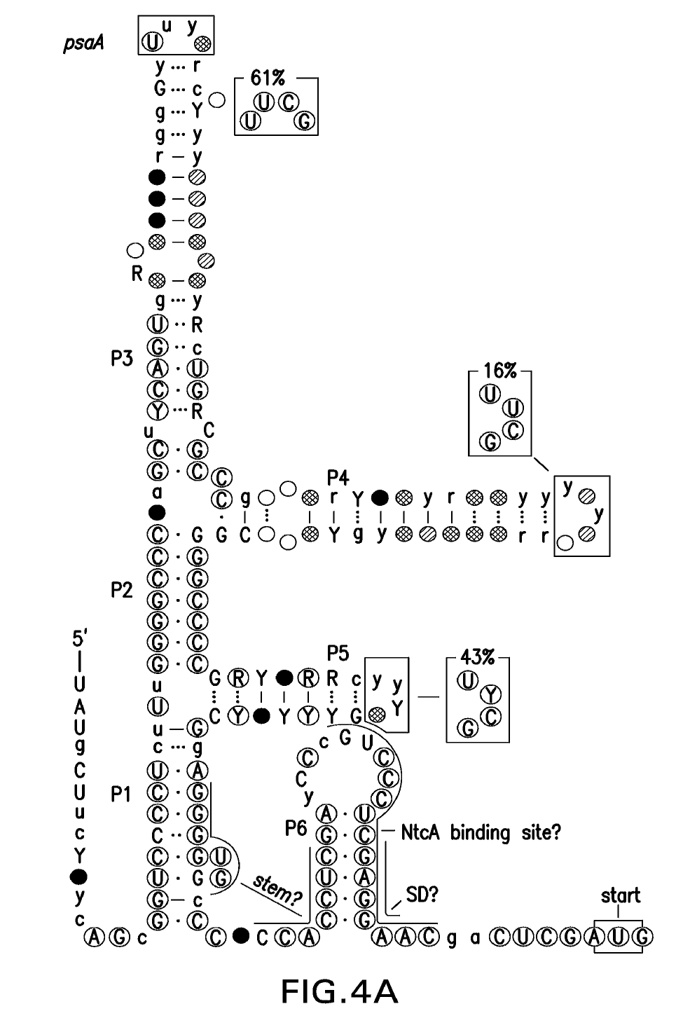

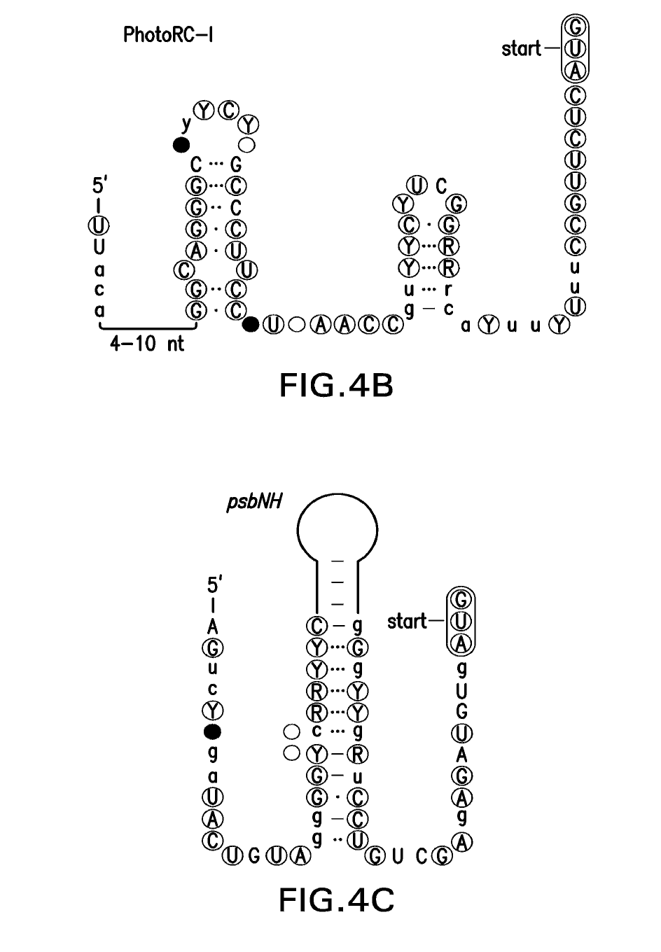

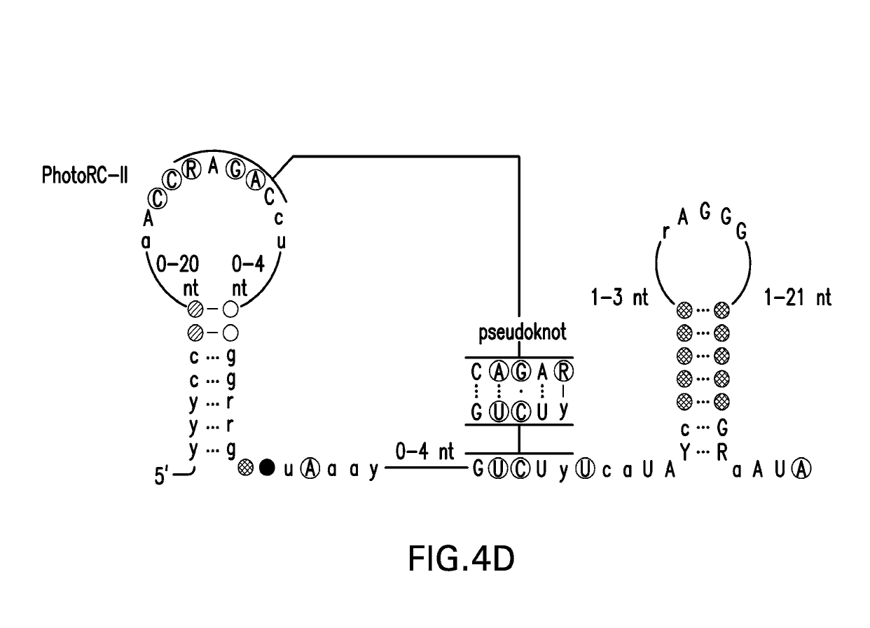

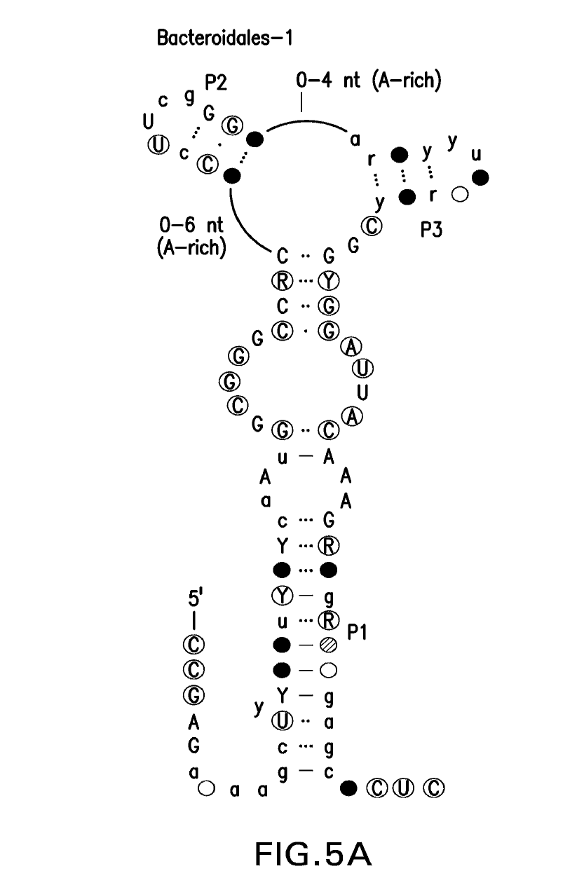

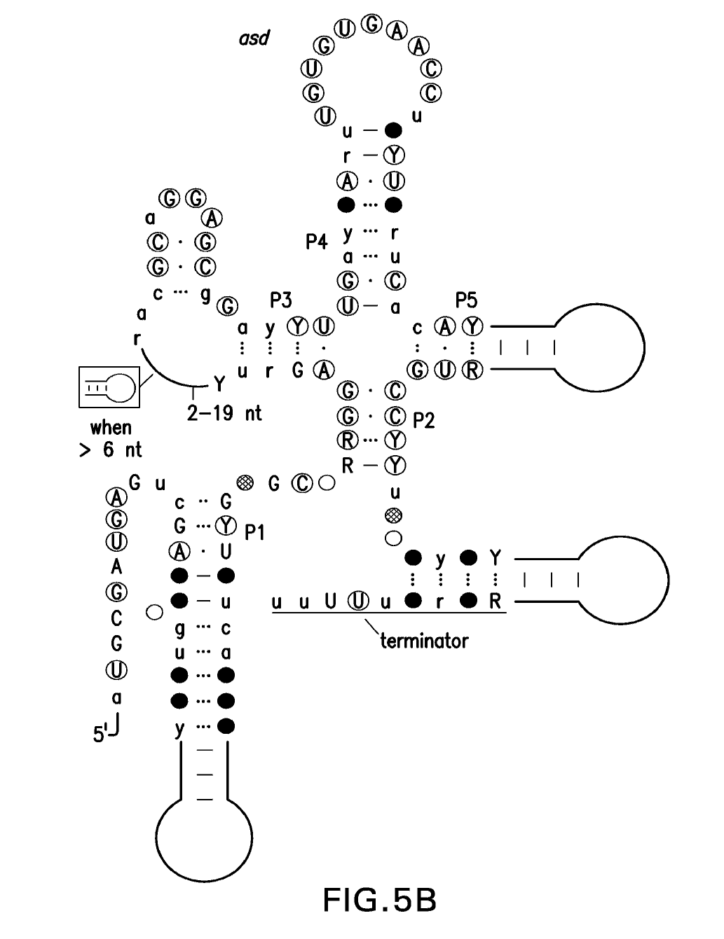

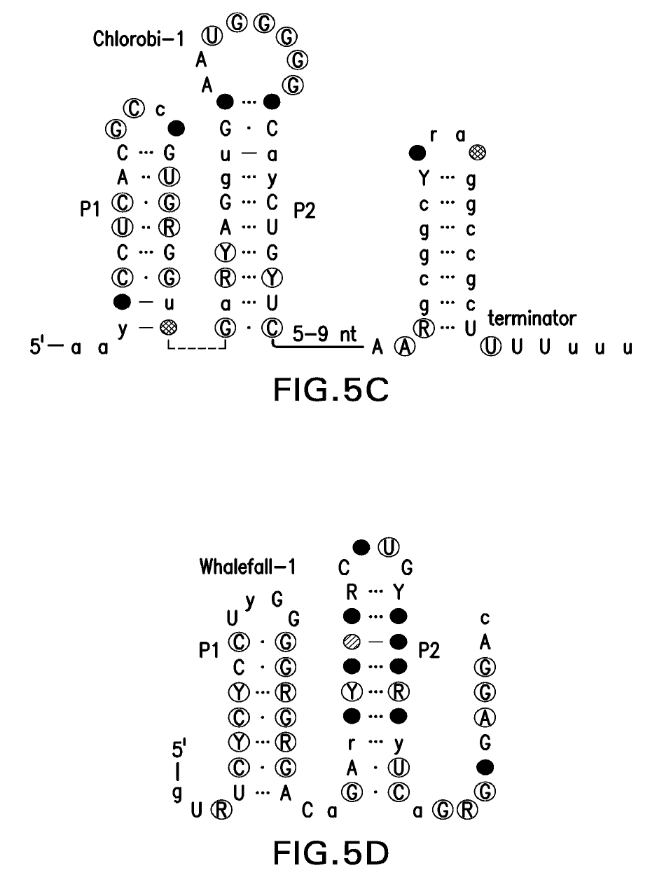

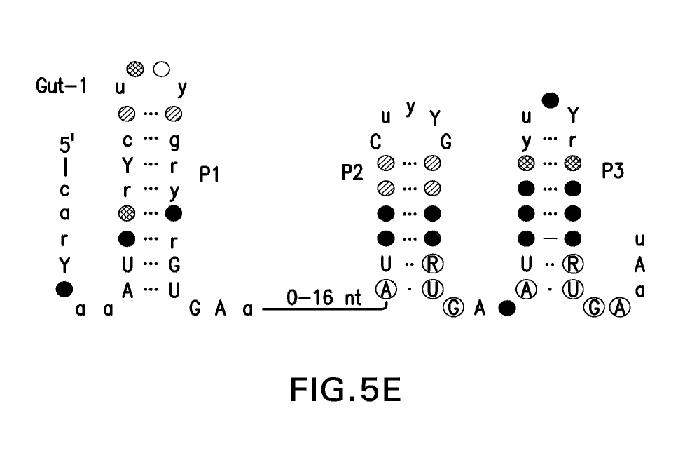

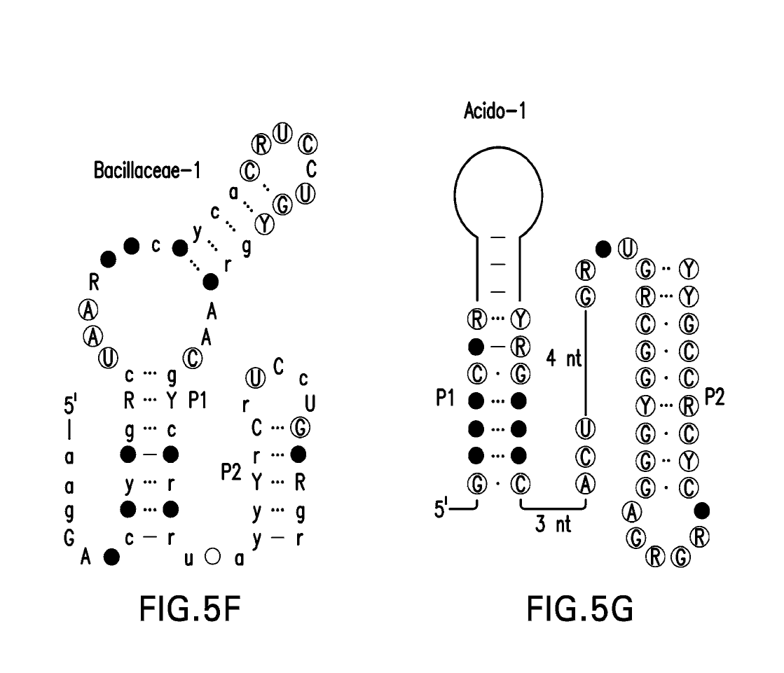

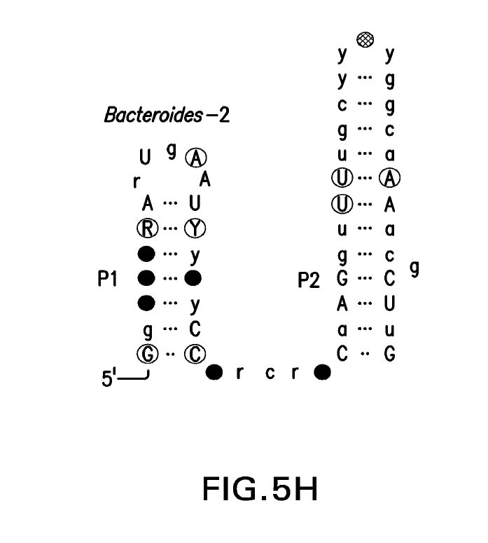

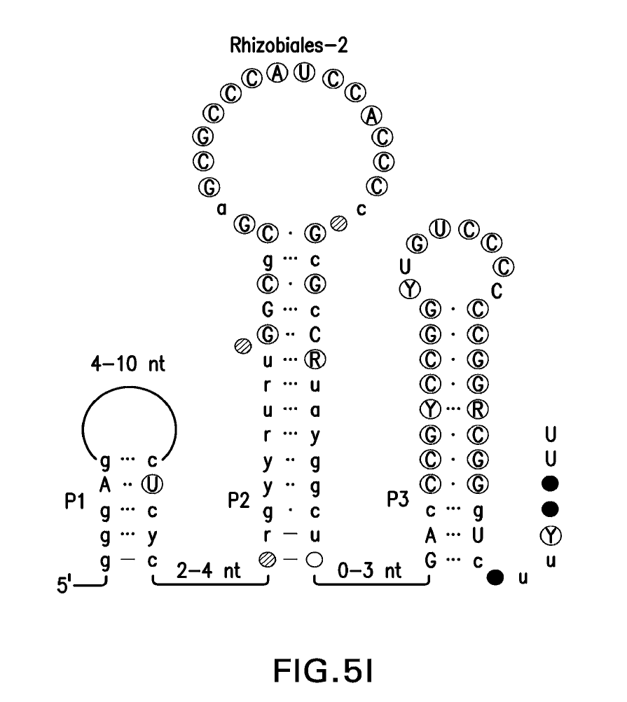

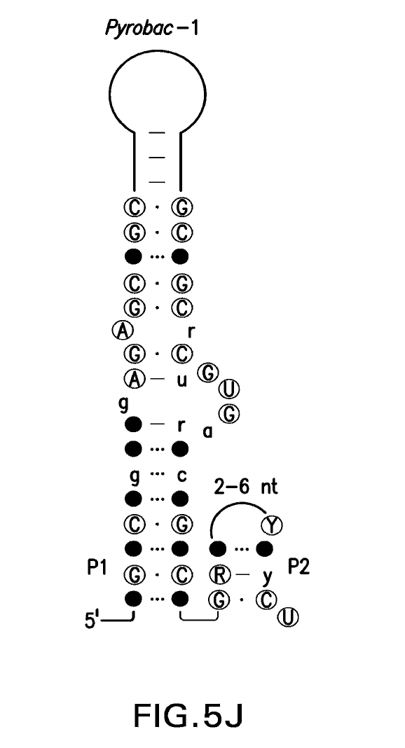

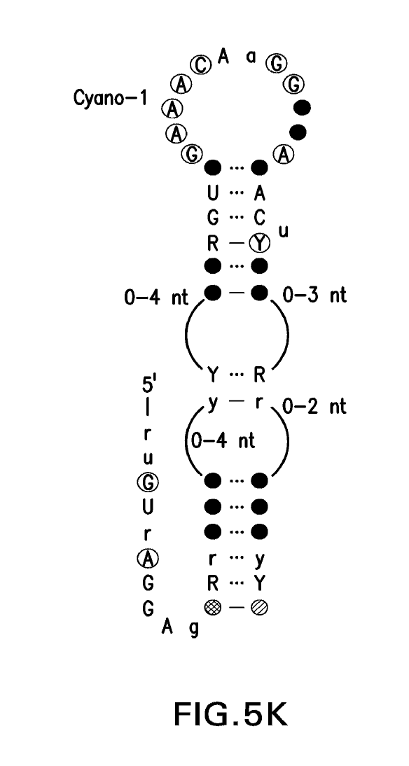

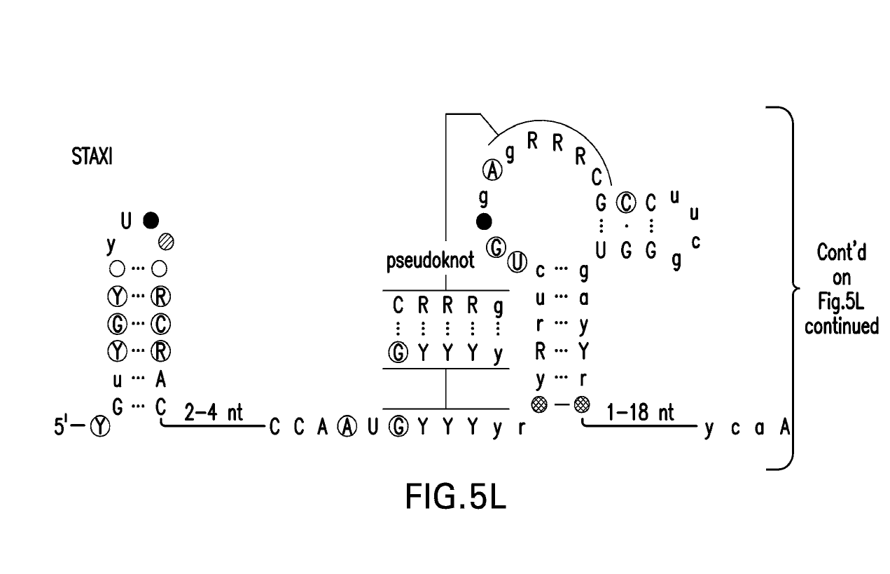

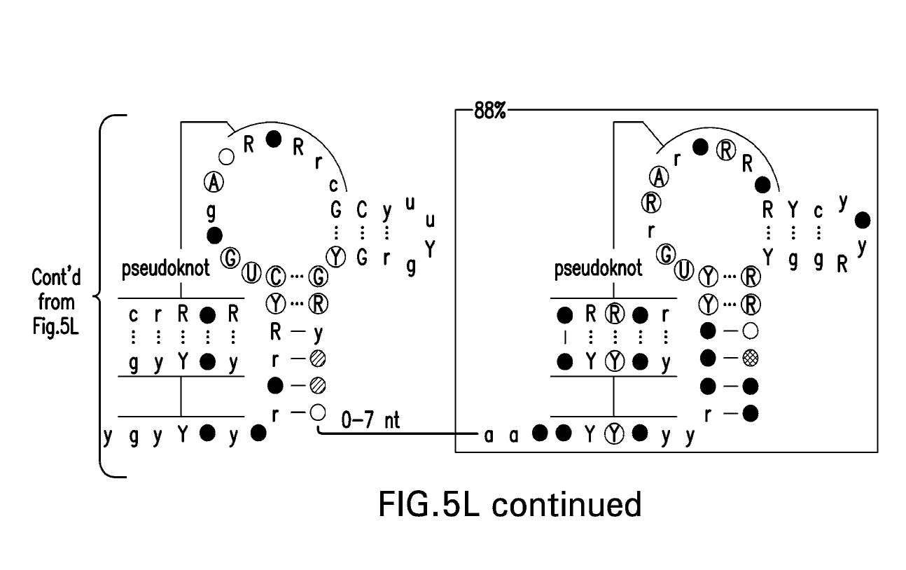

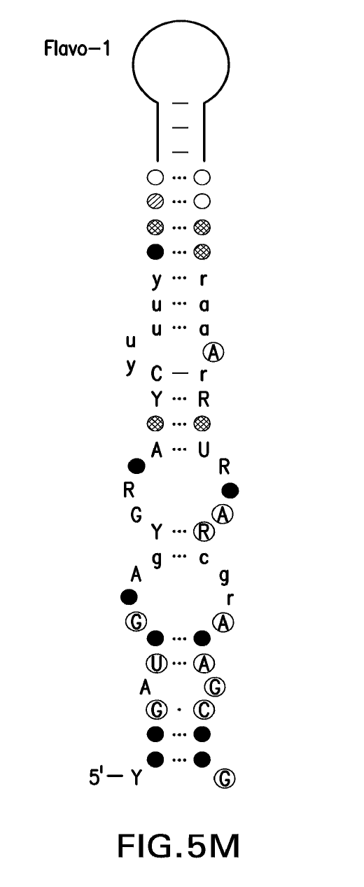

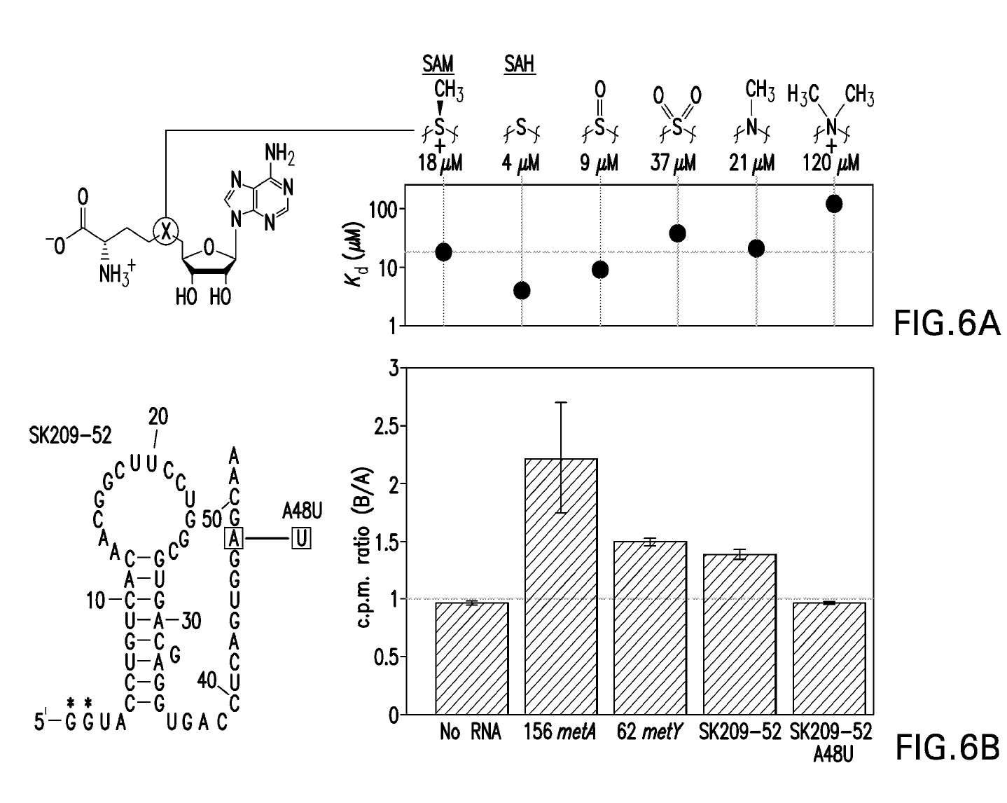

The riboswitch can be activated by a trigger molecule, wherein the riboswitch produces a signal when activated by the trigger molecule. The riboswitch can have, for example, one of the consensus structures of FIG. 1, FIG. 2, FIG. 3, FIG. 4, or FIG. 5.

In some forms, the riboswitch can comprise an aptamer domain and an expression platform domain wherein the aptamer domain is derived from a naturally-occurring glutamine-responsive riboswitch, S-adenosylmethionine-repsonsive riboswitch, S-adenosylhomocysteine-repsonsive riboswitch, glutamine riboswitch, SAM/SAH riboswitch, glnA riboswitch, Downstream-peptide riboswitch, crcB riboswitch, pfl riboswitch, yjdF riboswitch, manA riboswitch, wcaG riboswitch, epsC riboswitch, ykkC-III riboswitch, psaA riboswitch, psbA riboswitch, PhotoRC-I riboswitch, PhotoRC-II riboswitch, or psbNH riboswitch. In some forms, the aptamer domain can be the aptamer domain of a naturally-occurring glutamine-responsive riboswitch, S-adenosylmethionine-repsonsive riboswitch, S-adenosylhomocysteine-repsonsive riboswitch, glutamine riboswitch, SAM/SAH riboswitch, glnA riboswitch, Downstream-peptide riboswitch, crcB riboswitch, pfl riboswitch, yjdF riboswitch, manA riboswitch, wcaG riboswitch, epsC riboswitch, riboswitch, psaA riboswitch, psbA riboswitch, PhotoRC-I riboswitch, PhotoRC-II riboswitch, or psbNH riboswitch.

The aptamer domain can have the consensus structure of an aptamer domain of the naturally-occurring riboswitch. In some forms, the aptamer domain can consist of only base pair conservative changes of the naturally-occurring riboswitch.

In some forms, the riboswitch changes conformation when activated by the compound of interest, wherein the change in conformation produces a signal via a conformation dependent label. In some forms, the riboswitch changes conformation when activated by the compound of interest, wherein the change in conformation causes a change in expression of an RNA linked to the riboswitch, wherein the change in expression produces a signal. In some forms, the signal is produced by a reporter protein expressed from the RNA linked to the riboswitch.

In some forms, the cell can be identified as being in need of altered gene expression. The cell can be a bacterial cell. The compound can kill or inhibit the growth of the bacterial cell. The compound and the cell can be brought into contact by administering the compound to a subject. The cell can be a bacterial cell in the subject, wherein the compound kills or inhibits the growth of the bacterial cell. The subject can have a bacterial infection. The compound can be administered in combination with another antimicrobial compound. The compound can inhibit bacterial growth in a biofilm.

Further disclosed are methods of killing or inhibiting the growth of bacteria. The method can comprise, for example, contacting the bacteria with a compound identified and/or confirmed by any of the methods disclosed herein. Further disclosed are methods of killing bacteria. The method can comprise, for example, contacting the bacteria with a compound identified and/or confirmed by any of the methods disclosed herein. The disclosed methods can be performed in a variety of ways and using different options or combinations of features and components. As an example, a gel-based assay or a chip-based assay can be used to determine if the test compound interacts with, modulates, inhibits, blocks, deactivates, and/or activates the riboswitch, such as a glutamine riboswitch. The test compound can interact in any manner, such as, for example, via van der Waals interactions, hydrogen bonds, electrostatic interactions, hydrophobic interactions, or a combination. The riboswitch, such as a glutamine riboswitch, can comprise an RNA cleaving ribozyme, for example. A fluorescent signal can be generated when a nucleic acid comprising a quenching moiety is cleaved. Molecular beacon technology can be employed to generate the fluorescent signal. The methods disclosed herein can be carried out using a high throughput screen.

Also disclosed are compositions and methods for selecting and identifying compounds that can activate, deactivate or block a riboswitch, such as a glutamine riboswitch. Activation of a riboswitch, such as a glutamine riboswitch, refers to the change in state of the riboswitch upon binding of a trigger molecule. A riboswitch, such as a glutamine riboswitch, can be activated by compounds other than the trigger molecule and in ways other than binding of a trigger molecule. The term trigger molecule is used herein to refer to molecules and compounds that can activate a riboswitch. This includes the natural or normal trigger molecule for the riboswitch and other compounds that can activate the riboswitch. Natural or normal trigger molecules are the trigger molecule for a given riboswitch in nature or, in the case of some non-natural riboswitches, the trigger molecule for which the riboswitch was designed or with which the riboswitch was selected (as in, for example, in vitro selection or in vitro evolution techniques). Non-natural trigger molecules can be referred to as non-natural trigger molecules.

Deactivation of a riboswitch refers to the change in state of the riboswitch, such as a glutamine riboswitch, when the trigger molecule is not bound. A riboswitch, such as a glutamine riboswitch, can be deactivated by binding of compounds other than the trigger molecule and in ways other than removal of the trigger molecule. Blocking of a riboswitch, such as a glutamine riboswitch, refers to a condition or state of the riboswitch where the presence of the trigger molecule does not activate the riboswitch. Activation of a riboswitch, such as a glutamine riboswitch, can be assessed in any suitable manner. For example, the riboswitch, such as a glutamine riboswitch, can be linked to a reporter RNA and expression, expression level, or change in expression level of the reporter RNA can be measured in the presence and absence of the test compound. As another example, the riboswitch, such as a glutamine riboswitch, can include a conformation dependent label, the signal from which changes depending on the activation state of the riboswitch, such as a glutamine riboswitch. Such a riboswitch preferably uses an aptamer domain from or derived from a naturally occurring riboswitch. As can be seen, assessment of activation of a riboswitch can be performed with the use of a control assay or measurement or without the use of a control assay or measurement. Methods for identifying compounds that deactivate a riboswitch can be performed in analogous ways.

Also disclosed are method of inhibiting growth of a cell, such as a bacterial cell, that is in a subject. The method can comprise administering to the subject an effective amount of a compound identified and/or confirmed in any of the methods described herein. This can result in the compound being brought into contact with the cell. The subject can have, for example, a bacterial infection, and the bacterial cells can be the cells to be inhibited by the compound. The bacteria can be any bacteria, such as bacteria from the genus Bacillus or Staphylococcus, for example. Bacterial growth can also be inhibited in any context in which bacteria are found. For example, bacterial growth in fluids, biofilms, and on surfaces can be inhibited. The compounds disclosed herein can be administered or used in combination with any other compound or composition. For example, the disclosed compounds can be administered or used in combination with another antimicrobial compound.

It is to be understood that the disclosed methods and compositions are not limited to specific examples unless otherwise specified, and, as such, can vary. It is also to be understood that the terminology used herein is for the purpose of describing particular embodiments only and is not intended to be limiting.

### Materials

Disclosed are materials, compositions, and components that can be used for, can be used in conjunction with, can be used in preparation for, or are products of the disclosed methods and compositions. These and other materials are disclosed herein, and it is understood that when combinations, subsets, interactions, groups, etc. of these materials are disclosed that while specific reference to each of various individual and collective combinations and permutation of these compounds can not be explicitly disclosed, each is specifically contemplated and described herein. For example, if a riboswitch or aptamer domain is disclosed and discussed and a number of modifications that can be made to a number of molecules including the riboswitch or aptamer domain are discussed, each and every combination and permutation of riboswitch or aptamer domain and the modifications that are possible are specifically contemplated unless specifically indicated to the contrary. Thus, if a class of molecules A, B, and C are disclosed as well as a class of molecules D, E, and F and an example of a combination molecule, A-D is disclosed, then even if each is not individually recited, each is individually and collectively contemplated. Thus, in this example, each of the combinations A-E, A-F, B-D, B-E, B-F, C-D, C-E, and C-F are specifically contemplated and should be considered disclosed from disclosure of A, B, and C; D, E, and F; and the example combination A-D. Likewise, any subset or combination of these is also specifically contemplated and disclosed. Thus, for example, the sub-group of A-E, B-F, and C-E are specifically contemplated and should be considered disclosed from disclosure of A, B, and C; D, E, and F; and the example combination A-D. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods, and that each such combination is specifically contemplated and should be considered disclosed.

### A. Riboswitches

Riboswitches are expression control elements that are part of an RNA molecule to be expressed and that change state when bound by a trigger molecule. Riboswitches typically can be dissected into two separate domains: one that selectively binds the target (aptamer domain) and another that influences genetic control (expression platform domain). It is the dynamic interplay between these two domains that results in metabolite-dependent allosteric control of gene expression. Disclosed are isolated and recombinant riboswitches, recombinant constructs containing such riboswitches, heterologous sequences operably linked to such riboswitches, and cells and transgenic organisms harboring such riboswitches, riboswitch recombinant constructs, and riboswitches operably linked to heterologous sequences. The heterologous sequences can be, for example, sequences encoding proteins or peptides of interest, including reporter proteins or peptides. Preferred riboswitches are, or are derived from, naturally occurring riboswitches.

The disclosed riboswitches, including the derivatives and recombinant forms thereof, generally can be from any source, including naturally occurring riboswitches and riboswitches designed de novo. Any such riboswitches can be used in or with the disclosed methods. However, different types of riboswitches can be defined and some such sub-types can be useful in or with particular methods (generally as described elsewhere herein). Types of riboswitches include, for example, naturally occurring riboswitches, derivatives and modified forms of naturally occurring riboswitches, chimeric riboswitches, and recombinant riboswitches. A naturally occurring riboswitch is a riboswitch having the sequence of a riboswitch as found in nature. Such a naturally occurring riboswitch can be an isolated or recombinant form of the naturally occurring riboswitch as it occurs in nature. That is, the riboswitch has the same primary structure but has been isolated or engineered in a new genetic or nucleic acid context. Chimeric riboswitches can be made up of, for example, part of a riboswitch of any or of a particular class or type of riboswitch and part of a different riboswitch of the same or of any different class or type of riboswitch; part of a riboswitch of any or of a particular class or type of riboswitch and any non-riboswitch sequence or component. Recombinant riboswitches are riboswitches that have been isolated or engineered in a new genetic or nucleic acid context.

Riboswitches can have single or multiple aptamer domains. Aptamer domains in riboswitches having multiple aptamer domains can exhibit cooperative binding of trigger molecules or can not exhibit cooperative binding of trigger molecules (that is, the aptamers need not exhibit cooperative binding). In the latter case, the aptamer domains can be said to be independent binders. Riboswitches having multiple aptamers can have one or multiple expression platform domains. For example, a riboswitch having two aptamer domains that exhibit cooperative binding of their trigger molecules can be linked to a single expression platform domain that is regulated by both aptamer domains. Riboswitches having multiple aptamers can have one or more of the aptamers joined via a linker. Where such aptamers exhibit cooperative binding of trigger molecules, the linker can be a cooperative linker.

Aptamer domains can be said to exhibit cooperative binding if they have a Hill coefficient n between x and x−1, where x is the number of aptamer domains (or the number of binding sites on the aptamer domains) that are being analyzed for cooperative binding. Thus, for example, a riboswitch having two aptamer domains (such as glycine-responsive riboswitches) can be said to exhibit cooperative binding if the riboswitch has Hill coefficient between 2 and 1. It should be understood that the value of x used depends on the number of aptamer domains being analyzed for cooperative binding, not necessarily the number of aptamer domains present in the riboswitch. This makes sense because a riboswitch can have multiple aptamer domains where only some exhibit cooperative binding.

Disclosed are chimeric riboswitches containing heterologous aptamer domains and expression platform domains. That is, chimeric riboswitches are made up an aptamer domain from one source and an expression platform domain from another source. The heterologous sources can be from, for example, different specific riboswitches, different types of riboswitches, or different classes of riboswitches. The heterologous aptamers can also come from non-riboswitch aptamers. The heterologous expression platform domains can also come from non-riboswitch sources.

Modified or derivative riboswitches can be produced using in vitro selection and evolution techniques. In general, in vitro evolution techniques as applied to riboswitches involve producing a set of variant riboswitches where part(s) of the riboswitch sequence is varied while other parts of the riboswitch are held constant. Activation, deactivation or blocking (or other functional or structural criteria) of the set of variant riboswitches can then be assessed and those variant riboswitches meeting the criteria of interest are selected for use or further rounds of evolution. Useful base riboswitches for generation of variants are the specific and consensus riboswitches disclosed herein. Consensus riboswitches can be used to inform which part(s) of a riboswitch to vary for in vitro selection and evolution.

Also disclosed are modified riboswitches with altered regulation. The regulation of a riboswitch can be altered by operably linking an aptamer domain to the expression platform domain of the riboswitch (which is a chimeric riboswitch). The aptamer domain can then mediate regulation of the riboswitch through the action of, for example, a trigger molecule for the aptamer domain. Aptamer domains can be operably linked to expression platform domains of riboswitches in any suitable manner, including, for example, by replacing the normal or natural aptamer domain of the riboswitch with the new aptamer domain. Generally, any compound or condition that can activate, deactivate or block the riboswitch from which the aptamer domain is derived can be used to activate, deactivate or block the chimeric riboswitch.

Also disclosed are inactivated riboswitches. Riboswitches can be inactivated by covalently altering the riboswitch (by, for example, crosslinking parts of the riboswitch or coupling a compound to the riboswitch). Inactivation of a riboswitch in this manner can result from, for example, an alteration that prevents the trigger molecule for the riboswitch from binding, that prevents the change in state of the riboswitch upon binding of the trigger molecule, or that prevents the expression platform domain of the riboswitch from affecting expression upon binding of the trigger molecule.

Also disclosed are biosensor riboswitches. Biosensor riboswitches are engineered riboswitches that produce a detectable signal in the presence of their cognate trigger molecule. Useful biosensor riboswitches can be triggered at or above threshold levels of the trigger molecules. Biosensor riboswitches can be designed for use in vivo or in vitro. For example, biosensor riboswitches operably linked to a reporter RNA that encodes a protein that serves as or is involved in producing a signal can be used in vivo by engineering a cell or organism to harbor a nucleic acid construct encoding the riboswitch/reporter RNA. An example of a biosensor riboswitch for use in vitro is a riboswitch that includes a conformation dependent label, the signal from which changes depending on the activation state of the riboswitch. Such a biosensor riboswitch preferably uses an aptamer domain from or derived from a naturally occurring riboswitch. Biosensor riboswitches can be used in various situations and platforms. For example, biosensor riboswitches can be used with solid supports, such as plates, chips, strips and wells.

Also disclosed are modified or derivative riboswitches that recognize new trigger molecules. New riboswitches and/or new aptamers that recognize new trigger molecules can be selected for, designed or derived from known riboswitches. This can be accomplished by, for example, producing a set of aptamer variants in a riboswitch, assessing the activation of the variant riboswitches in the presence of a compound of interest, selecting variant riboswitches that were activated (or, for example, the riboswitches that were the most highly or the most selectively activated), and repeating these steps until a variant riboswitch of a desired activity, specificity, combination of activity and specificity, or other combination of properties results.

In general, any aptamer domain can be adapted for use with any expression platform domain by designing or adapting a regulated strand in the expression platform domain to be complementary to the control strand of the aptamer domain. Alternatively, the sequence of the aptamer and control strands of an aptamer domain can be adapted so that the control strand is complementary to a functionally significant sequence in an expression platform. For example, the control strand can be adapted to be complementary to the Shine-Dalgarno sequence of an RNA such that, upon formation of a stem structure between the control strand and the SD sequence, the SD sequence becomes inaccessible to ribosomes, thus reducing or preventing translation initiation. Note that the aptamer strand would have corresponding changes in sequence to allow formation of a P1 stem in the aptamer domain. In the case of riboswitches having multiple aptamers exhibiting cooperative binding, one the P1 stem of the activating aptamer (the aptamer that interacts with the expression platform domain) need be designed to form a stem structure with the SD sequence.

As another example, a transcription terminator can be added to an RNA molecule (most conveniently in an untranslated region of the RNA) where part of the sequence of the transcription terminator is complementary to the control strand of an aptamer domain (the sequence will be the regulated strand). This will allow the control sequence of the aptamer domain to form alternative stem structures with the aptamer strand and the regulated strand, thus either forming or disrupting a transcription terminator stem upon activation or deactivation of the riboswitch. Any other expression element can be brought under the control of a riboswitch by similar design of alternative stem structures.

For transcription terminators controlled by riboswitches, the speed of transcription and spacing of the riboswitch and expression platform elements can be important for proper control. Transcription speed can be adjusted by, for example, including polymerase pausing elements (e.g., a series of uridine residues) to pause transcription and allow the riboswitch to form and sense trigger molecules.

Disclosed are regulatable gene expression constructs comprising a nucleic acid molecule encoding an RNA comprising a riboswitch operably linked to a coding region, wherein the riboswitch regulates expression of the RNA, wherein the riboswitch and coding region are heterologous. The riboswitch can comprise an aptamer domain and an expression platform domain, wherein the aptamer domain and the expression platform domain are heterologous. The riboswitch can comprise an aptamer domain and an expression platform domain, wherein the aptamer domain comprises a P1 stem, wherein the P1 stem comprises an aptamer strand and a control strand, wherein the expression platform domain comprises a regulated strand, wherein the regulated strand, the control strand, or both have been designed to form a stem structure. The riboswitch can comprise two or more aptamer domains and an expression platform domain, wherein at least one of the aptamer domains and the expression platform domain are heterologous. The riboswitch can comprise two or more aptamer domains and an expression platform domain, wherein at least one of the aptamer domains comprises a P1 stem, wherein the P1 stem comprises an aptamer strand and a control strand, wherein the expression platform domain comprises a regulated strand, wherein the regulated strand, the control strand, or both have been designed to form a stem structure.

Riboswitches can be referred to in different ways. For example, riboswitches can be identified by their trigger molecule (or main or natural trigger molecule): glutamine riboswitch or SAM/SAH riboswitch, for example. Riboswitches can be identified by their responsiveness to a trigger molecule: glutamine-responsive riboswitch or SAH-responsive riboswitch, for example. Riboswitches can be identified by the aptamer in the riboswitch: glnA riboswitch, Downstream-peptide riboswitch, or crcB riboswitch, for example. Examples of riboswitches include glutamine riboswitches, SAM/SAH riboswitches, glnA riboswitches, Downstream-peptide riboswitches, crcB riboswitches, pfl riboswitches, yjdF riboswitches, manA riboswitches, wcaG riboswitches, epsC riboswitches, ykkC-III riboswitches, psaA riboswitches, psbA riboswitches, PhotoRC-I riboswitches, PhotoRC-II riboswitches, psbNH riboswitches, glutamine-responsive riboswitches, SAM-responsive-riboswitches, and SAH-responsive riboswitches.

1. Aptamer Domains

Aptamers are nucleic acid segments and structures that can bind selectively to particular compounds and classes of compounds. Riboswitches have aptamer domains that, upon binding of a trigger molecule result in a change in the state or structure of the riboswitch. In functional riboswitches, the state or structure of the expression platform domain linked to the aptamer domain changes when the trigger molecule binds to the aptamer domain. Aptamer domains of riboswitches can be derived from any source, including, for example, natural aptamer domains of riboswitches, artificial aptamers, engineered, selected, evolved or derived aptamers or aptamer domains. Aptamers in riboswitches generally have at least one portion that can interact, such as by forming a stem structure, with a portion of the linked expression platform domain. This stem structure will either form or be disrupted upon binding of the trigger molecule.

Consensus aptamer domains of a variety of natural riboswitches are shown in FIG. 11 of U.S. Application Publication No. 2005-0053951 and elsewhere herein. These aptamer domains (including all of the direct variants embodied therein) can be used in riboswitches. The consensus sequences and structures indicate variations in sequence and structure. Aptamer domains that are within the indicated variations are referred to herein as direct variants. These aptamer domains can be modified to produce modified or variant aptamer domains. Conservative modifications include any change in base paired nucleotides such that the nucleotides in the pair remain complementary. Moderate modifications include changes in the length of stems or of loops (for which a length or length range is indicated) of less than or equal to 20% of the length range indicated. Loop and stem lengths are considered to be “indicated” where the consensus structure shows a stem or loop of a particular length or where a range of lengths is listed or depicted. Moderate modifications include changes in the length of stems or of loops (for which a length or length range is not indicated) of less than or equal to 40% of the length range indicated. Moderate modifications also include and functional variants of unspecified portions of the aptamer domain.

The P1 stem and its constituent strands can be modified in adapting aptamer domains for use with expression platforms and RNA molecules. Such modifications, which can be extensive, are referred to herein as P1 modifications. P1 modifications include changes to the sequence and/or length of the P1 stem of an aptamer domain.

Aptamer domains of the disclosed riboswitches can also be used for any other purpose, and in any other context, as aptamers. For example, aptamers can be used to control ribozymes, other molecular switches, and any RNA molecule where a change in structure can affect function of the RNA.

Examples of aptamer domains are any of the RNA motifs described herein. For example, glnA, Downstream-peptide, SAM/SAH, crcB, pfl, yjdF, manA, wcaG, epsC, ykkC-III, psaA, psbA, PhotoRC-I, PhotoRC-II, and psbNH motifs.

2. Expression Platform Domains

Expression platform domains arc a part of riboswitches that affect expression of the RNA molecule that contains the riboswitch. Expression platform domains generally have at least one portion that can interact, such as by forming a stem structure, with a portion of the linked aptamer domain. This stem structure will either form or be disrupted upon binding of the trigger molecule. The stem structure generally either is, or prevents formation of, an expression regulatory structure. An expression regulatory structure is a structure that allows, prevents, enhances or inhibits expression of an RNA molecule containing the structure. Examples include Shine-Dalgarno sequences, initiation codons, transcription terminators, and stability and processing signals.

B. Trigger Molecules

Trigger molecules are molecules and compounds that can activate a riboswitch. This includes the natural or normal trigger molecule for the riboswitch and other compounds that can activate the riboswitch. Natural or normal trigger molecules are the trigger molecule for a given riboswitch in nature or, in the case of some non-natural riboswitches, the trigger molecule for which the riboswitch was designed or with which the riboswitch was selected (as in, for example, in vitro selection or in vitro evolution techniques).

C. Glutamine Riboswitches (glnA and Downstream-Peptide Motifs)

The glnA motif and the Downstream-peptide motif (FIG. 29) are both approximately 60 nucleotides in length, comprise three base-paired regions, and share highly similar portions of conserved sequence and structure. However, there are some minor structural differences that distinguish the two motifs. The glnA motif includes an E-loop connecting the junction of stems P2 and P3 (J2/3) with J3/1. In contrast, the Downstream-peptide motif lacks an E-loop and P3 stem, but instead forms a pseudoknot. An additional difference between the two RNAs is their genetic placement. While the Downstream-peptide motif is found exclusively in the 5′ UTRs of short polypeptides of unknown function that are typically 17 to 100 amino acids in length, the glnA motif is frequently positioned upstream of a variety of genes involved in nitrogen metabolism including ammonium transporters, glutamine and glutamate synthetases, and nitrogen regulatory protein P_{II} (Weinberg et al., Genome Biol 2010; 11:R31).

The two motifs share various qualities with previously characterized riboswitches, including size, complexity, degree of sequence conservation, and genetic context. Considering the nature of the genes downstream of the glnA motif, it was speculated that L-glutamine could be the ligand for this riboswitch aptamer. Glycine and lysine are known ligands for riboswitches (Sudarsan et al. An mRNA structure in bacteria that controls gene expression by binding lysine. Genes Dev 2003; 17:2688-2697; Mandal et al. A glycine-dependent riboswitch that uses cooperative binding to control gene expression. Science 2004; 306:275-279), and establish a precedent for riboswitch aptamers recognizing amino acids. Furthermore, glutamine is an attractive riboswitch ligand given its involvement in nitrogen regulation processes.

Nitrogen is often a limiting nutrient in marine environments which makes accurately monitoring internal nitrogen levels particularly important for aquatic bacteria (Goldman JC. Identification of nitrogen as a growth-limiting nutrient in wastewaters and coastal marine waters through continuous culture algal assays. Water Res 1976; 10:97-104). Although glutamine is known to be a key indicator of the state of nitrogen metabolism in proteobacteria and firmicutes (Jiang et al. Enzymological characterization of the signal-transducing uridylyltransferase/uridylyl-removing enzyme (EC 2.7.7.59) of Escherichia coli and its interaction with the PII protein. Biochemistry 1998; 37:12782-12794; Forchhammer K. Glutamine signalling in bacteria. Front Biosci 2007; 12:358-370), other compounds are thought to control this set of pathways in cyanobacteria (Muro-Pastor et al. Cyanobacteria perceive nitrogen status by sensing intracellular 2-oxoglutarate levels. J Biol Chem 2001; 276:38320-38328; Vázquez-Bermudez et al. Carbon supply and 2-oxoglutarate effects on expression of nitrate reductase and nitrogen-regulated genes in Synechococcus sp. strain PCC 7942. FEMS Microbiol Lett 2003; 221:155-159; Forchhammer K. Global carbon/nitrogen control by PII signal transduction in cyanobacteria: from signals to targets. FEMS Microbiol Rev 2004; 28:319-333).

### D. Constructs, Vectors and Expression Systems

The disclosed riboswitches, such as glutamine riboswitches, can be used with any suitable expression system. Recombinant expression is usefully accomplished using a vector, such as a plasmid. The vector can include a promoter operably linked to riboswitch-encoding sequence and RNA to be expression (e.g., RNA encoding a protein). The vector can also include other elements required for transcription and translation. As used herein, vector refers to any carrier containing exogenous DNA. Thus, vectors are agents that transport the exogenous nucleic acid into a cell without degradation and include a promoter yielding expression of the nucleic acid in the cells into which it is delivered. Vectors include but are not limited to plasmids, viral nucleic acids, viruses, phage nucleic acids, phages, cosmids, and artificial chromosomes. A variety of prokaryotic and eukaryotic expression vectors suitable for carrying riboswitch-regulated constructs can be produced. Such expression vectors include, for example, pET, pET3d, pCR2.1, pBAD, pUC, and yeast vectors. The vectors can be used, for example, in a variety of in vivo and in vitro situation.

Viral vectors include adenovirus, adeno-associated virus, herpes virus, vaccinia virus, polio virus, AIDS virus, neuronal trophic virus, Sindbis and other RNA viruses, including these viruses with the HIV backbone. Also useful are any viral families which share the properties of these viruses which make them suitable for use as vectors. Retroviral vectors, which are described in Verma (1985), include Murine Maloney Leukemia virus, MMLV, and retroviruses that express the desirable properties of MMLV as a vector. Typically, viral vectors contain, nonstructural early genes, structural late genes, an RNA polymerase III transcript, inverted terminal repeats necessary for replication and encapsidation, and promoters to control the transcription and replication of the viral genome. When engineered as vectors, viruses typically have one or more of the early genes removed and a gene or gene/promoter cassette is inserted into the viral genome in place of the removed viral DNA.

A “promoter” is generally a sequence or sequences of DNA that function when in a relatively fixed location in regard to the transcription start site. A “promoter” contains core elements required for basic interaction of RNA polymerase and transcription factors and can contain upstream elements and response elements.

“Enhancer” generally refers to a sequence of DNA that functions at no fixed distance from the transcription start site and can be either 5′ (Laimins, 1981) or 3′ (Lusky et al., 1983) to the transcription unit. Furthermore, enhancers can be within an intron (Banerji et al., 1983) as well as within the coding sequence itself (Osborne et al., 1984). They are usually between 10 and 300 bp in length, and they function in cis. Enhancers function to increase transcription from nearby promoters. Enhancers, like promoters, also often contain response elements that mediate the regulation of transcription. Enhancers often determine the regulation of expression.

Expression vectors used in eukaryotic host cells (yeast, fungi, insect, plant, animal, human or nucleated cells) can also contain sequences necessary for the termination of transcription which can affect mRNA expression. These regions are transcribed as polyadenylated segments in the untranslated portion of the mRNA encoding tissue factor protein. The 3′ untranslated regions also include transcription termination sites. It is preferred that the transcription unit also contain a polyadenylation region. One benefit of this region is that it increases the likelihood that the transcribed unit will be processed and transported like mRNA. The identification and use of polyadenylation signals in expression constructs is well established. It is preferred that homologous polyadenylation signals be used in the transgene constructs.

The vector can include nucleic acid sequence encoding a marker product. This marker product is used to determine if the gene has been delivered to the cell and once delivered is being expressed. Preferred marker genes are the E. Coli lacZ gene which encodes β-galactosidase and green fluorescent protein.

In some embodiments the marker can be a selectable marker. When such selectable markers are successfully transferred into a host cell, the transformed host cell can survive if placed under selective pressure. There are two widely used distinct categories of selective regimes. The first category is based on a cell's metabolism and the use of a mutant cell line which lacks the ability to grow independent of a supplemented media. The second category is dominant selection which refers to a selection scheme used in any cell type and does not require the use of a mutant cell line. These schemes typically use a drug to arrest growth of a host cell. Those cells which have a novel gene would express a protein conveying drug resistance and would survive the selection. Examples of such dominant selection use the drugs neomycin, (Southern and Berg, 1982), mycophenolic acid, (Mulligan and Berg, 1980) or hygromycin (Sugden et al., 1985).

Gene transfer can be obtained using direct transfer of genetic material, in but not limited to, plasmids, viral vectors, viral nucleic acids, phage nucleic acids, phages, cosmids, and artificial chromosomes, or via transfer of genetic material in cells or carriers such as cationic liposomes. Such methods are well known in the art and readily adaptable for use in the method described herein. Transfer vectors can be any nucleotide construction used to deliver genes into cells (e.g., a plasmid), or as part of a general strategy to deliver genes, e.g., as part of recombinant retrovirus or adenovirus (Ram et al. Cancer Res. 53:83-88, (1993)). Appropriate means for transfection, including viral vectors, chemical transfectants, or physico-mechanical methods such as electroporation and direct diffusion of DNA, are described by, for example, Wolff, J. A., et al., Science, 247, 1465-1468, (1990); and Wolff, J. A. Nature, 352, 815-818, (1991).

1. Viral Vectors

Preferred viral vectors are Adenovirus, Adeno-associated virus, Herpes virus, Vaccinia virus, Polio virus, AIDS virus, neuronal trophic virus, Sindbis and other RNA viruses, including these viruses with the HIV backbone. Also preferred are any viral families which share the properties of these viruses which make them suitable for use as vectors. Preferred retroviruses include Murine Maloney Leukemia virus, MMLV, and retroviruses that express the desirable properties of MMLV as a vector. Retroviral vectors are able to carry a larger genetic payload, i.e., a transgene or marker gene, than other viral vectors, and for this reason are a commonly used vector. However, they are not useful in non-proliferating cells. Adenovirus vectors are relatively stable and easy to work with, have high titers, and can be delivered in aerosol formulation, and can transfect non-dividing cells. Pox viral vectors are large and have several sites for inserting genes, they are thermostable and can be stored at room temperature. A preferred embodiment is a viral vector which has been engineered so as to suppress the immune response of the host organism, elicited by the viral antigens. Preferred vectors of this type will carry coding regions for Interleukin 8 or 10.

Viral vectors have higher transaction (ability to introduce genes) abilities than do most chemical or physical methods to introduce genes into cells. Typically, viral vectors contain, nonstructural early genes, structural late genes, an RNA polymerase III transcript, inverted terminal repeats necessary for replication and encapsidation, and promoters to control the transcription and replication of the viral genome. When engineered as vectors, viruses typically have one or more of the early genes removed and a gene or gene/promoter cassette is inserted into the viral genome in place of the removed viral DNA. Constructs of this type can carry up to about 8 kb of foreign genetic material. The necessary functions of the removed early genes are typically supplied by cell lines which have been engineered to express the gene products of the early genes in trans.

i. Retroviral Vectors

A retrovirus is an animal virus belonging to the virus family of Retroviridae, including any types, subfamilies, genus, or tropisms. Retroviral vectors, in general, are described by Verma, I. M., Retroviral vectors for gene transfer. In Microbiology-1985, American Society for Microbiology, pp. 229-232, Washington, (1985), which is incorporated by reference herein. Examples of methods for using retroviral vectors for gene therapy are described in U.S. Pat. Nos. 4,868,116 and 4,980,286; PCT applications WO 90/02806 and WO 89/07136; and Mulligan, (Science 260:926-932 (1993)); the teachings of which are incorporated herein by reference.

A retrovirus is essentially a package which has packed into it nucleic acid cargo. The nucleic acid cargo carries with it a packaging signal, which ensures that the replicated daughter molecules will be efficiently packaged within the package coat. In addition to the package signal, there arc a number of molecules which arc needed in cis, for the replication, and packaging of the replicated virus. Typically a retroviral genome, contains the gag, pol, and env genes which are involved in the making of the protein coat. It is the gag, pol, and env genes which are typically replaced by the foreign DNA that it is to be transferred to the target cell. Retrovirus vectors typically contain a packaging signal for incorporation into the package coat, a sequence which signals the start of the gag transcription unit, elements necessary for reverse transcription, including a primer binding site to bind the tRNA primer of reverse transcription, terminal repeat sequences that guide the switch of RNA strands during DNA synthesis, a purine rich sequence 5′ to the 3′ LTR that serve as the priming site for the synthesis of the second strand of DNA synthesis, and specific sequences near the ends of the LTRs that enable the insertion of the DNA state of the retrovirus to insert into the host genome. The removal of the gag, pol, and env genes allows for about 8 kb of foreign sequence to be inserted into the viral genome, become reverse transcribed, and upon replication be packaged into a new retroviral particle. This amount of nucleic acid is sufficient for the delivery of a one to many genes depending on the size of each transcript. It is preferable to include either positive or negative selectable markers along with other genes in the insert.

Since the replication machinery and packaging proteins in most retroviral vectors have been removed (gag, pol, and env), the vectors are typically generated by placing them into a packaging cell line. A packaging cell line is a cell line which has been transfected or transformed with a retrovirus that contains the replication and packaging machinery, but lacks any packaging signal. When the vector carrying the DNA of choice is transfected into these cell lines, the vector containing the gene of interest is replicated and packaged into new retroviral particles, by the machinery provided in cis by the helper cell. The genomes for the machinery are not packaged because they lack the necessary signals.

ii. Adenoviral Vectors

The construction of replication-defective adenoviruses has been described (Berkner et al., J. Virology 61:1213-1220 (1987); Massie et al., Mol. Cell. Biol. 6:2872-2883 (1986); Haj-Ahmad et al., J. Virology 57:267-274 (1986); Davidson et al., J. Virology 61:1226-1239 (1987); Zhang “Generation and identification of recombinant adenovirus by liposome-mediated transfection and PCR analysis” BioTechniques 15:868-872 (1993)). The benefit of the use of these viruses as vectors is that they are limited in the extent to which they can spread to other cell types, since they can replicate within an initial infected cell, but arc unable to form new infectious viral particles. Recombinant adenoviruses have been shown to achieve high efficiency gene transfer after direct, in vivo delivery to airway epithelium, hepatocytes, vascular endothelium, CNS parenchyma and a number of other tissue sites (Morsy, J. Clin. Invest. 92:1580-1586 (1993); Kirshenbaum, J. Clin. Invest. 92:381-387 (1993); Roessler, J. Clin. Invest. 92:1085-1092 (1993); Moullier, Nature Genetics 4:154-159 (1993); La Salle, Science 259:988-990 (1993); Gomez-Foix, J. Biol. Chem. 267:25129-25134 (1992); Rich, Human Gene Therapy 4:461-476 (1993); Zabner, Nature Genetics 6:75-83 (1994); Guzman, Circulation Research 73:1201-1207 (1993); Bout, Human Gene Therapy 5:3-10 (1994); Zabner, Cell 75:207-216 (1993); Caillaud, Eur. J. Neuroscience 5:1287-1291 (1993); and Ragot, J. Gen. Virology 74:501-507 (1993)). Recombinant adenoviruses achieve gene transduction by binding to specific cell surface receptors, after which the virus is internalized by receptor-mediated endocytosis, in the same manner as wild type or replication-defective adenovirus (Chardonnet and Dales, Virology 40:462-477 (1970); Brown and Burlingham, J. Virology 12:386-396 (1973); Svensson and Persson, J. Virology 55:442-449 (1985); Seth, et al., J. Virol. 51:650-655 (1984); Seth, et al., Mol. Cell. Biol. 4:1528-1533 (1984); Varga et al., J. Virology 65:6061-6070 (1991); Wickham et al., Cell 73:309-319 (1993)).

A preferred viral vector is one based on an adenovirus which has had the E1 gene removed and these virons are generated in a cell line such as the human 293 cell line. In another preferred embodiment both the E1 and E3 genes are removed from the adenovirus genome.

Another type of viral vector is based on an adeno-associated virus (AAV). This defective parvovirus is a preferred vector because it can infect many cell types and is nonpathogenic to humans. AAV type vectors can transport about 4 to 5 kb and wild type AAV is known to stably insert into chromosome 19. Vectors which contain this site specific integration property are preferred. An especially preferred embodiment of this type of vector is the P4.1 C vector produced by Avigen, San Francisco, Calif., which can contain the herpes simplex virus thymidine kinase gene, HSV-tk, and/or a marker gene, such as the gene encoding the green fluorescent protein, GFP.

The inserted genes in viral and retroviral usually contain promoters, and/or enhancers to help control the expression of the desired gene product. A promoter is generally a sequence or sequences of DNA that function when in a relatively fixed location in regard to the transcription start site. A promoter contains core elements required for basic interaction of RNA polymerase and transcription factors, and can contain upstream elements and response elements.

2. Viral Promoters and Enhancers

Preferred promoters controlling transcription from vectors in mammalian host cells can be obtained from various sources, for example, the genomes of viruses such as: polyoma, Simian Virus 40 (SV40), adenovirus, retroviruses, hepatitis-B virus and most preferably cytomegalovirus, or from heterologous mammalian promoters, e.g. beta actin promoter. The early and late promoters of the SV40 virus are conveniently obtained as an SV40 restriction fragment which also contains the SV40 viral origin of replication (Fiers et al., Nature, 273: 113 (1978)). The immediate early promoter of the human cytomegalovirus is conveniently obtained as a HindIII E restriction fragment (Greenway, P. J. et al., Gene 18: 355-360 (1982)). Of course, promoters from the host cell or related species also are useful herein.

Enhancer generally refers to a sequence of DNA that functions at no fixed distance from the transcription start site and can be either 5′ (Laimins, L. et al., Proc. Natl. Acad. Sci. 78: 993 (1981)) or 3′ (Lusky, M. L., et al., Mol. Cell. Bio. 3: 1108 (1983)) to the transcription unit. Furthermore, enhancers can be within an intron (Banerji, J. L. et al., Cell 33: 729 (1983)) as well as within the coding sequence itself (Osborne, T. F., et al., Mol. Cell. Bio. 4: 1293 (1984)). They are usually between 10 and 300 bp in length, and they function in cis. Enhancers function to increase transcription from nearby promoters. Enhancers also often contain response elements that mediate the regulation of transcription. Promoters can also contain response elements that mediate the regulation of transcription. Enhancers often determine the regulation of expression of a gene. While many enhancer sequences are now known from mammalian genes (globin, elastase, albumin, α-fetoprotein and insulin), typically one will use an enhancer from a eukaryotic cell virus. Preferred examples are the SV40 enhancer on the late side of the replication origin (bp 100-270), the cytomegalovirus early promoter enhancer, the polyoma enhancer on the late side of the replication origin, and adenovirus enhancers.

The promoter and/or enhancer can be specifically activated either by light or specific chemical events which trigger their function. Systems can be regulated by reagents such as tetracycline and dexamethasone. There are also ways to enhance viral vector gene expression by exposure to irradiation, such as gamma irradiation, or alkylating chemotherapy drugs.

It is preferred that the promoter and/or enhancer region be active in all eukaryotic cell types. A preferred promoter of this type is the CMV promoter (650 bases). Other preferred promoters are SV40 promoters, cytomegalovirus (full length promoter), and retroviral vector LTF.

It has been shown that all specific regulatory elements can be cloned and used to construct expression vectors that are selectively expressed in specific cell types such as melanoma cells. The glial fibrillary acetic protein (GFAP) promoter has been used to selectively express genes in cells of glial origin.

Expression vectors used in eukaryotic host cells (yeast, fungi, insect, plant, animal, human or nucleated cells) can also contain sequences necessary for the termination of transcription which can affect mRNA expression. These regions are transcribed as polyadenylated segments in the untranslated portion of the mRNA encoding tissue factor protein. The 3′ untranslated regions also include transcription termination sites. It is preferred that the transcription unit also contain a polyadenylation region. One benefit of this region is that it increases the likelihood that the transcribed unit will be processed and transported like mRNA. The identification and use of polyadenylation signals in expression constructs is well established. It is preferred that homologous polyadenylation signals be used in the transgene constructs. In a preferred embodiment of the transcription unit, the polyadenylation region is derived from the SV40 early polyadenylation signal and consists of about 400 bases. It is also preferred that the transcribed units contain other standard sequences alone or in combination with the above sequences improve expression from, or stability of, the construct.

3. Markers

The vectors can include nucleic acid sequence encoding a marker product. This marker product is used to determine if the gene has been delivered to the cell and once delivered is being expressed. Preferred marker genes are the E. Coli lacZ gene which encodes β-galactosidase and green fluorescent protein.

In some embodiments the marker can be a selectable marker. Examples of suitable selectable markers for mammalian cells are dihydrofolate reductase (DHFR), thymidine kinase, neomycin, neomycin analog G418, hydromycin, and puromycin. When such selectable markers are successfully transferred into a mammalian host cell, the transformed mammalian host cell can survive if placed under selective pressure. There are two widely used distinct categories of selective regimes. The first category is based on a cell's metabolism and the use of a mutant cell line which lacks the ability to grow independent of a supplemented media. Two examples are: CHO DHFR⁻ cells and mouse LTK⁻ cells. These cells lack the ability to grow without the addition of such nutrients as thymidine or hypoxanthine. Because these cells lack certain genes necessary for a complete nucleotide synthesis pathway, they cannot survive unless the missing nucleotides are provided in a supplemented media. An alternative to supplementing the media is to introduce an intact DHFR or TK gene into cells lacking the respective genes, thus altering their growth requirements. Individual cells which were not transformed with the DHFR or TK gene will not be capable of survival in non-supplemented media.

The second category is dominant selection which refers to a selection scheme used in any cell type and does not require the use of a mutant cell line. These schemes typically use a drug to arrest growth of a host cell. Those cells which would express a protein conveying drug resistance and would survive the selection. Examples of such dominant selection use the drugs neomycin, (Southern P. and Berg, P., J. Molec. Appl. Genet. 1: 327 (1982)), mycophenolic acid, (Mulligan, R. C. and Berg, P. Science 209: 1422 (1980)) or hygromycin, (Sugden, B. et al., Mol. Cell. Biol. 5: 410-413 (1985)). The three examples employ bacterial genes under eukaryotic control to convey resistance to the appropriate drug G418 or neomycin (geneticin), xgpt (mycophenolic acid) or hygromycin, respectively. Others include the neomycin analog G418 and puramycin.

### E. Biosensor Riboswitches

Also disclosed are biosensor riboswitches. Biosensor riboswitches are engineered riboswitches that produce a detectable signal in the presence of their cognate trigger molecule. Useful biosensor riboswitches can be triggered at or above threshold levels of the trigger molecules. Biosensor riboswitches can be designed for use in vivo or in vitro. For example, biosensor riboswitches operably linked to a reporter RNA that encodes a protein that serves as or is involved in producing a signal can be used in vivo by engineering a cell or organism to harbor a nucleic acid construct encoding the riboswitch/reporter RNA. An example of a biosensor riboswitch for use in vitro is a riboswitch that includes a conformation dependent label, the signal from which changes depending on the activation state of the riboswitch. Such a biosensor riboswitch preferably uses an aptamer domain from or derived from a naturally occurring riboswitch, such as from a glutamine riboswitch.

### F. Reporter Proteins and Peptides

For assessing activation of a riboswitch, or for biosensor riboswitches, a reporter protein or pcptidc can be used. The reporter protein or pcptidc can be encoded by the RNA the expression of which is regulated by the riboswitch. The examples describe the use of some specific reporter proteins. The use of reporter proteins and peptides is well known and can be adapted easily for use with riboswitches. The reporter proteins can be any protein or peptide that can be detected or that produces a detectable signal. Preferably, the presence of the protein or peptide can be detected using standard techniques (e.g., radioimmunoassay, radio-labeling, immunoassay, assay for enzymatic activity, absorbance, fluorescence, luminescence, and Western blot). More preferably, the level of the reporter protein is easily quantifiable using standard techniques even at low levels. Useful reporter proteins include luciferases, green fluorescent proteins and their derivatives, such as firefly luciferase (FL) from Photinus pyralis, and Renilla luciferase (RL) from Renilla reniformis.

### G. Conformation Dependent Labels

Conformation dependent labels refer to all labels that produce a change in fluorescence intensity or wavelength based on a change in the form or conformation of the molecule or compound (such as a riboswitch) with which the label is associated. Examples of conformation dependent labels used in the context of probes and primers include molecular beacons, Amplifluors, FRET probes, cleavable FRET probes, TaqMan probes, scorpion primers, fluorescent triplex oligos including but not limited to triplex molecular beacons or triplex FRET probes, fluorescent water-soluble conjugated polymers, PNA probes and QPNA probes. Such labels, and, in particular, the principles of their function, can be adapted for use with riboswitches. Several types of conformation dependent labels are reviewed in Schweitzer and Kingsmore, Curr. Opin. Biotech. 12:21-27 (2001).

Stem quenched labels, a form of conformation dependent labels, are fluorescent labels positioned on a nucleic acid such that when a stem structure forms a quenching moiety is brought into proximity such that fluorescence from the label is quenched. When the stem is disrupted (such as when a riboswitch containing the label is activated), the quenching moiety is no longer in proximity to the fluorescent label and fluorescence increases. Examples of this effect can be found in molecular beacons, fluorescent triplex oligos, triplex molecular beacons, triplex FRET probes, and QPNA probes, the operational principles of which can be adapted for use with riboswitches.

Stem activated labels, a form of conformation dependent labels, arc labels or pairs of labels where fluorescence is increased or altered by formation of a stem structure. Stem activated labels can include an acceptor fluorescent label and a donor moiety such that, when the acceptor and donor are in proximity (when the nucleic acid strands containing the labels form a stem structure), fluorescence resonance energy transfer from the donor to the acceptor causes the acceptor to fluoresce. Stem activated labels are typically pairs of labels positioned on nucleic acid molecules (such as riboswitches) such that the acceptor and donor are brought into proximity when a stem structure is formed in the nucleic acid molecule. If the donor moiety of a stem activated label is itself a fluorescent label, it can release energy as fluorescence (typically at a different wavelength than the fluorescence of the acceptor) when not in proximity to an acceptor (that is, when a stem structure is not formed). When the stem structure forms, the overall effect would then be a reduction of donor fluorescence and an increase in acceptor fluorescence. FRET probes are an example of the use of stem activated labels, the operational principles of which can be adapted for use with riboswitches.

### H. Detection Labels

To aid in detection and quantitation of riboswitch activation, deactivation or blocking, or expression of nucleic acids or protein produced upon activation, deactivation or blocking of riboswitches, detection labels can be incorporated into detection probes or detection molecules or directly incorporated into expressed nucleic acids or proteins. As used herein, a detection label is any molecule that can be associated with nucleic acid or protein, directly or indirectly, and which results in a measurable, detectable signal, either directly or indirectly. Many such labels are known to those of skill in the art. Examples of detection labels suitable for use in the disclosed method are radioactive isotopes, fluorescent molecules, phosphorescent molecules, enzymes, antibodies, and ligands.

Examples of suitable fluorescent labels include fluorescein isothiocyanate (FITC), 5,6-carboxymethyl fluorescein, Texas red, nitrobenz-2-oxa-1,3-diazol-4-yl (NBD), coumarin, dansyl chloride, rhodamine, amino-methyl coumarin (AMCA), Eosin, Erythrosin, BODIPY®, Cascade Blue®, Oregon Green®, pyrene, lissamine, xanthenes, acridines, oxazines, phycoerythrin, macrocyclic chelates of lanthanide ions such as quantum Dye™, fluorescent energy transfer dyes, such as thiazole orange-ethidium heterodimer, and the cyanine dyes Cy3, Cy3.5, Cy5, Cy5.5 and Cy7. Examples of other specific fluorescent labels include 3-Hydroxypyrene 5,8,10-Tri Sulfonic acid, 5-Hydroxy Tryptaminc (5-HT), Acid Fuchsin, Alizarin Complcxon, Alizarin Red, Allophycocyanin, Aminocoumarin, Anthroyl Stearate, Astrazon Brilliant Red 4G, Astrazon Orange R, Astrazon Red 6B, Astrazon Yellow 7 GLL, Atabrine, Auramine, Aurophosphine, Aurophosphine G, BAO 9 (Bisaminophenyloxadiazole), BCECF, Berberine Sulphate, Bisbenzamide, Blancophor FFG Solution, Blancophor SV, Bodipy F1, Brilliant Sulphoflavin FF, Calcien Blue, Calcium Green, Calcofluor RW Solution, Calcofluor White, Calcophor White ABT Solution, Calcophor White Standard Solution, Carbostyryl, Cascade Yellow, Catecholamine, Chinacrine, Coriphosphine O, Coumarin-Phalloidin, CY3.1 8, CY5.1 8, CY7, Dans (1-Dimethyl Amino Naphaline 5 Sulphonic Acid), Dansa (Diamino Naphtyl Sulphonic Acid), Dansyl NH—CH₃, Diamino Phenyl Oxydiazole (DAO), Dimethylamino-5-Sulphonic acid, Dipyrrometheneboron Difluoride, Diphenyl Brilliant Flavine 7GFF, Dopamine, Erythrosin ITC, Euchrysin, FIF (Formaldehyde Induced Fluorescence), Flazo Orange, Fluo 3, Fluorescamine, Fura-2, Genacryl Brilliant Red B, Genacryl Brilliant Yellow 10GF, Genacryl Pink 3G, Genacryl Yellow 5GF, Gloxalic Acid, Granular Blue, Haematoporphyrin, Indo-1, Intrawhite Cf Liquid, Leucophor PAF, Leucophor SF, Leucophor WS, Lissamine Rhodamine B200 (RD200), Lucifer Yellow CH, Lucifer Yellow VS, Magdala Red, Marina Blue, Maxilon Brilliant Flavin 10 GFF, Maxilon Brilliant Flavin 8 GFF, MPS (Methyl Green Pyronine Stilbene), Mithramycin, NBD Amine, Nitrobenzoxadidole, Noradrenaline, Nuclear Fast Red, Nuclear Yellow, Nylosan Brilliant Flavin EBG, Oxadiazole, Pacific Blue, Pararosaniline (Feulgen), Phorwite AR Solution, Phorwite BKL, Phorwite Rev, Phorwite RPA, Phosphine 3R, Phthalocyanine, Phycoerythrin R, Polyazaindacene Pontochrome Blue Black, Porphyrin, Primuline, Procion Yellow, Pyronine, Pyronine B, Pyrozal Brilliant Flavin 7GF, Quinacrine Mustard, Rhodamine 123, Rhodamine 5 GLD, Rhodamine 6G, Rhodamine B, Rhodamine B 200, Rhodamine B Extra, Rhodamine BB, Rhodamine BG, Rhodamine WT, Serotonin, Sevron Brilliant Red 2B, Sevron Brilliant Red 4G, Sevron Brilliant Red B, Sevron Orange, Sevron Yellow L, SITS (Primuline), SITS (Stilbene Isothiosulphonic acid), Stilbene, Snarf 1, sulpho Rhodamine B Can C, Sulpho Rhodamine G Extra, Tetracycline, Thiazine Red R, Thioflavin S, Thioflavin TCN, Thioflavin 5, Thiolyte, Thiozol Orange, Tinopol CBS, True Blue, Ultralite, Uranine B, Uvitex SFC, Xylene Orange, and XRITC.

Useful fluorescent labels are fluorescein (5-carboxyfluorescein-N-hydroxysuccinimide ester), rhodamine (5,6-tetramethyl rhodamine), and the cyanine dyes Cy3, Cy3.5, Cy5, Cy5.5 and Cy7. The absorption and emission maxima, respectively, for these fluors arc: FITC (490 nm; 520 nm), Cy3 (554 nm; 568 nm), Cy3.5 (581 nm; 588 nm), Cy5 (652 nm: 672 nm), Cy5.5 (682 nm; 703 nm) and Cy7 (755 nm; 778 nm), thus allowing their simultaneous detection. Other examples of fluorescein dyes include 6-carboxyfluorescein (6-FAM), 2′,4′,1,4,-tetrachlorofluorescein (TET), 2′,4′,5′,7′,1,4-hexachlorofluorescein (HEX), 2′,7′-dimethoxy-4′,5′-dichloro-6-carboxyrhodamine (JOE), 2′-chloro-5′-fluoro-7′,8′-fused phenyl-1,4-dichloro-6-carboxyfluorescein (NED), and 2′-chloro-7′-phenyl-1,4-dichloro-6-carboxyfluorescein (VIC). Fluorescent labels can be obtained from a variety of commercial sources, including Amersham Pharmacia Biotech, Piscataway, N.J.; Molecular Probes, Eugene, Oreg.; and Research Organics, Cleveland, Ohio.

Additional labels of interest include those that provide for signal only when the probe with which they are associated is specifically bound to a target molecule, where such labels include: “molecular beacons” as described in Tyagi & Kramer, Nature Biotechnology (1996) 14:303 and EP 0 070 685 B1. Other labels of interest include those described in U.S. Pat. No. 5,563,037; WO 97/17471 and WO 97/17076.

Labeled nucleotides are a useful form of detection label for direct incorporation into expressed nucleic acids during synthesis. Examples of detection labels that can be incorporated into nucleic acids include nucleotide analogs such as BrdUrd (5-bromodeoxyuridine, Hoy and Schimke, Mutation Research 290:217-230 (1993)), aminoallyldeoxyuridine (Henegariu et al., Nature Biotechnology 18:345-348 (2000)), 5-methylcytosine (Sano et al., Biochim. Biophys. Acta 951:157-165 (1988)), bromouridine (Wansick et al., J. Cell Biology 122:283-293 (1993)) and nucleotides modified with biotin (Langer et al., Proc. Natl. Acad. Sci. USA 78:6633 (1981)) or with suitable haptens such as digoxygenin (Kerkhof, Anal. Biochem. 205:359-364 (1992)). Suitable fluorescence-labeled nucleotides are Fluorescein-isothiocyanate-dUTP, Cyanine-3-dUTP and Cyanine-5-dUTP (Yu et al., Nucleic Acids Res., 22:3226-3232 (1994)). A preferred nucleotide analog detection label for DNA is BrdUrd (bromodeoxyuridine, BrdUrd, BrdU, BUdR, Sigma-Aldrich Co). Other useful nucleotide analogs for incorporation of detection label into DNA are AA-dUTP (aminoallyl-deoxyuridine triphosphate, Sigma-Aldrich Co.), and 5-methyl-dCTP (Roche Molecular Biochemicals). A useful nucleotide analog for incorporation of detection label into RNA is biotin-16-UTP (biotin-16-uridine-5′-triphosphate, Roche Molecular Biochemicals). Fluorescein, Cy3, and Cy5 can be linked to dUTP for direct labeling. Cy3.5 and Cy7 are available as avidin or anti-digoxygenin conjugates for secondary detection of biotin- or digoxygenin-labeled probes.

Detection labels that are incorporated into nucleic acid, such as biotin, can be subsequently detected using sensitive methods well-known in the art. For example, biotin can be detected using streptavidin-alkaline phosphatase conjugate (Tropix, Inc.), which is bound to the biotin and subsequently detected by chemiluminescence of suitable substrates (for example, chemiluminescent substrate CSPD: disodium, 3-(4-methoxyspiro-[1,2,-dioxetane-3-2′-(5′-chloro)tricyclo [3.3.1.1³′⁷]decane]-4-yl) phenyl phosphate; Tropix, Inc.). Labels can also be enzymes, such as alkaline phosphatase, soybean peroxidase, horseradish peroxidase and polymerases, that can be detected, for example, with chemical signal amplification or by using a substrate to the enzyme which produces light (for example, a chemiluminescent 1,2-dioxetane substrate) or fluorescent signal.

Molecules that combine two or more of these detection labels are also considered detection labels. Any of the known detection labels can be used with the disclosed probes, tags, molecules and methods to label and detect activated or deactivated riboswitches or nucleic acid or protein produced in the disclosed methods. Methods for detecting and measuring signals generated by detection labels are also known to those of skill in the art. For example, radioactive isotopes can be detected by scintillation counting or direct visualization; fluorescent molecules can be detected with fluorescent spectrophotometers; phosphorescent molecules can be detected with a spectrophotometer or directly visualized with a camera; enzymes can be detected by detection or visualization of the product of a reaction catalyzed by the enzyme; antibodies can be detected by detecting a secondary detection label coupled to the antibody. As used herein, detection molecules are molecules which interact with a compound or composition to be detected and to which one or more detection labels are coupled.

### I. Sequence Similarities

It is understood that as discussed herein the use of the terms homology and identity mean the same thing as similarity. Thus, for example, if the use of the word homology is used between two sequences (non-natural sequences, for example) it is understood that this is not necessarily indicating an evolutionary relationship between these two sequences, but rather is looking at the similarity or relatedness between their nucleic acid sequences. Many of the methods for determining homology between two evolutionarily related molecules are routinely applied to any two or more nucleic acids or proteins for the purpose of measuring sequence similarity regardless of whether they are evolutionarily related or not.

In general, it is understood that one way to define any known variants and derivatives or those that might arise, of the disclosed riboswitches, aptamers, expression platforms, genes and proteins herein, is through defining the variants and derivatives in terms of homology to specific known sequences. This identity of particular sequences disclosed herein is also discussed elsewhere herein. In general, variants of riboswitches, aptamers, expression platforms, genes and proteins herein disclosed typically have at least, about 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99 percent homology to a stated sequence or a native sequence. Those of skill in the art readily understand how to determine the homology of two proteins or nucleic acids, such as genes. For example, the homology can be calculated after aligning the two sequences so that the homology is at its highest level. Another way of calculating homology can be performed by published algorithms.

Optimal alignment of sequences for comparison can be conducted by the local homology algorithm of Smith and Waterman Adv. Appl. Math. 2: 482 (1981), by the homology alignment algorithm of Needleman and Wunsch, J. Mol. Biol. 48: 443 (1970), by the search for similarity method of Pearson and Lipman, Proc. Natl. Acad. Sci. U.S.A. 85: 2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by inspection.

The same types of homology can be obtained for nucleic acids by for example the algorithms disclosed in Zuker, M. Science 244:48-52, 1989, Jaeger et al. Proc. Natl. Acad. Sci. USA 86:7706-7710, 1989, Jaeger et al. Methods Enzymol. 183:281-306, 1989 which are herein incorporated by reference for at least material related to nucleic acid alignment. It is understood that any of the methods typically can be used and that in certain instances the results of these various methods can differ, but the skilled artisan understands if identity is found with at least one of these methods, the sequences would be said to have the stated identity.

For example, as used herein, a sequence recited as having a particular percent homology to another sequence refers to sequences that have the recited homology as calculated by any one or more of the calculation methods described above. For example, a first sequence has 80 percent homology, as defined herein, to a second sequence if the first sequence is calculated to have 80 percent homology to the second sequence using the Zuker calculation method even if the first sequence does not have 80 percent homology to the second sequence as calculated by any of the other calculation methods. As another example, a first sequence has 80 percent homology, as defined herein, to a second sequence if the first sequence is calculated to have 80 percent homology to the second sequence using both the Zuker calculation method and the Pearson and Lipman calculation method even if the first sequence does not have 80 percent homology to the second sequence as calculated by the Smith and Waterman calculation method, the Needleman and Wunsch calculation method, the Jaeger calculation methods, or any of the other calculation methods. As yet another example, a first sequence has 80 percent homology, as defined herein, to a second sequence if the first sequence is calculated to have 80 percent homology to the second sequence using each of calculation methods (although, in practice, the different calculation methods will often result in different calculated homology percentages).

### J. Hybridization and Selective Hybridization

The term hybridization typically means a sequence driven interaction between at least two nucleic acid molecules, such as a primer or a probe and a riboswitch or a gene. Sequence driven interaction means an interaction that occurs between two nucleotides or nucleotide analogs or nucleotide derivatives in a nucleotide specific manner. For example, G interacting with C or A interacting with T are sequence driven interactions. Typically sequence driven interactions occur on the Watson-Crick face or Hoogsteen face of the nucleotide. The hybridization of two nucleic acids is affected by a number of conditions and parameters known to those of skill in the art. For example, the salt concentrations, pH, and temperature of the reaction all affect whether two nucleic acid molecules will hybridize.

Parameters for selective hybridization between two nucleic acid molecules are well known to those of skill in the art. For example, in some embodiments selective hybridization conditions can be defined as stringent hybridization conditions. For example, stringency of hybridization is controlled by both temperature and salt concentration of either or both of the hybridization and washing steps. For example, the conditions of hybridization to achieve selective hybridization can involve hybridization in high ionic strength solution (6×SSC or 6×SSPE) at a temperature that is about 12-25° C. below the Tm (the melting temperature at which half of the molecules dissociate from their hybridization partners) followed by washing at a combination of temperature and salt concentration chosen so that the washing temperature is about 5° C. to 20° C. below the Tm. The temperature and salt conditions are readily determined empirically in preliminary experiments in which samples of reference DNA immobilized on filters are hybridized to a labeled nucleic acid of interest and then washed under conditions of different stringencies. Hybridization temperatures are typically higher for DNA-RNA and RNA-RNA hybridizations. The conditions can be used as described above to achieve stringency, or as is known in the art (Sambrook et al., Molecular Cloning: A Laboratory Manual, 2nd Ed., Cold Spring Harbor Laboratory, Cold Spring Harbor, N.Y., 1989; Kunkel et al. Methods Enzymol. 1987:154:367, 1987 which is herein incorporated by reference for material at least related to hybridization of nucleic acids). A preferable stringent hybridization condition for a DNA:DNA hybridization can be at about 68° C. (in aqueous solution) in 6×SSC or 6×SSPE followed by washing at 68° C. Stringency of hybridization and washing, if desired, can be reduced accordingly as the degree of complementarity desired is decreased, and further, depending upon the G-C or A-T richness of any area wherein variability is searched for. Likewise, stringency of hybridization and washing, if desired, can be increased accordingly as homology desired is increased, and further, depending upon the G-C or A-T richness of any area wherein high homology is desired, all as known in the art.

Another way to define selective hybridization is by looking at the amount (percentage) of one of the nucleic acids bound to the other nucleic acid. For example, in some embodiments selective hybridization conditions would be when at least about, 60, 65, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 percent of the limiting nucleic acid is bound to the non-limiting nucleic acid. Typically, the non-limiting nucleic acid is in for example, 10 or 100 or 1000 fold excess. This type of assay can be performed at under conditions where both the limiting and non-limiting nucleic acids are for example, 10 fold or 100 fold or 1000 fold below their k_{d}, or where only one of the nucleic acid molecules is 10 fold or 100 fold or 1000 fold or where one or both nucleic acid molecules are above their k_{d}.

Another way to define selective hybridization is by looking at the percentage of nucleic acid that gets enzymatically manipulated under conditions where hybridization is required to promote the desired enzymatic manipulation. For example, in some embodiments selective hybridization conditions would be when at least about, 60, 65, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 percent of the nucleic acid is enzymatically manipulated under conditions which promote the enzymatic manipulation, for example if the enzymatic manipulation is DNA extension, then selective hybridization conditions would be when at least about 60, 65, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 percent of the nucleic acid molecules are extended. Preferred conditions also include those suggested by the manufacturer or indicated in the art as being appropriate for the enzyme performing the manipulation.

Just as with homology, it is understood that there are a variety of methods herein disclosed for determining the level of hybridization between two nucleic acid molecules. It is understood that these methods and conditions can provide different percentages of hybridization between two nucleic acid molecules, but unless otherwise indicated meeting the parameters of any of the methods would be sufficient. For example if 80% hybridization was required and as long as hybridization occurs within the required parameters in any one of these methods it is considered disclosed herein.

It is understood that those of skill in the art understand that if a composition or method meets any one of these criteria for determining hybridization either collectively or singly it is a composition or method that is disclosed herein.

### K. Nucleic Acids

There are a variety of molecules disclosed herein that are nucleic acid based, including, for example, riboswitches, aptamers, and nucleic acids that encode riboswitches and aptamers. The disclosed nucleic acids can be made up of for example, nucleotides, nucleotide analogs, or nucleotide substitutes. Non-limiting examples of these and other molecules are discussed herein. It is understood that for example, when a vector is expressed in a cell, that the expressed mRNA will typically be made up of A, C, G, and U. Likewise, it is understood that if a nucleic acid molecule is introduced into a cell or cell environment through for example exogenous delivery, it is advantageous that the nucleic acid molecule be made up of nucleotide analogs that reduce the degradation of the nucleic acid molecule in the cellular environment.

So long as their relevant function is maintained, riboswitches, aptamers, expression platforms and any other oligonucleotides and nucleic acids can be made up of or include modified nucleotides (nucleotide analogs). Many modified nucleotides are known and can be used in oligonucleotides and nucleic acids. A nucleotide analog is a nucleotide which contains some type of modification to either the base, sugar, or phosphate moieties. Modifications to the base moiety would include natural and synthetic modifications of A, C, G, and T/U as well as different purine or pyrimidine bases, such as uracil-5-yl, hypoxanthin-9-yl (I), and 2-aminoadenin-9-yl. A modified base includes but is not limited to 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 8-azaguanine and 8-azaadenine, 7-deazaguanine and 7-deazaadenine and 3-deazaguanine and 3-deazaadenine Additional base modifications can be found for example in U.S. Pat. No. 3,687,808, Englisch et al., Angewandte Chemie, International Edition, 1991, 30, 613, and Sanghvi, Y. S., Chapter 15, Antisense Research and Applications, pages 289-302, Crooke, S. T. and Lebleu, B. ed., CRC Press, 1993. Certain nucleotide analogs, such as 5-substituted pyrimidines, 6-azapyrimidines and N-2, N-6 and O-6 substituted purines, including 2-aminopropyladenine, 5-propynyluracil and 5-propynylcytosine. 5-methylcytosine can increase the stability of duplex formation. Other modified bases are those that function as universal bases. Universal bases include 3-nitropyrrole and 5-nitroindole. Universal bases substitute for the normal bases but have no bias in base pairing. That is, universal bases can base pair with any other base. Base modifications often can be combined with for example a sugar modification, such as 2′-O-methoxyethyl, to achieve unique properties such as increased duplex stability. There are numerous United States patents such as U.S. Pat. Nos. 4,845,205; 5,130,302; 5,134,066; 5,175,273; 5,367,066; 5,432,272; 5,457,187; 5,459,255; 5,484,908; 5,502,177; 5,525,711; 5,552,540; 5,587,469; 5,594,121, 5,596,091; 5,614,617; and 5,681,941, which detail and describe a range of base modifications. Each of these patents is herein incorporated by reference in its entirety, and specifically for their description of base modifications, their synthesis, their use, and their incorporation into oligonucleotides and nucleic acids.

Nucleotide analogs can also include modifications of the sugar moiety. Modifications to the sugar moiety would include natural modifications of the ribose and deoxyribose as well as synthetic modifications. Sugar modifications include but are not limited to the following modifications at the 2′ position: OH; F; O-, S-, or N-alkyl; O-, S-, or N-alkenyl; O-, S- or N-alkynyl; or O-alkyl-O-alkyl, wherein the alkyl, alkenyl and alkynyl can be substituted or unsubstituted C1 to C10, alkyl or C₂ to C₁₀ alkenyl and alkynyl. 2′ sugar modifications also include but are not limited to —O[(CH₂)ₙ O]ₘ CH₃, —O(CH₂)ₙ OCH₃, —O(CH₂)ₙ NH₂, —O(CH₂)ₙ CH₃, —O(CH₂)ₙ—ONH₂, and —O(CH₂)ₙON[(CH₂)ₙ CH₃)]₂, where n and m are from 1 to about 10.

Other modifications at the 2′ position include but arc not limited to: C1 to C 10 lower alkyl, substituted lower alkyl, alkaryl, aralkyl, O-alkaryl or O-aralkyl, SH, SCH₃, OCN, Cl, Br, CN, CF₃, OCF₃, SOCH₃, SO₂ CH₃, ONO₂, NO₂, N₃, NH₂, heterocycloalkyl, heterocycloalkaryl, aminoalkylamino, polyalkylamino, substituted silyl, an RNA cleaving group, a reporter group, an intercalator, a group for improving the pharmacokinetic properties of an oligonucleotide, or a group for improving the pharmacodynamic properties of an oligonucleotide, and other substituents having similar properties. Similar modifications can also be made at other positions on the sugar, particularly the 3′ position of the sugar on the 3′ terminal nucleotide or in 2′-5′ linked oligonucleotides and the 5′ position of 5′ terminal nucleotide. Modified sugars would also include those that contain modifications at the bridging ring oxygen, such as CH₂ and S, Nucleotide sugar analogs can also have sugar mimetics such as cyclobutyl moieties in place of the pentofuranosyl sugar. There are numerous United States patents that teach the preparation of such modified sugar structures such as 4,981,957; 5,118,800; 5,319,080; 5,359,044; 5,393,878; 5,446,137; 5,466,786; 5,514,785; 5,519,134; 5,567,811; 5,576,427; 5,591,722; 5,597,909; 5,610,300; 5,627,053; 5,639,873; 5,646,265; 5,658,873; 5,670,633; and 5,700,920, each of which is herein incorporated by reference in its entirety, and specifically for their description of modified sugar structures, their synthesis, their use, and their incorporation into nucleotides, oligonucleotides and nucleic acids.

Nucleotide analogs can also be modified at the phosphate moiety. Modified phosphate moieties include but are not limited to those that can be modified so that the linkage between two nucleotides contains a phosphorothioate, chiral phosphorothioate, phosphorodithioate, phosphotriester, aminoalkylphosphotriester, methyl and other alkyl phosphonates including 3′-alkylene phosphonate and chiral phosphonates, phosphinates, phosphoramidates including 3′-amino phosphoramidate and aminoalkylphosphoramidates, thionophosphoramidates, thionoalkylphosphonates, thionoalkylphosphotriesters, and boranophosphates. It is understood that these phosphate or modified phosphate linkages between two nucleotides can be through a 3′-5′ linkage or a 2′-5′ linkage, and the linkage can contain inverted polarity such as 3′-5′ to 5′-3′ or 2′-5′ to 5′-2′. Various salts, mixed salts and free acid forms are also included. Numerous United States patents teach how to make and use nucleotides containing modified phosphates and include but are not limited to, 3,687,808; 4,469,863; 4,476,301; 5,023,243; 5,177,196; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,306; 5,550,111; 5,563,253; 5,571,799; 5,587,361; and 5,625,050, each of which is herein incorporated by reference its entirety, and specifically for their description of modified phosphates, their synthesis, their use, and their incorporation into nucleotides, oligonucleotides and nucleic acids.

It is understood that nucleotide analogs need only contain a single modification, but can also contain multiple modifications within one of the moieties or between different moieties.

Nucleotide substitutes are molecules having similar functional properties to nucleotides, but which do not contain a phosphate moiety, such as peptide nucleic acid (PNA). Nucleotide substitutes are molecules that will recognize and hybridize to (base pair to) complementary nucleic acids in a Watson-Crick or Hoogsteen manner, but which are linked together through a moiety other than a phosphate moiety. Nucleotide substitutes are able to conform to a double helix type structure when interacting with the appropriate target nucleic acid.

Nucleotide substitutes are nucleotides or nucleotide analogs that have had the phosphate moiety and/or sugar moieties replaced. Nucleotide substitutes do not contain a standard phosphorus atom. Substitutes for the phosphate can be for example, short chain alkyl or cycloalkyl internucleoside linkages, mixed heteroatom and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; and others having mixed N, O, S and CH₂ component parts. Numerous United States patents disclose how to make and use these types of phosphate replacements and include but are not limited to 5,034,506; 5,166,315; 5,185,444; 5,214,134; 5,216,141; 5,235,033; 5,264,562; 5,264,564; 5,405,938; 5,434,257; 5,466,677; 5,470,967; 5,489,677; 5,541,307; 5,561,225; 5,596,086; 5,602,240; 5,610,289; 5,602,240; 5,608,046; 5,610,289; 5,618,704; 5,623,070; 5,663,312; 5,633,360; 5,677,437; and 5,677,439, each of which is herein incorporated by reference its entirety, and specifically for their description of phosphate replacements, their synthesis, their use, and their incorporation into nucleotides, oligonucleotides and nucleic acids.

It is also understood in a nucleotide substitute that both the sugar and the phosphate moieties of the nucleotide can be replaced, by for example an amide type linkage (aminoethylglycine) (PNA). U.S. Pat. Nos. 5,539,082; 5,714,331; and 5,719,262 teach how to make and use PNA molecules, each of which is herein incorporated by reference. (See also Nielsen et al., Science 254:1497-1500 (1991)). Oligonucleotides and nucleic acids can be comprised of nucleotides and can be made up of different types of nucleotides or the same type of nucleotides. For example, one or more of the nucleotides in an oligonucleotide can be ribonucleotides, 2′-O-methyl ribonucleotides, or a mixture of ribonucleotides and 2′-O-methyl ribonucleotides; about 10% to about 50% of the nucleotides can be ribonucleotides, 2′-O-methyl ribonucleotides, or a mixture of ribonucleotides and 2′-O-methyl ribonucleotides; about 50% or more of the nucleotides can be ribonucleotides, 2′-O-methyl ribonucleotides, or a mixture of ribonucleotides and 2′-O-methyl ribonucleotides; or all of the nucleotides are ribonucleotides, 2′-O-methyl ribonucleotides, or a mixture of ribonucleotides and 2′-O-methyl ribonucleotides. Such oligonucleotides and nucleic acids can be referred to as chimeric oligonucleotides and chimeric nucleic acids.

### L. Solid Supports

Solid supports are solid-state substrates or supports with which molecules (such as trigger molecules) and riboswitches (or other components used in, or produced by, the disclosed methods) can be associated. Riboswitches and other molecules can be associated with solid supports directly or indirectly. For example, analytes (e.g., trigger molecules, test compounds) can be bound to the surface of a solid support or associated with capture agents (e.g., compounds or molecules that bind an analyte) immobilized on solid supports. As another example, riboswitches can be bound to the surface of a solid support or associated with probes immobilized on solid supports. An array is a solid support to which multiple riboswitches, probes or other molecules have been associated in an array, grid, or other organized pattern.

Solid-state substrates for use in solid supports can include any solid material with which components can be associated, directly or indirectly. This includes materials such as acrylamide, agarose, cellulose, nitrocellulose, glass, gold, polystyrene, polyethylene vinyl acetate, polypropylene, polymethacrylate, polyethylene, polyethylene oxide, polysilicates, polycarbonates, teflon, fluorocarbons, nylon, silicon rubber, polyanhydrides, polyglycolic acid, polylactic acid, polyorthoesters, functionalized silane, polypropylfumerate, collagen, glycosaminoglycans, and polyamino acids. Solid-state substrates can have any useful form including thin film, membrane, bottles, dishes, fibers, woven fibers, shaped polymers, particles, beads, microparticles, or a combination. Solid-state substrates and solid supports can be porous or non-porous. A chip is a rectangular or square small piece of material. Preferred forms for solid-state substrates are thin films, beads, or chips. A useful form for a solid-state substrate is a microtiter dish. In some embodiments, a multiwell glass slide can be employed.

An array can include a plurality of riboswitches, trigger molecules, other molecules, compounds or probes immobilized at identified or predefined locations on the solid support. Each predefined location on the solid support generally has one type of component (that is, all the components at that location are the same). Alternatively, multiple types of components can be immobilized in the same predefined location on a solid support. Each location will have multiple copies of the given components. The spatial separation of different components on the solid support allows separate detection and identification.

Although useful, it is not required that the solid support be a single unit or structure. A set of riboswitches, trigger molecules, other molecules, compounds and/or probes can be distributed over any number of solid supports. For example, at one extreme, each component can be immobilized in a separate reaction tube or container, or on separate beads or microparticles.

Methods for immobilization of oligonucleotides to solid-state substrates are well established. Oligonucleotides, including address probes and detection probes, can be coupled to substrates using established coupling methods. For example, suitable attachment methods are described by Pease et al., Proc. Natl. Acad. Sci. USA 91(11):5022-5026 (1994), and Khrapko et al., Mol Biol (Mosk) (USSR) 25:718-730 (1991). A method for immobilization of 3′-amine oligonucleotides on casein-coated slides is described by Stimpson et al., Proc. Natl. Acad. Sci. USA 92:6379-6383 (1995). A useful method of attaching oligonucleotides to solid-state substrates is described by Guo et al., Nucleic Acids Res. 22:5456-5465 (1994).

Each of the components (for example, riboswitches, trigger molecules, or other molecules) immobilized on the solid support can be located in a different predefined region of the solid support. The different locations can be different reaction chambers. Each of the different predefined regions can be physically separated from each other of the different regions. The distance between the different predefined regions of the solid support can be either fixed or variable. For example, in an array, each of the components can be arranged at fixed distances from each other, while components associated with beads will not be in a fixed spatial relationship. In particular, the use of multiple solid support units (for example, multiple beads) will result in variable distances.

Components can be associated or immobilized on a solid support at any density. Components can be immobilized to the solid support at a density exceeding 400 different components per cubic centimeter. Arrays of components can have any number of components. For example, an array can have at least 1,000 different components immobilized on the solid support, at least 10,000 different components immobilized on the solid support, at least 100,000 different components immobilized on the solid support, or at least 1,000,000 different components immobilized on the solid support.

### M. Kits

The materials described above as well as other materials can be packaged together in any suitable combination as a kit useful for performing, or aiding in the performance of the disclosed method. It is useful if the kit components in a given kit are designed and adapted for use together in the disclosed method. For example disclosed are kits for detecting compounds, the kit comprising one or more biosensor riboswitches. The kits also can contain reagents and labels for detecting activation of the riboswitches.

### N. Mixtures

Disclosed are mixtures formed by performing or preparing to perform the disclosed method. For example, disclosed are mixtures comprising riboswitches and trigger molecules.

Whenever the method involves mixing or bringing into contact compositions or components or reagents, performing the method creates a number of different mixtures. For example, if the method includes 3 mixing steps, after each one of these steps a unique mixture is formed if the steps are performed separately. In addition, a mixture is formed at the completion of all of the steps regardless of how the steps were performed. The present disclosure contemplates these mixtures, obtained by the performance of the disclosed methods as well as mixtures containing any disclosed reagent, composition, or component, for example, disclosed herein.

### O. Systems

Disclosed are systems useful for performing, or aiding in the performance of, the disclosed method. Systems generally comprise combinations of articles of manufacture such as structures, machines, devices, and the like, and compositions, compounds, materials, and the like. Such combinations that are disclosed or that are apparent from the disclosure arc contemplated. For example, disclosed and contemplated arc systems comprising biosensor riboswitches, a solid support and a signal-reading device.

### P. Data Structures and Computer Control

Disclosed are data structures used in, generated by, or generated from, the disclosed method. Data structures generally are any form of data, information, and/or objects collected, organized, stored, and/or embodied in a composition or medium. Riboswitch structures and activation measurements stored in electronic form, such as in RAM or on a storage disk, is a type of data structure.

The disclosed method, or any part thereof or preparation therefor, can be controlled, managed, or otherwise assisted by computer control. Such computer control can be accomplished by a computer controlled process or method, can use and/or generate data structures, and can use a computer program. Such computer control, computer controlled processes, data structures, and computer programs are contemplated and should be understood to be disclosed herein.

### Methods

Disclosed are methods of identifying compounds that activate, deactivate or block a riboswitch. For example, compounds that activate a riboswitch can be identified by bringing into contact a test compound and a riboswitch and assessing activation of the riboswitch. If the riboswitch is activated, the test compound is identified as a compound that activates the riboswitch. Activation of a riboswitch can be assessed in any suitable manner. For example, the riboswitch can be linked to a reporter RNA and expression, expression level, or change in expression level of the reporter RNA can be measured in the presence and absence of the test compound. As another example, the riboswitch can include a conformation dependent label, the signal from which changes depending on the activation state of the riboswitch. Such a riboswitch preferably uses an aptamer domain from or derived from a naturally occurring riboswitch. As can be seen, assessment of activation of a riboswitch can be performed with the use of a control assay or measurement or without the use of a control assay or measurement. Methods for identifying compounds that deactivate a riboswitch can be performed in analogous ways.

Identification of compounds that block a riboswitch can be accomplished in any suitable manner. For example, an assay can be performed for assessing activation or deactivation of a riboswitch in the presence of a compound known to activate or deactivate the riboswitch and in the presence of a test compound. If activation or deactivation is not observed as would be observed in the absence of the test compound, then the test compound is identified as a compound that blocks activation or deactivation of the riboswitch.

Multiple different approaches can be used to detect binding RNAs, including, for example, allosteric ribozyme assays using gel-based and chip-based detection methods, and in-line probing assays. High throughput testing can also be accomplished by using, for example, fluorescent detection methods. For example, the natural catalytic activity of a glucosamine-6-phosphate sensing riboswitch that controls gene expression by activating RNA-cleaving ribozyme can be used. This ribozyme can be reconfigured to cleave separate substrate molecules with multiple turnover kinetics. Therefore, a fluorescent group held in proximity to a quenching group can be uncoupled (and therefore become more fluorescent) if a compound triggers ribozyme function. Second, molecular beacon technology can be employed. This creates a system that suppresses fluorescence if a compound prevents the beacon from docking to the riboswitch RNA. Either approach can be applied to any of the riboswitch classes by using RNA engineering strategies described herein.

High-throughput screening can also be used to reveal entirely new chemical scaffolds that also bind to riboswitch RNAs either with standard or non-standard modes of molecular recognition. Multiple different approaches can be used to detect metabolite binding RNAs, including allosteric ribozyme assays using gel-based and chip-based detection methods, and in-line probing assays. Also disclosed are compounds made by identifying a compound that activates, deactivates or blocks a riboswitch and manufacturing the identified compound. This can be accomplished by, for example, combining compound identification methods as disclosed elsewhere herein with methods for manufacturing the identified compounds. For example, compounds can be made by bringing into contact a test compound and a riboswitch, assessing activation of the riboswitch, and, if the riboswitch is activated by the test compound, manufacturing the test compound that activates the riboswitch as the compound.

Also disclosed are compounds made by checking activation, deactivation or blocking of a riboswitch by a compound and manufacturing the checked compound. This can be accomplished by, for example, combining compound activation, deactivation or blocking assessment methods as disclosed elsewhere herein with methods for manufacturing the checked compounds. For example, compounds can be made by bringing into contact a test compound and a riboswitch, assessing activation of the riboswitch, and, if the riboswitch is activated by the test compound, manufacturing the test compound that activates the riboswitch as the compound. Checking compounds for their ability to activate, deactivate or block a riboswitch refers to both identification of compounds previously unknown to activate, deactivate or block a riboswitch and to assessing the ability of a compound to activate, deactivate or block a riboswitch where the compound was already known to activate, deactivate or block the riboswitch.

Certain materials, compounds, compositions, and components disclosed herein can be obtained commercially or readily synthesized using techniques generally known to those of skill in the art. For example, the starting materials and reagents used in preparing the disclosed compounds and compositions are either available from commercial suppliers such as Aldrich Chemical Co., (Milwaukee, Wis.), Acros Organics (Morris Plains, N.J.), Fisher Scientific (Pittsburgh, Pa.), or Sigma (St. Louis, Mo.) or are prepared by methods known to those skilled in the art following procedures set forth in references such as Fieser and Fieser's Reagents for Organic Synthesis, Volumes 1-17 (John Wiley and Sons, 1991); Rodd's Chemistry of Carbon Compounds, Volumes 1-5 and Supplementals (Elsevier Science Publishers, 1989); Organic Reactions, Volumes 1-40 (John Wiley and Sons, 1991); March's Advanced Organic Chemistry, (John Wiley and Sons, 4th Edition); and Larock's Comprehensive Organic Transformations (VCH Publishers Inc., 1989).

It should be understood that particular contacts and interactions (such as hydrogen bond donation or acceptance) described herein for compounds interacting with riboswitches are preferred but are not essential for interaction of a compound with a riboswitch. For example, compounds can interact with riboswitches with less affinity and/or specificity than compounds having the disclosed contacts and interactions. Further, different or additional functional groups on the compounds can introduce new, different and/or compensating contacts with the riboswitches. For example, for glutamine riboswitches, large or small functional groups can be used. Such functional groups can have, and can be designed to have, contacts and interactions with other part of the riboswitch. Such contacts and interactions can compensate for contacts and interactions of the trigger molecules and core structure. Useful functional groups can be attached, for example, to the alpha-carbon of glutamine. Modifications to the side chain, carboxy group, primary amino group, or a combination, of glutamine can be used or avoided.

Also disclosed are methods of killing or inhibiting the growth of bacteria. The method can comprise contacting the bacteria with a compound identified by any of the methods disclosed herein. The method can comprise selecting a compound identified by any of the methods disclosed herein and contacting the bacteria with the selected compound. Also disclosed are methods of inhibiting gene expression. The method can comprise bringing into contact a compound and a cell, wherein the compound is identified by any of the disclosed methods. Also disclosed are methods of inhibiting gene expression. The method can comprise bringing into contact a compound and a cell, wherein the compound is identified by any of the disclosed methods. The method can comprise selecting a compound identified by any of the methods disclosed herein and bringing into contact the compound and a cell.

Also disclosed are methods comprising: (a) testing a compound identified by any of the disclosed methods for inhibition of gene expression of a gene encoding an RNA comprising a glutamine riboswitch, wherein the inhibition is via the riboswitch; and (b) inhibiting gene expression by bringing into contact a cell and a compound that inhibited gene expression in step (a). The cell can comprise a gene encoding an RNA comprising a target riboswitch, wherein the target riboswitch is a glutamine riboswitch, wherein the compound inhibits expression of the gene by binding to the target riboswitch.

Also disclosed are methods for activating, deactivating or blocking a riboswitch. Such methods can involve, for example, bringing into contact a riboswitch and a compound or trigger molecule that can activate, deactivate or block the riboswitch. Riboswitches function to control gene expression through the binding or removal of a trigger molecule. Compounds can be used to activate, deactivate or block a riboswitch. The trigger molecule for a riboswitch (as well as other activating compounds) can be used to activate a riboswitch. Compounds other than the trigger molecule generally can be used to deactivate or block a riboswitch. Riboswitches can also be deactivated by, for example, removing trigger molecules from the presence of the riboswitch. Thus, the disclosed method of deactivating a riboswitch can involve, for example, removing a trigger molecule (or other activating compound) from the presence or contact with the riboswitch. A riboswitch can be blocked by, for example, binding of an analog of the trigger molecule that does not activate the riboswitch. The method can comprise selecting a compound or trigger molecule that can activate, deactivate or block a riboswitch and bringing into contact the riboswitch and the selected compound or trigger molecule. The method can comprise selecting a compound identified by any of the disclosed methods that can activate, deactivate or block a riboswitch and bringing into contact the riboswitch and the selected compound.

Also disclosed arc methods for altering expression of an RNA molecule, or of a gene encoding an RNA molecule, where the RNA molecule includes a riboswitch, by bringing a compound into contact with the RNA molecule. Riboswitches function to control gene expression through the binding or removal of a trigger molecule. Thus, subjecting an RNA molecule of interest that includes a riboswitch to conditions that activate, deactivate or block the riboswitch can be used to alter expression of the RNA. Expression can be altered as a result of, for example, termination of transcription or blocking of ribosome binding to the RNA. Binding of a trigger molecule can, depending on the nature of the riboswitch, reduce or prevent expression of the RNA molecule or promote or increase expression of the RNA molecule. The method can comprise selecting a compound that can activate, deactivate or block a riboswitch and bringing into contact an RNA molecule comprising the riboswitch and the selected compound. The method can comprise selecting a compound identified by any of the disclosed methods that can activate, deactivate or block a riboswitch and bringing into contact an RNA molecule comprising the riboswitch and the selected compound.

Also disclosed are methods for regulating expression of a naturally occurring gene or RNA that contains a riboswitch by activating, deactivating or blocking the riboswitch. If the gene is essential for survival of a cell or organism that harbors it, activating, deactivating or blocking the riboswitch can result in death, stasis or debilitation of the cell or organism. For example, activating a naturally occurring riboswitch in a naturally occurring gene that is essential to survival of a microorganism can result in death of the microorganism (if activation of the riboswitch turns off or represses expression). This is one basis for the use of the disclosed compounds and methods for antimicrobial and antibiotic effects. The compounds that have these antimicrobial effects are considered to be bacteriostatic or bacteriocidal. The method can comprise selecting a compound that can activate, deactivate or block a riboswitch and bringing into contact a gene or RNA that contains the riboswitch and the selected compound. The method can comprise selecting a compound identified by any of the disclosed methods that can activate, deactivate or block a riboswitch and bringing into contact a gene or RNA that contains the riboswitch and the selected compound.

Also disclosed are methods for selecting and identifying compounds that can activate, deactivate or block a riboswitch. Activation of a riboswitch refers to the change in state of the riboswitch upon binding of a trigger molecule. A riboswitch can be activated by compounds other than the trigger molecule and in ways other than binding of a trigger molecule. The term trigger molecule is used herein to refer to molecules and compounds that can activate a riboswitch. This includes the natural or normal trigger molecule for the riboswitch and other compounds that can activate the riboswitch. Natural or normal trigger molecules are the trigger molecule for a given riboswitch in nature or, in the case of some non-natural riboswitches, the trigger molecule for which the riboswitch was designed or with which the riboswitch was selected (as in, for example, in vitro selection or in vitro evolution techniques). Non-natural trigger molecules can be referred to as non-natural trigger molecules.

Also disclosed are methods of killing or inhibiting bacteria or microorganisms, comprising contacting the bacteria or microorganisms with a compound disclosed herein or identified by the methods disclosed herein. The method can comprise selecting a compound identified by any of the methods disclosed herein and bringing into contact bacteria or microorganisms and the selected compound. The method can comprise selecting a compound identified by any of the methods disclosed herein and bringing into contact bacteria or microorganisms and the selected compound. The method can comprise selecting a compound that can activate, deactivate or block a riboswitch and bringing into contact bacteria or microorganisms and the selected compound. The method can comprise selecting a compound identified by any of the disclosed methods that can activate, deactivate or block a riboswitch and bringing into contact bacteria or microorganisms and the selected compound. The method can comprise selecting a compound that can activate, deactivate or block a riboswitch and bringing into contact bacteria or microorganisms that contain the riboswitch and the selected compound. The method can comprise selecting a compound identified by any of the disclosed methods that can activate, deactivate or block a riboswitch and bringing into contact bacteria or microorganisms that contain the riboswitch and the selected compound.

Also disclosed are methods of identifying compounds that activate, deactivate or block a riboswitch. For examples, compounds that activate a riboswitch can be identified by bringing into contact a test compound and a riboswitch and assessing activation of the riboswitch. If the riboswitch is activated, the test compound is identified as a compound that activates the riboswitch. Activation of a riboswitch can be assessed in any suitable manner. For example, the riboswitch can be linked to a reporter RNA and expression, expression level, or change in expression level of the reporter RNA can be measured in the presence and absence of the test compound. As another example, the riboswitch can include a conformation dependent label, the signal from which changes depending on the activation state of the riboswitch. Such a riboswitch preferably uses an aptamer domain from or derived from a naturally occurring riboswitch. As can be seen, assessment of activation of a riboswitch can be performed with the use of a control assay or measurement or without the use of a control assay or measurement. Methods for identifying compounds that deactivate a riboswitch can be performed in analogous ways.

In addition to the methods disclosed elsewhere herein, identification of compounds that block a riboswitch can be accomplished in any suitable manner. For example, an assay can be performed for assessing activation or deactivation of a riboswitch in the presence of a compound known to activate or deactivate the riboswitch and in the presence of a test compound. If activation or deactivation is not observed as would be observed in the absence of the test compound, then the test compound is identified as a compound that blocks activation or deactivation of the riboswitch.

Also disclosed are methods of detecting compounds using biosensor riboswitches. The method can include bringing into contact a test sample and a biosensor riboswitch and assessing the activation of the biosensor riboswitch. Activation of the biosensor riboswitch indicates the presence of the trigger molecule for the biosensor riboswitch in the test sample. Biosensor riboswitches are engineered riboswitches that produce a detectable signal in the presence of their cognate trigger molecule. Useful biosensor riboswitches can be triggered at or above threshold levels of the trigger molecules. Biosensor riboswitches can be designed for use in vivo or in vitro. For example, biosensor riboswitches operably linked to a reporter RNA that encodes a protein that serves as or is involved in producing a signal can be used in vivo by engineering a cell or organism to harbor a nucleic acid construct encoding the riboswitch/reporter RNA. An example of a biosensor riboswitch for use in vitro is a glutamine riboswitch that includes a conformation dependent label, the signal from which changes depending on the activation state of the riboswitch. Such a biosensor riboswitch preferably uses an aptamer domain from or derived from a naturally occurring glutamine riboswitch.

Also disclosed are compounds made by identifying a compound that activates, deactivates or blocks a riboswitch and manufacturing the identified compound. This can be accomplished by, for example, combining compound identification methods as disclosed elsewhere herein with methods for manufacturing the identified compounds. For example, compounds can be made by bringing into contact a test compound and a riboswitch, assessing activation of the riboswitch, and, if the riboswitch is activated by the test compound, manufacturing the test compound that activates the riboswitch as the compound.

Also disclosed are compounds made by checking activation, deactivation or blocking of a riboswitch by a compound and manufacturing the checked compound. This can be accomplished by, for example, combining compound activation, deactivation or blocking assessment methods as disclosed elsewhere herein with methods for manufacturing the checked compounds. For example, compounds can be made by bringing into contact a test compound and a riboswitch, assessing activation of the riboswitch, and, if the riboswitch is activated by the test compound, manufacturing the test compound that activates the riboswitch as the compound. Checking compounds for their ability to activate, deactivate or block a riboswitch refers to both identification of compounds previously unknown to activate, deactivate or block a riboswitch and to assessing the ability of a compound to activate, deactivate or block a riboswitch where the compound was already known to activate, deactivate or block the riboswitch.

Disclosed is a method of detecting a compound of interest, the method comprising bringing into contact a sample and a glutamine riboswitch, wherein the riboswitch is activated by the compound of interest, wherein the riboswitch produces a signal when activated by the compound of interest, wherein the riboswitch produces a signal when the sample contains the compound of interest. The riboswitch can change conformation when activated by the compound of interest, wherein the change in conformation produces a signal via a conformation dependent label. The riboswitch can change conformation when activated by the compound of interest, wherein the change in conformation causes a change in expression of an RNA linked to the riboswitch, wherein the change in expression produces a signal. The signal can be produced by a reporter protein expressed from the RNA linked to the riboswitch.

Disclosed is a method comprising (a) testing a compound for inhibition of gene expression of a gene encoding an RNA comprising a riboswitch, wherein the inhibition is via the riboswitch, and (b) inhibiting gene expression by bringing into contact a cell and a compound that inhibited gene expression in step (a), wherein the cell comprises a gene encoding an RNA comprising a riboswitch, wherein the compound inhibits expression of the gene by binding to the riboswitch.

### A. Identification of Antimicrobial Compounds

Riboswitches arc a class of structured RNAs that have evolved for the purpose of binding small organic molecules. The natural binding pocket of riboswitches can be targeted with metabolite analogs or by compounds that mimic the shape-space of the natural metabolite. The small molecule ligands of riboswitches provide useful sites for derivitization to produce drug candidates. Distribution of some riboswitches is shown in Table 1 of U.S. Application Publication No. 2005-0053951. Once a class of riboswitch has been identified and its potential as a drug target assessed, such as the glutamine riboswitch, candidate molecules can be identified.

The emergence of drug-resistant stains of bacteria highlights the need for the identification of new classes of antibiotics. Anti-riboswitch drugs represent a mode of anti-bacterial action that is of considerable interest for the following reasons. Riboswitches control the expression of genes that are critical for fundamental metabolic processes. Therefore manipulation of these gene control elements with drugs yields new antibiotics. These antimicrobial agents can be considered to be bacteriostatic, or bacteriocidal. Riboswitches also carry RNA structures that have evolved to selectively bind metabolites, and therefore these RNA receptors make good drug targets as do protein enzymes and receptors. Furthermore, it has been shown that two antimicrobial compounds (discussed above) kill bacteria by deactivating the antibiotics resistance to emerge through mutation of the RNA target.

### B. Methods of Using Antimicrobial Compounds

Disclosed herein are in vivo and in vitro anti-bacterial methods. By “anti-bacterial” is meant inhibiting or preventing bacterial growth, killing bacteria, or reducing the number of bacteria. Thus, disclosed is a method of inhibiting or preventing bacterial growth comprising contacting a bacterium with an effective amount of one or more compounds disclosed herein. Additional structures for the disclosed compounds are provided herein.

Disclosed herein is also a method of inhibiting growth of a cell, such as a bacterial cell or a microbial cell, that is in a subject, the method comprising administering an effective amount of a compound as disclosed herein to the subject. This can result in the compound being brought into contact with the cell. The subject can have, for example, a bacterial infection, and the bacterial cells can be inhibited by the compound. The bacteria can be any bacteria, such as cyanobacteria or bacteria from the genus Bacillus or Staphylococcus, for example. Bacterial growth can also be inhibited in any context in which bacteria are found. For example, bacterial growth in fluids, biofilms, and on surfaces can be inhibited. The compounds disclosed herein can be administered or used in combination with any other compound or composition. For example, the disclosed compounds can be administered or used in combination with another antimicrobial compound.

The bacteria can be any bacteria, such as bacteria from the genus Bacillus, Acinetobacter, Actinobacillus, Clostridium, Desullitobacterium, Enterococcus, Erwinia, Escherichia, Exiguobacterium, Fusobacterium, Geobacillus, Haemophilus, Klebsiella, kliomarina, Lactobacillus, Lactococcus, Leuconostoc, Listeria, Moorella, Mycobacterium, Oceanobacillus, Oenococcus, Pasteurella, Pediococcus, Pseudomonas, Shewanella, Shigella, Solibacter, Staphylococcus, Streptococcus, Therinoanaerobacter, Therinotoga, and Vibrio, for example. The bacteria can be, for example, Actinobacillus pleuropneumoniae, Bacillus anthracia, Bacillus cereus, Bacillus clausii, Bacillus halodurans, Bacillus licheniformis, Bacillus subtilis, Bacillus thuringiensis, Clostridium acetobutylicum, Clostridium dificile, Clostridium perfringens, Clostridium tetani, Clostridium themzocellum, Desulfitobacterium hafniense, Enterococcus faecalis, Erwinia carotovora, Escherichia coli, Exiguobacterium sp., Fusobacterium nucleatum, Geobacillus kaustophilus, Haemophilus ducreyi, Haemophilus influenzae, Haemophilus somnus, Idiomarina loihiensis, Lactobacillus acidophilus, Lactobacillus casei, Lactobacillus delbrueckii, Lactobacillus gasseri, Lactobacillus johnsonii, Lactobacillus plantarum, Lactococcus lactis, Leuconostoc mesenteroides, Listeria innocua, Listeria monocytogenes, Moorella thermoacetica, Oceanobacillus iheyensis, Oenococcus oeni, Pasteurella multocida, Pediococcus pentosaceus, Shewanella oneidensis, Shigella flexneri, Solibacter usitatus, Staphylococcus aureus, Staphylococcus epidermidis, Thermoanaerobacter tengcongensis, Thermotoga maritima, Vibrio cholerae, Vibrio fischeri, Vibrio parahaemolyticus, or Vibrio vulnificus.

Particularly useful are cyanobacteria. For example, the bacteria can be any bacteria, such as bacteria from the genus Acaryochloris, Adrianema, Albrightia, Alternantia, Ammatoidea, Anabaena, Anabaenopsis, Aphanizomenon, Aphanocapsa, Aphanothece, Arthronema, Arthrospira, Asterocapsa, Aulosira, Bacularia, Baradlaia, Blennothrix, Borzia, Borzinema, Brachytrichia, Brachytrichiopsis, Brasilonema, Calothrix, Camptylonemopsis, Capsosira, Chamaecalyx, Chamaesiphon, Chlorogloea, Chlorogloeopsis, Chondrocystis, Chondrogloea, Chroococcidiopsis, Chroococcidium, Chroococcopsis, Chroococcus, Chroogloeocystis, Clastidium, Coccopedia, Coelomoron, Coelosphaeriopsis, Coelosphaerium, Coleodesmium, Coleofasciculus, Colteronema, Crinalium, Crocosphaera, Cronbergia, Cuspidothrix, Cyanoaggregatum, Cyanoarbor, Cyanobacterium, Cyanobium, Cyanobotrys, Cyanocatena, Cyanocatenula, Cyanocomperia, Cyanocystis, Cyanoderma, Cyanodermatium, Cyanodictyon, Cyanogranis, Cyanokybus, Cyanonephron, Cyanophanon, Cyanosaccus, Cyanosarcina, Cyanospira, Cyanostylon, Cyanotetras, Cyanothamnos, Cyanothece, Cylindrospermopsis, Cylindrospermum, Dalmatella, Dasygloea, Dermocarpella, Desmosiphon, Dichothrix, Dolichospermum, Doliocatella, Dzensia, Entophysalis, Epigloeosphaera, Epilithia, Ercegovicia, Eucapsis, Fischerella, Fischerellopsis, Fortiea, Gardnerula, Geitleria, Geitleribactron, Geitlerinema, Geminocystis, Glaucospira, Gloeobacter, Gloeocapsa, Gloeocapsopsis, Gloeothece, Gloeotrichia, Gomontiella, Gomphosphaeria, Halomicronema, Halothece, Handeliella, Hapalosiphon, Hassallia, Herpyzonema, Heterocyanococcus, Heteroleibleinia, Homoeoptyche, Homoeothrix, Hormathonema, Hormoscilla, Hormothece, Hydrococcus, Hydrocoleum, Hydrocoryne, Hyella, Hyphomorpha, Isactis, Isocystis, Iyengariella, Jaaginema, Johannesbaptistia, Katagnymene, Komvophoron, Kyrtuthrix, Leibleinia, Lemmermanniella, Leptolyngbya, Leptopogon, Letestuinerna, Liinnococcus, Lfinnothrix, Lithococcus, Lithomyxa, Loefgrenia, Loriella, Lyngbya, Lyngbyopsis, Macrospermum, Mantellum, Mastidocladus, Mastigocladopsis, Mastigocoleopsis, Mastigocoleus, Matteia, Merismopedia, Microchaete, Microcoleus, Microcrocis, Microcystis, Mojavia, Myxobaktron, Myxohyella, Myxosarcina, Nematoplaca, Nephrococcus, Nodularia, Nostoc, Nostochopsis, Onkonema, Ophiothrix, Oscillatoria, Palikiella, Pannus, Paracapsa, Parenchymorpha, Parthasarathiella, Pascherinema, Petalonema, Phormidesmis, Phormidiochaete, Phormidium, Placoma, Planktocyanocapsa, Planktolyngbya, Planktothricoides, Planktothrix, Plectonema, Pleurocapsa, Podocapsa, Polychlamydum, Porphyrosiphon, Prochlorococcus, Prochloron, Prochlorothrix, Proterendothrix, Pseudanabaena, Pseudocapsa, Pseudoncobyrsa, Pseudophormidium, Pseudoscillatoria, Pseudoscytonema, Pulvinularia, Radaisia, Radiocystis, Raphidiopsis, Rexia, Rhabdoderma, Rhabdogloea, Rhodostichus, Richelia, Rivularia, Ronzeria, Rubidibacter, Sacconenza, Schizothrix, Schmidleinema, Scytonema, Scytonematopsis, Sequenzaea, Sinaiella, Siphononema, Siphonosphaera, Sirocoleum, Snowella, Sokolovia, Solentia, Spelaeopogon, Sphaerocavum, Sphaerospermopsis, Spirirestis, Spirulina, Stanieria, Starria, Stauromatonema, Stichosiphon, Stigonema, Streptostemon, Symphyonema, Symphyonemopsis, Symploca, Symplocastrum, Synechococcus, Synechocystis, Tapinothrix, Thalpophila, Thermosynechoccous, Thiochaete, Tolypothrix, Trichocoleus, Trichodesmium, Trichormus, Tryponema, Tubiella, Tychonema, Umezakia, Voukiella, Westiella, Westiellopsis, Wollea, Wolskyella, Woronichinia, Xenococcus, Xenotholos, and Yonedaella.

Bacterial growth can also be inhibited in any context in which bacteria are found. For example, bacterial growth in fluids, biofilms, and on surfaces can be inhibited. The compounds disclosed herein can be administered or used in combination with any other compound or composition. For example, the disclosed compounds can be administered or used in combination with another antimicrobial compound.

“Inhibiting bacterial growth” is defined as reducing the ability of a single bacterium to divide into daughter cells, or reducing the ability of a population of bacteria to form daughter cells. The ability of the bacteria to reproduce can be reduced by about 10%, about 20%, about 30%, about 40%, about 50%, about 60%, about 70%, about 80%, about 90%, or 100% or more.

Also provided is a method of killing a bacterium or population of bacteria comprising contacting the bacterium with one or more of the compounds disclosed and described herein.

“Killing a bacterium” is defined as causing the death of a single bacterium, or reducing the number of a plurality of bacteria, such as those in a colony. When the bacteria are referred to in the plural form, the “killing of bacteria” is defined as cell death of a given population of bacteria at the rate of 10% of the population, 20% of the population, 30% of the population, 40% of the population, 50% of the population, 60% of the population, 70% of the population, 80% of the population, 90% of the population, or less than or equal to 100% of the population.

The compounds and compositions disclosed herein have anti-bacterial activity in vitro or in vivo, and can be used in conjunction with other compounds or compositions, which can be bacteriocidal as well.

By the term “therapeutically effective amount” of a compound as provided herein is meant a nontoxic but sufficient amount of the compound to provide the desired reduction in one or more symptoms. As will be pointed out below, the exact amount of the compound required will vary from subject to subject, depending on the species, age, and general condition of the subject, the severity of the disease that is being treated, the particular compound used, its mode of administration, and the like. Thus, it is not possible to specify an exact “effective amount.” However, an appropriate effective amount may be determined by one of ordinary skill in the art using only routine experimentation.

The compositions and compounds disclosed herein can be administered in vivo in a pharmaceutically acceptable carrier. By “pharmaceutically acceptable” is meant a material that is not biologically or otherwise undesirable, i.e., the material may be administered to a subject without causing any undesirable biological effects or interacting in a deleterious manner with any of the other components of the pharmaceutical composition in which it is contained. The carrier would naturally be selected to minimize any degradation of the active ingredient and to minimize any adverse side effects in the subject, as would be well known to one of skill in the art.

The compositions or compounds disclosed herein can be administered orally, parenterally (e.g., intravenously), by intramuscular injection, by intraperitoneal injection, transdermally, extracorporeally, topically or the like, including topical intranasal administration or administration by inhalant. As used herein, “topical intranasal administration” means delivery of the compositions into the nose and nasal passages through one or both of the nares and can comprise delivery by a spraying mechanism or droplet mechanism, or through aerosolization of the nucleic acid or vector. Administration of the compositions by inhalant can be through the nose or mouth via delivery by a spraying or droplet mechanism. Delivery can also be directly to any area of the respiratory system (e.g., lungs) via intubation. The exact amount of the compositions required will vary from subject to subject, depending on the species, age, weight and general condition of the subject, the severity of the allergic disorder being treated, the particular nucleic acid or vector used, its mode of administration and the like. Thus, it is not possible to specify an exact amount for every composition. However, an appropriate amount can be determined by one of ordinary skill in the art using only routine experimentation given the teachings herein.

Parenteral administration of the composition or compounds, if used, is generally characterized by injection. Injectables can be prepared in conventional forms, either as liquid solutions or suspensions, solid forms suitable for solution of suspension in liquid prior to injection, or as emulsions. A more recently revised approach for parenteral administration involves use of a slow release or sustained release system such that a constant dosage is maintained. See, e.g., U.S. Pat. No. 3,610,795, which is incorporated by reference herein.

The compositions and compounds disclosed herein can be used therapeutically in combination with a pharmaceutically acceptable carrier. Suitable carriers and their formulations arc described in Remington: The Science and Practice of Pharmacy (19th ed.) ed. A. R. Gennaro, Mack Publishing Company, Easton, Pa. 1995. Typically, an appropriate amount of a pharmaceutically-acceptable salt is used in the formulation to render the formulation isotonic. Examples of the pharmaceutically-acceptable carrier include, but are not limited to, saline, Ringer's solution and dextrose solution. The pH of the solution is preferably from about 5 to about 8, and more preferably from about 7 to about 7.5. Further carriers include sustained release preparations such as semipermeable matrices of solid hydrophobic polymers containing the antibody, which matrices are in the form of shaped articles, e.g., films, liposomes or microparticles. It will be apparent to those persons skilled in the art that certain carriers may be more preferable depending upon, for instance, the route of administration and concentration of composition being administered.

Pharmaceutical carriers are known to those skilled in the art. These most typically would be standard carriers for administration of drugs to humans, including solutions such as sterile water, saline, and buffered solutions at physiological pH. The compositions can be administered intramuscularly or subcutaneously. Other compounds will be administered according to standard procedures used by those skilled in the art.

Pharmaceutical compositions may include carriers, thickeners, diluents, buffers, preservatives, surface active agents and the like in addition to the molecule of choice.

Pharmaceutical compositions may also include one or more active ingredients such as antimicrobial agents, antiinflammatory agents, anesthetics, and the like.

The pharmaceutical composition may be administered in a number of ways depending on whether local or systemic treatment is desired, and on the area to be treated. Administration may be topically (including ophthalmically, vaginally, rectally, intranasally), orally, by inhalation, or parenterally, for example by intravenous drip, subcutaneous, intraperitoneal or intramuscular injection. The disclosed antibodies can be administered intravenously, intraperitoneally, intramuscularly, subcutaneously, intracavity, or transdermally.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like. Formulations for topical administration may include ointments, lotions, creams, gels, drops, suppositories, sprays, liquids and powders. Conventional pharmaceutical carriers, aqueous, powder or oily bases, thickeners and the like may be necessary or desirable.

Compositions for oral administration include powders or granules, suspensions or solutions in water or non-aqueous media, capsules, sachets, or tablets. Thickeners, flavorings, diluents, emulsifiers, dispersing aids or binders may be desirable.

Some of the compositions may potentially be administered as a pharmaceutically acceptable acid- or base-addition salt, formed by reaction with inorganic acids such as hydrochloric acid, hydrobromic acid, perchloric acid, nitric acid, thiocyanic acid, sulfuric acid, and phosphoric acid, and organic acids such as formic acid, acetic acid, propionic acid, glycolic acid, lactic acid, pyruvic acid, oxalic acid, malonic acid, succinic acid, maleic acid, and fumaric acid, or by reaction with an inorganic base such as sodium hydroxide, ammonium hydroxide, potassium hydroxide, and organic bases such as mono-, di-, trialkyl and awl amines and substituted ethanolamines.

Therapeutic compositions as disclosed herein may also be delivered by the use of monoclonal antibodies as individual carriers to which the compound molecules are coupled. The therapeutic compositions of the present disclosure may also be coupled with soluble polymers as targetable drug carriers. Such polymers can include, but are not limited to, polyvinyl-pyrrolidone, pyran copolymer, polyhydroxypropylmethacryl-amidephenol, polyhydroxyethylaspartamidephenol, or polyethyl-eneoxidepolylysine substituted with palmitoyl residues. Furthermore, the therapeutic compositions of the present disclosure may be coupled to a class of biodegradable polymers useful in achieving controlled release of a drug, for example, polylactic acid, polyepsilon caprolactone, polyhydroxy butyric acid, polyorthoesters, polyacetals, polydihydro-pyrans, polycyanoacrylates and cross-linked or amphipathic block copolymers of hydrogels.

Preferably at least about 3%, more preferably about 10%, more preferably about 20%, more preferably about 30%, more preferably about 50%, more preferably 75% and even more preferably about 100% of the bacterial infection is reduced due to the administration of the compound. A reduction in the infection is determined by such parameters as reduced white blood cell count, reduced fever, reduced inflammation, reduced number of bacteria, or reduction in other indicators of bacterial infection. To increase the percentage of bacterial infection reduction, the dosage can increase to the most effective level that remains non-toxic to the subject.

As used throughout, “subject” refers to an individual. Preferably, the subject is a mammal such as a non-human mammal or a primate, and, more preferably, a human.

“Subjects” can include domesticated animals (such as cats, dogs, etc.), livestock (e.g., cattle, horses, pigs, sheep, goats, etc.), laboratory animals (e.g., mouse, rabbit, rat, guinea pig, etc.) and fish.

A “bacterial infection” is defined as the presence of bacteria in a subject or sample. Such bacteria can be an outgrowth of naturally occurring bacteria in or on the subject or sample, or can be due to the invasion of a foreign organism.

The compounds disclosed herein can be used in the same manner as antibiotics. Uses of antibiotics are well established in the art. One example of their use includes treatment of animals. When needed, the disclosed compounds can be administered to the animal via injection or through feed or water, usually with the professional guidance of a veterinarian or nutritionist. They are delivered to animals either individually or in groups, depending on the circumstances such as disease severity and animal species. Treatment and care of the entire herd or flock may be necessary if all animals are of similar immune status and all are exposed to the same disease-causing microorganism.

Another example of a use for the compounds includes reducing a microbial infection of an aquatic animal, comprising the steps of selecting an aquatic animal having a microbial infection, providing an antimicrobial solution comprising a compound as disclosed, chelating agents such as EDTA, TRIENE, adding a pH buffering agent to the solution and adjusting the pH thereof to a value of between about 7.0 and about 9.0, immersing the aquatic animal in the solution and leaving the aquatic animal therein for a period that is effective to reduce the microbial burden of the animal, removing the aquatic animal from the solution and returning the animal to water not containing the solution. The immersion of the aquatic animal in the solution containing the EDTA, a compound as disclosed, and TRIENE and pH buffering agent may be repeated until the microbial burden of the animal is eliminated. (U.S. Pat. No. 6,518,252).

Other uses of the compounds disclosed herein include, but are not limited to, dental treatments and purification of water (this can include municipal water, sewage treatment systems, potable and non-potable water supplies, and hatcheries, for example).

### EXAMPLES

### A. Example 1

### Bacterial Aptamers that Selectively Bind Glutamine

This example demonstrates that the glnA and Downstream-peptide motifs are structural variants of a novel aptamer class responsive to glutamine, providing the first evidence that this amino acid is an important signaling molecule in the regulation of nitrogen metabolism in cyanobacteria.

1. Results

Given its characteristics revealed by bioinformatics, it was hypothesized and later determined that glnA motif RNAs are representatives of a new-found riboswitch aptamer class. Because these RNAs are encoded upstream of several genes involved in nitrogen metabolism, a collection of potential ligands and analogs related to this set of metabolic pathways was tested. In-line probing assays revealed that a 67 nucleotide glnA representative from Synechococcus elongatus, termed 67 glnA (FIG. 30A), binds most tightly to L-glutamine with an apparent dissociation constant (K_{D}) of approximately 575 (FIG. 30B, 30C). The shape of the binding curve matches that expected for a one-to-one interaction between the RNA and its ligand.

With the exception of D-glutamine, which is bound by the 67 glnA aptamer with approximately 1/10^{th} the affinity of its more common isomer, all other compounds tested were rejected by the aptamer even at concentrations as high as 10 mM. The compounds tested include the natural amino acid L-asparagine, the glutamine analogues L-glutamine t-butyl ester, L-theanine, O-acetyl-L-serine, L-homoglutamine, L-β-homo glutamine, (S)-2-amino-5-oxo-hexanoic acid, 5-amino-5-oxopentanoic acid, and the dipeptide Ala-Gln. Ligand-binding specificity was also assessed for a second representative of the glnA motif from a marine metagenomic sequence. This RNA binds to L-glutamine with a K_{D} of approximately 150 μM, whereas putrescine, L-lysine, 2-oxoglutarate, γ-aminobutyric acid (GABA), glutaric acid, succinate, succinic semialdehyde, agmatine, pyridoxal phosphate, and glutamate are all rejected at 1 mM (data not shown). Despite the lower K_{D} value of this RNA, further experiments were conducted with the 67 glnA RNA due to more pronounced positions of modulating cleavage intensity in our in-line probing assasy.

To ensure that the binding observed was not the result of non-specific interactions between the RNA and glutamine, 67 glnA mutant RNA constructs M1 and M2 (FIG. 30A) containing two consecutive disruptive mutations in either the P2 or P3 stems, respectively, were prepared. As expected, no modulation of these structurally disrupted RNAs upon addition of glutamine to in-line probing reactions, indicating that the banding pattern changes seen with the wild-type RNA with glutamine arc caused by selective interactions. Constructs M3 and M4 (FIG. 30A) in which the mismatched base pairs were restored with compensatory mutations were then tested. These RNAs regain structural modulation in response to glutamine addition, and exhibit K_{D} values similar to that of the 67 glnA RNA.

There are several reasons why glutamate, rather than or in addition to glutamine, could be the natural metabolite ligand for glnA motif aptamers. First, the chemical structures of glutamine and glutamate differ only by a side chain amino or hydroxyl group, respectively. Both these groups at a minimum could serve as a single hydrogen bond donor source. Second, some representatives of the glnA motif are found upstream of genes directly involved in glutamate synthesis. Third, the concentrations of both glutamine and glutamate are exceptionally high in bacteria. In Escherichia coli, glutamine is present in the bacteria at a concentration of approximately 4 mM, whereas the intracellular level of glutamate is approximately 100 mM (Bennett et al. Absolute metabolite concentrations and implied enzyme active site occupancy in Escherichia coli. Nat Chem Biol 2009; 5:593-599). For a riboswitch to be selective for glutamine, it would need to discriminate against glutamate by more than 20 fold, given the exceptionally high concentration of this competing amino acid.

As noted above, binding by glutamate was assessed in the assays described above without observing binding at 1 mM amino acid concentration. However, to investigate glutamate binding at physiologically relevant concentrations, in-line probing assays using the 67 glnA RNA and 100 mM glutamate as the primary buffering agent were conducted. Although no structural modulation was observed in the presence of this high concentration of glutamate, the addition of 1 mM glutamine to an in-line probing assay containing 100 mM glutamate produced the pattern of RNA cleavage products expected for glutamine binding.

Interestingly, over half of the glnA RNAs are arranged in tandem orientations, where two or sometimes three aptamers are found grouped together with only small segments of intervening sequence in between (FIG. 31A). Of the double glnA aptamer arrangements, over half share a similar intervening sequence and an addition portion of conserved nucleotides 3′ of the aptamers that has the potential to base-pair with the intervening sequence (FIG. 31B). Multiple aptamers arrangements have been observed previously and can serve various biological purposes. For example, two aptamers sensitive to different ligands can influence the expression of the same gene, functioning similarly to two-input Boolean logic gates (Sudarsan et al. Tandem riboswitch architectures exhibit complex gene control functions. Science 2006; 314:300-304). Multiple aptamers that recognize the same compound can be utilized to achieve sharper, more digital responses to changing ligand concentrations either by using multiple terminator stems (Welz et al. Ligand binding and gene control characteristics of tandem riboswitches in Bacillus anthracis. RNA 2007; 13:573-582) or cooperativity (Mandal et al. A glycine-dependent riboswitch that uses cooperative binding to control gene expression. Science 2004; 306:275-279; Kwon et al. Chemical basis of glycine riboswitch cooperativity. RNA 2008; 14:25-34). In the case of glnA RNAs, the amount of intervening sequence between the aptamers is often apparently too small to allow for the use of multiple expression platforms, therefore they can function in a cooperative fashion. To test this, several tandem glnA constructs were made and tested via in-line probing. These constructs included a three aptamer arrangement and a representative from the group of RNAs shown in FIG. 31B, both with and without the conserved portion of 3′ sequence. The in-line probing analysis of these tandem constructs did not reveal any evidence of cooperative binding (data not shown), such as a steeper dose-response curve than that exhibited by non-cooperative riboswitches. The data does not rule out cooperative function, since various factors such as inadequate construct length or non-physiological assay conditions could confound the assays. Despite the fact that the genetic contexts of glnA motif and Downstream-peptide motif RNAs are distinct, it was speculated that the Downsteam-peptide motif can also bind glutamine due to sequence and structural similarities of the aptamer families. Again in-line probing on a representative member of the Downstream-peptide motif from Synechococcus sp. CC9902 (83 DP RNA) was used and it was determined that the RNA binds glutamine with an apparent K_{D} of approximately 5 mM (FIG. 32). Unlike with the 67 glnA RNA, no binding was detected between the 83 DP RNA and D-glutamine at concentrations up to 10 mM. Additionally, the RNA does not appreciably bind to any of the other compounds tested with the 67 glnA RNA at 10 mM concentrations.

The predicted pseudoknot was validated by examining the ligand-binding functions of disruptive and compensatory mutations. Specifically, construct M5 was designed as a mutant 83 DP RNA with two disruptive mutations in the pseudoknot. As with the 67 glnA disruption mutation constructs, M5 RNA does not bind to glutamine. By contrast, a construct with compensatory mutations that restore base pairing within the pseudoknot (M6 RNA) binds to glutamine with a K_{D} value similar to that of the DP RNA.

2. Discussion

The glnA motif and the Downstream-peptide motif share a variety of structural features and are able to bind glutamine while strongly discriminating against a variety of structurally related analogues. Mutational studies indicated the interactions between glutamine and the RNAs examined in this study are specific, because small disruptions of various base-paired regions affect the ability of the RNAs to bind ligand. Additionally, these experiments support the accuracy of the secondary structural models because the compensatory mutations used in constructs M3, M4, and M6 indicate that the structure rather than the precise sequence in these putative stems is important. All of the disclosed findings indicate that these RNAs are subtypes of a novel glutamine riboswitch class.

Highly conserved nucleotides in loops and bulges are often indicative of positions essential for forming riboswitch aptamer binding pockets. Several residues in the three stem junction of the glnA aptamer and analogous positions on the Downstream-peptide aptamer could be involved in the formation of the aptamer binding pocket. Additionally, the high degree of conservation of the nucleotides in the P1 stem indicates that some of these residues directly participate in ligand binding as well.

Neither of the glutamine aptamer subtypes bind to any other compounds tested in the in-line probing assays. This observation, in conjunction with the high sequence similarity between the two related RNAs, indicates that the binding pockets of both subtypes are similar. The fact that these RNAs reject glutamate, L-homoglutamine, and (S)-2-amino-5-oxo-hexanoic acid indicates that the aptamers are highly sensitive to the length and composition of the amino acid side chain. This is an important characteristic for a receptor that must be responsive to a single amino acid, given that there are many other natural amino acids in all cells. Because the aptamers tested were both sensitive to the removal of the amino group in the side chain, the RNA likely makes one or more hydrogen bonds with the ligand at this position. Similarly, removal of the amino group attached to the α-carbon also causes a loss of binding, as is evident by the in-line probing results with the compound 5-amino-5-oxopentanoic acid. The loss of hydrogen bonds and/or ionic interactions can be responsible for this compound's inability to serve as an aptamer ligand.

Large chemical groups on either the N or C termini of the amino acid are also not tolerated because neither Ala-Gln nor L-glutamine t-butyl ester are bound by the aptamers. The addition of bulky chemical groups to glutamine likely results in steric clashes with the RNA, which may indicate that the aptamers form a highly-enclosed pocket as do many other riboswitch aptamers. In the case of L-glutamine t-butyl ester, it is also possible that the removal of the negative charge from the C terminal oxygen can causes a loss of a favorable ionic interaction.

The 67 glnA RNA can bind D-glutamine, albeit with a reduced affinity when compared to the more biologically prevalent L-isomer. This can indicate that the reversal of two groups bonded to the α-carbon of the ligand causes the loss of only weak contacts between the compound and the RNA. Alternatively, if important bonds are broken, this could be partially mitigated by the formation of other fortuitous interactions when the chirality of the ligand is reversed.

No binding was detected between the DP RNA and D-glutamine. This finding can seemingly contradict the claim that the binding pockets of the glnA and the Downstream-peptide motif are likely similar. However, because the 67 glnA RNA binds to L-glutamine 10-fold more tightly compared to D-glutamine, it is possible that concentrations higher than 10 mM (the highest concentration we tested) would be necessary to detect an interaction between the 83 DP RNA and the D-isomer.

The high frequency of tandem glnA RNA arrangements could mean that these aptamers are often employed to achieve a form of riboswitch-mediated gene control not feasible with a single aptamer. For examples, cooperative binding by two aptamers can yield a more digital gene control element (Mandal et al. A glycine-dependent riboswitch that uses cooperative binding to control gene expression. Science 2004; 306:275-279; Welz R, Breaker R R. Ligand binding and gene control characteristics of tandem riboswitches in Bacillus anthracis. RNA 2007; 13:573-582). Although no evidence of cooperativity was observed in the in-line probing assays, the tandem glnA RNAs can function in this manner using additional portions of sequence elements not included in the constructs. They may require other cellular factors or conditions for proper function.

The K_{D} values for the different glutamine aptamers tested are all relatively high in comparison to those of characterized riboswitch aptamer classes, which range from about 10 μM for the cyclic di-GMP-I aptamer (Smith et al. Structural basis of ligand binding by a c-di-GMP riboswitch. Nat Struct Mol Biol 2009; 16:1218-1223) to more than 100 μM for glmS ribozymes (Winkler et al. Control of gene expression by a natural metabolite-responsive ribozyme. Nature 2004; 428:281-286; Cochrane et al. Structural and chemical basis for glucosamine 6-phosphate binding and activation of the glmS ribozyme. Biochemistry 2009; 48:3239-3246). Considering the high concentrations of glutamine in E. coli and likely other bacterial species, it isn't surprising that members of this aptamer class exhibit poor affinity. Regardless, the correlation of an aptamer's K_{D} with the concentrations of ligand needed to trigger riboswitch is not possible with many riboswitches because kinetically-driven riboswitches do not reach thermodynamic equilibrium for ligand binding (Wickiser et al. The kinetics of ligand binding by an adenine-sensing riboswitch. Biochemistry 2005; 44:13404-13414; Wickiser et al. The speed of RNA transcription and metabolite binding kinetics operate an FMN riboswitch. Mol Cell 2005; 18:49-60; Gilbert et al. Thermodynamic and kinetic characterization of ligand binding to the purine riboswitch aptamer domain. J Mol Biol 2006; 359:754-768).

As mentioned previously, riboswitches not only require an aptamer but an expression platform to translate ligand binding of the aptamer into a change in gene expression. There are cases where the glnA and downstream-peptide aptamers are positioned close to predicted terminator stems and ribosomal binding sites, which can be part of riboswitch expression platforms. However, mechanisms for how the aptamers interact with these portions of the sequence are not obvious. It is not uncommon for expression platforms to be hard to identify, and nevertheless the glnA and Downstream-peptide RNAs are suspected to be components of full riboswitches.

Disclosed herein, it is shown that the glnA and Downstream-peptide motifs are naturally occurring aptamers that selectively bind L-glutamine. These elements are often positioned 5′ of several genes involved with nitrogen metabolism in cyanobacteria, which implicates glutamine as an important signaling molecule in the pathways of these organisms. The presence of glutamine-responsive riboswitches can explain why the glutamine-sensing regulatory proteins responsible for nitrogen regulation in other bacterial taxa are absent in cyanobacteria (Forchhammer K. Glutamine signalling in bacteria. Front Biosci 2007; 12:358-370). The discovery of these RNAs expands the scope of metabolites that are recognized by natural aptamers, introducing glutamine as the third amino acid to be sensed by natural RNAs along with glycine and lysine. As the amount of available genomic sequence data continues to expand, it is expected that many additional metabolite-sensing aptamers will be discovered, including a greater diversity of classes that sense amino acids.

3. Materials and Methods

i. Chemicals and DNA oligonucleotides

The compounds L-glutamine, D-glutamine, Ala-Gln, L-glutamine t-butyl ester, L-theanine, O-acetyl-L-serine, asparagine, putrescine, lysine, 2-oxoglutarate, glutaric acid, succinatc, succinic scmialdchydc, GABA, agmatinc, pyridoxal phosphate, and glutamate were all obtained from Sigma-Aldrich. L-homoglutamine and (S)-2-amino-5-oxo-hexanoic acid were ordered from Toronto Research Chemicals Inc. L-β-homoglutamine was obtained from the PepTech Corporation, and 5-amino-5-oxopentanoic acid was purchased from ChemBridge.

The following DNA oligonucleotides were ordered from Sigma-Genosys: primer 1,5′-TAATACGACTCACTATAGGGTAATCGTTGGCCCAGTTTATCTGGGTGGAA (SEQ ID NO:1); primer 2,5′-TGAGAGGCGCGTTGCTTCAGGCCAAAGACCTTACTT CCACCCAGATAAA (SEQ ID NO:2); primer 3,5′-TAATACGACTCACTATAGGGT AATCGTTGGCCCAGTTTATCAAGGTGGAA (SEQ ID NO:3); primer 4,5′-TAATA CGACTCACTATAGGGTAATCGTTGGCCTTGTTTATCAAGGTGGAA (SEQ ID NO:4); primer 5,5′-TGAGAGGCGCGTTGCTTCAGGCCAAAGACCTTACTTCCA CCTTGATAAA (SEQ ID NO:5); primer 6,5′-TGAGAGGCGCGTTGCTTCAGCCC AAAGTCCTTACTTCCACCCAGATAAA (SEQ ID NO:6); primer 7,5′-TGAGAGGCG CGTTGCTTCAGCTCAAAGTGCTTACTTCCACCCAGATAAA (SEQ ID NO:7); primer 8,5′-TAATACGACTCACTATAGGGTATTCTTGGTCCACGTTGAGCTTCC AATCGAAGCTGCA (SEQ ID NO:8); primer 9,5′-TCCTTCATTGCCCACGCCCCCG TTGCTTGGCATGGGTCTGACTGCAGCTTCGATTGGA (SEQ ID NO:9); primer 10, 5′-TCCTTCATTGCCCTCGCCCCCGTTGCTTGGCCTGGGTCTGACTGCAGCTTC GATTGGAAGCT (SEQ ID NO:10); primer 11, 5′-TCCTTCATTGCCCTAGCC CCCGTTGCTTGGCCAGGGTCTGACTGCAGCTTCGATTGGAAGCT (SEQ ID NO:11).

ii. Transcription and Purification of RNAs

Pairs of oligonucleotides were used in primer extension reactions to make full-length double-stranded DNA (dsDNA) products to use as templates for in vitro transcription reactions. Primers 1 and 2 were used for 67 glnA, 2 and 3 for Ml, 4 and 5 for M3, 1 and 6 for M2, 1 and 7 for M4, 8 and 9 for 83 DP, 8 and 10 for M5, and 8 and 11 for M6. A 100 μl solution containing 300 pmoles of each primer, 50 mM Tris-HCl (pH 8.3 at 23° C.), 75 mM KCl, 3 mM MgCl₂, 10 μM dithiothreitol (DTT), 1 mM of each of the four deoxynucleoside triphosphates (dNTPs), and 8 U/μl of SuperScript II Reverse Transcriptase (Invitrogen) was heated for 2 hours at 42° C. The full length dsDNA was then purified using the QIAquick PCR Purification Kit (QIAgen) following the manufacturer's protocol and was eluted in a volume of 50

20 μl of dsDNA was used as a template in a 100 μl in vitro transcription reaction containing 80 mM HEPES (pH 7.5 at 23° C.), 40 mM DTT, 24 mM MgCl₂, 2 mM spermidine, 2.5 mM each of the four ribonucleoside 5′ triphosphates (NTPs), and 10 units/μl bacteriophage T7 RNA polymerase. Samples were heated for 2 hours at 37° C. and purified via denaturing (8M urea) 6% polyacrylamide gel electrophoresis (PAGE). A band containing the RNA was cut from the gel and soaked in a solution of 10 mM Tris-HCl (pH 7.5 at 23° C.), 200 mM NaCl and 1 mM EDTA (pH 8.0 at 23° C.). The RNAs were then concentrated by adding 2.5 volumes of cold (−20° C.) ethanol, centrifuging for 20 minutes at 17,900 g. The resulting pellet was dried, resuspended in water, and stored at −20° C. until use.

iii. In-Line Probing Assays

RNAs were 5′-³²P radiolabeled and subjected to in-line probing analyses, which have been described in detail previously (Soukup et al. Relationship between internucleotide linkage geometry and the stability of RNA. RNA 1999; 5:1308-1325; Regulski E E, Breaker R R. In-line probing analysis of riboswitches. Methods Mol Biol 2008; 419:53-67). Briefly, 5′ triphosphates were removed from the RNAs using alkaline phosphatase (Roche) according to the manufacturer's protocol. The dephosphorylated RNAs were 5′-³²P radiolabeled by incubation with [γ-³²P] ATP and T4 polynucleotide kinase (New England Biolabs), following the manufacturer's directions. Denaturing PAGE (6%) was subsequently employed to purify the RNAs as described above. The radiolabeled RNAs were then incubated at 23° C. in a solution containing 75 mM Tris-HCl (pH 8.3 at 23° C.), 20 mM MgCl₂, 100 mM KCl, and various different potential ligands (see results section for full list) at concentrations ranging from 1 μM to 10 mM in most cases. When RNAs were subjected to in-line probing with 100 mM L-glutamate, the amino acid replaced the Tris-HCl as the buffering agent. HCl and NaOH were used to adjust this modified in-line probing solution to pH 8.3.

After incubating for approximately 40 hours, the products of in-line probing reactions were separated by denaturing 10% PAGE. The gels were then dried and imaged using a Storm 820 PhosphorImager (GE Healthcare). The relative intensities of the various degradation products were quantified using SAFA v1.1 software (Das et al. SAFA: Semi-automated footprinting analysis software for high-throughput quantification of nucleic acid footprinting experiments. RNA 2005; 11:344-354). The bands which modulated the most intensely were used to make K_{D} estimates. In the cases where the concentrations of ligand used were insufficiently high to saturate RNA binding, the data was normalized to K_{D} values that best explained the data assuming a standard one-to-one interaction.

### A. Example 2

### Comparative Genomics Reveals 104 Candidate Structured RNAs from Bacteria, Archaea and their Metagenomes

1. Introduction

Structured noncoding RNAs perform many functions that are essential for protein synthesis, RNA processing, and gene regulation. Structured RNAs can be detected by comparative genomics, in which homologous sequences are identified and inspected for mutations that conserve RNA secondary structure.

By applying a comparative genomics-based approach to genome and metagenome sequences from bacteria and archaea, 104 structured RNAs were identified. Three metabolite-binding RNA motifs were validated, including one that binds the coenzyme S-adenosylmethionine, and a further nine metabolite-binding RNAs were identified. New-found cis-regulatory RNAs are implicated in photosynthesis or nitrogen regulation in cyanobacteria, purine and one-carbon metabolism, stomach infection by Helicobacter, and many other physiological processes. A riboswitch termed crcB is represented in both bacteria and archaea. Another RNA motif controls gene expression from 3′ untranslated regions (UTRs) of mRNAs, which is unusual for bacteria. Many noncoding RNAs that act in trans are also revealed, and several of the noncoding RNA motifs are found mostly or exclusively in metagenome DNA sequences. This work greatly expands the variety of highly-structured noncoding RNAs known to exist in bacteria and archaea.

2. Results

i. Identification and Analysis of RNA Structures

Promising RNA motifs predicted by the automated bioinformatics procedure were subsequently evaluated manually (see Materials & Methods). As previously reported (Weinberg et al. 2007), promising motifs were identified by seeking RNAs that exhibit both regions of conserved nucleotide sequence and evidence of secondary structure. Evidence for the latter characteristic involved the identification of nucleotide variation between representatives of a motif that conserves a given structure. For example, one form of covariation involves mutations to two nucleotides that preserve a Watson-Crick base pair. Assessment of covariation can be complicated, since, for example, spurious evidence of covariation is sometimes a consequence of sequence misalignments. Therefore, final covariation assessments were performed manually.

Cis-regulatory RNAs in bacteria are typically located in 5′ UTRs. However, transcription start sites for most genes have not been experimentally established. Therefore, when a motif commonly resides upstream of coding regions, it can be assumed that it resides in 5′ UTRs, and is a cis-regulatory RNA. Additional analysis of the system and the scheme for naming motifs is described in Example 3.

ii. Riboswitches

Riboswitches (Roth et al. The Structural and Functional Diversity of Metabolite-Binding Riboswitches. Annu Rev Biochem 2009; Waters et al. Regulatory RNAs in bacteria. Cell 2009, 136:615-628; Montange et al. Riboswitches: emerging themes in RNA structure and function. Annu Rev Biophys 2008, 37:117-133) are RNAs that sense metabolites, and regulate gene expression in response to changes in metabolite concentrations. Typically, they form domains within 5′ UTRs of mRNAs, and their ligand binding triggers a folding change that modulates expression of the downstream gene. Therefore, it is useful to look for riboswitches located in 5′ UTRs. Most known riboswitches require complex secondary and tertiary structures to form tight and highly selective binding pockets for metabolite ligands. Therefore, motifs that have complex secondary structures and stretches of highly conserved nucleotide positions are indicative of a riboswitches.

A total of 12 RNA motifs were identified that exhibited these characteristics. Reported herein is the validation of a new SAM/SAH-binding riboswitch class, and analysis of other identified riboswitches. Details describing additional experimental validation and ligands tested with other riboswitches are presented in Example 3.

iii. SAM/SAH Riboswitch

The coenzyme SAM and its reaction by-product SAH are frequently targeted ligands for riboswitches. Three distinct superfamilies of SAM-binding riboswitches (Wang et al. Riboswitches that sense S-adenosylmethionine and S-adenosylhomocysteine. Biochem Cell Biol 2008, 86:157-168) and one SAH-binding riboswitch class (Wang et al. Riboswitches that sense S-adenosylhomocysteine and activate genes involved in coenzyme recycling. Mol Cell 2008, 29:691-702) have been validated previously. All discriminate against SAM or SAH by orders of magnitude, despite the fact that SAM differs from SAH only by a single methyl group and associated positive charge.

The current search produced a motif, termed SAM/SAH (FIG. 1A), that is found exclusively in the order Rhodobacterales of α-proteobacteria. The RNA motif is consistently found immediately upstream of metK genes, which encode SAM synthetase. Since known SAM-binding riboswitches are frequently upstream of metK genes (Wang et al. Riboswitches that sense S-adenosylmethionine and S-adenosylhomocysteine. Biochem Cell Bio12008, 86:157-168), the element's gene association indicates it may function as part of a novel SAM-sensing riboswitch class.

A SAM/SAH RNA from Roseobacter sp. SK209-2-6, called “SK209-52 RNA”, was subjected to in-line probing (Soukup et al. Relationship between internucleotide linkage geometry and the stability of RNA. RNA 1999, 5:1308-1325) in the presence of various concentrations of SAM or SAH (FIG. 1B, C). SK209-52 RNA binds SAH with an apparent dissociation constant (KD) of ˜4.3 μM and SAM with a K_{D} of ˜8.6 μM (FIG. 1D). Similar results were obtained with SAM/SAH RNA constructs from other species (data not shown). However, because SAM undergoes spontaneous demethylation, SAM samples contain at least some of the breakdown product SAH. Thus, apparent affinity for SAM could result from binding only of contaminating SAH (Wang et al. Riboswitches that sense S-adenosylhomocysteine and activate genes involved in coenzyme recycling. Mol Cell 2008, 29:691-702). However, binding assays based on equilibrium dialysis and molecular recognition experiments indicate that SAM/SAH RNAs do bind SAM (Example 3).

It is interesting to note that SAM/SAH aptamers, which are the smallest of the SAM and SAH aptamer classes, presumably cannot discriminate strongly against SAH. This lack of discrimination may mean that genes associated with this RNA are purposefully regulated by either SAM or SAH. However, SAM is more abundant in cells than SAH (Ueland P M: Pharmacological and biochemical aspects of S-adenosylhomocysteine and S-adenosylhomocysteine hydrolase. Pharmacol Rev 1982, 34:223-253). This fact, coupled with the frequent association of the RNA motif with metK gene contexts of SAM/SAH RNAs, indicates that their biological role is to function as part of a SAM-responsive riboswitch.

iv. crcB Motif

The crcB motif (FIG. 2) is detected in a wide variety of phyla in bacteria and archaea. Thus crcB RNAs join only one known riboswitch class (TPP) (Sudarsan et al. Metabolite-binding RNA domains are present in the genes of eukaryotes. RNA 2003, 9:644-647), and few other RNAs of any kind, that are present in more than one domain of life. The crcB motif consistently resides in the potential 5′ UTRs of genes, including those involved in DNA repair (mutS), K⁺ or Cl⁻ transport, or genes encoding formate hydrogen lyase. In many cases, predicted transcription terminators overlap the conserved crcB motif. Therefore, ligand-binding of the riboswitch that stabilizes the conserved structure at higher ligand concentrations can inhibit terminator stem formation and increase gene expression. The crcB motif can regulate genes in response to stress conditions that can damage DNA, and be mitigated by increased expression of other genes controlled by the RNAs (Example 3).

v. pfl Motif

The pfl motif (FIG. 2) is found in four bacterial phyla. As with crcB RNAs, predicted transcription terminators overlap the 3′ region of many pfl RNAs, thus gene expression can be increased in response to higher ligand concentrations. The genes most commonly associated with pfl RNAs are related to purine biosynthesis, or to synthesis of formyltetrahydrofolate (formyl-THF), which is used for purine biosynthesis. These genes include purH, fhs, pfl, glyA and folD. PurH formylates AICAR using formyl-THF as the donor. Formyl-THF can be synthesized by the product offhs using formate and THF as substrates. Formate, in turn, is produced in the reaction catalyzed by Pfl. The upregulation of Pfl to create formate for the synthesis of purines was observed previously (Derzelle et al. Proteome analysis of Streptococcus thermophilus grown in milk reveals pyruvate formate-lyase as the major upregulated protein. Appl Environ Microbiol 2005, 71:8597-8605). Formyl-THF can also be produced from THF and serine by the combined action of GlyA and FolD. Thus, the five genes most commonly regulated by pfl RNAs have a role in the synthesis of purines or formyl-THF. Most other genes apparently regulated by pfl RNAs encode enzymes that perform other steps in purine synthesis, or convert between THF or its 1-carbon adducts at least as a side effect, e.g., metH (Example 3).

vi. yjdF Motif

The yjdF motif (FIG. 2) is found in many Firmicutes, including Bacillus subtilis. In most cases, it resides in potential 5′ UTRs of homologs of the yjdF gene, whose function is unknown. However, in Streptococcus thermophilus, a yjdF RNA motif is associated with an operon whose protein products synthesize nicotinamide adenine dinucleotide (NAD'). Also, the S. thermophilus yjdF RNA lacks typical yjdF motif consensus features downstream of and including the P4 stem. Thus, the S. thermophilus RNAs can sense a distinct compound that structurally resembles the ligand bound by other yjdF RNAs. Alternatively, these RNAs have an alternate solution to form a similar binding site, as is observed with some SAM riboswitches (Weinberg et al. The aptamer core of SAM-IV riboswitches mimics the ligand-binding site of SAM-I riboswitches. RNA 2008, 14:822-828).

vii. manA and wcaG Motifs

The manA and wcaG motifs (FIG. 2) are found almost exclusively in marine metagenome sequences, but are each detected in T4-like phages that infect cyanobacteria. Also, two manA RNAs are found in γ-proteobacteria. Remarkably, many phages of cyanobacteria have incorporated genes involved in metabolism, including exopolysaccharide production and photosynthesis (Sullivan et al. Three Prochlorococcus cyanophage genomes: signature features and ecological interpretations. PLoS Biol 2005, 3:e144; Rohwer F, Thurber R V: Viruses manipulate the marine environment. Nature 2009, 459:207-212; Lindell et al. Genome-wide expression dynamics of a marine virus and host reveal features of co-evolution. Nature 2007, 449:83-86), and some of these cyanophages carry manA or wcaG RNAs. RNA domains corresponding to the manA motif are commonly located in potential 5′ UTRs of genes involved in mannose or fructose metabolism, nucleotide synthesis, ibpA chaperones, and photosynthetic genes. Distinctively, wcaG RNAs typically regulate genes related to production of exopolysaccharides or genes that are induced by high light conditions. Perhaps manA and wcaG RNAs are used by phages to modify their hosts' metabolism (Lindell et al. Genome-wide expression dynamics of a marine virus and host reveal features of co-evolution. Nature 2007, 449:83-86), though they may also be exploited by uninfected bacteria.

viii. epsC Motif

RNA domains corresponding to the epsC motif (FIG. 2) are found in potential 5′

UTRs of genes related to exopolysaccharide (EPS) synthesis such as epsC (Lemon et al. Biofilm development with an emphasis on Bacillus subtilis. Curr Top Microbiol Immunol 2008, 322:1-16), in B. subtilis and related species. Different species use different chemical subunits in their EPS (Leoff et al. Cell wall carbohydrate compositions of strains from the Bacillus cereus group of species correlate with phylogenetic relatedness. J Bacteriol 2008, 190:112-121), which acts in processes such as biofilm formation, capsule synthesis, and sporulation (Leoff et al. Cell wall carbohydrate compositions of strains from the Bacillus cereus group of species correlate with phylogenetic relatedness. J Bacteriol 2008, 190:112-121; Nakhamchik et al. Cyclic-di-GMP regulates extracellular polysaccharide production, biofilm formation, and rugose colony development by Vibrio vulnificus. Appl Environ Microbiol 2008, 74:4199-4209; Torres-Cabassa et al. Control of extracellular polysaccharide synthesis in Erwinia stewartii and Escherichia coli K-12: a common regulatory function. J Bacteriol 1987, 169:4525-4531). epsC RNA motifs can sense an intermediate in EPS synthesis that is common to all bacteria containing epsC RNAs. Signalling molecules also regulate EPS synthesis in some bacteria (Nakhamchik et al. Cyclic-di-GMP regulates extracellular polysaccharide production, biofilm formation, and rugose colony development by Vibrio vulnificus. Appl Environ Microbiol 2008, 74:4199-4209; Liang et al. The cyclic AMP receptor protein modulates colonial morphology in Vibrio cholerae. Appl Environ Microbiol 2007, 73:7482-7487), and are therefore also identified as riboswitch ligands.

The epsC motif was discovered independently by another group and named EAR. This motif has been shown to exhibit transcription antitermination activity likely by directly interacting with protein components of the transcription elongation complex, and therefore this RNA motif may not also function as a metabolite-binding RNA. Intriguingly, the JUMPstart sequence motif (Hobbs et al. The JUMPstart sequence: a 39 bp element common to several polysaccharide gene clusters. Mol Microbiol 1994, 12:855-856) is found in the 5′ UTRs of genes related to polysaccharide synthesis and also is associated with modification of transcriptional elongation (Marolda et al. Promoter region of the Escherichia coli O7-specific lipopolysaccharide gene cluster: structural and functional characterization of an upstream untranslated mRNA sequence. J Bacteriol 1998, 180:3070-3079; Nieto et al. Suppression of transcription polarity in the Escherichia coli haemolysin operon by a short upstream element shared by polysaccharide and DNA transfer determinants. Mol Microbiol 1996, 19:705-713; Leeds et al. Enhancing transcription through the Escherichia coli hemolysin operon, hlyCABD: RfaH and upstream JUMPStart DNA sequences function together via a postinitiation mechanism. J Bacteriol 1997, 179:3519-3527; Wang et al. Expression of the O antigen gene cluster is regulated by RfaH through the JUMPstart sequence. FEMS Microbiol Lett 1998, 165:201-206). A conserved stem-loop structure among JUMPstart elements was detected (Example 3).

ix. ykkC-III Motif

The previously identified ykkC (Barrick et al. New RNA motifs suggest an expanded scope for riboswitches in bacterial genetic control. Proc Natl Acad Sci USA 2004, 101:6421-6426) and mini-ykkC (Weinberg et al., 2007) motifs are associated with genes related to those associated with ykkC-III, but these RNAs have distinct conserved sequence and structural features. The new-found ykkC-III motif (FIG. 2) is in potential 5′ UTRs of emrE and speB genes. emrE is the most common gene family associated with mini-ykkC and the second most common to be associated with ykkC, while speB is also associated with ykkC RNAs in many cases. Although a perfectly conserved ACGA sequence in ykkC-III is similar to the less rigidly conserved ACGR terminal loops of mini-ykkC RNAs, the structural contexts arc different (Example 3). All three RNA motifs have characteristics of gene control elements that regulate similar genes, and can respond to changing concentrations of the same metabolite. However, unlike mini-ykkC whose small and repetitive hairpin architecture is suggestive of protein binding, both ykkC and ykkC-III exhibit more complex structural features that are indicative of direct metabolite binding.

x. glnA and Downstream-Peptide Motifs

The glnA and Downstream peptide motifs carry similar sequence and structural features (FIG. 3; Example 1), although the genes they are associated with are very different. Many genes presumably regulated by glnA RNAs are clearly involved in nitrogen metabolism, and include nitrogen regulatory protein P_{H}, glutamine synthetase, glutamate synthase and ammonium transporters. Another associated gene is PMT1479, which was the most repressed gene when Prochlorococcus marinus was starved for nitrogen (Tolonen et al. Global gene expression of Prochlorococcus ecotypes in response to changes in nitrogen availability. Mol Syst Biol 2006, 2:53). Some glnA RNA motifs occur in tandem, which is an arrangement previously associated with more-digital gene regulation (Mandal et al. A glycine-dependent riboswitch that uses cooperative binding to control gene expression. Science 2004, 306:275-279; Welz et al. Ligand binding and gene control characteristics of tandem riboswitches in Bacillus anthracis. Rna 2007, 13:573-582).

The Downstream-peptide motif is found in potential 5′ UTRs of cyanobacterial ORFs whose products are typically 17-100 amino acids long, and are predicted not to belong to a known protein family. A pattern of synonymous mutations and insertions or deletions was observed in multiples of three nucleotides, supporting the prediction of a short conserved coding sequence. A previously predicted noncoding RNA called “yfr6” (Axmann et al. Identification of cyanobacterial non-coding RNAs by comparative genome analysis. Genome Biology 2005, 6:R73) is ˜250 nucleotides in length and contains a short ORF. The 5′ UTRs of these ORFs correspond to Downstream-peptide RNAs. While only two full-length yfr6 RNAs were found, 634 Downstream-peptide RNAs are detected, indicating that only the 5′ UTR is conserved. Experiments on yfr6 showed that transcription starts ˜20 nucleotides 5′ to the proposed Downstream-peptide motif (Axmann et al. Identification of cyanobacterial non-coding RNAs by comparative genome analysis. Genome Biology 2005, 6:R73). Also, a Downstream-peptide RNA resides in the potential 5′ UTR of a gene that appears to be down-regulated in response to nitrogen starvation (Axmann et al. Identification of cyanobacterial non-coding RNAs by comparative genome analysis. Genome Biology 2005, 6:R73). A conserved amino acid sequence in predicted proteins associated with Downstream-peptide RNAs indicates a regulatory mechanism (Example 3). The structural resemblance between glnA and Downstream-peptide RNA motifs makes sense because both sense glutamine. Both elements down-regulate genes in response to nitrogen depletion.

xi. Cyanobacterial photosystem regulatory motifs

a. psaA Motif

Representatives of the psaA motif (FIG. 4) occur in the potential 5′ UTRs of Photosystem-I psaAB operons in certain cyanobacteria. The motif includes three hairpins that often include UNCG tetraloops (Pace et al. Probing RNA structure, function, and history by comparative analysis. In: The RNA World, 2nd edition Edited by Gesteland R F, Cech T R, Atkins J F. Cold Spring Harbor, N.Y.: Cold Spring Harbor Laboratory Press; 1999). While the regulation of psaAB genes in species with psaA RNAs has not been studied, multiple psa genes in Synechocystis sp. PCC 6803 are regulated in response to light via DNA elements that are presumably transcription factor binding sites (Muramatsu et al. Coordinated high-light response of genes encoding subunits of photosystem I is achieved by AT-rich upstream sequences in the cyanobacterium Synechocystis sp. strain PCC 6803. J Bacteriol 2007, 189:2750-2758). Photosynthetic organisms up-regulate photosystem-I (psa) genes under low light conditions to maximize energy output, but must reduce their expression under sustained high light conditions, to avoid damage from free radicals (Muramatsu et al. Characterization of high-light-responsive promoters of the psaAB genes in Synechocystis sp. PCC 6803. Plant Cell Physiol 2006, 47:878-890). psaA RNAs could be involved in this regulation, although this RNA element has not been found upstream of psa genes other than psaAB.

b. PhotoRC-I, PhotoRC-II and psbNH Motifs

Two distinct RNA structures (FIG. 4) are associated with genes belonging to the photosynthetic reaction center family of proteins that can be psbA PhotoRC-I RNAs are present in known cyanobacteria and in marine environmental samples, while PhotoRC-II RNAs are detected only in marine samples and a cyanophage. These motifs and psbNH are further described in Example 3.

xii. Other Motifs

a. L17 Downstream Element

The L17 downstream element (FIG. 16) is located downstream (within the potential 3′ UTRs) of genes that encode ribosomal protein L17. In many cases, there are no annotated genes located immediately downstream of the element. Although the motif can actually be transcribed in the opposite orientation, the structure as shown is more stable because it carries many G-U base pairs and GNRA tetraloops (Pace et al. Probing RNA structure, function, and history by comparative analysis. In: The RNA World, 2nd edition Edited by Gesteland R F, Cech T R, Atkins J F. Cold Spring Harbor, N.Y.: Cold Spring Harbor Laboratory Press; 1999). These structures would be far less stable in the corresponding RNA transcribed from the complementary DNA template. The expression of ribosomal proteins is frequently regulated by a feedback mechanism where the protein binds an RNA structure in the 5′ UTR of its mRNA (Zengel et al. Diverse mechanisms for regulating ribosomal protein synthesis in \taxonEscherichia coli. Prog Nucleic Acid Res Mol Biol 1994, 47:331-370). Thus, the L17 downstream element could function in the 3′ UTR and be part of a feedback regulation system for L17 production. Regulation of a gene by a structured RNA domain located in the 3′ UTR is highly unusual in bacteria. However, precedents include an element in a ribosomal protein operon that regulates both upstream and downstream genes (Mattheakis et al. Retroregulation of the synthesis of ribosomal proteins L14 and L24 by feedback repressor S8 in Escherichia coli. Proc Natl Acad Sci USA 1989, 86:448-452), and regulation of upstream genes is observed in a phage (Guarneros et al. Posttranscriptional control of bacteriophage lambda gene expression from a site distal to the gene. Proc Natl Acad Sci USA 1982, 79:238-242) and proposed in Listeria (Toledo-Arana et al. The Listeria transcriptional landscape from saprophytism to virulence. Nature 2009, 459:950-956).

b. hopC Motif

The hopC motif (FIG. 16) is found in Helicobacter species in the potential 5′ UTRs of hopC/alpA gene and co-transcribed hopB/alpB genes. Previous studies established that expression of the hopCB operon is increased in response to low pH (McGowan et al. Promoter analysis of Helicobacter pylori genes with enhanced expression at low pH. Mol Microbiol 2003, 48:1225-1239). The experimentally determined 5′ UTRs of a hopCB operon mRNA (McGowan et al. Promoter analysis of Helicobacter pylori genes with enhanced expression at low pH. Mol Microbiol 2003, 48:1225-1239) contains a hopC motif RNA. HopCB is needed for optimal binding to human epithelial cells (Odenbreit et al. Role of the alpAB proteins and lipopolysaccharide in adhesion of Helicobacter pylori to human gastric tissue. Int J Med Microbiol 2002, 292:247-256), and is presumably involved in infection of the human stomach.

c. msiK Motif

The msiK motif is always found in the potential 5′ UTRs of msiK genes (Hurtubise et al. A cellulase/xylanase-negative mutant of Streptomyces lividans 1326 defective in cellobiose and xylobiose uptake is mutated in a gene encoding a protein homologous to ATP-binding proteins. Mol Microbiol 1995, 17:367-377; Parche et al. Sugar transport systems of Bifidobacterium longum NCC2705. J Mol Microbiol Biotechnol 2007, 12:9-19), which encode the ATPase subunit for ABC-type transporters of at least two complex sugars (Schlösser et al. The Streptomyces ATP-binding component MsiK assists in cellobiose and maltose transport. J Bacteriol 1997, 179:2092-2095), and probably many more (Bertram et al. In silico and transcriptional analysis of carbohydrate uptake systems of Streptomyces coelicolor A3(2). J Bacteriol 2004, 186:1362-1373). The motif is comprised of an 11-nucleotide bulge within a long hairpin. The 3′ side of the basal pairing region includes a predicted ribosome binding site, which may be part of the regulatory mechanism. Existing data indicate that msiK genes are not regulated in response to changing levels of glucose (Hurtubise et al. A cellulase/xylanase-negative mutant of Streptomyces lividans 1326 defective in cellobiose and xylobiose uptake is mutated in a gene encoding a protein homologous to ATP-binding proteins. Mol Microbiol 1995, 17:367-377; Schlösser et al. The Streptomyces ATP-binding component MsiK assists in cellobiose and maltose transport. J Bacteriol 1997, 179:2092-2095), so perhaps the RNA participates in a feedback inhibition loop by binding MsiK proteins (Example 3).

d. pan Motif

The pan motif (FIG. 19) is found in three phyla, and is present in the genetically tractable organism B. subtilis. Each pan RNA consists of a stem interrupted by two highly conserved bulged A residues. Most pan RNAs occur in tandem, and their simple structure and dimeric arrangement is suggestive of a dimeric protein binding motif. The RNAs are located upstream of operons containing panB, panC or aspartate decarboxylase genes, which are involved in synthesizing pantothenate (vitamin B₅).

e. rmf Motif

The rmf motif is found in the potential 5′ UTRs of rmf genes in Pseudomonas species. These genes encode ribosome modulation factor, which acts in the stringent response to depletion of nutrients and other stressors (Niven et al. Ribosome modulation factor. In: Bacterial physiology: a molecular approach Edited by El-Sharoud WM. Berlin: Springer-Verlag; 2008). Since Rmf interacts with rRNA, the protein Rmf can bind to the 5′ UTR of its mRNA. Alternately, since the RNA is relatively far from the rmf start codon, rmf RNAs can be non-coding RNAs that are expressed separately from the adjacent coding region.

f. SAM-Chlorobi Motif

The SAM-Chlorobi motif is found in the potential 5′ UTRs of operons containing all predicted metK and ahcY genes within the phylum Chlorobi. As noted above, metK encodes SAM synthetase, and in most other organisms metK homologs are controlled by changing SAM concentrations that are detected by SAM-responsive riboswitches. In contrast, ahcY encodes S-adenosylhomocysteine (SAH) hydrolase, and this gene is known to be controlled by SAH-responsive riboswitches in some organisms (Wang et al. Riboswitches that sense S-adenosylhomocysteine and activate genes involved in coenzyme recycling. Mol Cell 2008, 29:691-702). Sequences conforming to a strong promoter sequences (Bayley et al. Analysis of cepA and other Bacteroides fragilis genes reveals a unique promoter structure. FEMS Microbiol Lett 2000, 193:149-154; Chen et al. Characterization of strong promoters from an environmental Flavobacterium hibernum strain by using a green fluorescent protein-based reporter system. Appl Environ Microbiol 2007, 73:1089-1100) imply that SAM-Chlorobi RNAs are transcribed (Example 3). However, preliminary analysis of several SAM-Chlorobi RNA constructs using in-line probing did not reveal binding to SAM or SAH (Example 3).

g. STAXI Motif

The Ssbp, Topoisomerase, Antirestriction, XerDC Integrase (STAXI) motif is composed mainly of a pseudoknot structure repeated at least two and usually three times (FIG. 5). Tandem STAXI motifs are frequently nearby to genes that encode proteins that bind or manipulate DNA, including single-stranded DNA binding proteins (Ssbp), integrases and topoisomerases, or antirestriction proteins. Also, they are occasionally located nearby c4 antisense RNAs (Citron et al. The c4 repressors of bacteriophages P1 and P7 are antisense RNAs. Cell 1990, 62:591-598) (Example 3). Since genes proximal to STAXI representatives encode DNA manipulation proteins, it was possible that the STAXI motif represented a single-stranded DNA that adopted a local structure when duplex DNA is separated, as occurs during DNA replication, repair, or when bound by some proteins. However, the UUCG tetraloops that frequently occur within the STAXI motif repeats are known to stabilize RNA, whereas the corresponding TTCG are not particularly stabilizing for DNA structures (Antao et al. Thermodynamic parameters for loop formation in RNA and DNA hairpin tetraloops. Nucleic Acids Res 1992, 20:819-824). This indicates that the motif serves its function as an RNA structure.

xiii. Noncoding RNAs

Several motifs that are likely expressed as noncoding RNAs unaffiliated with mRNAs also were identified (FIG. 5, Table 1). Gut-1 and whalefall-1 RNAs are found only in environmental sequences and Bacteroides-2 is found in only one sequenced organism (Example 3). Thus, bacteria from multiple environmental samples express noncoding RNAs that are not represented in any cultivated organisms whose genomes have been sequenced (Weinberg et al. Extraordinary structured noncoding RNAs revealed by bacterial metagenome analysis. Nature 2009; Shi et al. Metatranscriptomics reveals unique microbial small RNAs in the ocean's water column. Nature 2009, 459:266-269). Similarly, Acido-1 and Dictyoglomi-1 RNAs are found in phyla in which few genome sequences are available. Further observations regarding all noncoding RNA motifs can be found in Example 3.

TABLE 1
List of Motifs

| Motif | RNA? | cis-reg? | Switch? | Taxa |
|---|---|---|---|---|
| 6S-flavo | Y | N | N | Bacteroidetes |
| aceE | ? | y | ? | γ-proteobacteria |
| Acido-1 | y | n | n | Acidobacteria |
| Acido-Lenti-1 | y | n | n | Acidobacteria, Lentisphaerae |
| Actino-pnp | Y | Y | N | Actinomycetales |
| AdoCbl-variant | Y | Y | Y | marine |
| asd | Y | ? | ? | Lactobacillales |
| atoC | y | y | ? | δ-proteobacteria |
| Bacillaceae-1 | Y | n | n | Bacillaceae |
| Bacillus-plasmid | y | ? | n | Bacillus |
| Bacteroid-trp | y | y | n | Bacteroidetes |
| Bacteroidales-1 | Y | ? | ? | Bacteroidales |
| Bacteroides-1 | y | ? | n | Bacteroides |
| Bacteroides-2 | ? | n | n | Bacteroides |
| Burkholderiales-1 | ? | ? | n | Burkholderiales |
| c4 antisense RNA | Y | N | N | Proteobacteria, phages |
| c4-a1b1 | Y | N | N | γ-proteobacteria, phages |
| Chlorobi-1 | Y | n | n | Chlorobi |
| Chlorobi-RRM | y | y | n | Chlorobi |
| Chloroflexi-1 | y | ? | n | Chloroflexus aggregans |
| Clostridiales-1 | y | n | n | Clostridiales, human gut |
| COG2252 | ? | y | n | Pseudomonadales |
| Collinsella-1 | y | n | n | Actinobacteria, human gut |
| crcB | Y | Y | Y | Widespread, bacteria and archaea |
| Cyano-1 | y | n | n | Cyanobacteria, marine |
| Cyano-2 | Y | n | n | Cyanobacteria, marine |
| Desulfotalea-1 | ? | n | n | Proteobacteria |
| Dictyoglomi-1 | y | ? | ? | Dictyoglomi |
| Downstream-peptide | Y | y | y | Cyanobacteria, marine |
| epsC | Y | y | y | Bacillales |
| fixA | ? | y | n | Pseudomonas |
| Flavo-1 | y | n | n | Bacteroidetes |
| flg-Rhizobiales | y | y | n | Rhizobiales |
| flpD | y | ? | n | Euroarchaeota |
| gabT | Y | y | ? | Pseudomonas |
| Gamma-cis-1 | ? | y | n | γ-proteobacteria |
| glnA | Y | Y | y | Cyanobacteria, marine |
| GUCCY-hairpin | ? | ? | n | Bacteroidetes, Proteobacteria |
| Gut-1 | Y | n | n | human gut only |
| gyrA | y | y | n | Pseudomonas |
| hopC | y | Y | ? | Helicobacter |
| icd | ? | y | n | Pseudomonas |
| JUMPstart | y | Y | n | γ-proteobacteria |
| L17 downstream element | y | y | n | Lactobacillales, Listeria |
| lactis-plasmid | y | ? | n | Lactobacillales |
| Lacto-int | ? | ? | n | Lactobacillales, phages |
| Lacto-rpoB | Y | y | n | Lactobacillales |
| Lacto-usp | Y | ? | ? | Lactobacillales |
| Leu/phe leader | Y | Y | Y | Lactococcus lactis |
| livK | y | y | ? | Pseudomonadales |
| Lnt | y | y | ? | Chlorobi |
| manA | Y | Y | y | marine, γ-proteobacteria, cyanophage |
| Methylobacterium-1 | Y | n | n | Methylobacterium, marine |
| Moco-II | y | Y | ? | Proteobacteria |
| mraW | y | y | ? | Actinomycetales |
| msiK | Y | Y | ? | Actinobacteria |
| Nitrosococcus-1 | ? | n | n | Nitrosococcus, Clostridia |
| nuoG | y | y | ? | Enterobacteriales (incl. E. coli K12) |
| Ocean-V | y | n | n | marine only |
| Ocean-VI | ? | ? | ? | marine only |
| pan | Y | Y | ? | Chloroflexi, Firmicutes, δ-proteobacteria |
| Pedo-repair | y | ? | n | Pedobacter |
| pfl | Y | Y | Y | several phyla |
| pheA | ? | y | n | Actinobacteria |
| PhotoRC-I | y | y | n | Cyanobacteria, marine |
| PhotoRC-II | Y | y | n | marine, cyanophage |
| Polynucleobacter-1 | y | y | ? | Burkholderiales, fresh water/estuary |
| potC | y | y | ? | marine only |
| psaA | Y | y | ? | Cyanobacteria |
| psbNH | y | y | n | Cyanobacteria, marine |
| Pseudomon-1 | y | n | n | Pseudomonadales |
| Pseudomon-2 | ? | n | n | |
| Pseudomon-GGDEF | ? | y | ? | Pseudomonas |
| Pseudomon-groES | y | y | ? | Pseudomonas |
| Pseudomon-Rho | y | Y | n | Pseudomonas |
| Pyrobac-1 | y | n | n | Pyrobaculum |
| Pyrobac-HINT | ? | y | n | |
| radC | Y | y | ? | Proteobacteria |
| Rhizobiales-1 | ? | n | N | Rhizobiales |
| Rhizobiales-2 | y | ? | n | Rhizobiales |
| Rhodopirellula-1 | ? | y | ? | Proteobacteria, Planctomycetes |
| rmf | Y | y | ? | Pseudomonadales |
| rne-II | Y | y | N | Pseudomonadales |
| SAM-Chlorobi | y | Y | ? | Chlorobi |
| SAM-I-IV-variant | Y | Y | Y | several phyla, marine |
| SAM-II long loops | Y | Y | Y | Bacteroidetes, marine |
| SAM/SAH | Y | Y | Y | Rhodobacterales |
| sanguinis-hairpin | ? | n | n | Streptococcus |
| sbcD | y | ? | n | Burkholderiales |
| ScRE | ? | y | n | Streptococcus |
| Soil-1 | ? | n | n | soil only |
| Solibacter-1 | ? | n | n | Solibacter usitatus |
| STAXI | y | ? | n | Enterobacteriales |
| sucA-II | y | y | ? | Pseudomonadales |
| sucC | Y | Y | ? | γ-proteobacteria |
| Termite-flg | Y | y | n | termite hind gut only |
| Termite-leu | y | ? | ? | termite hind gut only |
| traJ-II | Y | Y | n | Proteobacteria, Enterococcus faecium |
| Transposase-resistance | ? | y | n | several phyla |
| TwoAYGGAY | y | n | n | human gut, γ-proteobacteria, Clostridiales |
| wcaG | Y | y | y | marine, cyanophage |
| Whalefall-1 | Y | n | n | whalefall only |
| yjdF | Y | Y | Y | Firmicutes |
| ykkC-III | y | Y | y | Actinobacteria, δ-proteobacteria |

Columns are as follows.
Column “RNA?”: is this motif likely to represent a biological RNA?
Column “cis-reg”: is the motif cis-regulatory?
Column “switch?”: is the motif a riboswitch?
Column “Taxa”: common taxon/taxa carrying this motif.
Notation meaning:
“Y” = certainly,
“y” = probably,
“?” = ambiguous,
“n” = probably not,
“N” = no.
Many of the motifs are discussed in Example 3.

xiv. Expansion of Representatives of Previously Characterized Structured RNAs

Existing homology search methods for RNAs frequently fail to detect representatives of known RNA classes whose sequences have diverged extensively. However, the computational pipeline occasionally reveals examples of such RNAs. Details regarding RNA representatives that expand the collection of 6S RNAs, AdoCbl riboswitches, SAM-II riboswitches, and SAM-I/SAM-IV riboswitches are provided in Example 3. The RNAs that expand the collection of the superfamily of SAM-I (Winkler et al. An mRNA structure that controls gene expression by binding S-adenosylmethionine Nat. Struct. Biol. 2003, 10:701-707) and SAM-IV (Weinberg et al. The aptamer core of SAM-IV riboswitches mimics the ligand-binding site of SAM-I riboswitches. Rna 2008, 14:822-828) riboswitches (FIG. 24) are typically found in metagenome sequences. These variant SAM-I/SAM-IV riboswitches share many of the structural features of both families (FIG. 24), but lack an internal loop in the P2 stem, which is present in SAM-I/SAM-IV riboswitches (Example 3).

3. Conclusions

Numerous structured RNA motifs have been identified in the genomic and metagenomic DNA sequence data from bacteria and archaea. The RNA motifs exhibit a great diversity of conserved sequences and structural features, and their genomic locations are indicative of a wide variety of mechanisms of action (e.g., cis vs. trans) and expected biological roles. The disclosed findings indicate that the bacterial and archaeal domains of life will continue to be a rich source of novel structured RNAs.

Although some of the RNAs identified perform the same function as previously validated RNA classes (e.g. 6S-Flavo RNA, SAM/SAH riboswitches), the vast majority of the identified RNA motifs perform novel functions. Given that many of these RNAs are specific to certain lineages or uncultivated environmental samples, technologies that more rapidly make available DNA sequence information from additional lineages of bacteria and archaea are likely to accelerate the discovery of more classes of structured RNAs. This discovery rate can also be increased by improvements in computational analysis methods. These findings should yield a diverse collection of structured noncoding RNAs that will reveal a more complete understanding of the roles that RNAs perform in microbial cells.

4. Materials and Methods

i. DNA Sequence Sources and Gene Annotations

The microbial subsets of RefSeq (Pruitt et al. NCBI Reference Sequence (RefSeq): a curated non-redundant sequence database of genomes, transcripts and proteins. Nucleic Acids Res. 2005, 33:501-504) version 25 or 32 were searched, along with metagenome sequences from acid mine drainage (Tyson et al. Community structure and metabolism through reconstruction of microbial genomes from the environment. Nature 2004, 428:37-43), soil and whale fall (Tringe et al. Comparative metagenomics of microbial communities. Science 2005, 308:554-557), human gut (Gill et al. Metagenomic analysis of the human distal gut microbiome. Science 2006, 312:1355-1359; Kurokawa et al. Comparative metagenomics revealed commonly enriched gene sets in human gut microbiomes. DNA Res. 2007, 14:169-181), mouse gut (Turnbaugh et al. An obesity-associated gut microbiome with increased capacity for energy harvest. Nature 2006, 444:1027-1031), gutless sea worms (Woykc et al. Symbiosis insights through metagenomic analysis of a microbial consortium. Nature 2006, 443:950-955), sludge (Garcia et al. Metagenomic analysis of two enhanced biological phosphorus removal (EBPR) sludge communities. Nat Biotechno12006, 24:1263-1269), Global Ocean Survey scaffolds (Rusch et al.: The Sorcerer II Global Ocean Sampling expedition: northwest Atlantic through eastern tropical Pacific. PLoS Biol 2007, 5:e77; Venter et al. Environmental genome shotgun sequencing of the Sargasso Sea. Science 2004, 304:66-74), other marine sequences (DeLong et al. Community genomics among stratified microbial assemblages in the ocean's interior. Science 2006, 311:496-503) and termite hindgut (Warnecke et al. Metagenomic and functional analysis of hindgut microbiota of a wood-feeding higher termite. Nature 2007, 450:560-565). Locations and identities of protein-coding genes were derived from RefSeq or IMG/M (Markowitz et al. IMG/M: a data management and analysis system for metagenomes. Nucleic Acids Res 2008, 36:D534-538) annotations, or from “predicted proteins” (Yooseph et al. The Sorcerer 11 Global Ocean Sampling expedition: expanding the universe of protein families. PLoS Biol 2007, 5:e16) in Global Ocean Survey sequences. However, genes in some sequences (Kurokawa et al. Comparative metagenomics revealed commonly enriched gene sets in human gut microbiomes. DNA Res. 2007, 14:169-181; DeLong et al. Community genomics among stratified microbial assemblages in the ocean's interior. Science 2006, 311:496-503; Warnecke et al. Metagenomic and functional analysis of hindgut microbiota of a wood-feeding higher termite. Nature 2007, 450:560-565) were predicted using MetaGene (dated Oct. 12, 2006) with default parameters (Noguchi et al. MetaGene: prokaryotic gene finding from environmental genome shotgun sequences. Nucleic Acids Res 2006, 34:5623-5630). Conserved protein domains were annotated using the Conserved Domain Database version 2.08 (Marchler-Bauer et al. CDD: a Conserved Domain Database for protein classification. Nucleic Acids Research 2005, 33:192-196).

Annotations for tRNAs and rRNAs were derived from the sources noted above, or were predicted using tRNAscan-SE (Lowe et al. tRNAscan-SE: a program for improved detection of transfer RNA genes in genomic sequence. Nucleic Acids Research 1997, 25:955-964) run in bacterial mode. To detect additional rRNAs, annotated rRNAs whose descriptions read “ribosomal RNA” or “#S rRNA” (# represents any number) were used in WU-BLAST queries with command-line flags-hspsepQmax=4000-E 1e-20-W 8 (Yao et al. A computational pipeline for high-throughput discovery of cis-regulatory noncoding RNA in prokaryotes. PLoS Comput. Biol. 2007, 3:e126). Other RNAs were detected with Rfam (Gardner et al. Rfam: updates to the RNA families database. Nucleic Acids Res 2009, 37:D136-140), and WU-BLAST as described previously (Yao et al. A computational pipeline for high-throughput discovery of cis-regulatory noncoding RNA in prokaryotes. PLoS Comput. Biol. 2007, 3:e126). Published alignments of riboswitches were also used (Barrick et al. The distributions, mechanisms, and structures of metabolite-binding riboswitches. Genome Biol. 2007, 8:R239) as queries with RaveNnA global-mode searches (Weinberg et al. Sequence-based heuristics for faster annotation of non-coding RNA families. Bioinformatics 2006, 22:35-39; Eddy et al. RNA Sequence Analysis Using Covariance Models. Nucleic Acids Research 1994, 22:2079-2088), selecting hits manually based primarily on E-values.

iI. Automated Motif Identification

To reduce false positives in sequence comparisons, the pipeline was run separately on related taxa or metagenome sources (data not shown). For each run, InterGenic Regions (IGRs) of at least 30 nucleotides were extracted between protein-coding, tRNA and rRNA genes.

To generate clusters, an early version of a recently described algorithm was used (Tseng et al. Finding non-coding RNAs through genome-scale clustering. J Bioinform Comput Biol 2009, 7:373-388). Specifically, IGRs were compared using nucleotide NCBI BLAST (Altschul et al. Gapped BLAST and PSI-BLAST: a new generation of protein database search programs. Nucleic Acids Research 1997, 25:3389-3402) version 2.2.17 and parameters —W 7-G 2-E 2-q-2-m 8. Self matches were ignored. BLAST scores below a parameter S (see below) were considered insignificant and ignored. Each BLAST match defines two “nodes”, corresponding to the matching sequences. Nodes that overlap by at least five nucleotides are merged, along with their BLAST homologies. A cluster consists of all nodes that have direct or indirect (transitive) BLAST matches. Closely related sequences that span multiple distinct elements in an entire IGR can lead to spurious node merges. Therefore, homologies with BLAST scores above 100 are ignored.

If a node's length in nucleotides is L, and L<500, then the node is extended on either side by (500-L)/2 nucleotides, but is constrained to remain within the original IGR. CMfinder can easily tolerate nodes of 500 nucleotides. When L>1000, nodes are shrunk by (L-1000)/2 nucleotides around the center. The L>1000 case is extremely rare. Only clusters with at least three members were reported.

For each pipeline run, a range of values was tried for the parameter S=35, 40, . . . , 85, and determined how many known RNAs were detected with each value. Based on these data, a set of S values was selected manually, and the union of clusters arising from each S was used as input to CMfinder (Yao et al. CMfinder—a covariance model based RNA motif finding algorithm. Bioinformatics 2006, 22:445-452). CMfinder was used to predict motifs exactly as before (Yao et al. A computational pipeline for high-throughput discovery of cis-regulatory noncoding RNA in prokaryotes. PLoS Comput. Biol. 2007, 3:e126). Automated homology searches were then performed as described (Yao et al. A computational pipeline for high-throughput discovery of cis-regulatory noncoding RNA in prokaryotes. PLoS Comput. Biol. 2007, 3:e126), except that covariance model scores used the null3 model (Nawrocki et al. Infernal 1.0: inference of RNA alignments. Bioinformatics 2009, 25:1335-1337). Motifs were scored using a previously established method (Yao et al. A computational pipeline for high-throughput discovery of cis-regulatory noncoding RNA in prokaryotes. PLoS Comput. Biol. 2007, 3:e126), and by using tools comprising Pfold (Knudsen et al. Pfold: RNA secondary structure prediction using stochastic context-free grammars. Nucleic Acids Res 2003, 31:3423-3428) to infer a phylogenetic tree, then running pscore (Yao Z: Genome scale search of noncoding RNAs: bacteria to vertebrates. Seattle, Wash.: University of Washington; 2008. Dissertation). Motifs that had no covarying base pair positions, that had an average G+C content less than 24%, that had representatives whose nucleotide coordinates overlapped the reverse-complements of other representatives on average by 30% or more of their nucleotides, or that had fewer than six positions that were at least 97% conserved (when sequences were weighted with the GSC algorithm) were automatically eliminated.

iii. Manual Analysis of Motifs

The manual analysis of each RNA motif proceeded essentially as described previously (Weinberg et al., 2007). For motifs that were likely to be cis-regulatory, papers referencing the locus tags of apparently regulated genes were routinely searched for using Google Scholar (http://scholar.google.com). Mutual information analysis (Barrick et al. The distributions, mechanisms, and structures of metabolite-binding riboswitches. Genome Biol. 2007, 8:R239) was also used to predict additional base pairing interactions. Motifs less likely to represent structured RNAs were rejected using previously established criteria (Weinberg et al., 2007). In motif consensus diagrams, covariation and levels of conservation were calculated using earlier protocols (Weinberg et al., 2007), but up to 10% non-canonical pairs were tolerated in alignment columns that correspond to conserved base-pairs.

iv. Assessing the Novelty of Motifs

To determine if the RNA structures were reported previously, the Rfam database (Gardner et al. Rfam: updates to the RNA families database. Nucleic Acids Res 2009, 37:D136-140), and various papers not yet incorporated into Rfam that performed detailed analysis or experiments on new-found candidate RNAs (Marchais et al. Single-pass classification of all noncoding sequences in a bacterial genome using phylogenetic profiles. Genome Res 2009, 19:1084-1092; Axmann et al. Identification of cyanobacterial non-coding RNAs by comparative genome analysis. Genome Biology 2005, 6:R73; Liu et al. Experimental discovery of sRNAs in Vibrio cholerae by direct cloning, 5S/tRNA depletion and parallel sequencing. Nucleic Acids Res 2009, 37:e46; Livny et al. Identification of 17 Pseudomonas aeruginosa sRNAs and prediction of sRNA-encoding genes in 10 diverse pathogens using the bioinformatic tool sRNAPredict2. Nucleic Acids Res 2006, 34:3484-3493; Sonnleitner et al. Detection of small RNAs in Pseudomonas aeruginosa by RNomics and structure-based bioinformatic tools. Microbiology 2008, 154:3175-3187; Gonzalez et al. Genome-wide search reveals a novel GacA-regulated small RNA in Pseudomonas species. BMC Genomics 2008, 9:167; Steglich et al. The challenge of regulation in a minimal photoautotroph: non-coding RNAs in Prochlorococcus. PLoS Genet. 2008, 4:e1000173; Ulve et al. Identification of chromosomal alpha-proteobacterial small RNAs by comparative genome analysis and detection in Sinorhizobium meliloti strain 1021. BMC Genomics 2007, 8:467; Valverde et al. Prediction of Sinorhizobium meliloti sRNA genes and experimental detection in strain 2011. BMC Genomics 2008, 9:416; del Val et al. Identification of differentially expressed small non-coding RNAs in the legume endosymbiont Sinorhizobium meliloti by comparative genomics. Mol Microbiol 2007, 66:1080-1091; Saito et al. Novel small RNA-encoding genes in the intergenic regions of Bacillus subtilis. Gene 2009, 428:2-8; Padalon-Brauch et al. Small RNAs encoded within genetic islands of Salmonella typhimurium show host-induced expression and role in virulence. Nucleic Acids Res 2008, 36:1913-1927; Pichon et al. Small RNA genes expressed from Staphylococcus aureus genomic and pathogenicity islands with specific expression among pathogenic strains. Proc Natl Acad Sci USA 2005, 102:14249-14254; Swiercz et al. Small non-coding RNAs in Streptomyces coelicolor. Nucleic Acids Res 2008, 36:7240-7251; Rasmussen et al. The Transcriptionally Active Regions in the Genome of Bacillus subtilis. Mol Microbiol 2009; Perkins et al. A strand-specific RNA-Seq analysis of the transcriptome of the typhoid bacillus Salmonella typhi. PLoS Genet. 2009, 5:e1000569; Tczuka et al. Identification and gene disruption of small noncoding RNAs in Streptomyces griseus. J Bacteriol 2009, 191:4896-4904; Yoder-Himes et al. Mapping the Burkholderia cenocepacia niche response via high-throughput sequencing. Proc Natl Acad Sci USA 2009, 106:3976-3981; Geissmann et al. A search for small noncoding RNAs in Staphylococcus aureus reveals a conserved sequence motif for regulation. Nucleic Acids Res 2009; Arnvig et al. Identification of small RNAs in Mycobacterium tuberculosis. Mol Microbiol 2009, 73:397-408; Georg et al. Evidence for a major role of antisense RNAs in cyanobacterial gene regulation. Mol Syst Biol 2009, 5:305) were searched. Although some raw predictions of a previous report (Livny et al. High-throughput, kingdom-wide prediction and annotation of bacterial non-coding RNAs. PLoS One 2008, 3:e3197) overlap some of the RNA motifs, these raw predictions have never been subjected to detailed evaluation. Additionally, extensive Google searches for genes associated with crcB RNAs revealed that one of the 358 raw predictions of conserved elements on the RibEx web server (Abreu-Goodger et al. RibEx: a web server for locating riboswitches and other conserved bacterial regulatory elements. Nucleic Acids Res 2005, 33:W690-692) overlaps several of the crcB RNAs disclosed herein. This conserved element was called RLE0038, and was not previously subjected to detailed evaluation. It has not yet been determined if there are other coinciding predictions on this web server because its data are not available in a machine-readable format.

v. In-Line Probing Experiments

RNA constructs were prepared by in vitro RNA transcription RNA using T7 RNA polymerase and the appropriate DNA templates that were created by overlap extension of synthetic DNA oligonucleotides using SuperScript II reverse transcriptase (Invitrogen) as instructed by the manufacturer. RNA transcripts were purified using denaturing (8 M urea) polyacrylamide gel electrophoresis (PAGE). RNAs were eluted from the gel, dephosphorylated using alkaline phosphatase and 5′ radiolabeled with [γ-32P] using methods reported previously (Wang et al. Riboswitches that sense S-adenosylhomocysteine and activate genes involved in coenzyme recycling. Mol Cell 2008, 29:691-702). 5′ 32P-labeled fragments resulting from in-line probing reactions were subjected to denaturing PAGE, imaged and analyzed as previously described (Wang et al. Riboswitches that sense S-adenosylhomocysteine and activate genes involved in coenzyme recycling. Mol Cell 2008, 29:691-702).

vi. Equilibrium Dialysis Experiments

Equilibrium dialysis experiments were conducted in a Dispo-Equilibrium Biodialyzer (The Nest Group, Inc., Southboro, Mass., USA), which is comprised of two chambers (A and B) separated by a 5,000 kDa MW cut-off membrane. Chamber A was loaded with 20 μl solution of 500 nM ³H-SAM, and Chamber B was loaded with 20 μM of the specified RNA in a buffer containing 50 mM MOPS (pH 7.2 at 20° C.), 20 mM MgCl₂, and 500 mM KCl. The chambers were equilibrated at 25° C. for 10 hours before a 3 μl aliquot was removed from each chamber. Radioactivity of the aliquots was measured by a liquid scintillation counter. Each experiment was repeated three times, and average B/A values and standard deviations were calculated.

### B. Example 3

### Discovery of 104 Structured RNAs from Bacterial and Archaeal Genomes and Metagenomes Using Comparative Genomics

1. Background

i. Applicability of the Computational Pipeline to Find Cis-Regulatory RNAs

A previous report aligned the potential 5′ UTRs of homologous protein-coding genes (Yao et al. A computational pipeline for high-throughput discovery of cis-regulatory noncoding RNA in prokaryotes. PLoS Comput. Biol. 2007, 3:e126; Weinberg et al., 2007). This pipeline was thus designed to detect RNA motifs that are frequently in the potential 5′ UTRs of homologous genes. These were called “gene-associated” motifs. By contrast, the new pipeline compares (by nucleotide BLAST) the sequences of IGRs without regard for the type of protein-coding gene residing nearby. The new pipeline is thus directed at finding RNA motifs that are not gene-associated, i.e., are “gene-independent” motifs. Using this new pipeline, we did indeed find many gene-independent motifs, but we additionally found many gene-associated motifs, e.g., the insiK motif It may seem surprising that gene-associated motifs like msiK were not detected by the previous pipeline, given that the previous pipeline was designed to find such motifs. The following factors can contribute to the increase in motifs discovered by the new pipeline, including gene-associated motifs:

a. Newly Released Genome Sequence Data Facilitates the Discovery of Motifs that are Relatively Uncommon.

For example, the msiK motif is derived from a very compelling alignment produced by our newer pipeline, whereas the previous pipeline produced an unpromising prediction. This is most likely due to the fact that several additional genomes of Actinobacteria are now available, which provided more msiK motif representatives and resulted in a more convincing consensus sequence and secondary structure model. Similarly, the SAM-Chlorobi motif exhibits covariation only with the 11 Chlorobi genomes now available. It was also observed that the older pipeline failed to detect SAM-III riboswitches (Fuchs et al. The S(MK) box is a new SAM-binding RNA for translational regulation of SAM synthetase. Nat. Struct. Mol. Biol. 2006, 13:226-233), because these riboswitches often contain long and variable-length loops that make identification of the surrounding stem difficult for CMfinder. The pipeline now easily finds SAM-III riboswitches because many genome sequences are now available that carry SAM-III riboswitches containing short loops.

b. In Some Instances, Too Many UTRs of a Given Gene Family are Available and Only a Few of these Carry the Motif.

For example, the previous pipeline originally identified SAM-TV riboswitches (Weinberg et al. The aptamer core of SAM-IV riboswitches mimics the ligand-binding site of SAM-I riboswitches. Rna 2008, 14:822-828) based on 3 UTRs out of 54 UTRs of the COG0520 family sequenced from in Actinobacteria at the time of our analysis. Thus, most input data in this sequence cluster did not contain the motif. In contrast, the sequence clustering method in the current pipeline will likely partition the three SAM-IV RNAs into a different cluster from the other COG0520 UTRs, which reduces spurious sequences in the cluster. It should be noted, however, that one drawback to BLAST-based sequence clustering is that the accuracy of BLAST searches accuracy may be limited. The frequent decision in the present work to group bacteria at the level of order, rather than the more-broad phylum or class, also can help to reduce spurious sequences in clusters.

c. The Use of Environmental Sequences Helped to Find RNAs that are not Well Represented in Organisms Whose Genomes have been Fully Sequenced.

For example, representatives of SAM-I/IV riboswitches are present in RefSeq, but these few representatives are diluted among unrelated phyla, making their discovery using comparative sequence analysis unlikely. Fortunately, SAM-I/IV riboswitches are common in environmental sequences. A pipeline independent of protein-coding genes is helpful for the analysis of environmental sequences, since gene annotation is difficult when only fragmentary sequences are available.

d. Some Protein Coding Regions are Poorly Annotated, and so Clustering of IGRs Based on Gene Homology is Hindered.

For example, the yjdF motif is almost always upstream of homologous yjdF genes, but these poorly annotated genes are not presented as a conserved domain in the Conserved Domain Database. Therefore, in the context of the previous pipeline, most yjdF motif representatives could not have been identified as residing upstream of homologous genes.

ii. Naming RNA Motifs

Relatively little is known about most of the new-found motifs, but it is believed that it is useful to give them a mnemonic name that reflects some current knowledge of the RNA, its source, or its associated genes. Thus, motifs present only in metagenome data are named after the environment from which they were identified, e.g., “whalefall-1 motif”. Similarly, some motifs are named after their exclusive or predominant taxon, e.g., “Bacteroidales-1 motif”. Cis-regulatory RNA motifs that appear to regulate a variety of heterologous gene families, are named after a single example gene, e.g., “crcB motif”. When the precise biological roles of these RNAs are better understood, it is recommended that the class be renamed to more accurately reflect their functions. For example, the SAM/SAH riboswitch identified in this work was originally named the metK-Rhodobacter motif, before its binding to ligands was confirmed and its riboswitch function was inferred by its gene association and its proximity to expression platforms.

2. Experimental Analysis of Sam Binding by SAM/SAH Riboswitches

It is difficult to draw definitive conclusions regarding SAM binding by aptamers that also tightly bind SAH. Since SAM can undergo spontaneous demethylation, all SAM samples will contain at least some SAH, and this contaminating by-product will increase with aging of the sample. Therefore, the K_{D} reported for SAM could largely reflect the binding of contaminating SAH.

To address this issue, two experiments were performed that indicate that SAM/SAH riboswitches do bind SAM. First, the binding of several close analogs of SAM were examined (FIG. 1A), and all but one of these analogs are bound by SK209-52 RNA with K_{D} values within 10 fold of that measured for SAH. This indicates that SAM/SAH RNAs cannot strongly discriminate against compounds like SAM that carry additional chemical groups on the thioether linkage of SAH. Therefore, these data indicate that SAM/SAH RNAs bind SAM with an affinity that is biologically relevant.

Another experiment used a strategy based on equilibrium dialysis that was previously applied to the analysis of SAH riboswitches (Wang et al. Riboswitches that sense S-adenosylhomocysteine and activate genes involved in coenzyme recycling. Mol Cell 2008, 29:691-702). For these experiments, SAM was obtained with radioactive ³H in its methyl group. When this ³H-SAM degrades spontaneously, it will lose the methyl group, resulting in non-radioactive SAH. In these experiments, two chambers called “A” and “B” are separated by a membrane with a 5,000 kDa molecular weight cutoff. Small molecules like SAM and SAH can pass through this membrane, but RNA molecules cannot. ³H-SAM is placed in chamber A, while SK209-52 RNA is placed in chamber B. If SK209-52 RNA binds SAM, more ³H-SAM will be found in chamber B than in chamber A, because of its association with the RNA. The relative amounts of radioactivity between chambers A and B will thus be indicative of SAM binding, but will not reflect SAH binding because the SAH in this experiment is not radioactive. As positive controls, known SAM-binding RNAs called 156 metA (Corbino et al. Evidence for a second class of S-adenosylmethionine riboswitches and other regulatory RNA motifs in alpha-proteobacteria. Genome Biol. 2005, 6:R70) and 62 metY (Poiata E, Meyer MM, Ames T D, Breaker R R: A variant riboswitch aptamer class for S-adenosylmethionine common in marine bacteria. Rna 2009, 15:2046-2056) were used. Finally, when a point mutation called “A48U” was applied to SK209-52 RNA, the mutated RNA exhibited a drastically reduced ability to bind SAM when compared to the wild-type RNA.

The results show that significantly more radioactivity is present in chamber B when the known SAM-binding RNAs or when SK209-52 RNA is applied to chamber A (FIG. 1B). Therefore, SK209-52 RNA is binding ³H-SAM. As expected, when the A48U mutant is applied to chamber A, the amounts of radioactivity in the two chambers are roughly equal, showing that this mutant has a greatly reduced ability to bind SAM.

3. Additional Discussion of RNA Motifs

The text below provides comments on each motif identified in the current study. Notable characteristics derived by examining the sequence and structural features, or derived by literature analysis of the associated genes is presented. All motif consensus diagrams are shown in FIGS. 9-28.

i. aceE Motif

The aceE motif is found in the potential 5′ UTRs of aceE genes in Pseudomonas species. The aceE gene encodes pyruvate dehydrogenase, which can use pyruvate to synthesize coenzyme A that then participates in the citric acid cycle. Growth of P. aeruginosa in anaerobic conditions with nitrite as the sole electron acceptor leads to lower levels of aceE expression. However, this condition also leads to lower expression of other genes related to the citric acid cycle (Platt et al. Proteomic, microarray, and signature-tagged mutagenesis analyses of anaerobic Pseudomonas aeruginosa at pH 6.5, likely representing chronic, late-stage cystic fibrosis airway conditions. J Bacteriol 2008, 190:2739-2758) that do not have predicted aceE RNAs. On the other hand, expression of accE in a P. acruginosa strain isolated from a cystic fibrosis patient differed from that of a strain isolated from a burn victim, yet other citric acid cycle genes were not differently regulated in this case (Sriramulu et al. Proteome analysis reveals adaptation of Pseudomonas aeruginosa to the cystic fibrosis lung environment. Proteomics 2005, 5:3712-3721).

ii. Acido-1 Motif

The Acido-1 motif consists of two hairpins, with high sequence conservation in the linker between the hairpins, and in the terminal loop of the 3′ hairpin. Given its lack of association with genes, the motif appears to act in trans. Although only four sequences are predicted to have the Acido-1 motif, there is significant covariation. The motif appears to be restricted to Acidobacteria.

iii. Acido-Lenti-1 Motif

The Acido-Lenti-1 motif is found in the phyla Acidobacteria and Lentisphaerae. In Lentisphaerae, it is sometimes located near group 11 introns.

iv. Actino-pnp Motif

Actino pnp motif representatives are predicted only in Actinobacteria. They are consistently in the potential 5′ UTRs of genes annotated as encoding a 3′-5′ exoribonuclease, such as polynucleotide phosphorylase or RNase PH. RNA leader structures have been reported upstream of polynucleotide phosphorylase genes in enterobacteria such as E. coli where they reduce gene expression when enzyme levels are high (Jarrige et al. PNPase autocontrols its expression by degrading a double-stranded structure in the pnp mRNA leader. Embo J 2001, 20:6845-6855). Since the enterobacterial pnp leader RNA does not appear to be structurally related to the Actino pnp motif, it is thought that the Actino pnp is a distinct structural solution to regulate expression of the enzyme.

v. asd motif

The asd motif is often, but not always, in potential 5′ UTRs of genes, which indicates a cis-regulatory role. However, in two cases, non-homologous genes are downstream of an asd RNA, in the wrong orientation for the RNA to be in their 5′ UTRs.

Also, downstream of the motif in Streptococus mutans is a conserved transcription terminator, followed by a strong promoter that is, in turn, followed by the asd gene (Cardineau et al. Nucleotide sequence of the asd gene of Streptococcus mutans. Identification of the promoter region and evidence for attenuator-like sequences preceding the structural gene. J Biol Chem 1987, 262:3344-3353). In S. mutans, no significant modulation in gene expression was observed in response to changing levels of amino acids for whose synthesis Asd participates (i.e., lysine, threonine, and methionine) (Cardineau et al. Nucleotide sequence of the asd gene of Streptococcus mutans. Identification of the promoter region and evidence for attenuator-like sequences preceding the structural gene. J Biol Chem 1987, 262:3344-3353). In Streptococcus pneumoniae D39, a CodY binding site was predicted in between an asd RNA and the downstream asd gene (Hendriksen et al. CodY of Streptococcus pneumoniae: link between nutritional gene regulation and colonization. J Bacterio12008, 190:590-601). CodY binds double-stranded DNA when there are high concentrations of branched-chain amino acids (BCAAs, i.e., leucine, isoleucine or valine). This binding event typically represses genes involved in synthesizing BCAAs, and repression was demonstrated using microarrays, protein expression and DNA binding. Thus, this asd gene is regulated in response to BCAAs, in a manner unrelated to the upstream asd RNA. If asd RNAs are cis-regulatory elements, they presumably sense a signal other than high BCAA concentrations.

Given these characteristics, the asd motif can correspond to a non-coding RNA at least in some instances. This is consistent with the fact that there is a transcription terminator downstream of it, and potential base pairing that can serve as an antiterminator that would respond to metabolite binding or other signals is not observed. Interestingly, genes upstream of asd RNAs are always transcribed in the same direction as the RNA, and the distance between these upstream genes and the asd RNA is always within about 200 base pairs, although it is not clear whether this observation is biologically relevant, or merely a coincidence.

vi. atoC Motif

Motif representatives are in potential 5′ UTRs of genes encoding domains with oxidoreductase activity, response regulators containing DNA-binding domains, or FolK (folate synthesis).

vii. Bacillaceae-1 motif

This RNA likely functions in trans and is found in many gene contexts. In several cases is adjacent to a ribosomal RNA operon. The terminal loops of its two hairpins both have the consensus RUCCU, which is indicative of binding to a homodimeric protein.

vIii. Bacillus-Plasmid Motif

The Bacillus-plasmid motif occurs in species within the genera Bacillus and Lactobacillus species, and is usually found in plasmids. In a notable exception, the motif is found upstream of the ydcS gene in B. subtilis. The motif consists of a single hairpin where the 5′ and 3′ regions of the terminal loop are highly conserved. The interior part of the terminal loop is not highly conserved and can be as long as 38 nucleotides. Bacillus-plasmid RNA motifs are typically upstream of genes annotated as repA or mobilization element genes, although the gene is typically 200-300 nucleotides 3′ of the RNA structure. Nonetheless, this arrangement is indicative of a cis-antisense RNA that can regulate plasmid copy number (Kim et al. Copy-number of broad host-range plasmid R1162 is regulated by a small RNA. Nucleic Acids Res 1986, 14:8027-8046), even though the motif does not resemble a known RNA of this type.

ix. Bacteroid-trp Leader Motif

This motif apparently controls trpB and trpE genes in Bacteroidetes, which are involved in tryptophan synthesis. The motif contains a region of two or more conserved tryptophan codons (UGG), and therefore is presumably a peptide leader that detects low levels of tryptophan by attenuation (Vitreschak et al. Attenuation regulation of amino acid biosynthetic operons in proteobacteria: comparative genomics analysis. FEMS Microbiol Lett 2004, 234:357-370). Although tryptophan attenuation leaders are known in Proteobacteria, none have been reported in Bacteroidetes. A consensus diagram of the bacteroid-trp leader was not created since it is a loosely conserved hairpin.

x. Bacteroidales-1 motif

Upstream sequences that conform to consensus promoters for Bacteroides (Bayley et al. Analysis of cepA and other Bacteroides fragilis genes reveals a unique promoter structure. FEMS Microbiol Lett 2000, 193:149-154) allow for prediction of an approximate transcription start site for this RNA.

xi. Bacteroides-1 Motif

The Bacteroides-1 motif may act in trans. However, it is typically downstream of genes that are associated with synthesis of exopolysaccharides. Therefore the RNA motif can regulate expression of the upstream genes by acting within their 3′ UTRs.

xii. Bacteroides-2 Motif

This RNA is found almost exclusively in human gut bacterial sequences, except for the one species Bacteroides capillosus ATCC 29799. The genome sequence of this species was released in 2007, and metagenomics data was essential for identification by our bioinformatics pipeline.

xiii. Burkholderiales-1 Motif

The Burkholderiales-1 motif is present in some species in the order Burkholderiales. It is sometimes present in many copies in the same genome (e.g., 33 copies in Polaromonas sp. JS666). The genes immediately downstream of Burkholderiales-1 RNAs usually are oriented in the opposite direction. This arrangement would be expected if the Burkholderiales-1 motif were the reverse complement of a rho-independent transcription terminator. However, the motif's reverse complement lacks the expected polyuridine stretch.

xiv. c4 antisense RNA motif

The c4 antisense RNA was previously identified in P1 and P7 phages of E. coli (Citron et al. The c4 repressors of bacteriophages P1 and P7 are antisense RNAs. Cell 1990, 62:591-598). A motif was identified within Pseudomonadales, and established many homologs in other Proteobacteria, as well as several phages, including phage P1. The predicted structure is supported by covariation, and is consistent with the structure that was proposed based on the RNA present in P1 phage (Citron et al. The c4 repressors of bacteriophages P1 and P7 are antisense RNAs. Cell 1990, 62:591-598). The alignment indicates that c4 antisense RNA is found in the genomes of many bacteria, presumably from phage integration events. It was also observe that the terminal loop of P2 is often the stable tetraloop GNRA, UNCG or CUUG: out of 492 unique C4 antisense RNA P2 sequences, 122 terminate in GNRA, 233 in UNCG and 19 in CUUG. The other sequences present in the terminal loop of P2 can also have high stability. In several cases, the 3′ half of the P1 stem overlaps the 5′ half of a predicted transcription terminator hairpin. It is possible that c4 antisense RNA sometimes functions as a cis-regulatory element, although these predicted transcription terminators may simply function constitutively.

xv. c4 antisense RNA a1b1 motif

The c4 antisense RNA (described above) is believed to regulate ant genes by binding to complementary RNA sites, one of which overlaps a potential ribosome-binding site (Citron et al. The c4 repressors of bacteriophages P1 and P7 are antisense RNAs. Cell 1990, 62:591-598). c4 antisense RNA has two regions, called a′ and b′, that can base pair with sites designated a1, b1 and a2, b2. The a2, b2 sites are upstream of the ant gene and downstream of the c4 RNA. The a1, b1 sites are upstream of the c4 antisense RNA itself. It was proposed that the a1, b1 sites can compete with the a2, b2 sites for binding c4 RNA, and thereby free the a2, b2 sites, in turn allowing ant expression (Citron et al. The c4 repressors of bacteriophages P1 and P7 are antisense RNAs. Cell 1990, 62:591-598).

A motif was found that encompasses the a1 site, and is immediate 5′ to the b1 site. The motif consists of two hairpins whose structure is well supported by covariation. A third stem is sometimes found that connects a region several nucleotides 5′ to P1 with a sequence overlapping the 3′ part of P2 (not shown). Although this stem exhibits covariation when it is found, it is absent from many sequences, including those in DNA isolated from purified phage particles. No conserved secondary structure was previously proposed for the a1, b1 sites, but conserved structures are known for other targets of antisense RNAs, such as the traJ-I RNA mentioned below (see traJ-II motif).

xvi. Chlorobi-1 Motif

Chlorobi-1 RNAs are found only in the phylum Chlorobi and consist of two hairpins, with most nucleotide conservation found in their terminal loops. All known Chlorobi-1 RNAs have predicted transcription terminators downstream.

xvii. Chlorobi-RRM Motif

The Chlorobi-RRM motif is consistently in the potential 5′ UTRs of genes predicted to encode an RNA-binding protein, which indicates that it serves an auto-regulatory role for the gene.

xviii. Chloroflexi-1 Motif

The Chloroflexi-1 motif is present in three copies in Chloroflexus aggregans, a species in the phylum Chloroflexi. Although there is good covariation, the few sequences available make it difficult to assess the significance of the covariation. The fact that the three representatives are located near to one another on the chromosome indicates that the motif can be associated with a repetitive element.

xix. Clostridiales-1 Motif

The Clostridiales-1 motif is a large four-stem structure that is very common in DNA sequences from microbes in the human gut, and is present in several bacteria in the order Clostridiales. The structure seems to be less conserved when predicted homologs are incorporated using sensitive “local” mode covariance model searches (Eddy S R: A memory-efficient dynamic programming algorithm for optimal alignment of a sequence to an RNA secondary structure. BMC Bioinformatics 2002, 3:18), but even within the sequences that are similar to one another, there is significant covariation.

xx. Collinsella-1 Motif

There are only six representatives identified for the Collinsella-1 motif Five are from environmental samples of the human gut, and the remainder is found in Collinsella aerofaciens ATCC 25986. The P3 stem is well supported by covariation, however the P1 and P2 stems is less supported.

xxi. crcB Motif

The structural characteristics and genetic distribution of this motif arc strongly indicative of riboswitch aptamer function. When considering ligands, two stress conditions were considered under which cells up-regulate some of the genes presumably controlled by crcB RNAs. However, these conditions do not appear to account for all genes associated with the riboswitch. Acidic pH stress typically induces K+ or Na+ transporters (Leaphart et al. Transcriptome profiling of Shewanella oneidensis gene expression following exposure to acidic and alkaline pH. J Bacteriol 2006, 188:1633-1642), though unfortunately also many other genes not associated with crcB RNAs. The response to oxidative stress involves upregulating two genes associated with crcB RNAs, iscU and GTP cyclohydrolase (Storz et al. Oxidative stress. In: Bacterial stress responses Edited by Storz G, Hengge-Aronis R. Washington, D.C.: ASM Press; 2000), but again others not relevant to crcB RNAs.

xxii. Cyano-1 Motif

Some Cyano-1 RNAs in Prochlorococcus marinus MED4 (RefSeq accession NC_{—}005072) are near to noncoding RNAs detected previously, though no conserved secondary structure was proposed for these regions (Steglich et al. The challenge of regulation in a minimal photoautotroph: non-coding RNAs in Prochlorococcus. PLoS Genet. 2008, 4:e1000173). Specifically, Yfr10 is 200 nt distant in one case, Yfr12 is 80 nt distant in one case, Yfr18 is 170 nt distant in one case, and a Cyano-1 RNA overlaps ˜60 nt of the 3′ end of the roughly 250-nt Yfr15.

xxiii. Cyano-2 Motif

The Cyano-2 RNA consists of two structured regions separated by an internal region that has no apparent conserved structure. The sequence GCGA within terminal loops is common, and may form GNRA tetraloops in the subset of Cyano-2 RNAs in which the three immediate-flanking nucleotides both upstream and downstream of the tetraloop can form Watson-Crick base pairs. The second structured region has a highly conserved bulge. This indicates that the motif represents two distinct structures that are functionally associated. Cyano-2 RNAs usually occur in regions without any predicted genes or RNAs for the upstream 1 Kb, which is uncommon among known RNAs.

xxiv. Desulfotalea-1 Motif

The Desulfotalea-1 motif has characteristics of a trans-acting RNA and, in many instances, is located near rRNA operons.

xxv. Dictyoglomi-1 Motif

The Dictyoglomi-1 motif is present in two copies in each of the two species sequenced from the phylum Dictyoglomi. The RNA is consistently in the potential 5′ UTRs of genes, but since it is far from the genes, it is unclear whether it represents a cis-regulatory element. The downstream genes are annotated as enzymes that hydrolyze glycosidic bonds. Dictyoglomi-1 RNAs conserve four E-loops, which are often associated with intermolecular interactions (Lee J C: Structural studies of ribosomal RNA based on cross-analysis of comparative models and three-dimensional crystal structures. Austin, Tex.: University of Texas; 2003. Dissertation). The structure of the Dictyoglomi-1 motif is compromised by the lack of availability of diverged homologs. Detection of diverged homologs may reveal covariation within a longer structure.

xXvi. Downstream-Peptide Motif

The gene P9301_{—}07111 in Prochlorococcus marinus str. MIT 9301 is apparently regulated by a Downstream-peptide RNA and is among the 20 most highly expressed genes in this organism, although nitrogen regulation was not tested in this case (Frias-Lopez et al. Microbial community gene expression in ocean surface waters. Proc Natl Acad Sci USA 2008, 105:3805-3810). The peptides encoded downstream of Downstream-peptide RNAs are difficult to align, and might not all be homologous. However, out of 429 ORFs that were not truncated by short sequencing reads, 360 encode a peptide with the amino acid motif YRG and 207 have the longer LTYRG. The YRG motif was indicated by previous analysis of ORFs associated with the yfr6 motif (Axmann et al. Identification of cyanobacterial non-coding RNAs by comparative genome analysis. Genome Biology 2005, 6:R73), which corresponds to a previously predicted noncoding RNA that overlaps Downstream-peptide RNAs. It is known that PII proteins (also called GlnB) contain a conserved YRGxxY (SEQ ID NO:30) motif and are involved in regulating genes in nitrogen metabolism (Forchhammer K: Global carbon/nitrogen control by PII signal transduction in cyanobacteria: from signals to targets. FEMS Microbiol Rev 2004, 28:319-333). 355 out of 429 Downstream-peptides contain this YRGxxY (SEQ ID NO:30) arrangement. In most bacteria, the second Y is uridylated, though in Cyanobacteria, a serine residue after the G is phosphorylated. The peptides associated with Downstream-peptide RNAs can function in a way that is related to the phosphorylation of P_{H} proteins, perhaps as a decoy.

A distinct ncRNA called yfr14 was detected that overlaps the reverse complement of yfr6 (Steglich et al. The challenge of regulation in a minimal photoautotroph: non-coding RNAs in Prochlorococcus. PLoS Genet. 2008, 4:c1000173). Therefore, these yfr14 RNAs in turn also overlap Downstream-peptide RNAs.

xxvii. Flavo-1 Motif

All but a few Flavo-1 RNAs are found in Flavobacteria, with others found in the same phylum, Bacteroidetes, or the related Spirochaetes.

xxviii. fixA Motif

The fixA motif is consistently located in potential 5′ UTRs of fixA genes in certain Pseudomonas species. The fixA gene and the downstream fixB gene encode an enzyme required for carnitine reduction under anaerobic conditions (Walt A, Kahn M L: The fixA and fixB genes are necessary for anaerobic carnitine reduction in Escherichia coli. J Bacteriol 2002, 184:4044-4047).

xxix. gabT Motif

The gabT motif is found in the potential 5′ UTRs of gabT genes in the genus Pseudomonas. The motif is located downstream of gabD genes. Thus, the gene organization is always gabD, then the RNA, then gabT. In microarray experiments in various Pseudomonas species, gabD and gabT genes that are associated with gabT RNAs were shown to be induced by agmitine, putrescine, GABA (Chou et al. Transcriptome analysis of agmatine and putrescine catabolism in Pseudomonas aeruginosa PAO1. J Bacteriol 2008, 190:1966-1975), lysine, delta-aminovalerate (Espinosa-Urgel et al. Expression of a Pseudomonas putida aminotransferase involved in lysine catabolism is induced in the rhizosphere. Appl Environ Microbiol 2001, 67:5219-5224) and iron depletion (Ochsner et al. GeneChip expression analysis of the iron starvation response in Pseudomonas aeruginosa: identification of novel pyoverdine biosynthesis genes. Mol Microbiol 2002, 45:1277-1287). In all cases, both gabD and gabT genes were induced by approximately the same amount, indicating that they form an operon. In the case of agmatine and putrescine, the region upstream of gabD—which does not contain the RNA motif—was fused to a lacZ reporter, and yielded approximately the same induction as the genes. So, the regulation in response to the above metabolites can be caused by an element in the region upstream of gabD.

GabT is annotated as a transaminase, and GabD as a dehydrogenase, but they appear to operate on multiple substrates in multiple pathways. In the catabolism of agmatine, putrescine and other metabolites, GabT catalyzes the transamination of gamma-aminobutanoate (GABA) to form succinate semialdehyde, which is then dehydrogenated to succinate by GabD, where it feeds into the citric acid cycle (Chou et al. Transcriptome analysis of agmatinc and putrescinc catabolism in Pseudomonas acruginosa PAO1. J Bacteriol 2008, 190:1966-1975). GabD was shown to catalyze the expected reaction in vitro, and both genes are induced by GABA, the substrate of GabT. However, the genes also play a role in lysine degradation. In this pathway, the gene product annotated as GabT transaminates delta-aminovalerate, which is dehydrogenated to glutarate by the annotated GabD. In support of this proposed activity, the combined proteins catalyze the expected two-step reaction in vitro (Yamanishi et al. Prediction of missing enzyme genes in a bacterial metabolic network. Reconstruction of the lysine-degradation pathway of Pseudomonas aeruginosa. Febs J2007, 274:2262-2273), and both are induced by delta-aminovalerate (Espinosa-Urgel M, Ramos J L: Expression of a Pseudomonas putida aminotransferase involved in lysine catabolism is induced in the rhizosphere. Appl Environ Microbiol 2001, 67:5219-5224), their starting metabolite.

If gabT RNAs are cis-regulatory elements, they are presumably regulating gabT in a manner independent of gabD. In most gabT RNAs, a second hairpin is located 3′ of the primary hairpin. This stem appears to overlap the Shine-Dalgarno sequence of the downstream gabT genes, although this part of the stem does have a few non-canonical pairs in some sequences. In two cases, this second hairpin is absent, and the apparent Shine-Dalgarno sequence is located six nucleotides 3′ of the primary hairpin. This arrangement indicates mechanisms by which the gabT gene can be regulated. Note that all gabT RNAs are upstream of gabT genes, so both gabT RNAs with and without the second hairpin should affect gene expression in the same direction (up or down) under similar changes in cellular conditions. Thus, for example, if the second hairpin sequesters the ribosome binding site given high concentrations of an effector molecule, the gabT RNAs lacking the second hairpin should also somehow sequester the ribosome binding site under these conditions.

xxx. Gamma-cis-1 Motif

The Gamma-cis-1 motif is found in a variety of γ-proteobacteria. The motif as depicted (FIG. 15) is a three-stem junction, but the pairing in the P2 stem is often weak. Overall, although there is some evidence of covariation among Gamma-cis-1 RNAs, it is not clear whether these sequences correspond to structured RNAs.

xxxi. GUCCY Hairpin Motif

The GUCCY hairpin is a short hairpin flanked by the consensus sequences GUC and CY. Due to its small size, there is a high risk of false positives in homology searches. Therefore, we were conservative in adding hits as homologs. Also, the difficulty in confidently assigning homologs makes it more difficult to conclude that the motif represents a conserved RNA. It was observed that there is an overrepresentation of genes that are classified as COG2827 nearby. COG2827 genes encode endonucleases containing a URI domain.

xxxii. Gut-1 Motif

The Gut-1 motif is detected only in environmental sequences from the human gut, and not in any sequenced organism.

xxxiii. gyrA Motif

The gyrA motif consists of two hairpins that are generally supported by covariation, and is present in the order Pseudomonadales. The motif is always found in the potential 5′ UTRs of gyrA genes, and therefore it is presumed that it is a regulator of these genes. However, gyrA has been regarded as a housekeeping gene whose expression is constant in many conditions (Vencato et al. Bioinformatics-enabled identification of the HrpL regulon and type Iii secretion system effector proteins of Pseudomonas syringae pv. phaseolicola 1448A. Mol Plant Microbe Interact 2006, 19:1193-1206). The gyrA gene encodes a subunit of DNA gyrase. Mutations in this gene are commonly associated with ciprofloxacin resistance in Pseudomonas (Bonomo et al. Mechanisms of multidrug resistance in Acinetobacter species and Pseudomonas aeruginosa. Clin Infect Dis 2006, 43 Suppl 2:S49-56). The gyrA motif is also sometimes present upstream of dnaJ genes, which encode chaperones.

xxxiv. hopC Motif

The method by which hopCB transcript abundance is regulated is unknown, but it was speculated that a homopolymeric tract of 13 thymidines can be involved (McGowan et al. Promoter analysis of Helicobacter pylori genes with enhanced expression at low pH. Mol Microbiol 2003, 48:1225-1239). This tract is located upstream of the transcription start site, and does not overlap the hopC motif.

xxxv. icd Motif

The icd motif is found in Pseudomondales, in the potential 5′ UTRs of icd genes, which encode isocitrate dehydrogenase. This arrangement indicates that it is a cis-regulatory element. However, the modest covariation makes it ambiguous as to whether the icd motif is a genuine RNA.

xxxvi. JUMPstart sequence motif

The JUMPstart sequence is a conserved 39 bp element upstream of operons whose protein products are involved in the synthesis of polysaccharides (Hobbs et al. The JUMPstart sequence: a 39 bp element common to several polysaccharide gene clusters. Mol Microbiol 1994, 12:855-856). Experiments on the promoter region of the Escherichia coli O7-specific lipopolysaccharide gene cluster confirmed that the conserved JUMPstart sequence is in the 5′ UTR of the mRNA (Marolda et al. Promoter region of the Escherichia coli O7-specific lipopolysaccharide gene cluster: structural and functional characterization of an upstream untranslated mRNA sequence. J Bacteriol 1998, 180:3070-3079). A stem-loop structure was found that is conserved in many JUMPstart sequences (FIG. 16). No conserved RNA structures were previously reported in JUMPstart sequences.

A major feature of the JUMPstart sequence is the ops (operon polarity suppressor) element, which has the consensus GGCGGUAG (Nieto et al. Suppression of transcription polarity in the Escherichia coli haemolysin operon by a short upstream element shared by polysaccharide and DNA transfer determinants. Mol Microbiol 1996, 19:705-713). The ops element enhances transcription of downstream genes—especially distal genes of the operon—when the protein factor RfaH is present (Marolda et al. Promoter region of the Escherichia coli O7-specific lipopolysaccharide gene cluster: structural and functional characterization of an upstream untranslated mRNA sequence. J Bacteriol 1998, 180:3070-3079; Nieto et al. Suppression of transcription polarity in the Escherichia coli haemolysin operon by a short upstream element shared by polysaccharide and DNA transfer determinants. Mol Microbiol 1996, 19:705-713; Leeds et al. Enhancing transcription through the Escherichia coli hemolysin operon, hlyCABD: RfaH and upstream JUMPStart DNA sequences function together via a postinitiation mechanism. J Bacteriol 1997, 179:3519-3527; Wang et al. Expression of the O antigen gene cluster is regulated by RfaH through the JUMPstart sequence. FEMS Microbiol Lett 1998, 165:201-206). Some JUMPstart representatives have an additional partial ops sequence. This partial ops sequence has the consensus GGUAG and overlaps the stem 5′-side and the loop. Deletion of either ops sequence reduced the RfaH-mediated transcription enhancement (Marolda et al. Promoter region of the Escherichia coli O7-specific lipopolysaccharide gene cluster: structural and functional characterization of an upstream untranslated mRNA sequence. J Bacteriol 1998, 180:3070-3079).

There is a diversity of JUMPstart sequences containing the stem loop structure, including most studied JUMPstart sequences. However, a sequence called “hly (pHlyl52)” contains a validated ops element (Wang et al. Expression of the O antigen gene cluster is regulated by RfaH through the JUMPstart sequence. FEMS Microbiol Lett 1998, 165:201-206), but lacks stems in the most typical location (FIG. 16). There is more flexibility in the sequence and structural features that define the motif than presently shown, for example by allowing more distance between the stem and the major ops element. Alternately, the stem-loop structure can function independently of RfaH-mediated transcription enhancement.

xxxvii. Lacto-int Motif

The Lacto-int motif is found upstream of phage integrase genes, though not always in their potential 5′ UTRs. It is present in purified phages and in bacterial genomes, where it is presumably associated with prophage sequences. The motif consists of two hairpins, of which the first is supported by covariation while the second is ambiguous. It is possible that the motif forms an inverted repeat in DNA to facilitate integration or excision.

xxxviii. Lacto-Plasmid Motif

Lacto-plasmid RNAs are typically, but not always, located on plasmids, and are apparently restricted to Lactobacillales. In addition to their location on plasmids, they are sometimes present in apparent prophages.

xxxix. Lacto-rpoB motif

The Lacto-rpoB motif is a hairpin with a highly conserved loop found in the order Lactobacillales. It is in the potential 5′ UTRs of rpoB genes, which encode the β subunit of RNA polymerase.

xl. lactis-Plasmid Motif

Lactis-plasmid representatives are located on plasmids in bacteria in the order Lactobacillales, mostly Lactococcus lactis. The RNA motifs are typically located nearby to repB genes (though not necessarily in the 5′ UTR), although this can simply reflect the limited size of the plasmids. repB genes are involved in the replication of plasmids. Like the Bacillus-plasmid motif, lactis-plasmid RNAs can regulate plasmid copy number (Kim et al. Copy-number of broad host-range plasmid R1162 is regulated by a small RNA. Nucleic Acids Res 1986, 14:8027-8046). However, according to Rfam (Gardner et al. Rfam: updates to the RNA families database. Nucleic Acids Res 2009, 37:D136-140), many of the plasmids containing a lactis-plasmid RNA also contain predicted ctRNA-pND324 RNAs (Rfam accession RF00238) (Duan et al. Involvement of antisense RNA in replication control of the lactococcal plasmid pND324. FEMS Microbiol Lett 1998, 164:419-426), so the lactis-plasmid RNAs may perform another function.

xli. Leu/phe-Leader Motif

Detected only in the species Lactococcus lactis, the leu/phe-leader motif is supported by substantial covariation, and includes an ORF encoding a short peptide. Leu/phe-leader RNAs arc consistently in the potential 5′ UTRs of genes. When the gene is leuB or leuC, the peptide includes are run of three leucine residues. A leucine peptide leader has already been identified in L. lactis, where low concentrations of leucine lead to stalling during translation and ultimately affect transcriptional attenuation (Kok J: Inducible gene expression and environmentally regulated genes in lactic acid bacteria. Antonie Van Leeuwenhoek 1996, 70:129-145). Other leucine peptide leaders were previously identified upstream of a predicted amino acid transporter. Two additional homologous RNAs were detected that had a run of three phenylalanine residues instead of the leucine residues. While one of these is upstream of a hypothetical protein, the other is upstream of aroH, which is predicted to encode 3-deoxy-7-phosphoheptulonate synthase, which catalyzes an early step in the synthesis of phenylalanine, tyrosine and tryptophan. This step can be regulated via phenylalanine levels. All leu/phe-leader RNAs identified have predicted transcription terminators where the 5′ part of the terminator stem overlaps the 3′ part of the P4 stem in the leader motif.

xlii. Lnt Motif

The Lnt motif is found in Chlorobi, where it is in the potential 5′ UTRs of genes predicted to encode apolipoprotein N-acyltransferases. The RNA structure consists of a single six-bp stem that is modestly supported by covariation. The terminal loop for this stem is not well conserved, but the region 3′ to it has significant sequence conservation. One representative of the Lnt motif has a G-U base pair, which on the reverse complement would be A-C. G-U wobble pairs are more energetically favorable, and therefore help identify the proper orientation for putative RNA motifs. This fact argues that the Lnt motif is more likely to be predicted on the correct strand. However, on the reverse strand, the motif is very close to the predicted start codon of bacteriochlorophyll A genes.

xliii. Methylobacterium-1 RNA Motif This motif is largely found in marine metagenome sequences, although it is also present in Methylobacterium sp. 4-46, a kind of α-proteobacteria. The motif consists of three hairpins.

xliv. Moco-II Motif

The previously discovered Moco RNA element is a riboswitch that is associated with genes involved in biosynthesis and utilization of molybdenum cofactor (Moco) and tungsten cofactor (Weinberg et al., 2007; Regulski et al. A widespread riboswitch candidate that controls bacterial genes involved in molybdenum cofactor and tungsten cofactor metabolism. Mol Microbiol 2008, 68:918-932). The newly found Moco-II motif is also associated with Moco-related genes, including a molybdenum-binding domain (MoeA) and nitrate reductase. However, only 8 representatives of the Moco-II motif are known. Seven representatives are in 6-proteobacteria, with one diverged example in the β-proteobacteria division. The Moco-II motif consists of a hairpin with a conserved internal loop, and the hairpin is typically adjacent to a transcription terminator, indicating an expression platform. The structure is supported by modest covariation, and by the motif's presence upstream of genes that are not homologous, but are functionally related via Moco.

xlv. mraW Motif

The mraW motif is found in a wide variety of Actinobacteria such as Mycobacterium. It is a hairpin with three moderately conserved stems, and poorly conserved internal loops. The terminal loop has a highly conserved CUUCCCC sequence. Motif representatives are always in front of a predicted mraW gene, and appear to control an operon with a highly conserved series of genes: mraW, a hypothetical membrane protein and ftsI, typically followed by one or more mur genes. ftsI and mur genes are known to be involved in peptidoglycan synthesis (Wijayarathna et al. Isolation of ftsI and murE genes involved in peptidoglycan synthesis from Corynebacterium glutamicum. Appl Microbiol Biotechnol 2001, 55:466-470), so presumably the mraW motif is involved in the regulation of this process.

xlvi. msiK Motif

An msiK null mutant was identified as S. lividans 10-164 (Hurtubise et al. A cellulase/xylanase-negative mutant of Streptomyces lividans 1326 defective in cellobiose and xylobiose uptake is mutated in a gene encoding a protein homologous to ATP-binding proteins. Mol Microbiol 1995, 17:367-377). Strain 10-164 grows poorly on cellobiose, maltose and other sugars, but its growth on glucose is similar to wild type (Schlösser et al. The Streptomyces ATP-binding component MsiK assists in cellobiose and maltose transport. J Bacteriol 1997, 179:2092-2095). It also imports glucose at wild-type levels, but has a reduced ability to import other sugars (Hurtubise et al. A cellulase/xylanase-negative mutant of Streptomyces lividans 1326 defective in cellobiose and xylobiose uptake is mutated in a gene encoding a protein homologous to ATP-binding proteins. Mol Microbiol 1995, 17:367-377). In wild-type S. lividans or S. reticuli cells, concentrations of the MsiK protein are highest when cells are grown on cellobiose, lower for other sugars, and very low when cells are grown on glucose. In contrast, S. lividans 10-164 expresses MsiK at very high levels for all sugars tested, including glucose (Schlösser et al. The Streptomyces ATP-binding component MsiK assists in cellobiose and maltose transport. J Bacteriol 1997, 179:2092-2095).

msiK RNA motis can directly or indirectly sense glucose levels. For this, the presence of this fundamental sugar would imply that the import of other sugars is not necessary. However, the above experimental results imply that glucose is imported into strain 10-164 cells, but does not repress MsiK expression. This indicates that msiK RNAs can sense a small molecule that indicates sufficient levels of some sugars, but whose concentrations are not increased by glucose. Alternatively, when MsiK protein levels are sufficient to supply an ATPase domain to the various ABC sugar importers, excess MsiK binds the msiK RNA in its 5′ UTR and thereby represses further MsiK expression. In this model, the mutated MsiK in strain 10-164 is unable to bind to the RNA, leading to constitutive expression. It has been hypothesized for a different ATPase that it can repress its expression only in the ATP-bound state, as this state is most likely when no substrate is being transported (Panagiotidis et al. The ATP-binding cassette subunit of the maltose transporter MalK antagonizes MalT, the activator of the Escherichia coli mal regulon. Mol Microbiol 1998, 30:535-546), so this can explain why the 10-164 mutation hinders both ATPase activity and the proposed RNA-binding function. A related model is that MsiK binds to another, unknown protein, which in turn binds to the msiK RNA. The RNA motif may be a binding site for a receptor protein that senses a change in cellular conditions).

xlvii. nuoG Motif

This motif is found in enterobacteria upstream of nuoG genes, which encode a subunit of ubiquinone reductase. The downstream genes also encode subunits of this enzyme and presumably belong to the same operon. Since the motif is very small, there is a risk that homologs were not detected that would reveal that the structure is not conserved, as these homologs can have insignificant E-values in homology searches. The motif is present in most sequenced enterobacteria including the genus Escherichia, but not the closely related Salmonella. When the region upstream of a predicted nuoG gene in Salmonella typhimurium LT2 (an arbitrarily selected organism) was inspected, sequences that loosely match the nuoG RNA motif were found, but could not fold into the consensus structure. However, again, since the motif is very small, it is unclear whether sequence-only matches in Salmonella are significant. It is possible that the Salmonella sequence is unrelated to the nuoG motif. Regardless, within the nuoG RNA motifs, considerable covariation is seen, despite sequence and length constraints that would reduce the possibility of spurious base pairing. Thus, it is ambiguous whether the nuoG motif represents an RNA structure.

xlviii. Ocean-V Motif

The Ocean-V motif is found in only three sequences from marine environmental DNA samples. Although it is difficult to confidently assess its assignment as a structured RNA, even among these three sequences there is some covariation. The Ocean-V motif is not detected in any sequenced organism.

xlix. Ocean-VI Motif

The Ocean-VI motif is found frequently in marine environmental sequences, but is not detected in any known sequenced organism. The putative stems are highly conserved, and as a result there is only modest covariation. Ocean-VT RNAs are sometimes located downstream of non-homologous genes involved in methionine metabolism (metA, metK), but the upstream gene is often in the opposite orientation, so it is not clear that there is any functional association with methionine.

I. pan Motif

Although most pan RNAs occur in tandem pairs, those in δ-proteobacteria typically occur singly (data not shown). Note that there can be a technical bias in favor of pan RNAs containing two hairpins, since they are easier to find in homology searches.

Ii. Pedo-Repair Motif

The Pedo-repair motif is found in five instances in Pedobacter sp. BAL39, and in no other available sequence. The Pedo-repair motif is a three-stem junction that is followed by an additional hairpin, which can be a rho-independent transcription terminator. There are additional stems that can be pseudoknots or stems involved in alternate structures. The motif is well supported by covariation, but the fact that it is present in only one species and only five sequences are available provides reluctantance to declare that it is certain to be a structured RNA. The motif is in the potential 5′ UTRs of operons that contain a radC gene, which is annotated as a DNA repair protein, or a mcrC gene, part of a predicted methyl-dependent restriction system.

Iii. pfl motif

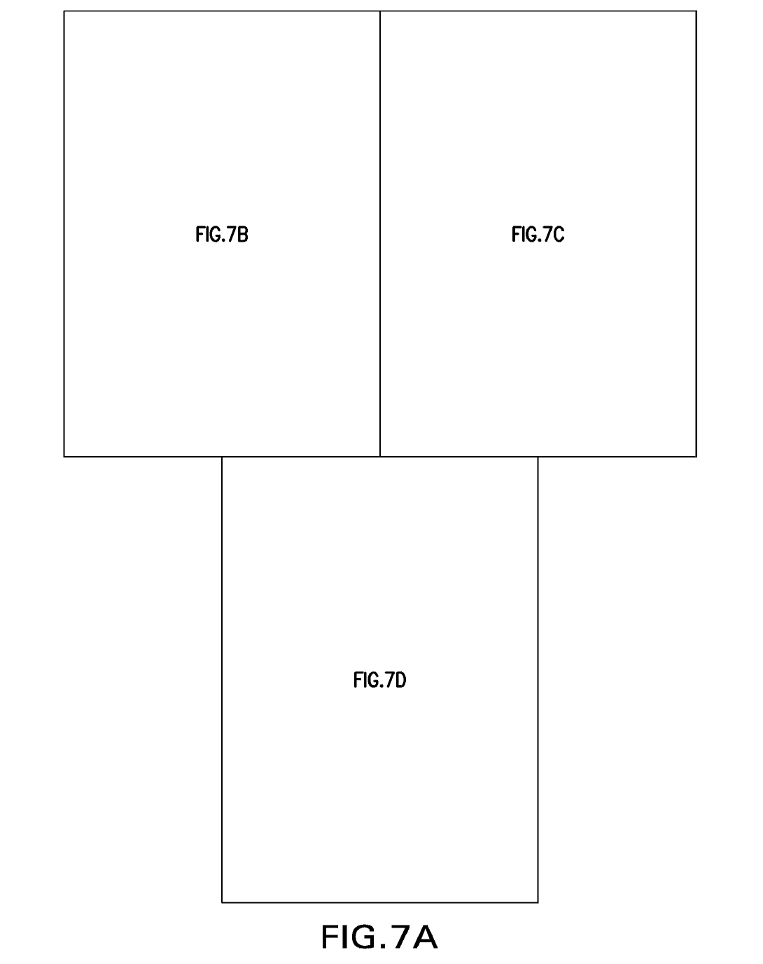

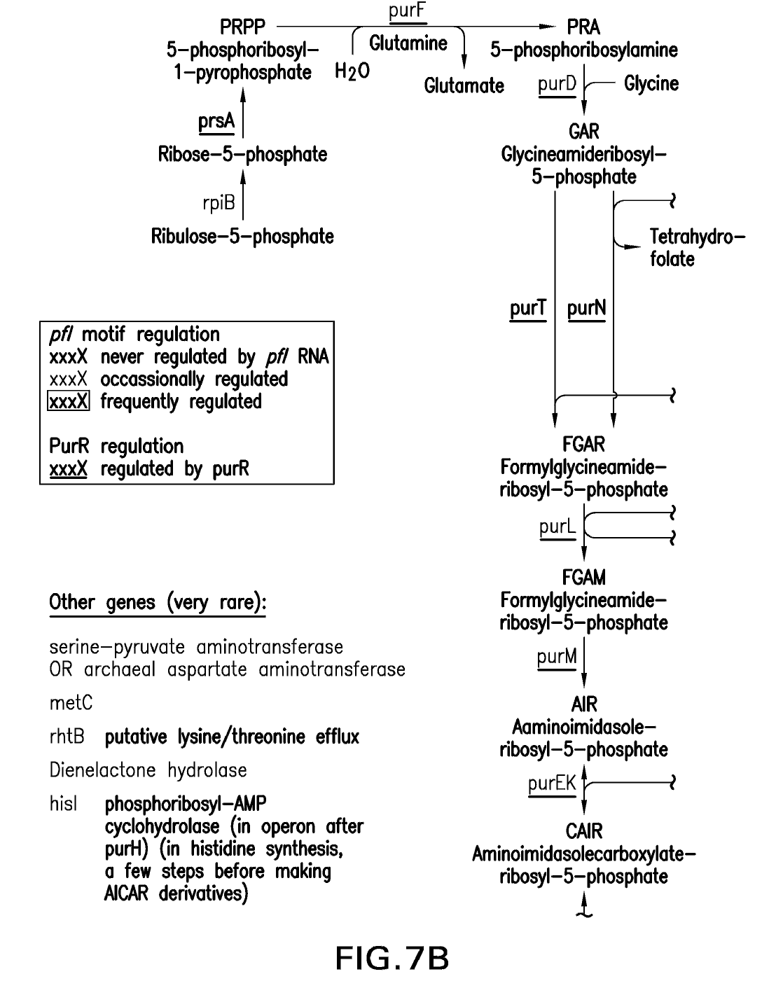

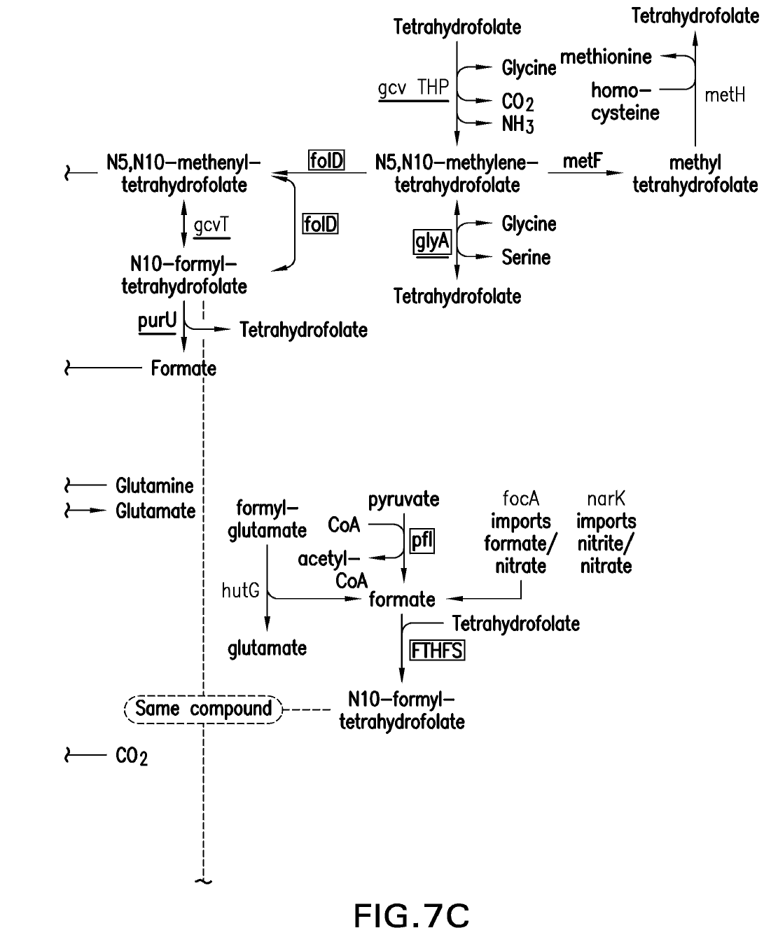

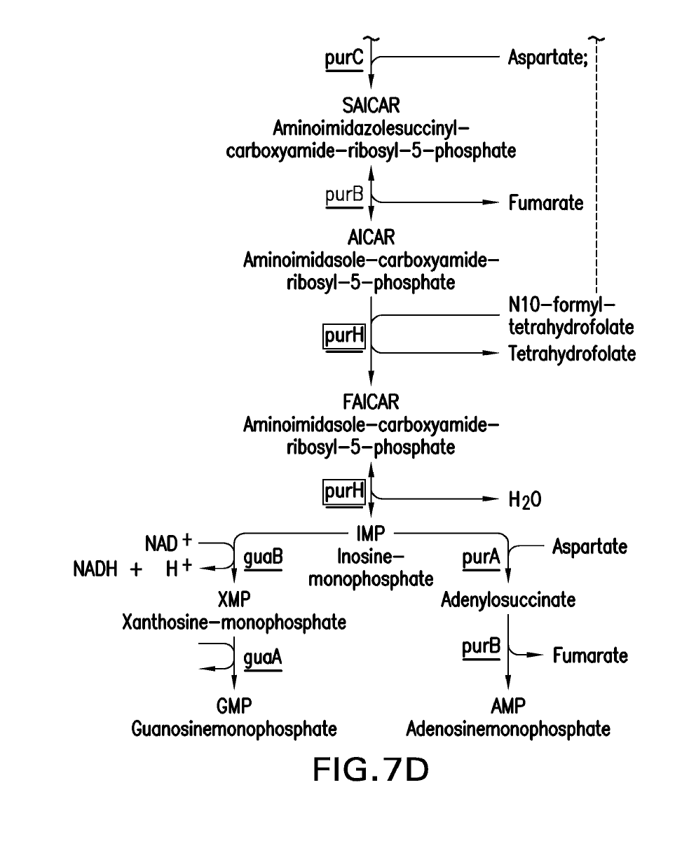

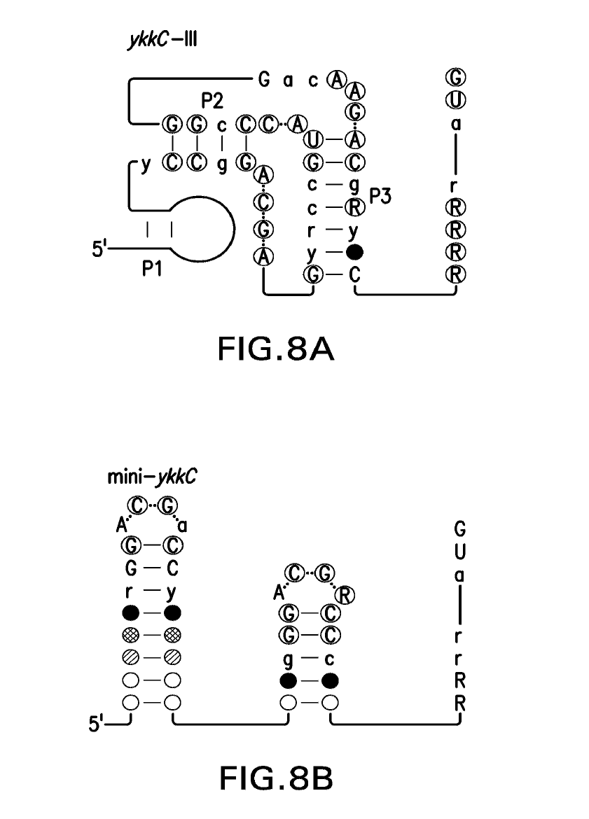

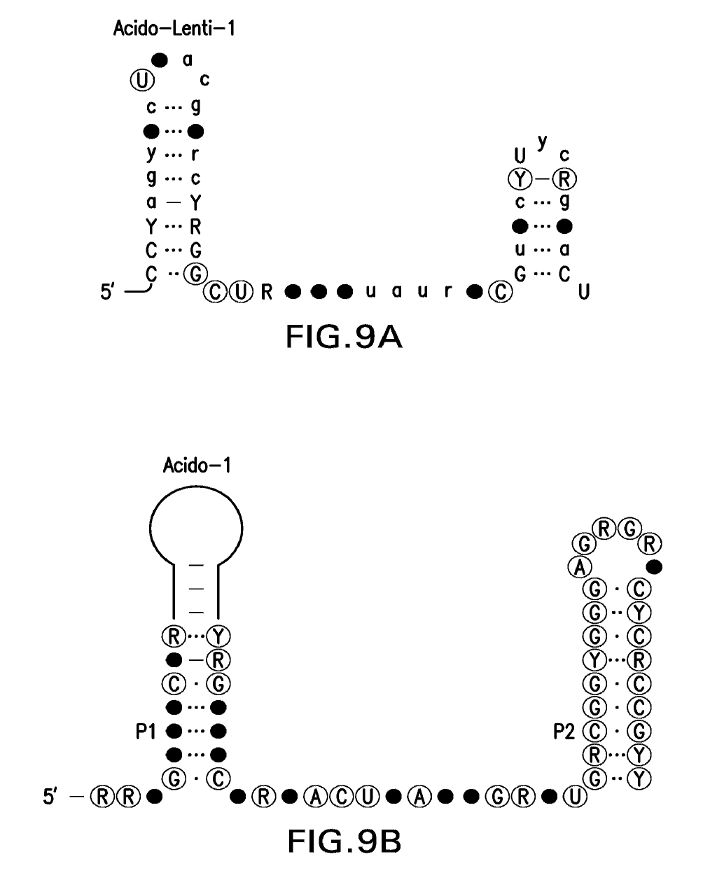

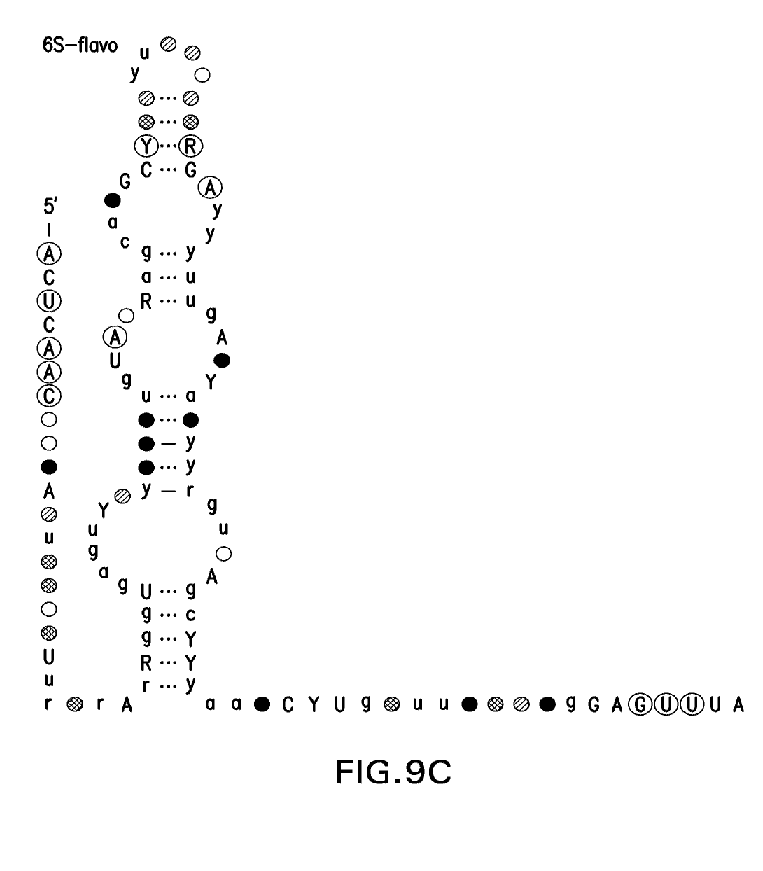

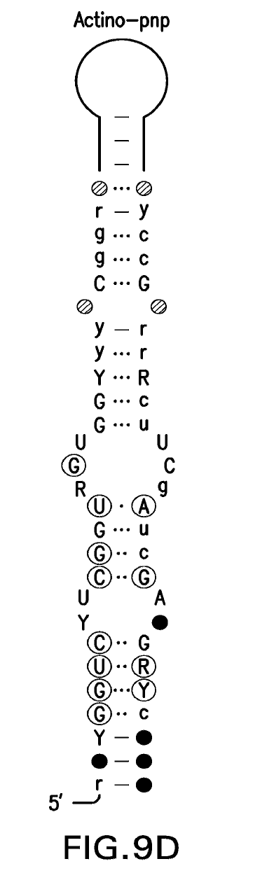

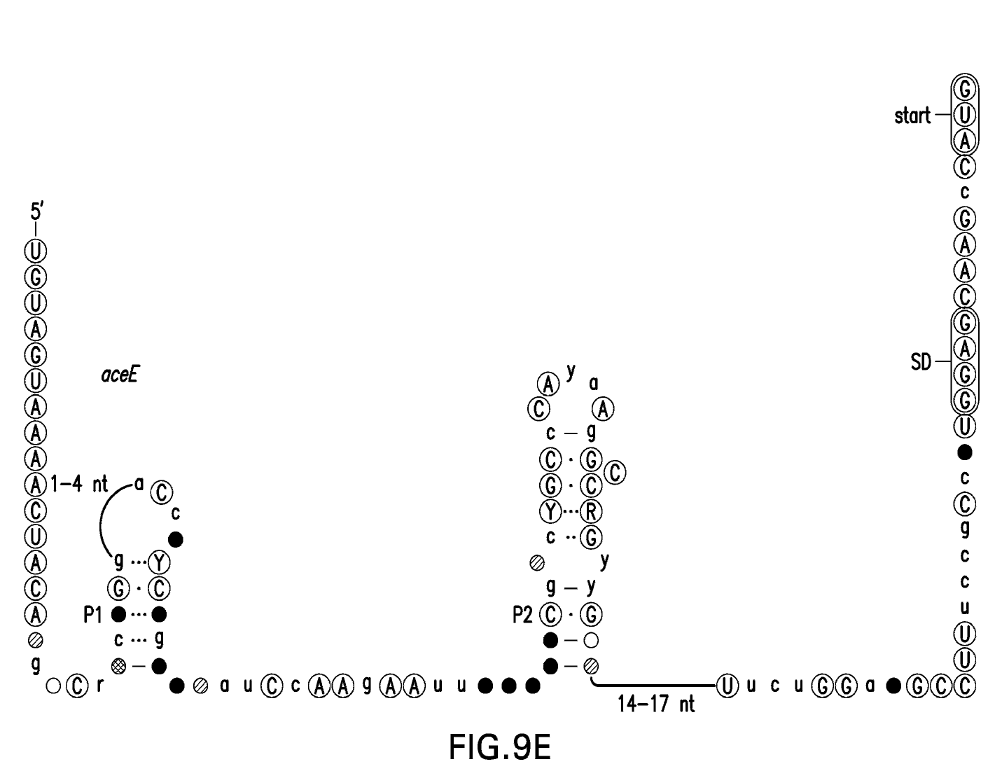

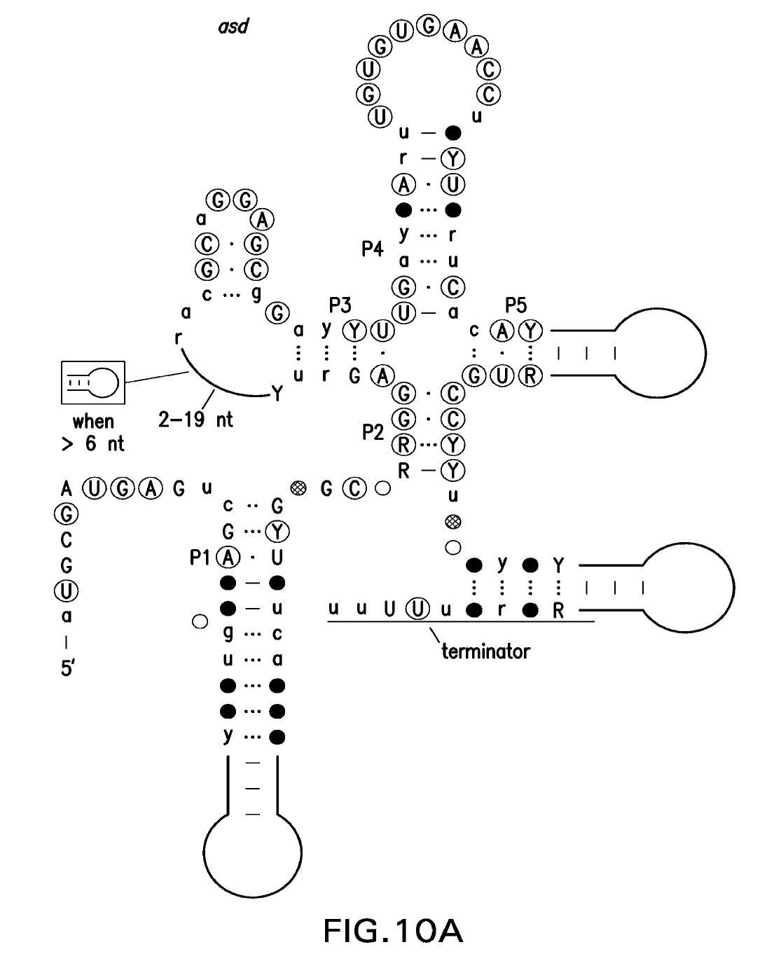

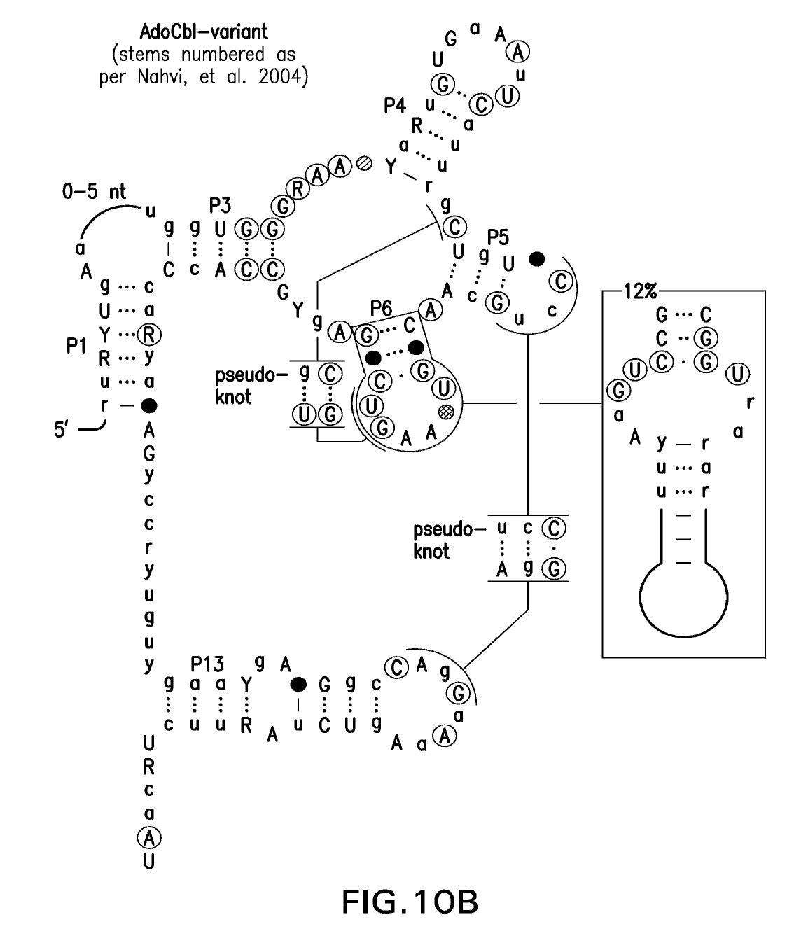

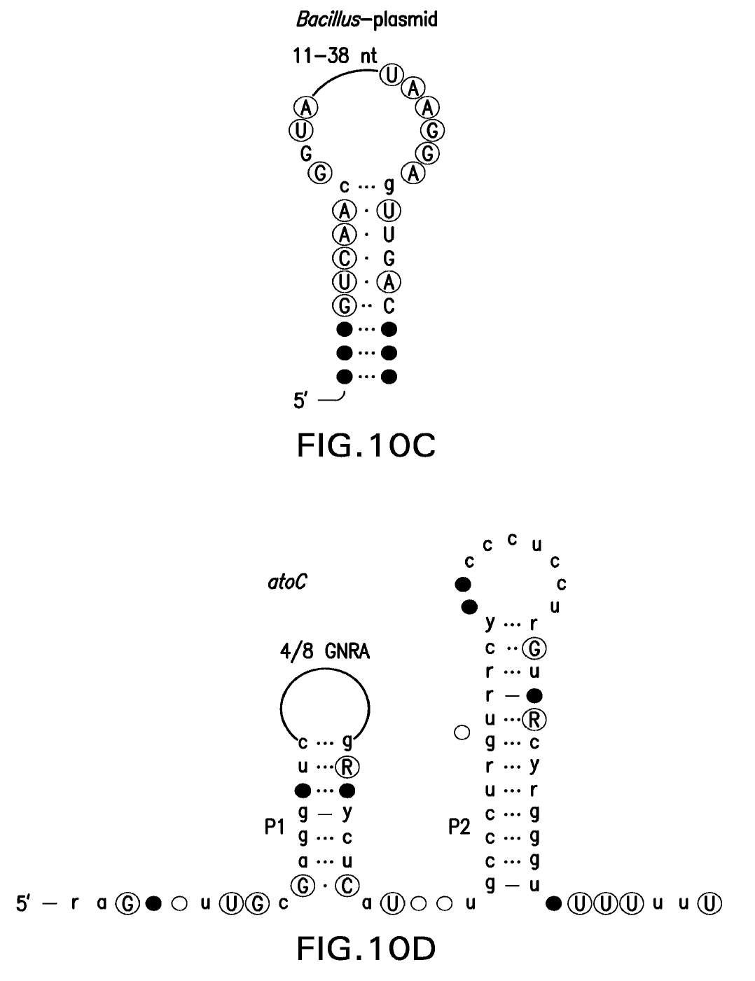

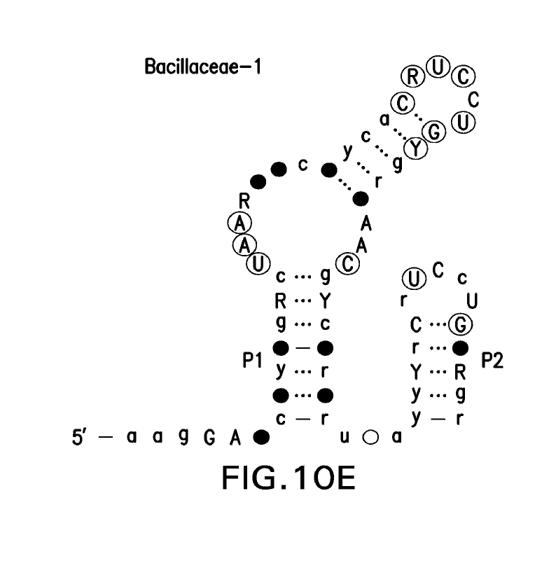

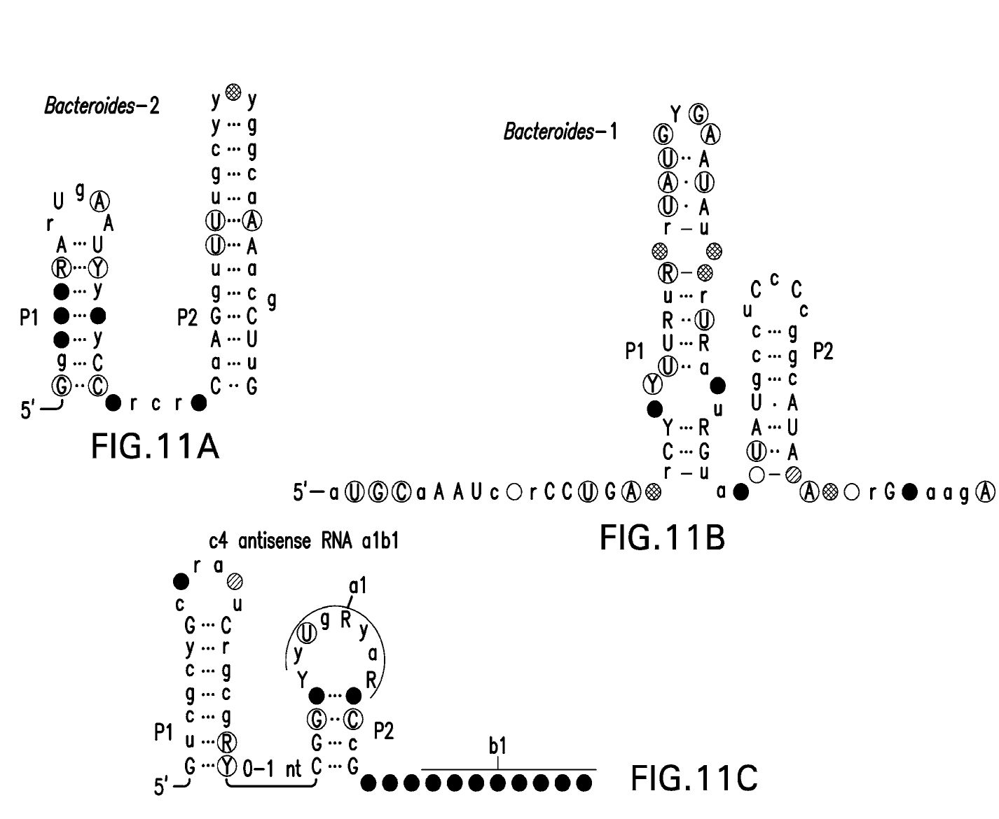

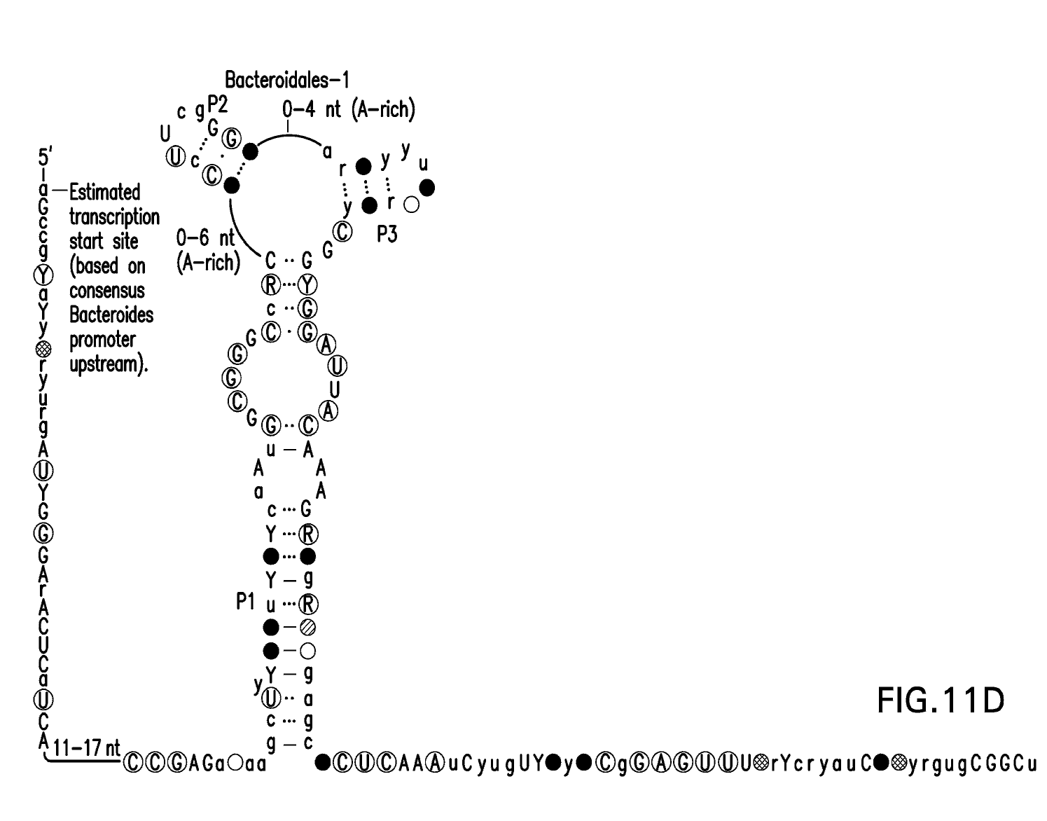

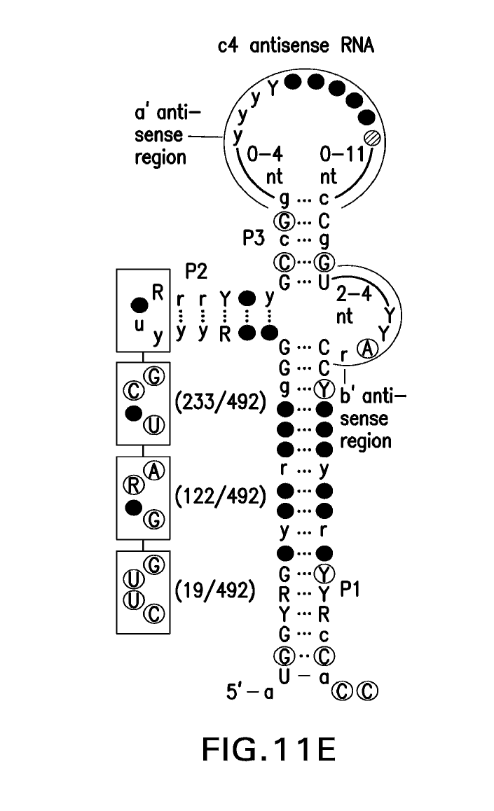

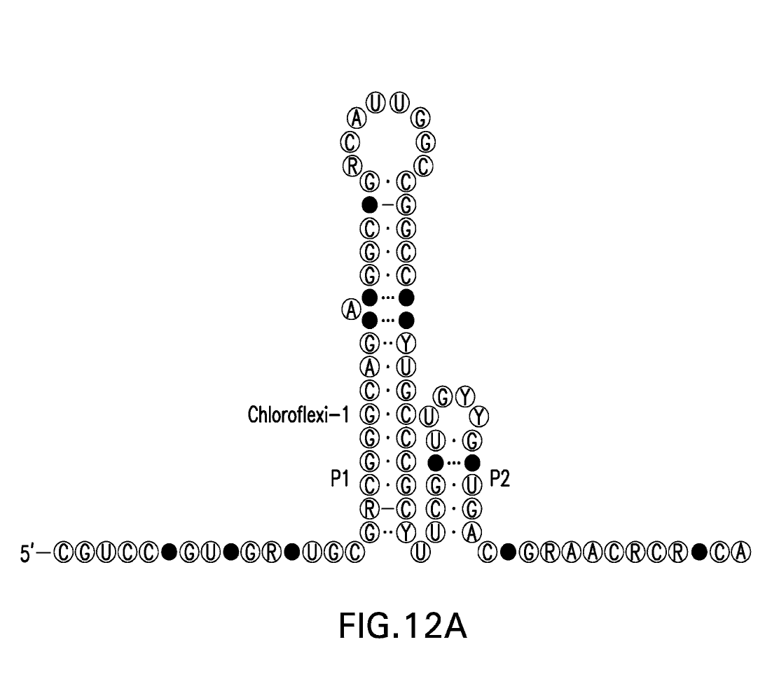

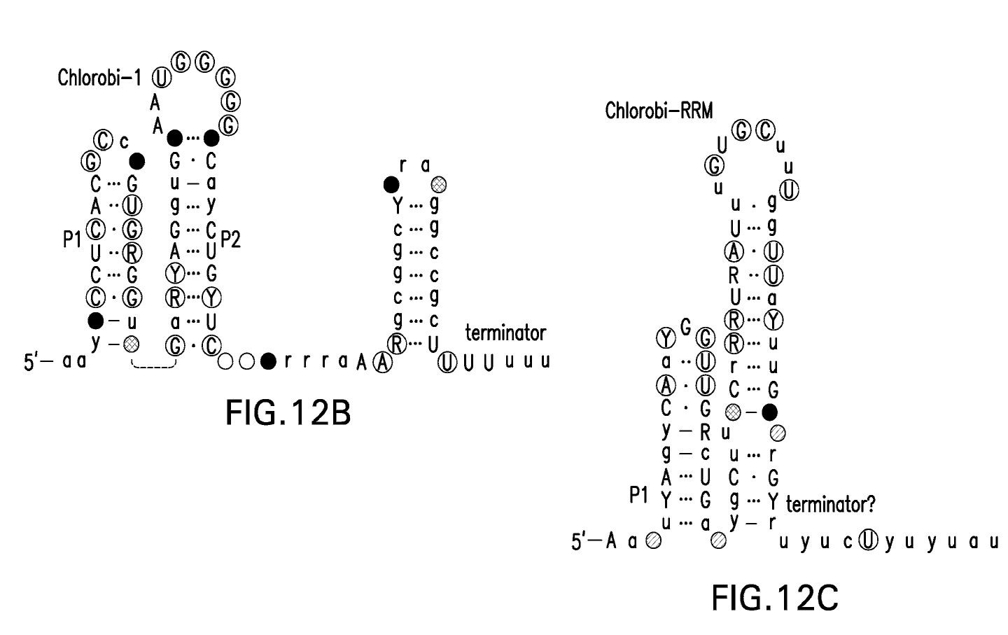

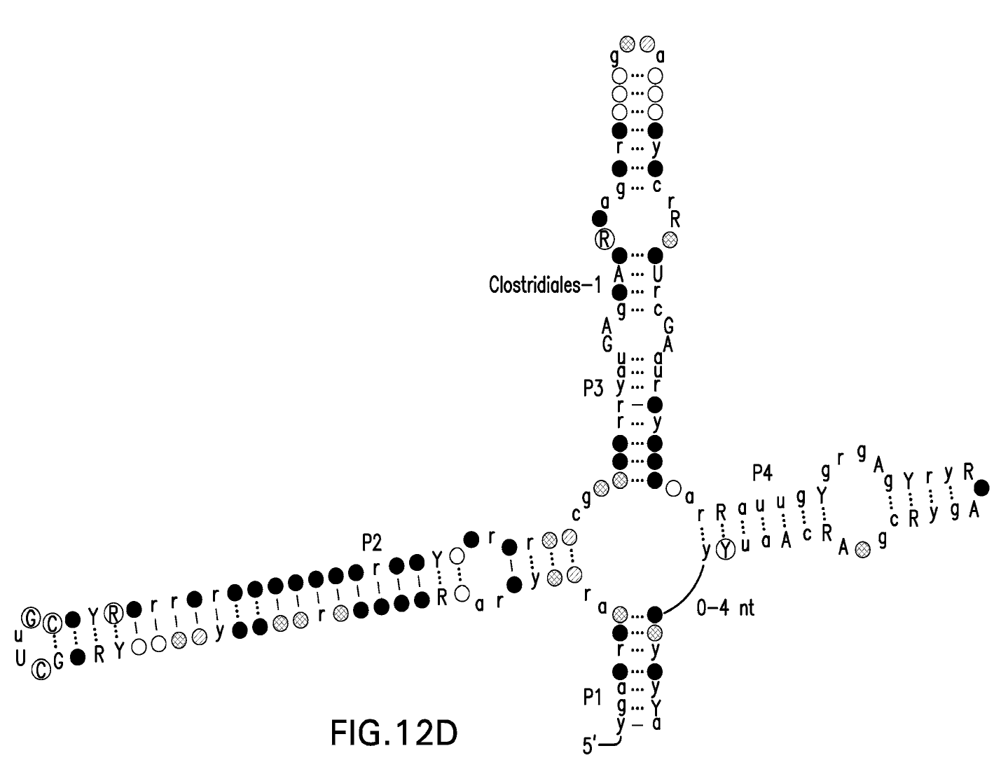

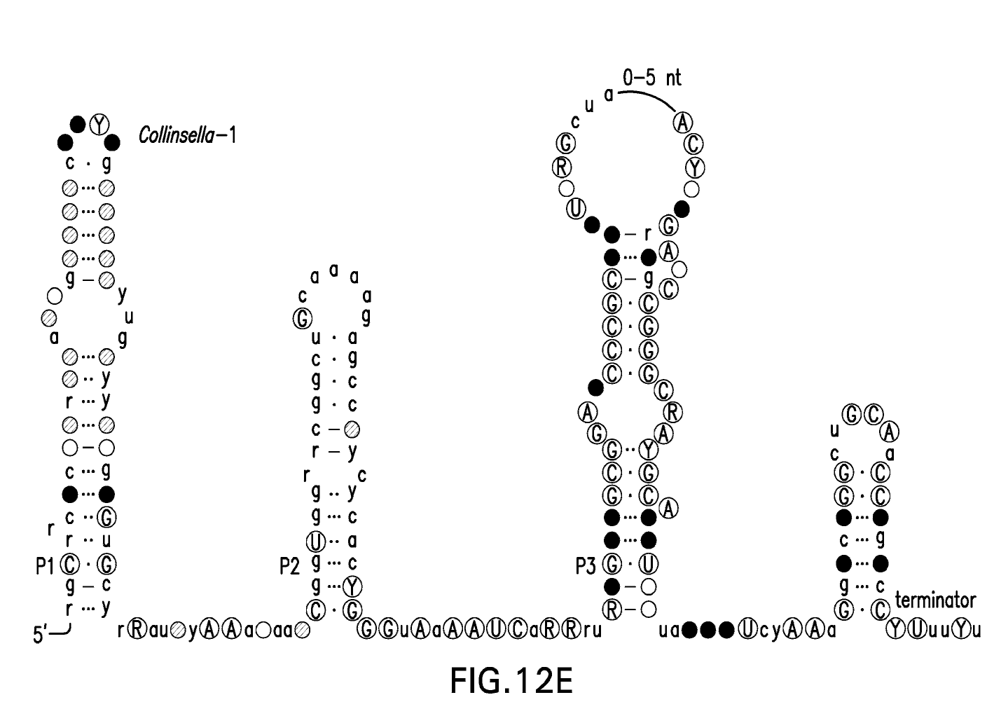

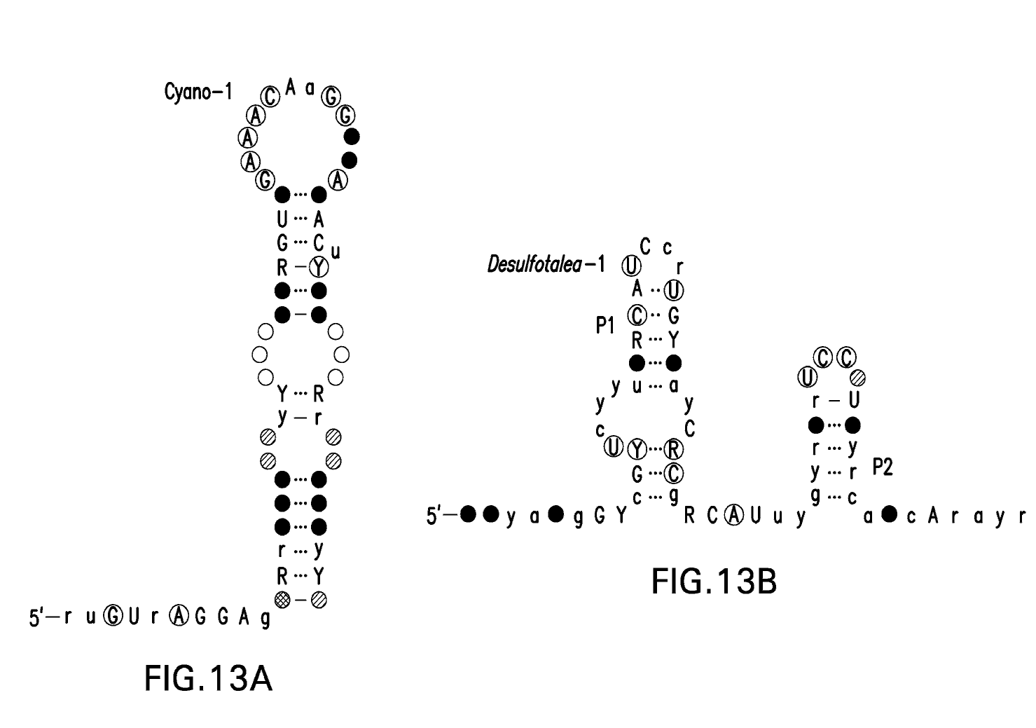

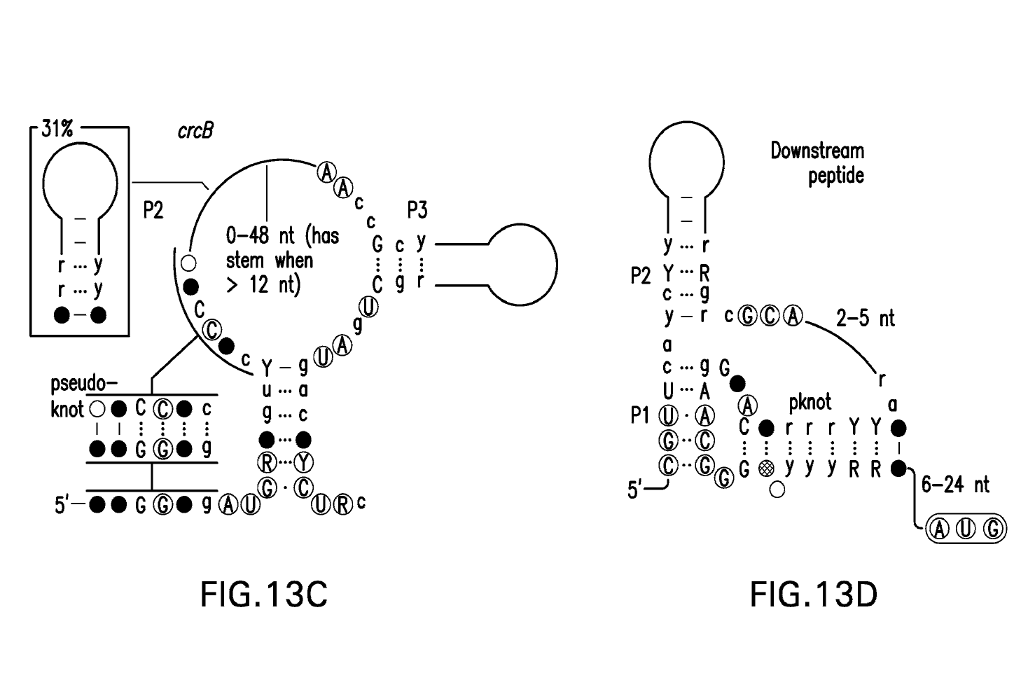

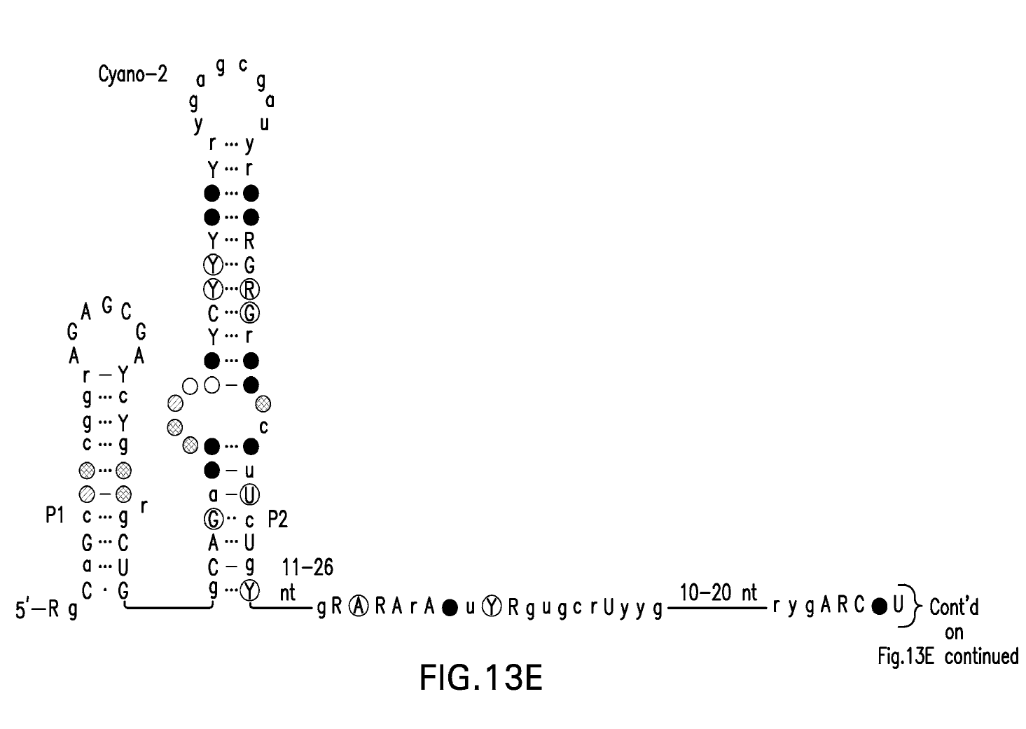

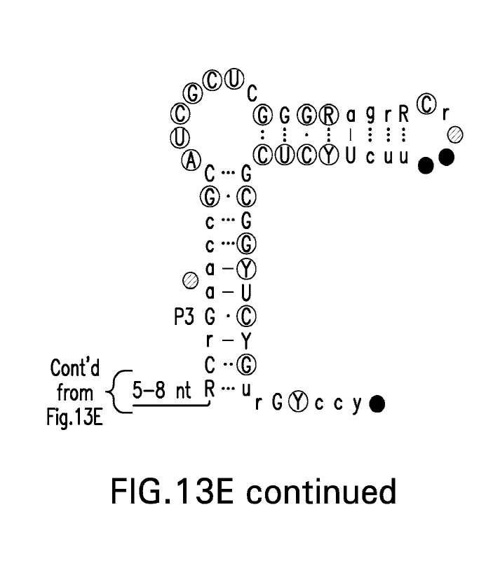

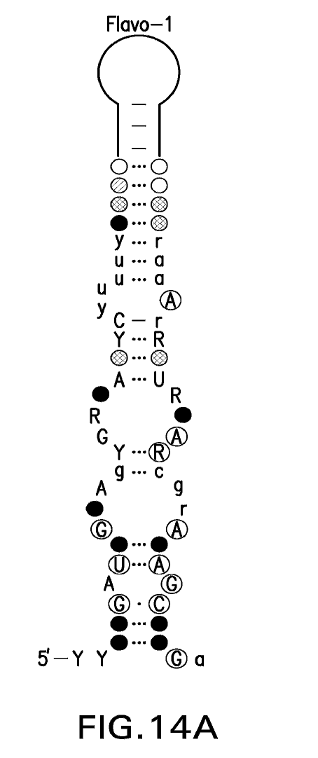

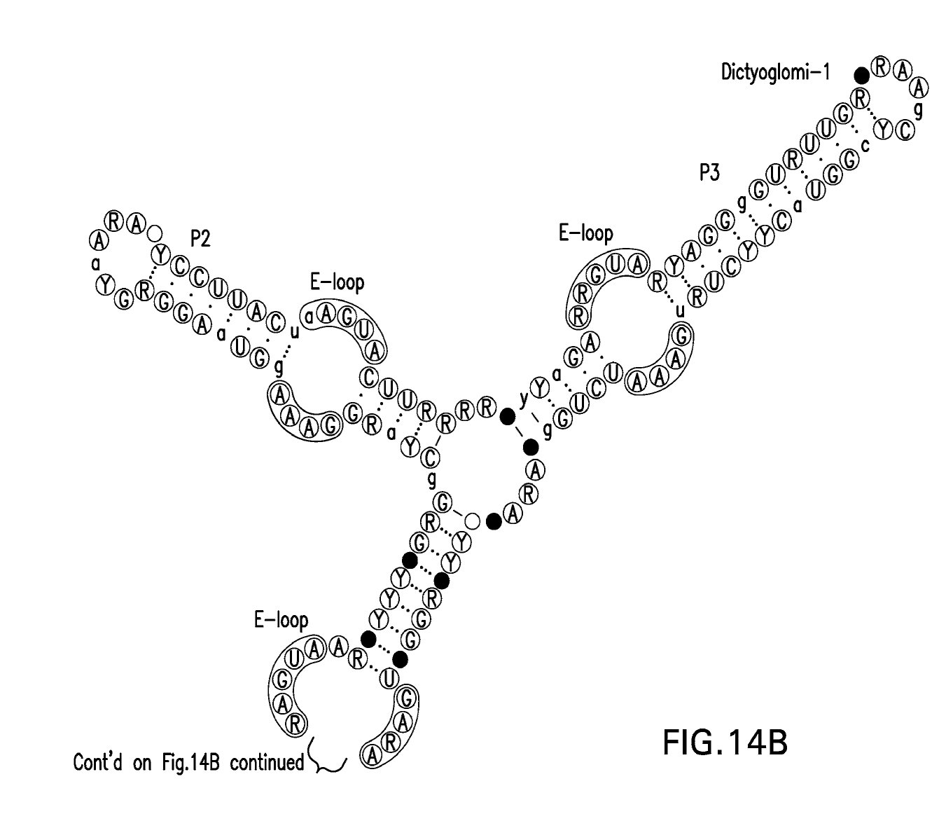

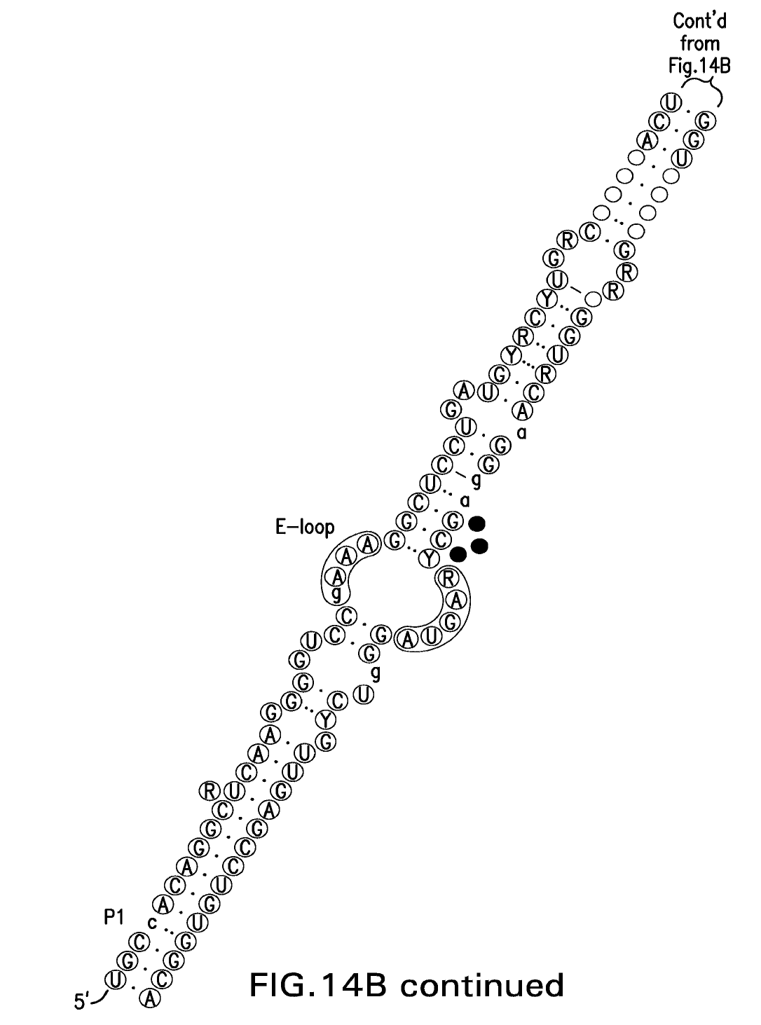

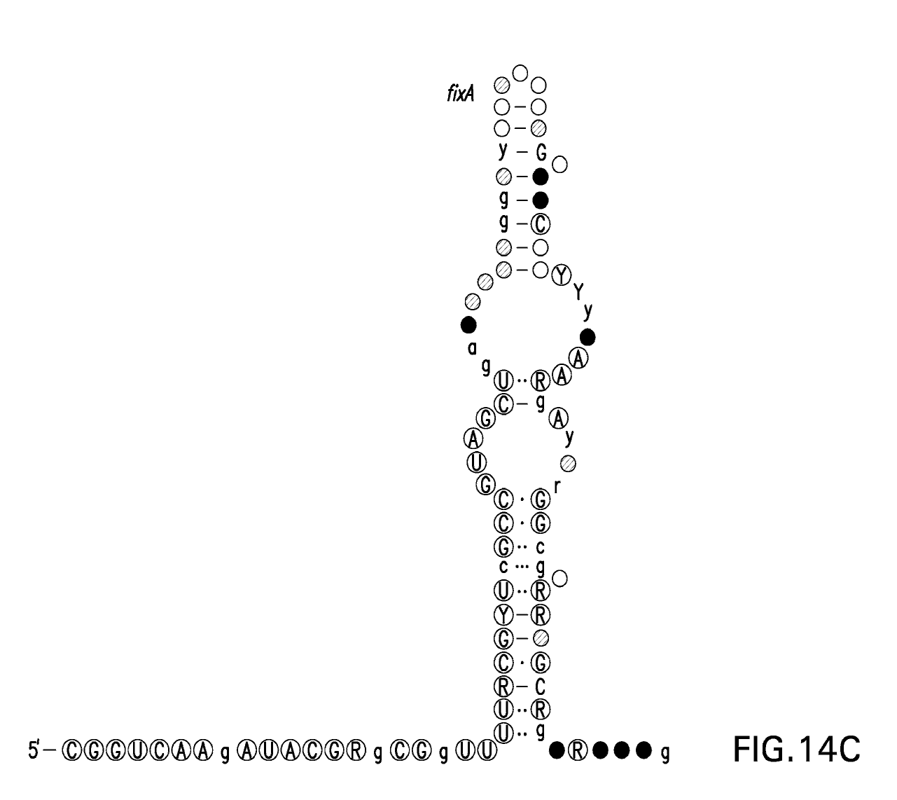

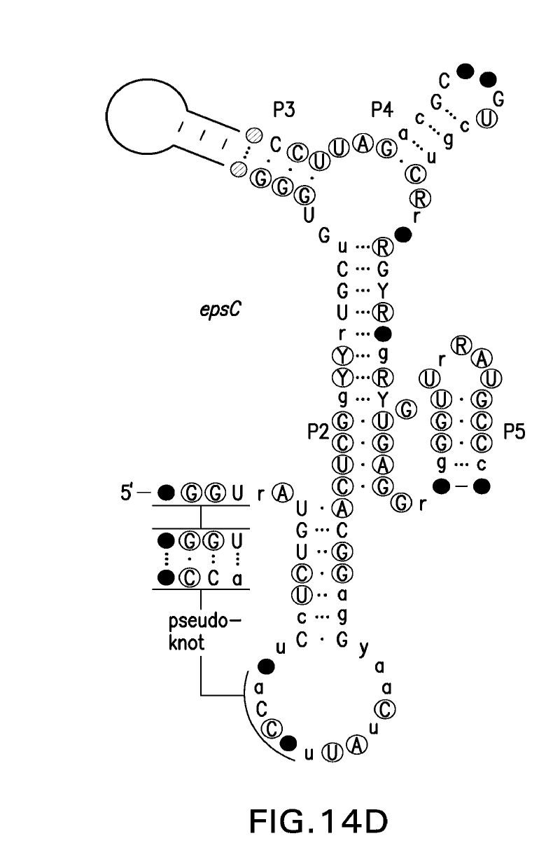

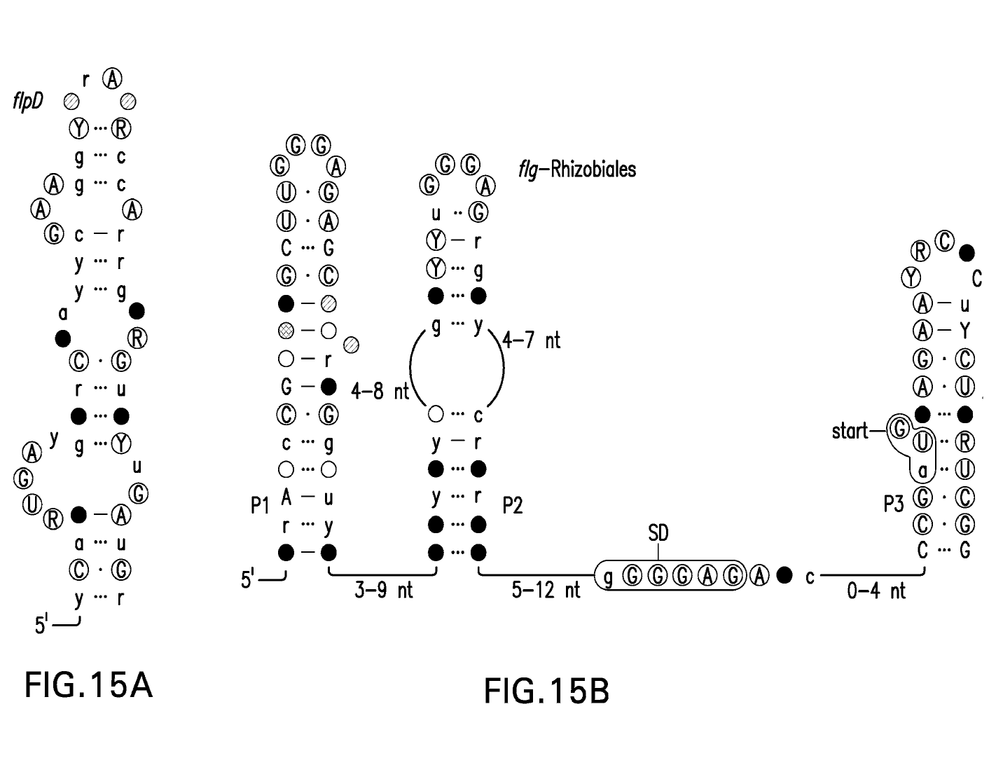

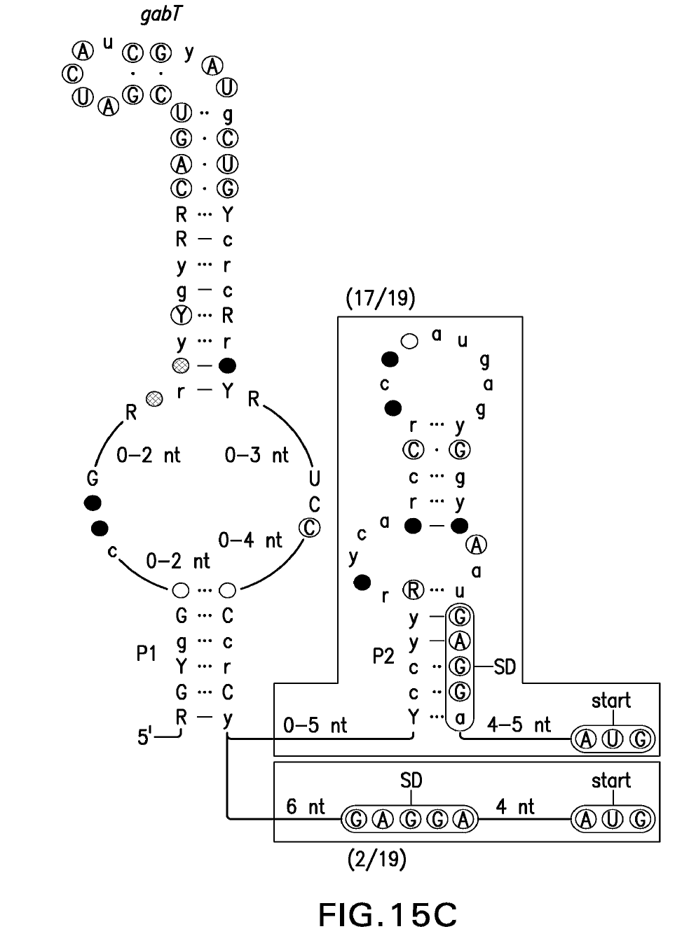

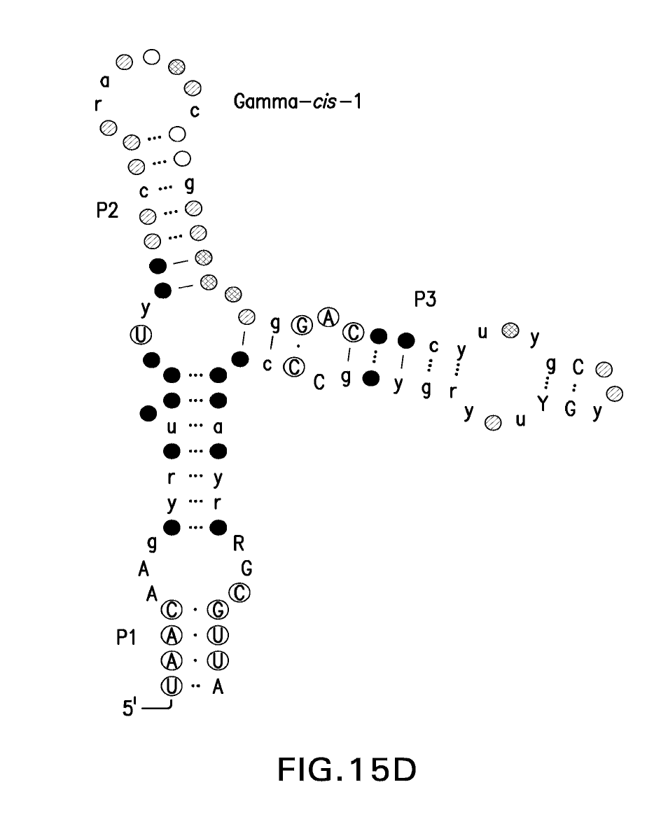

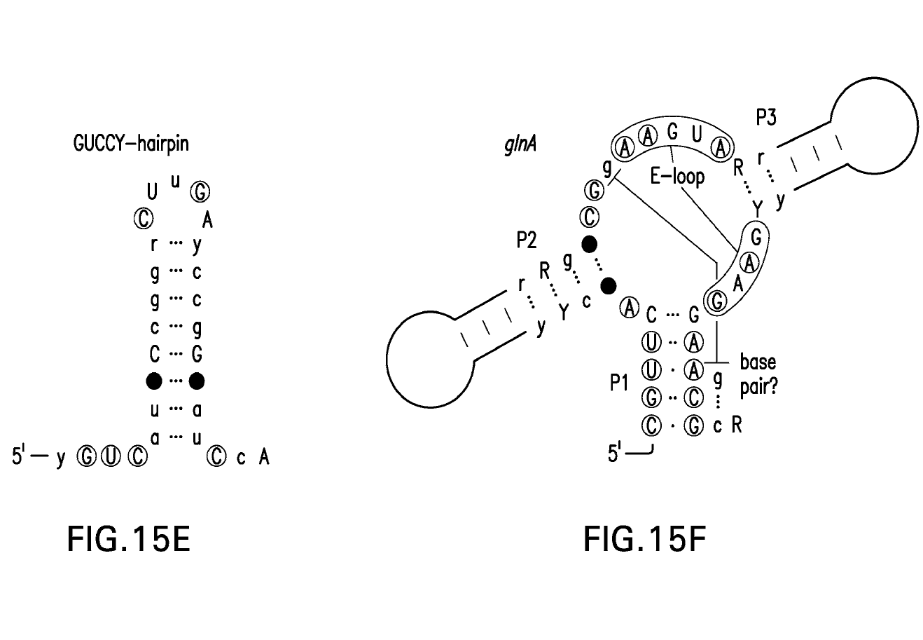

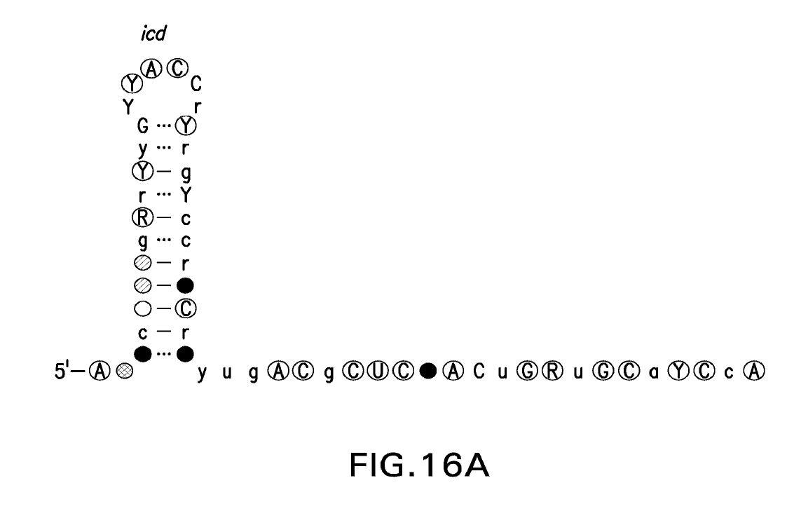

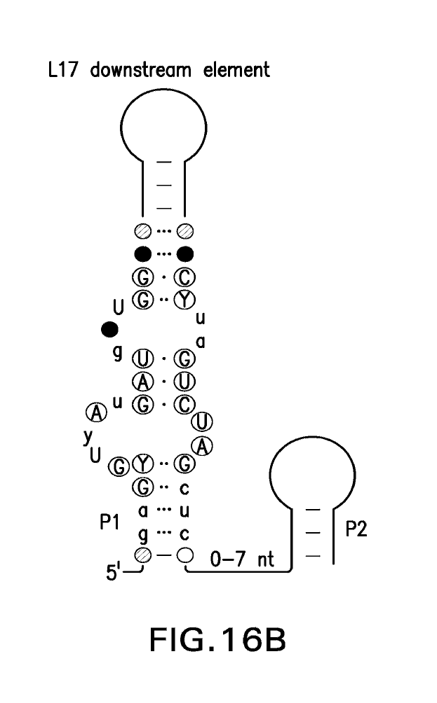

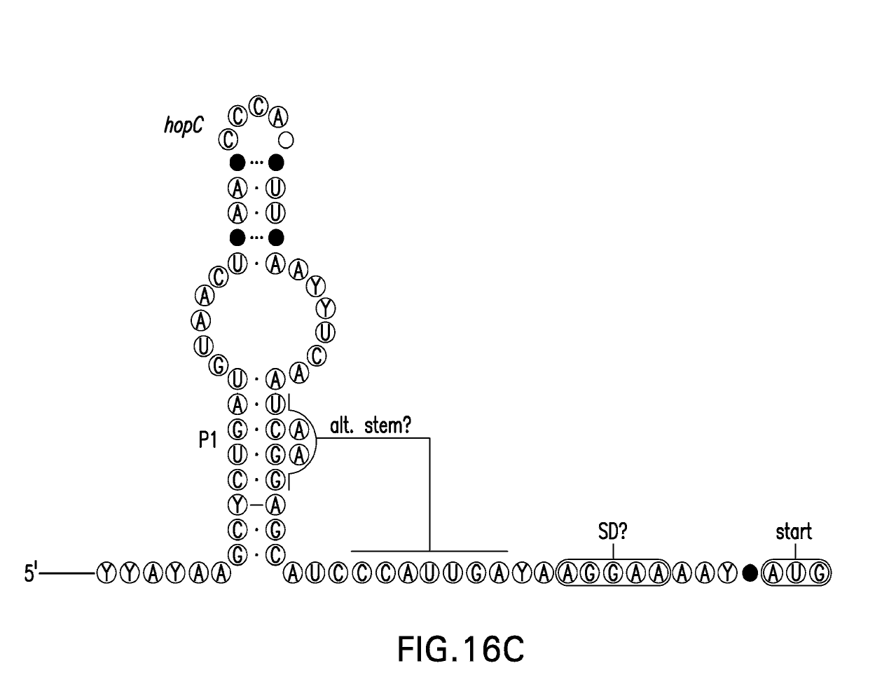

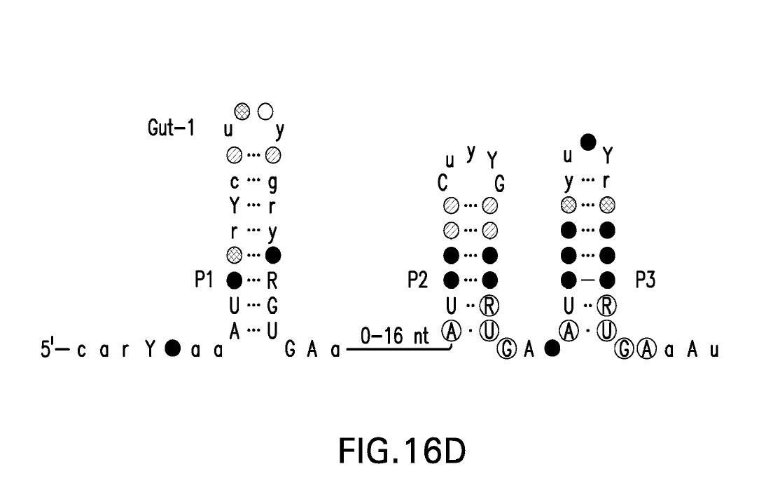

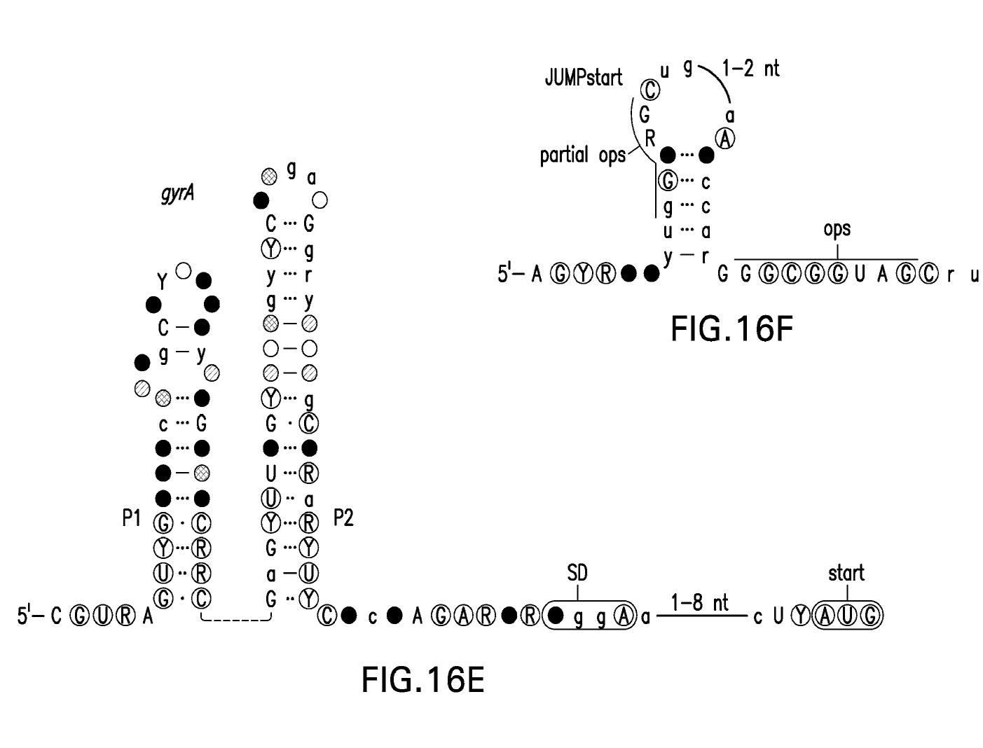

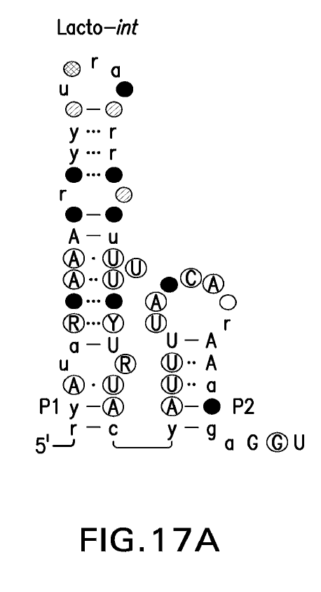

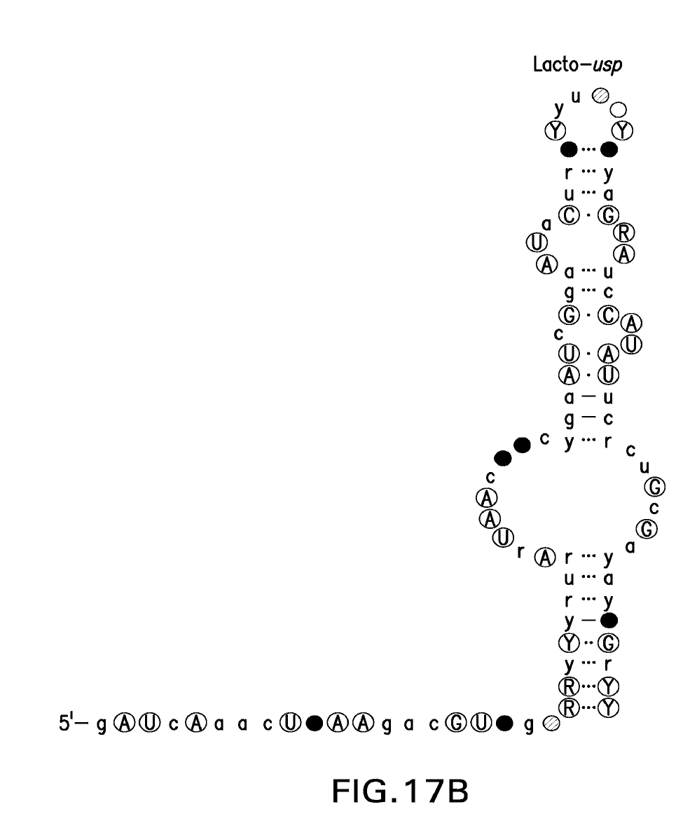

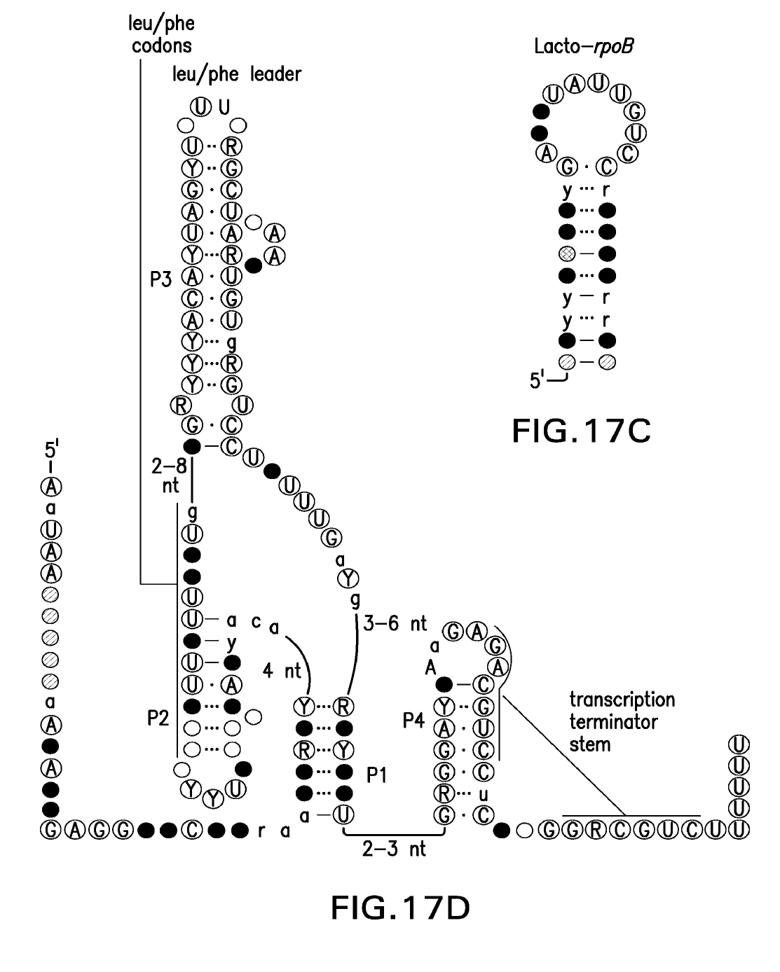

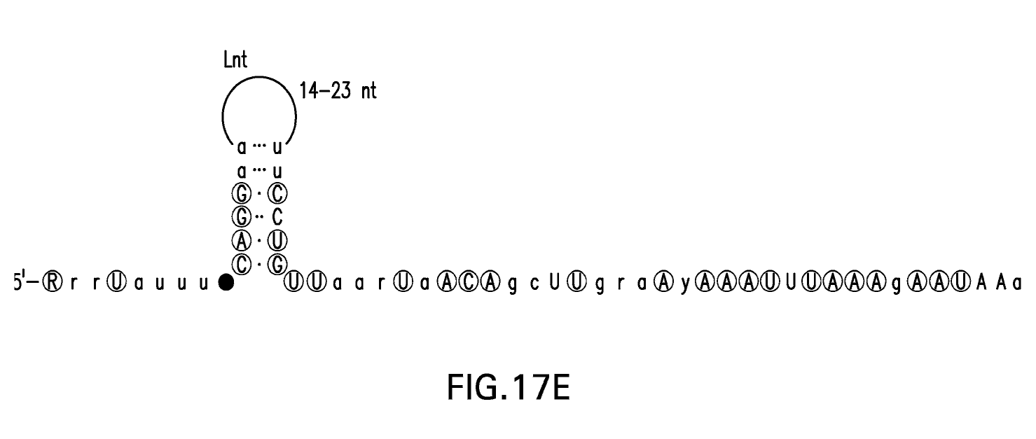

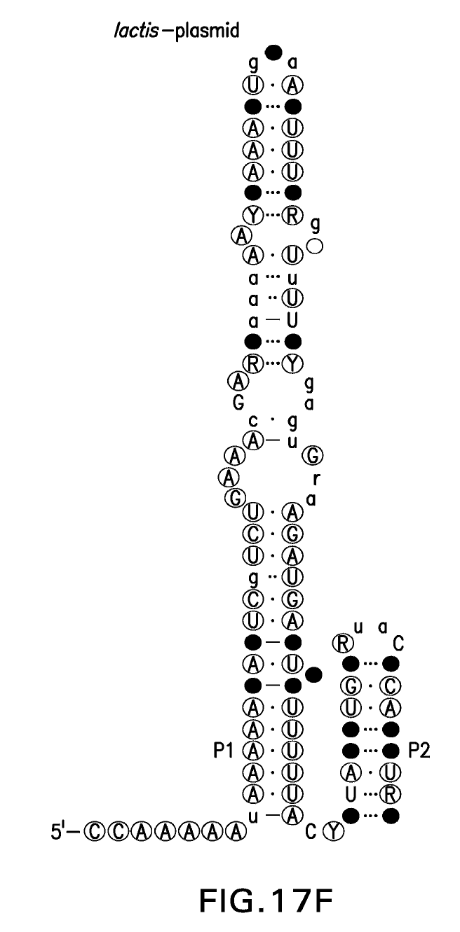

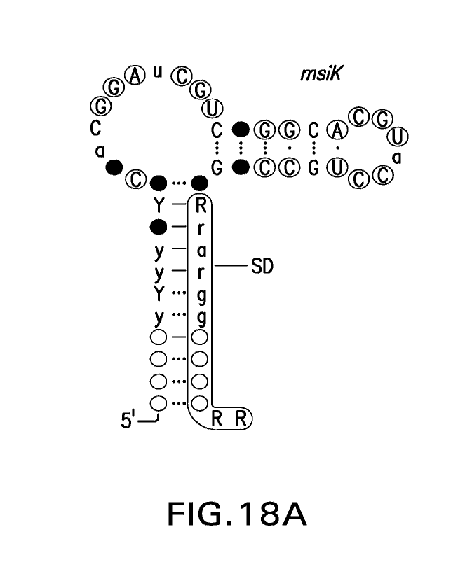

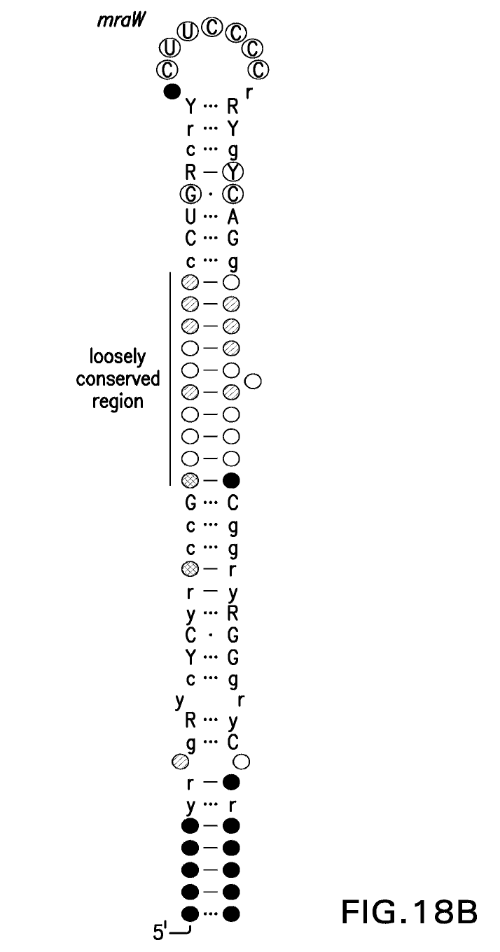

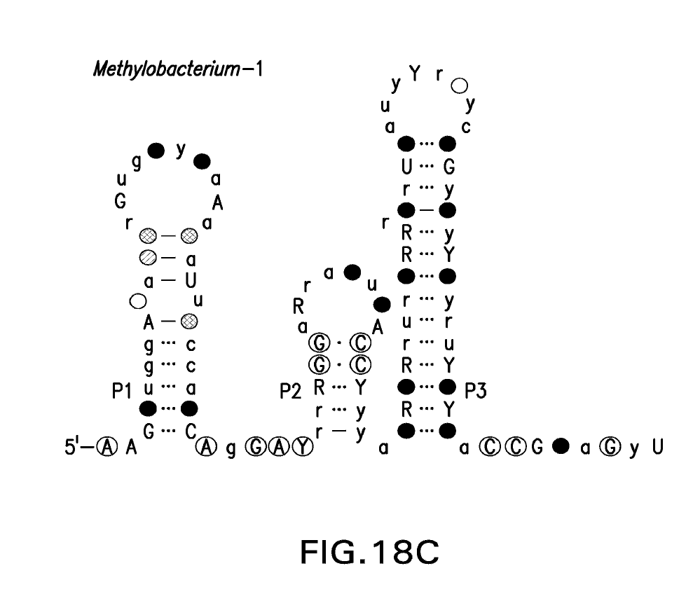

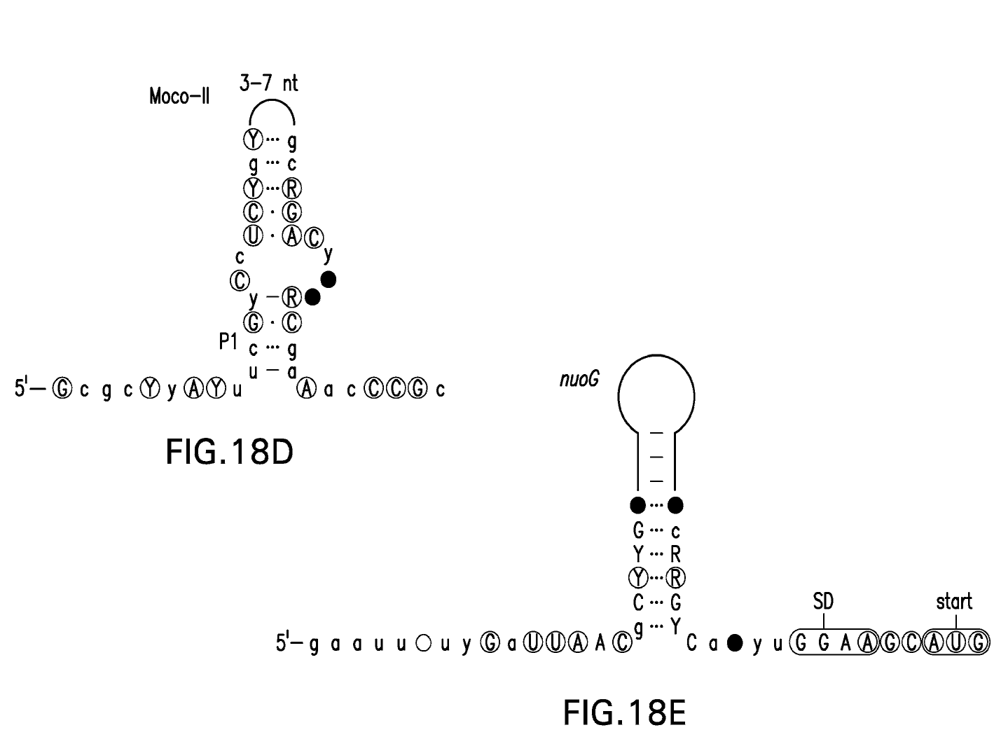

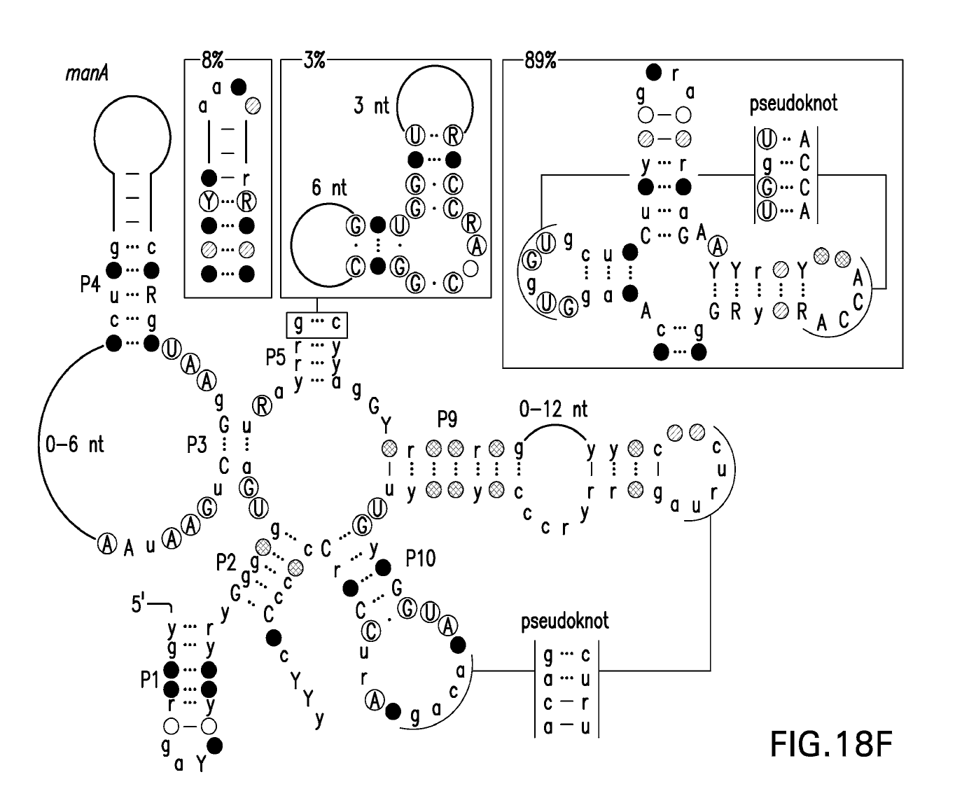

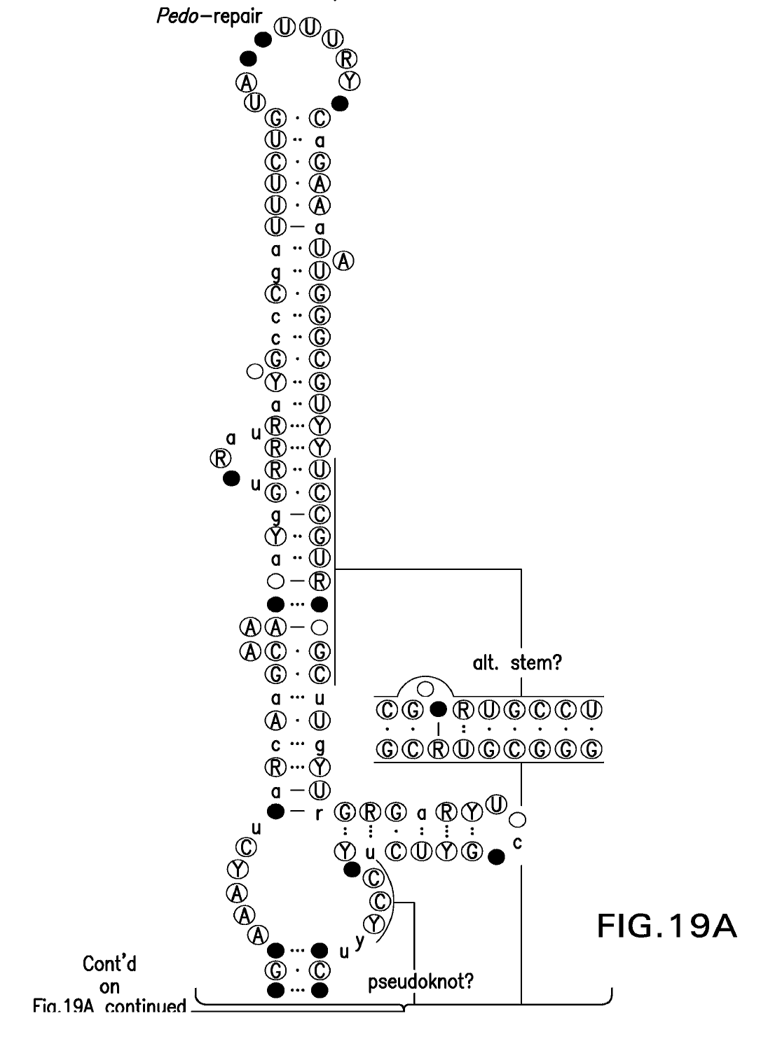

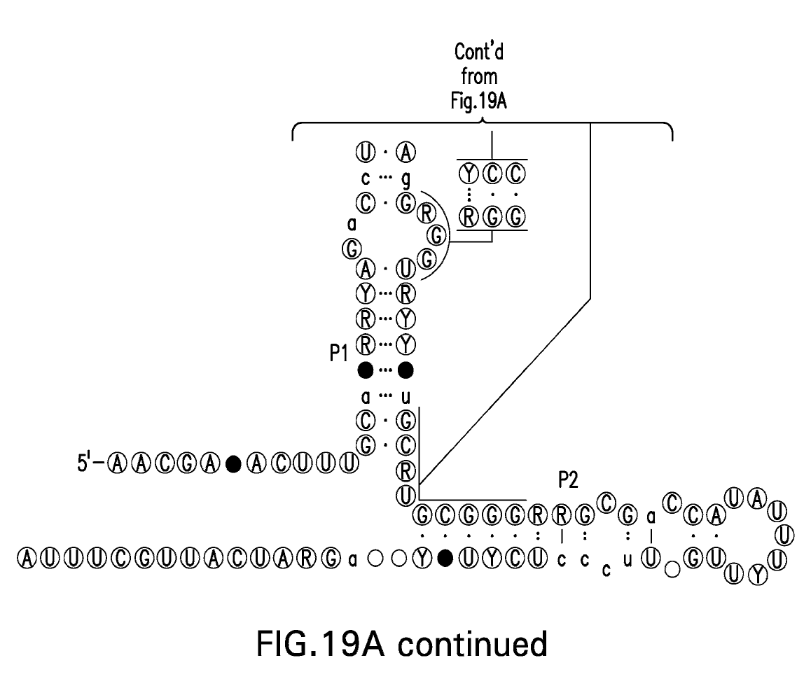

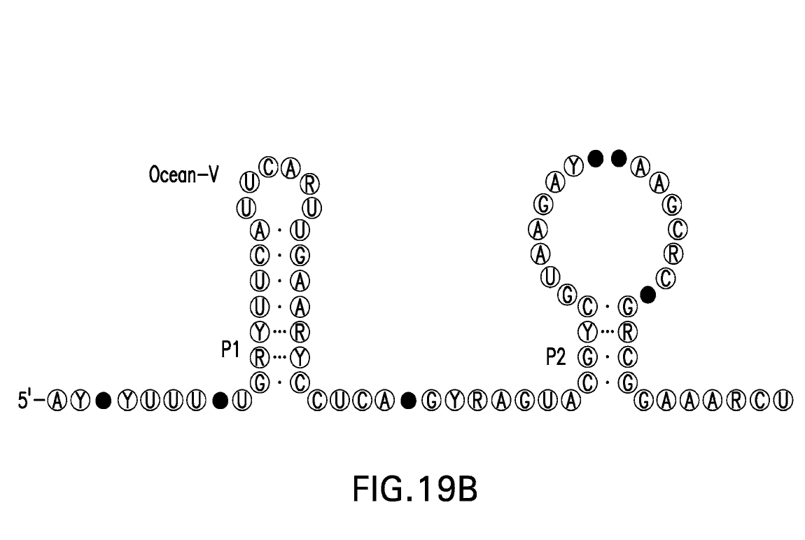

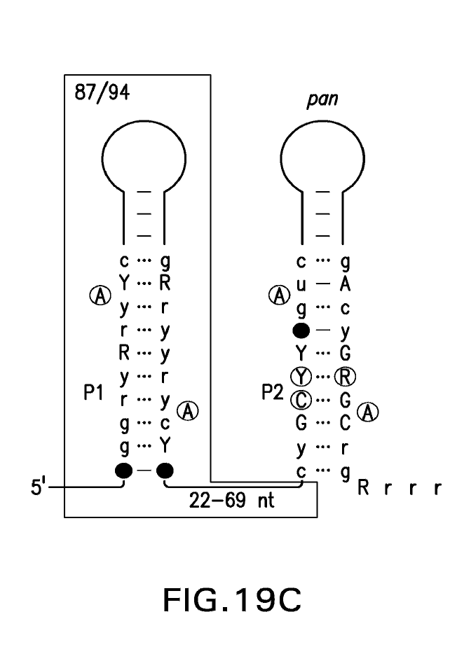

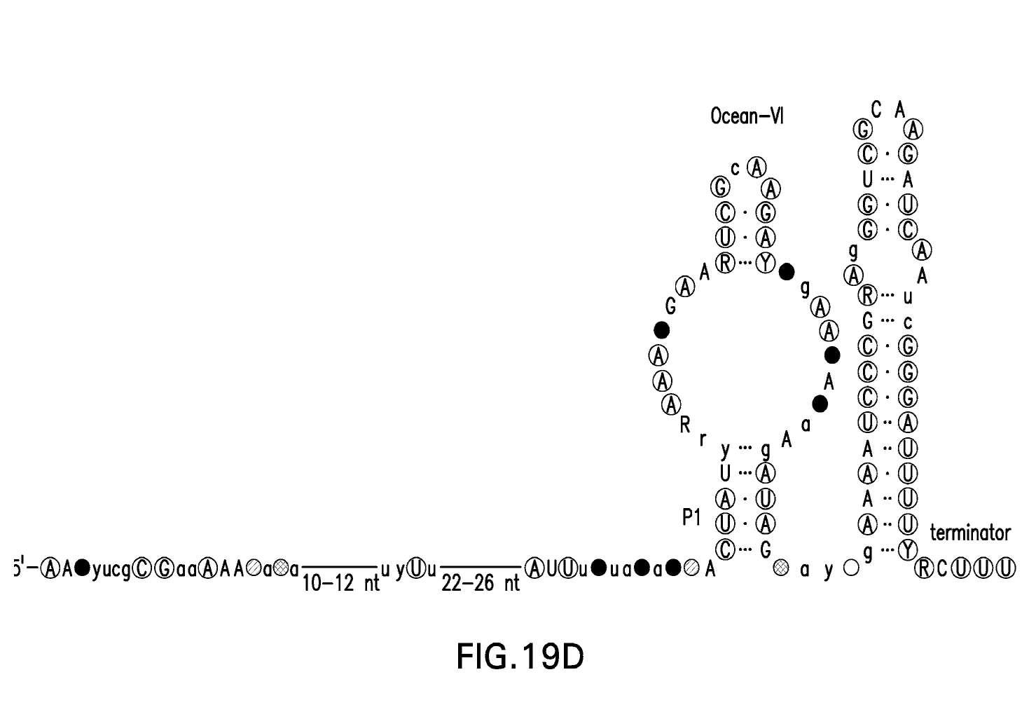

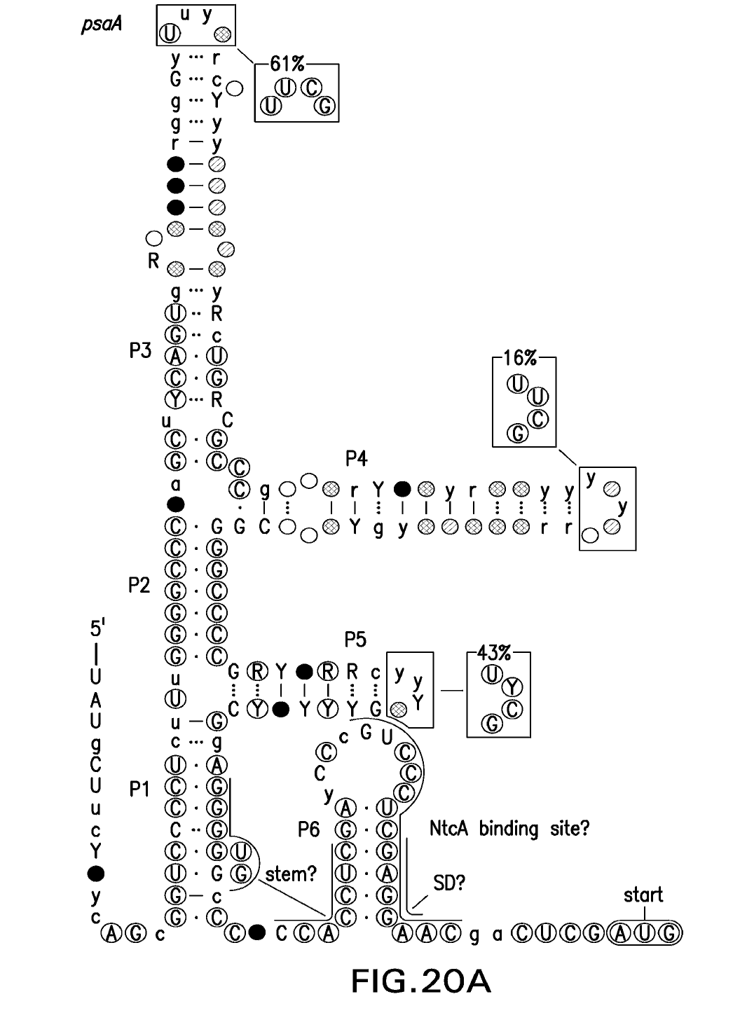

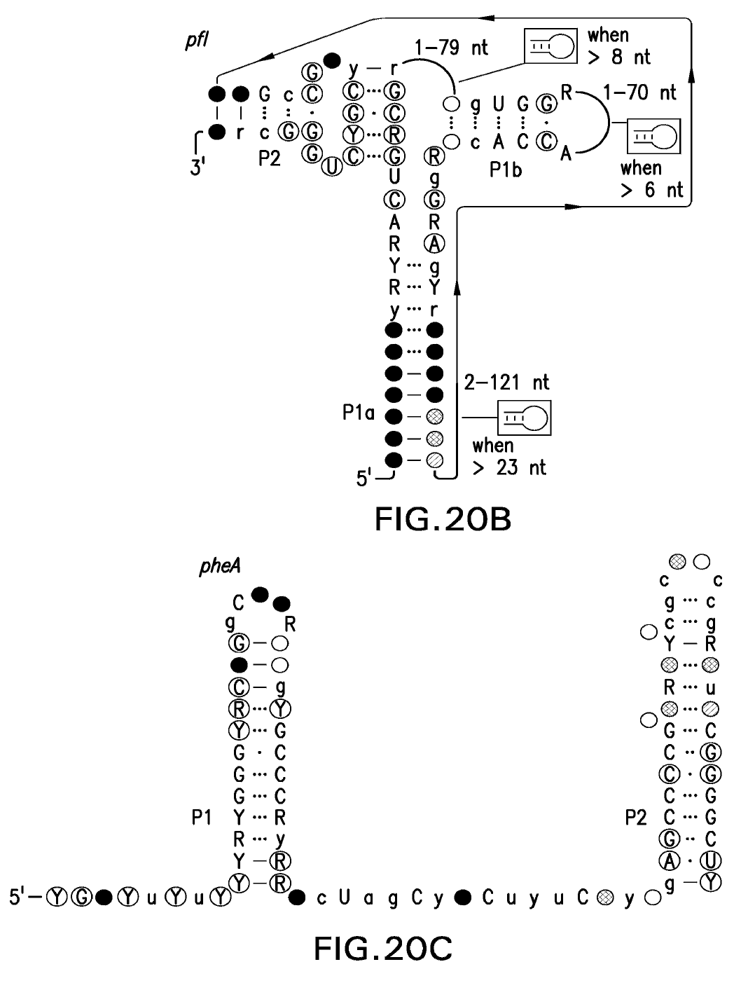

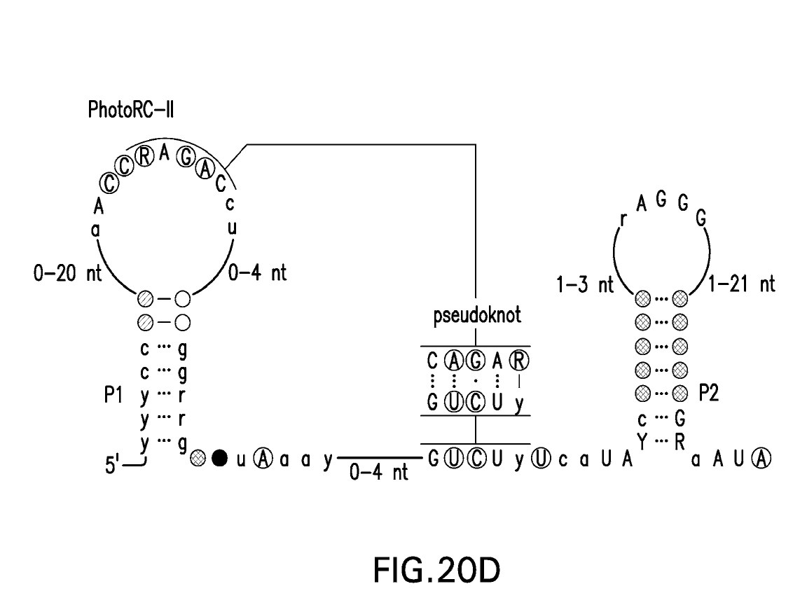

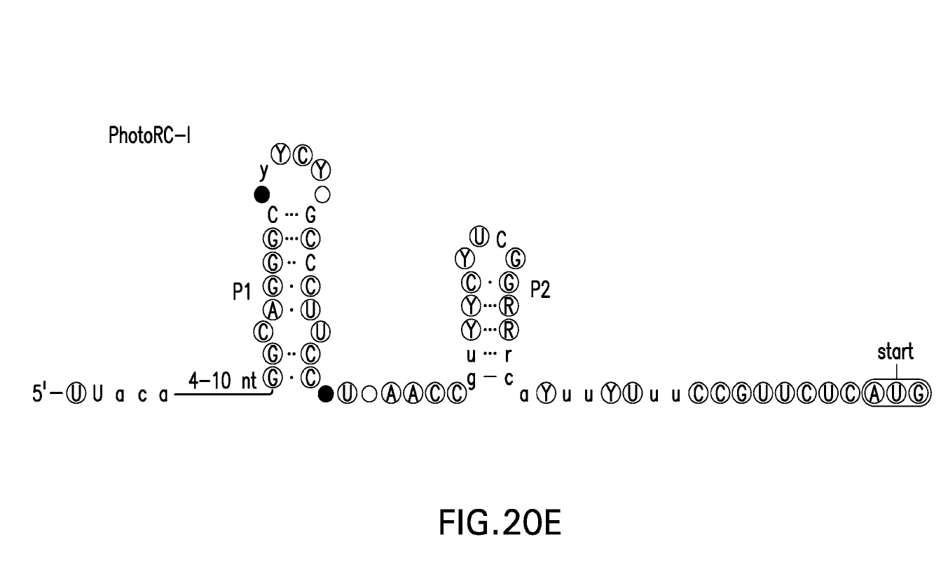

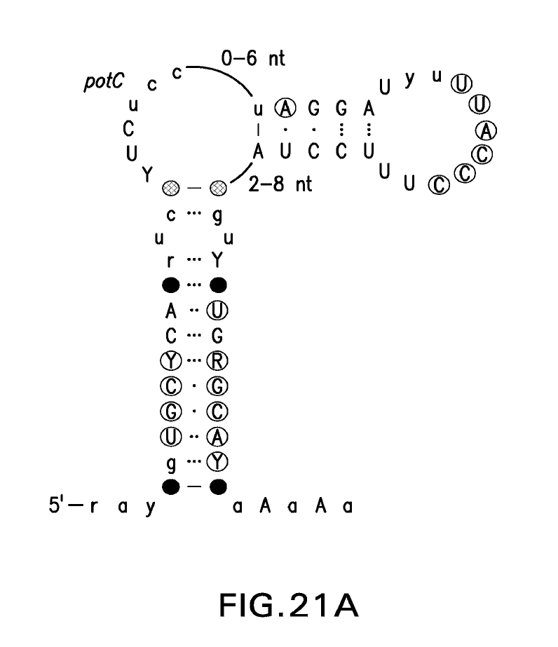

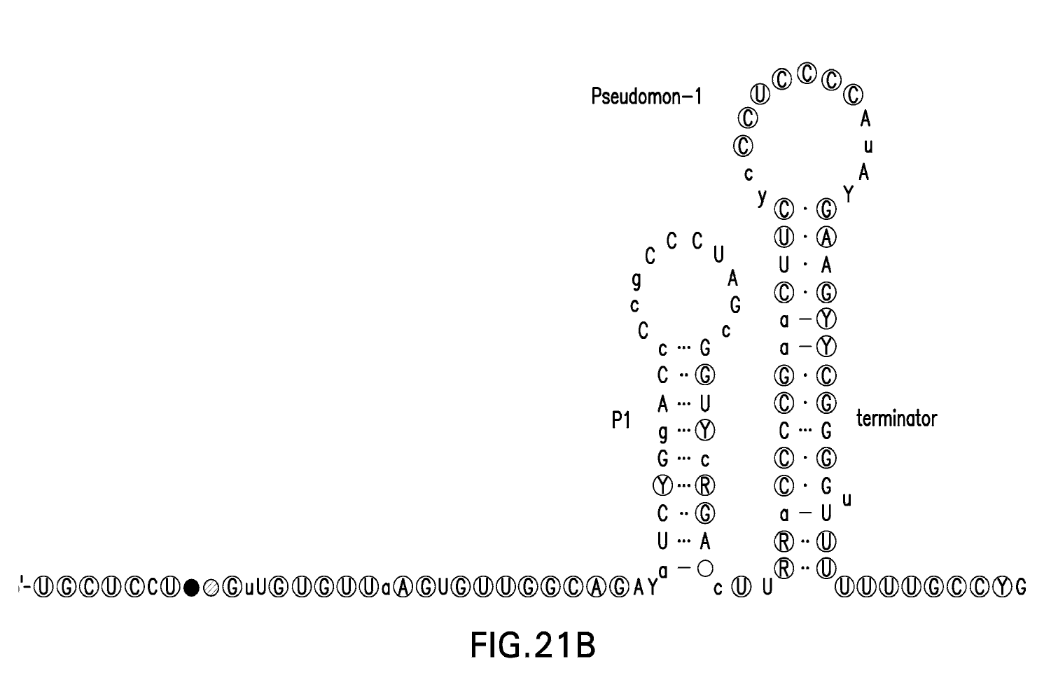

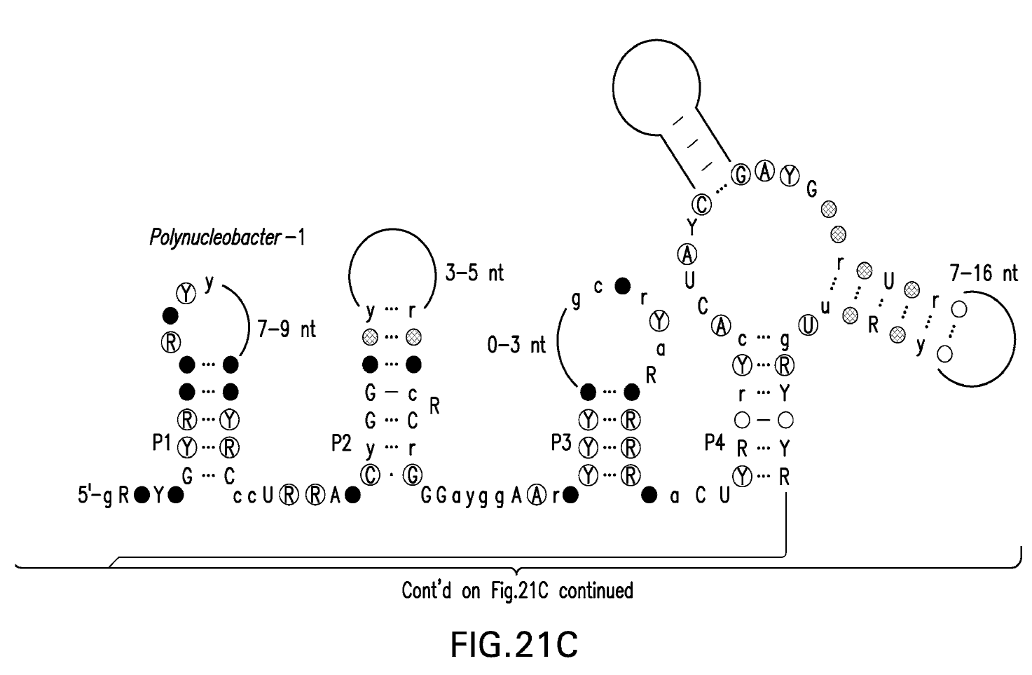

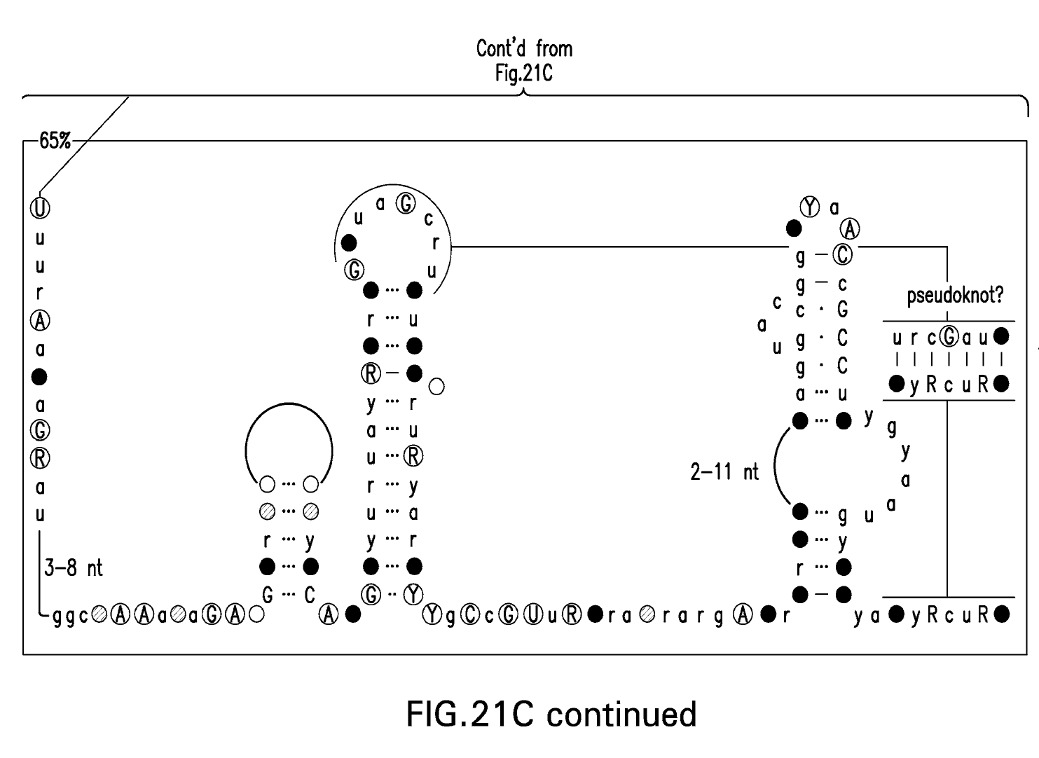

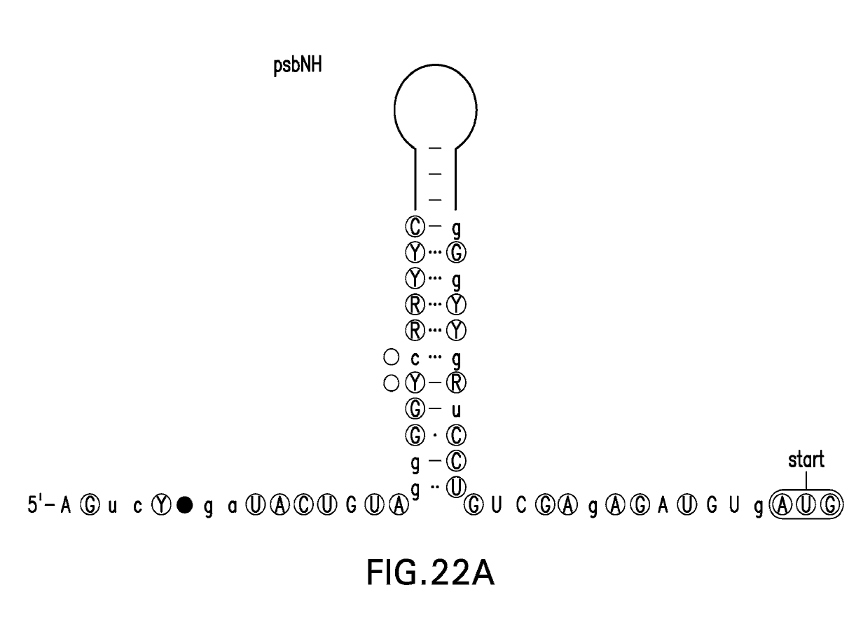

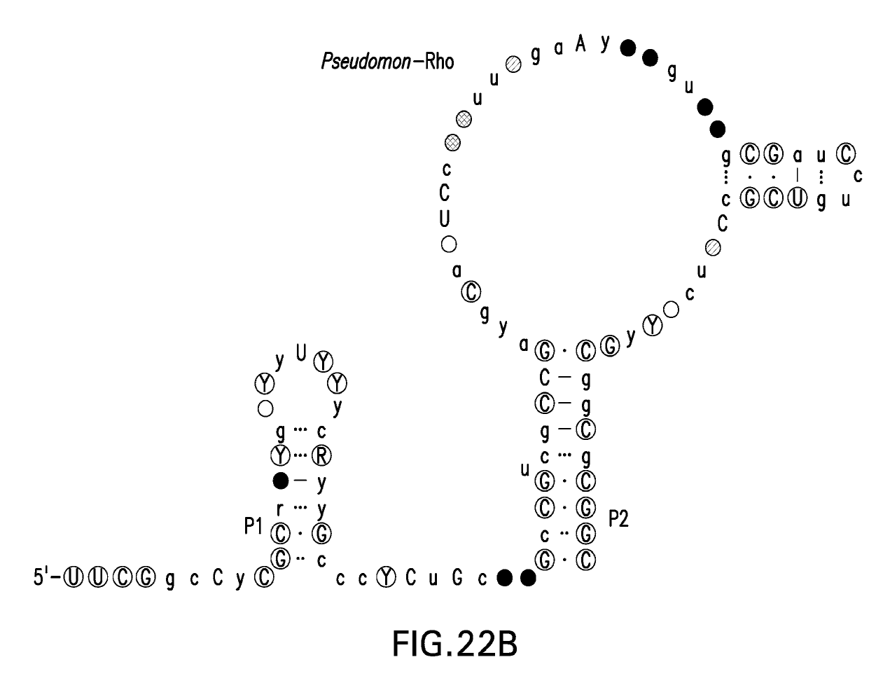

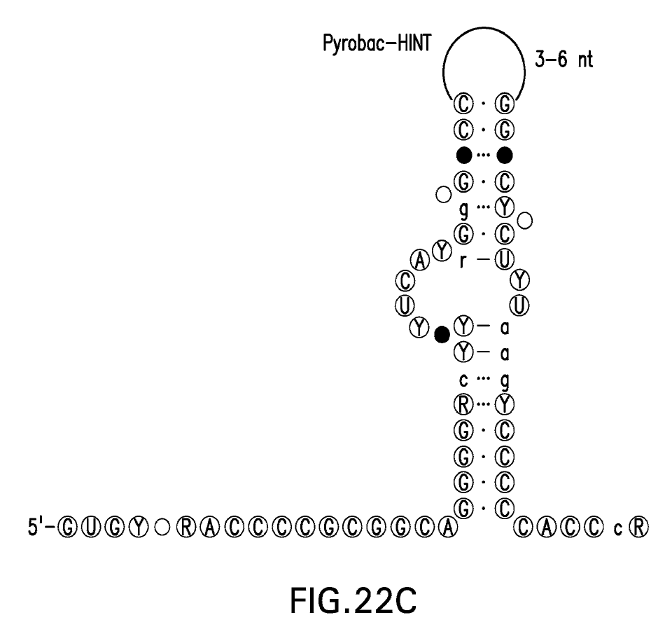

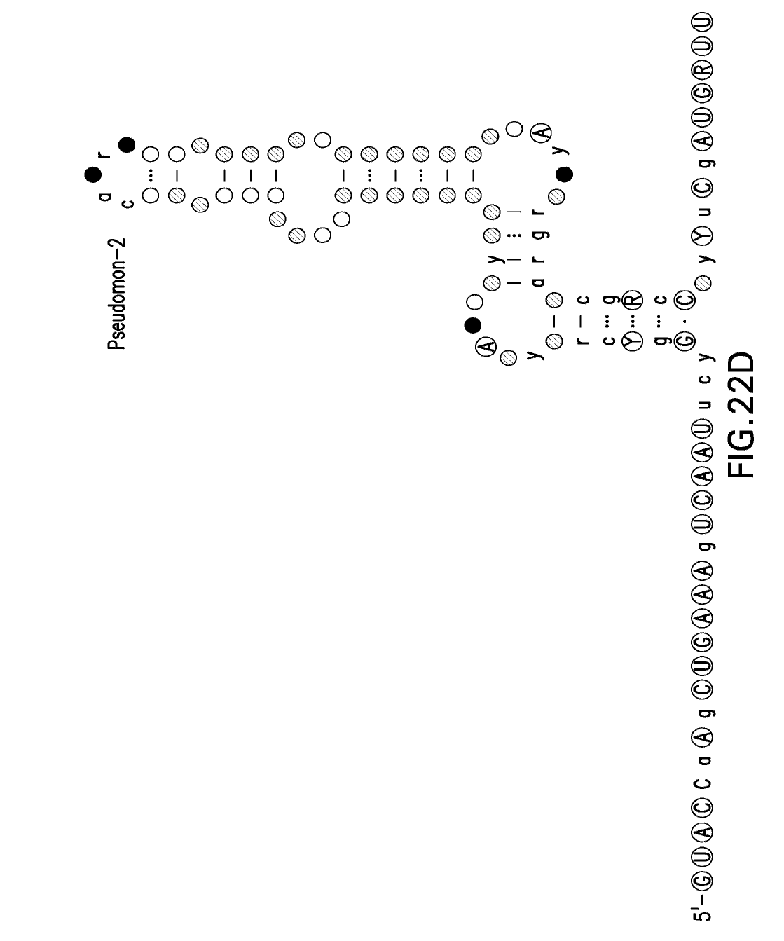

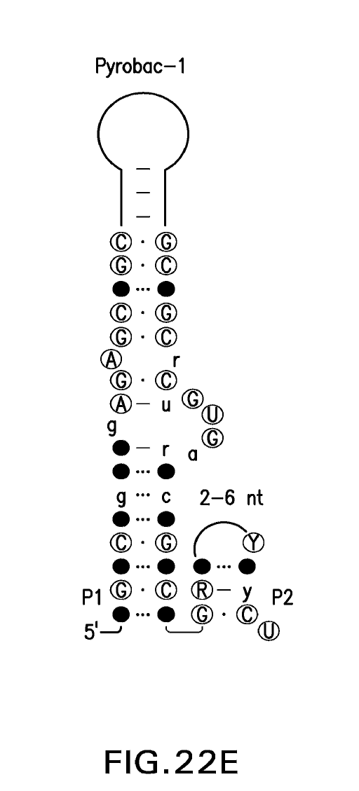

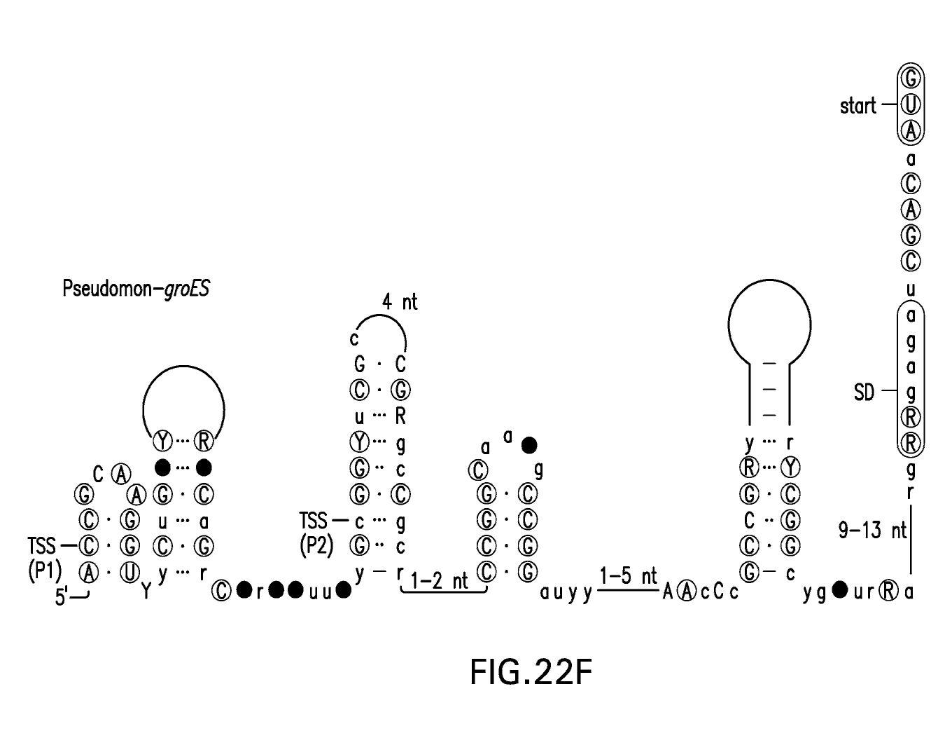

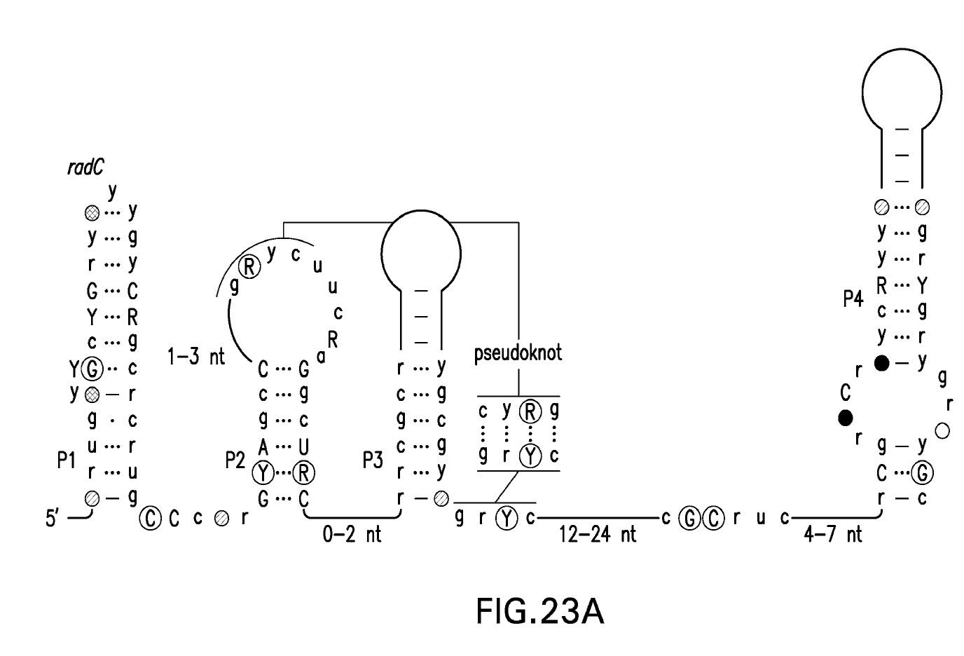

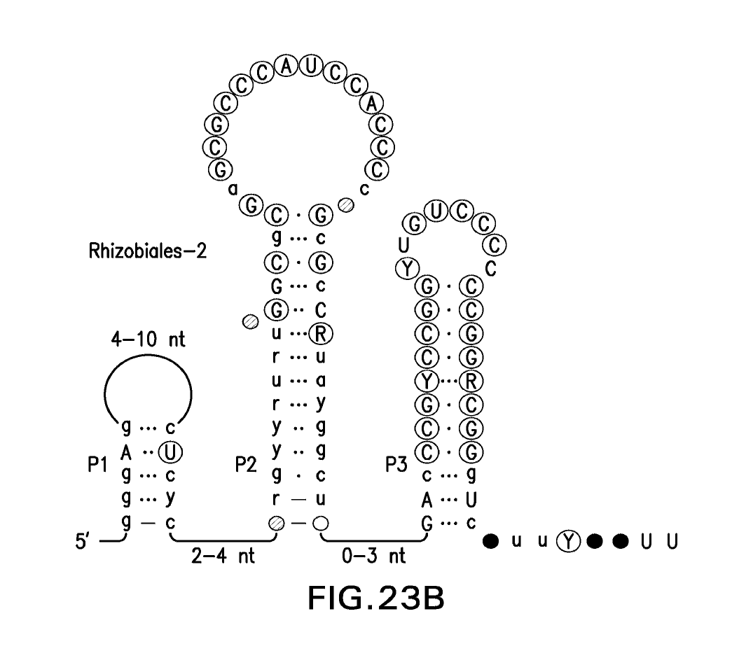

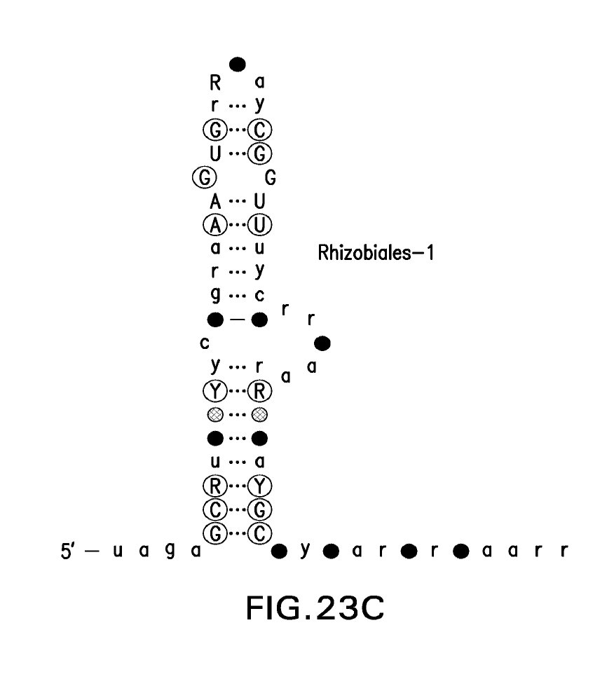

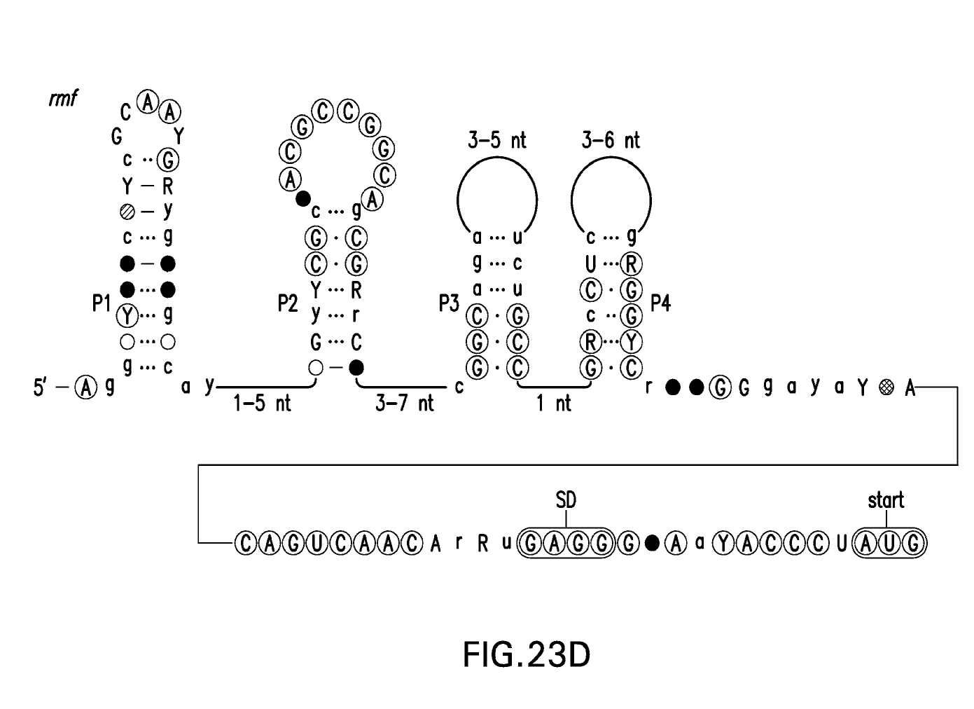

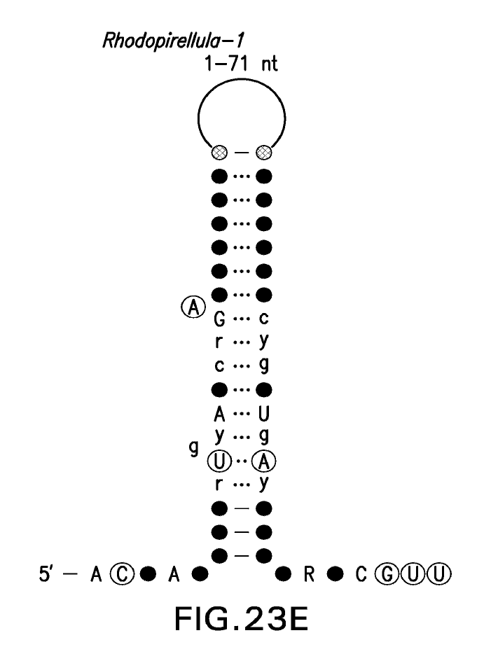

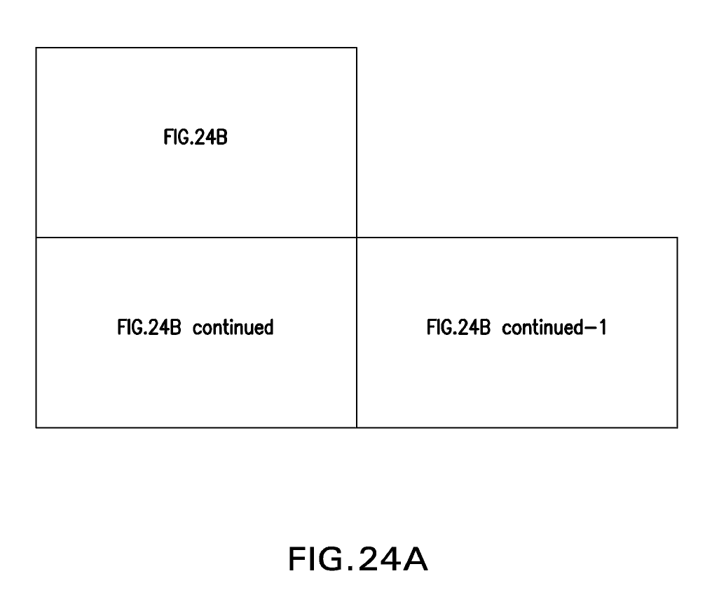

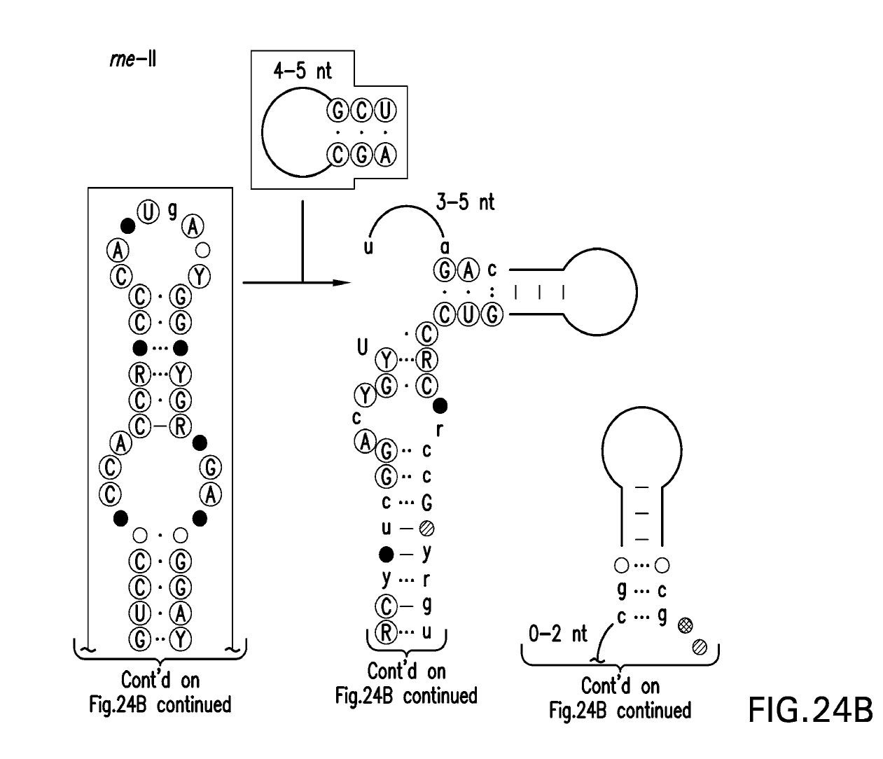

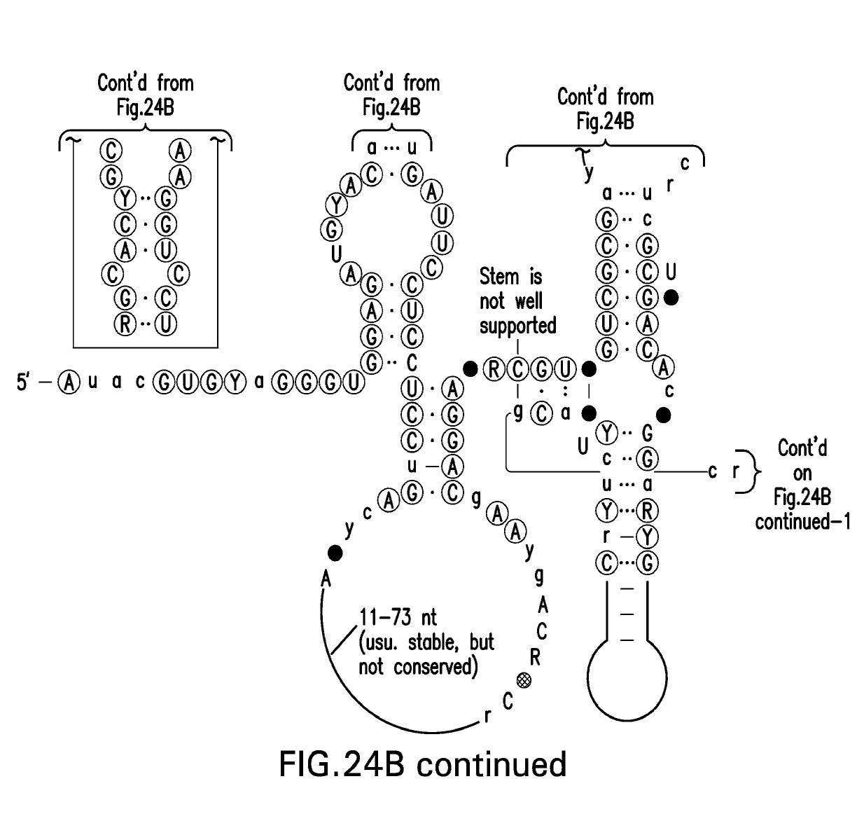

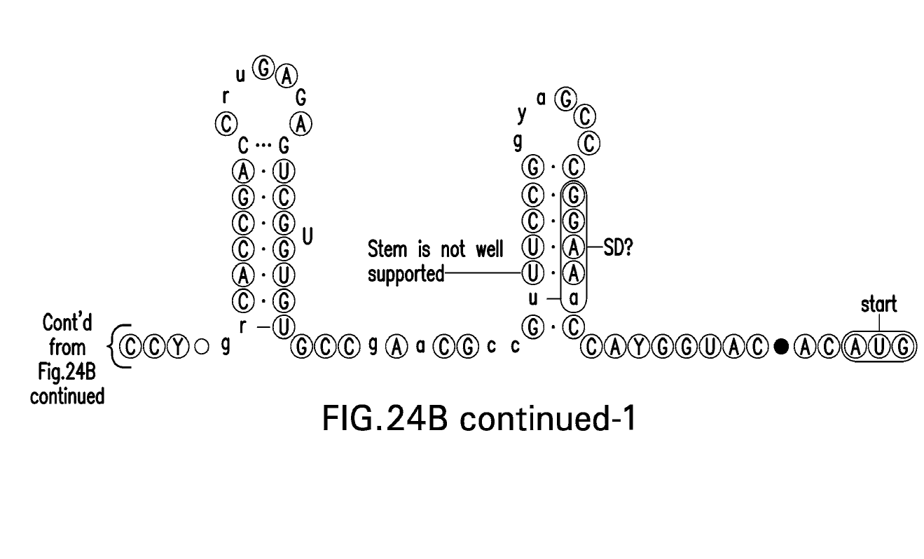

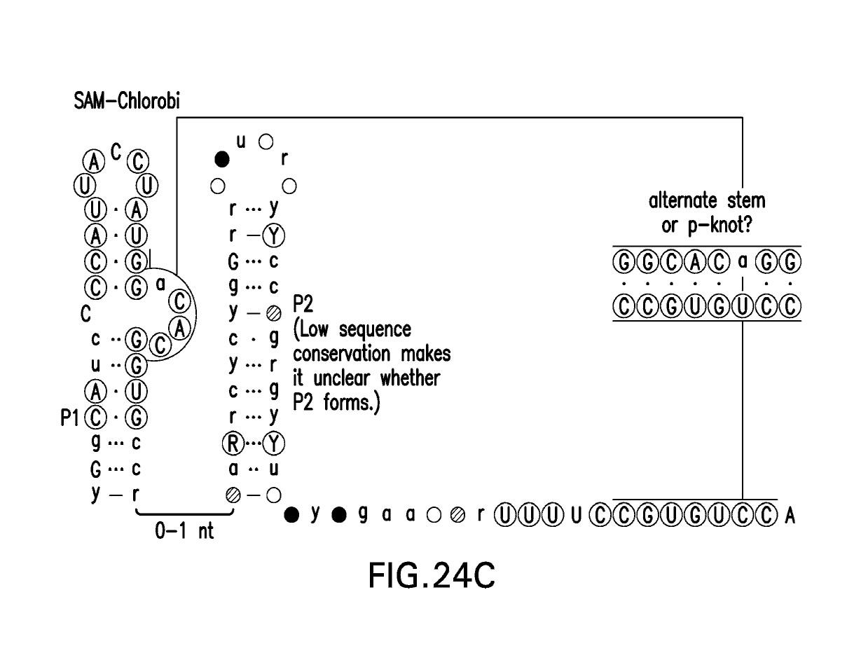

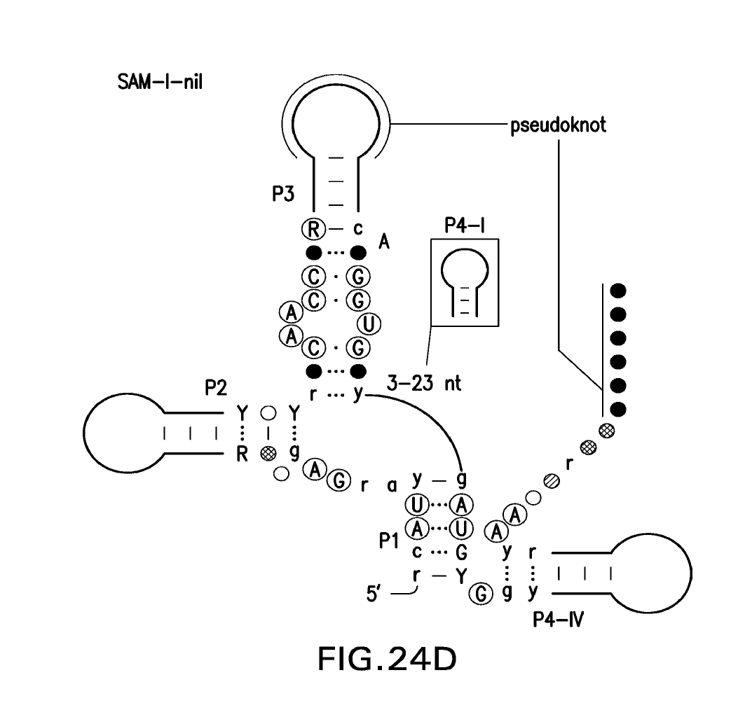

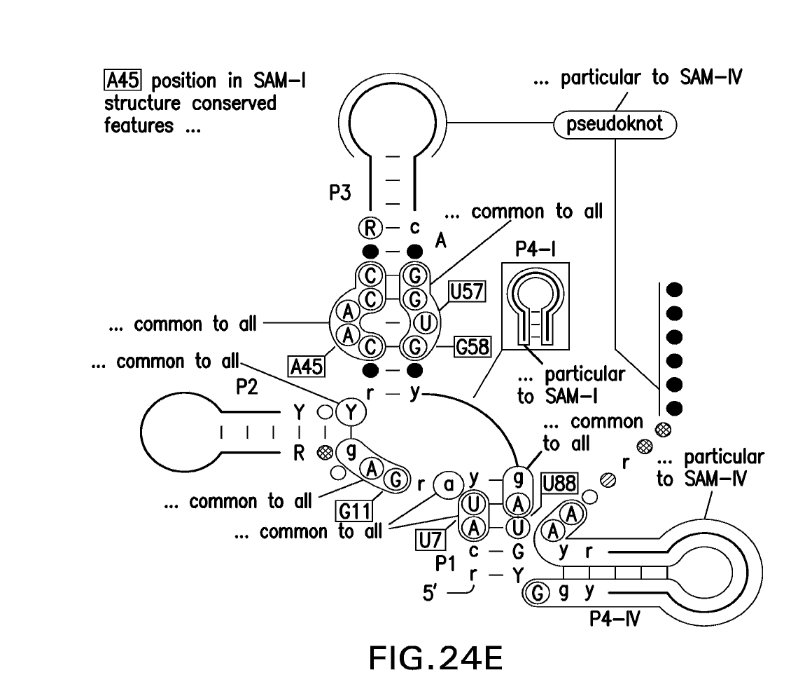

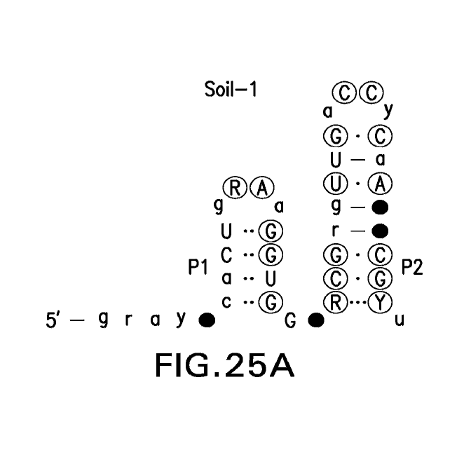

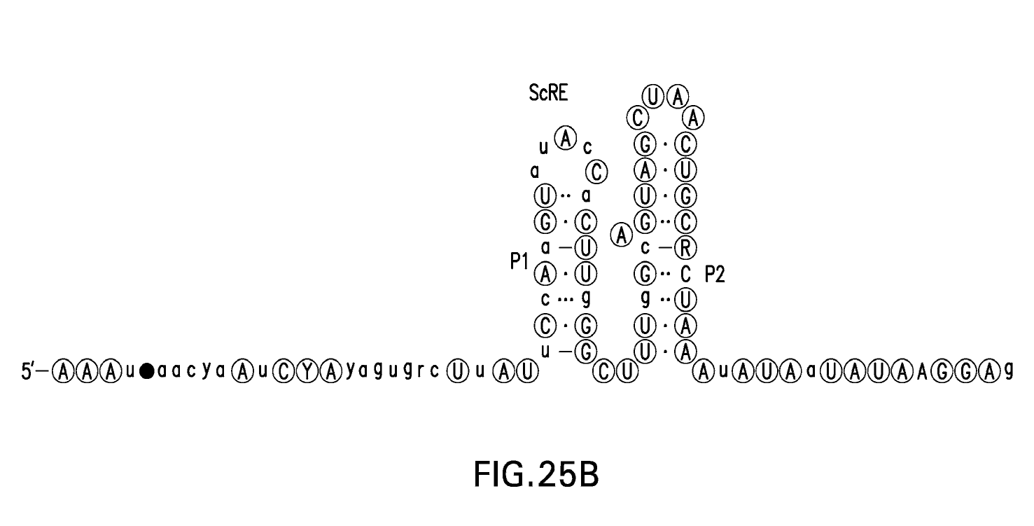

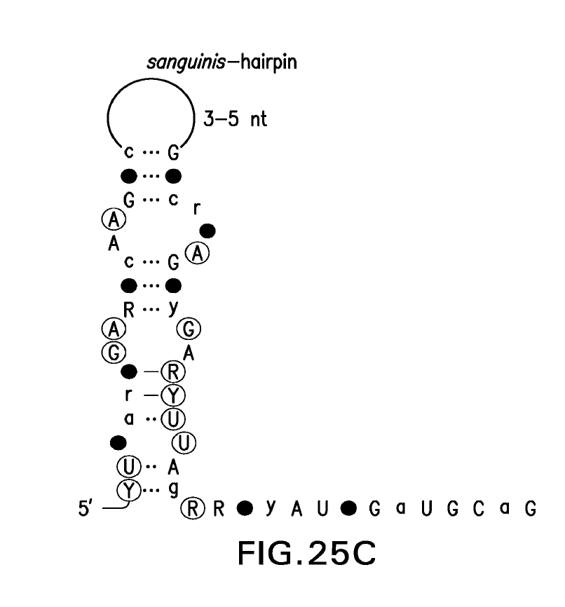

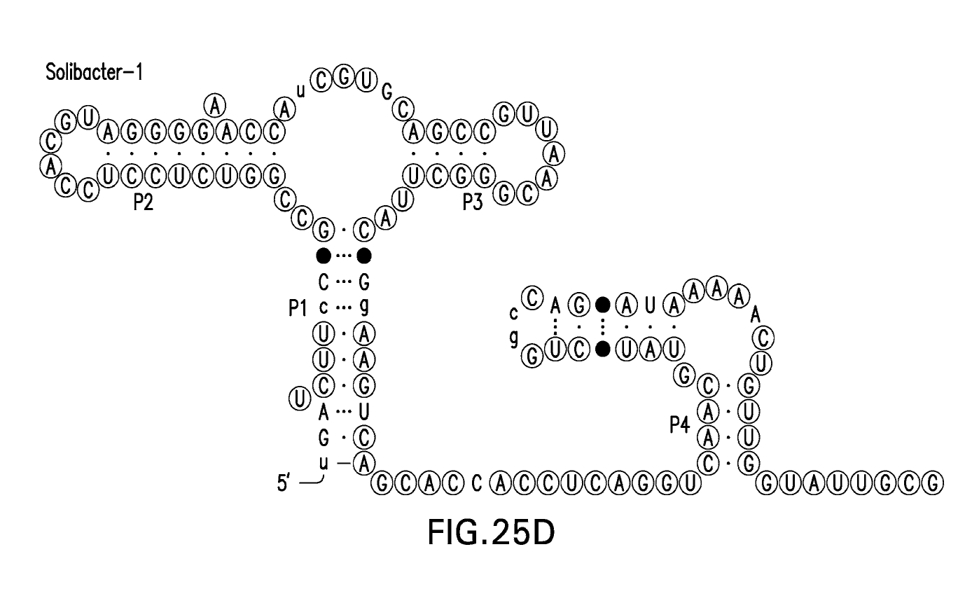

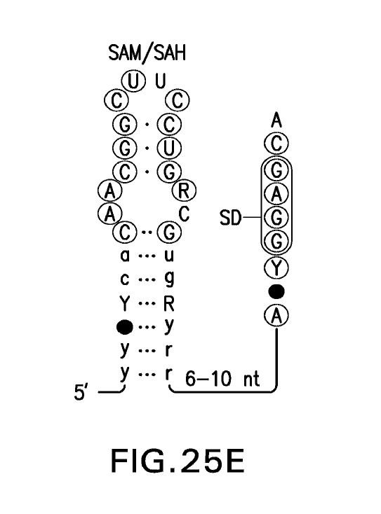

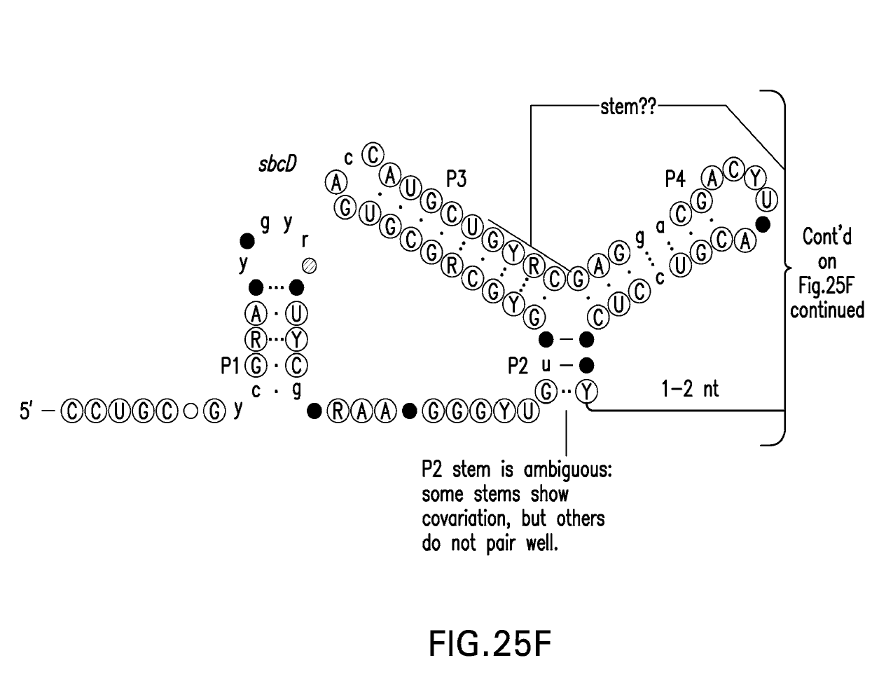

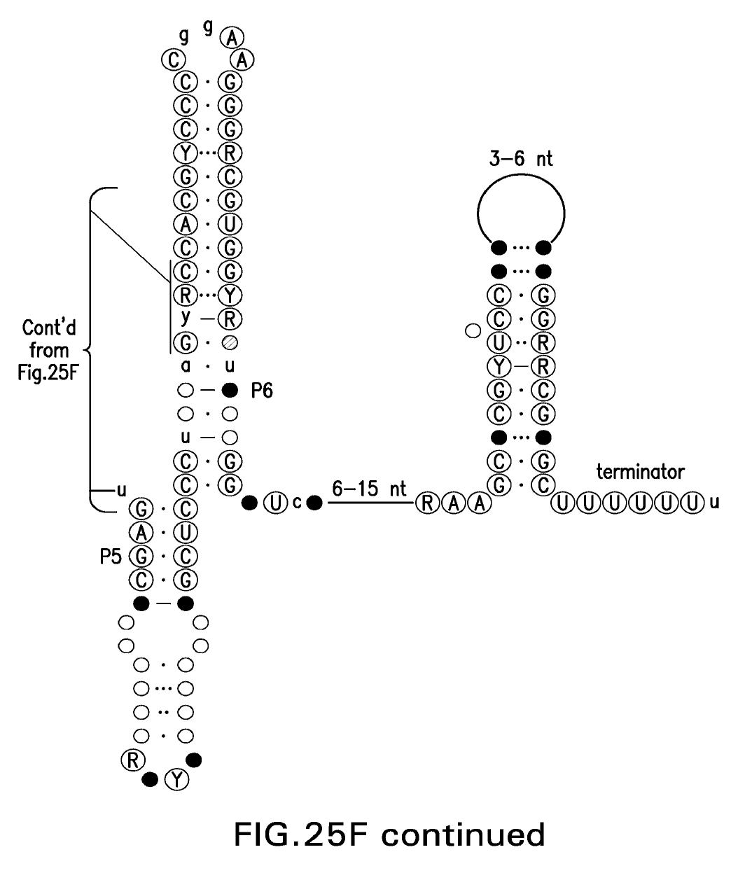

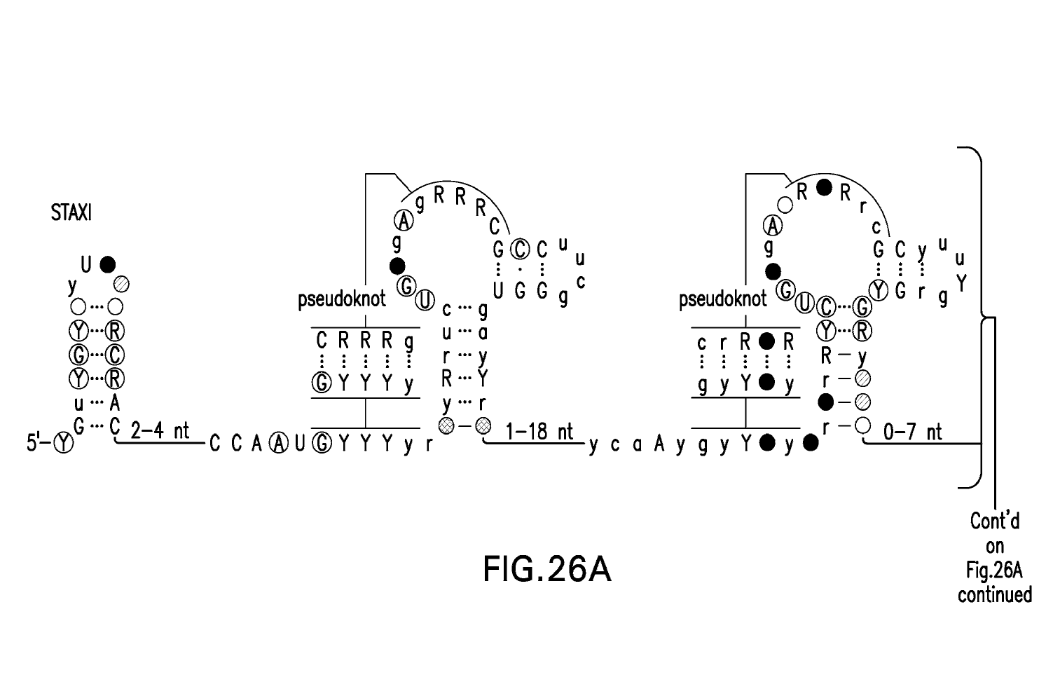

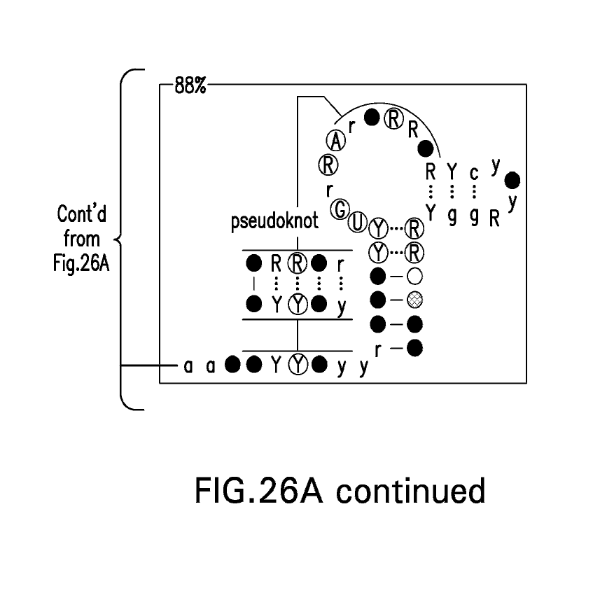

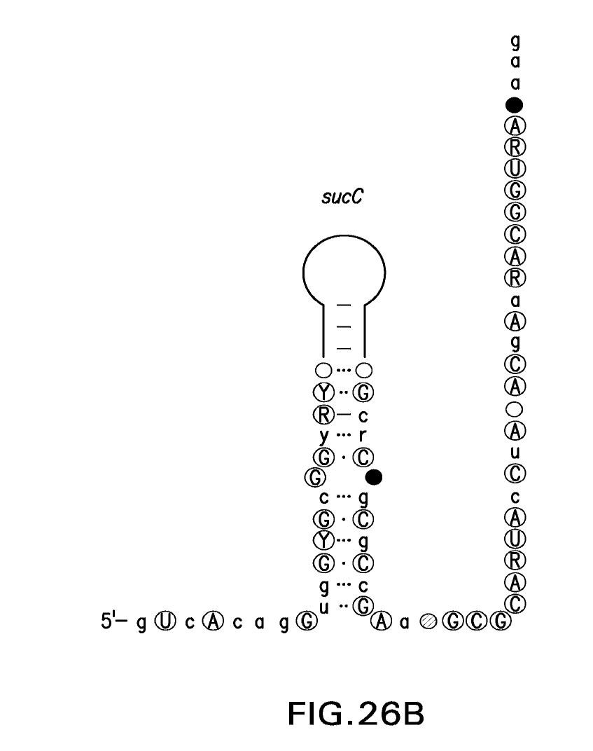

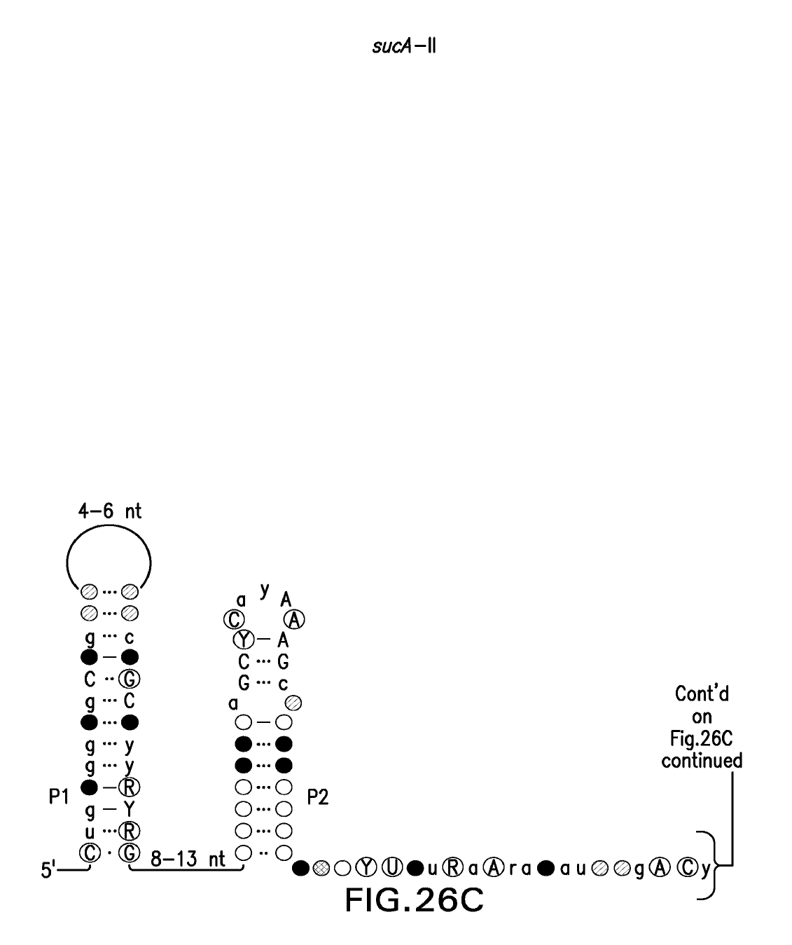

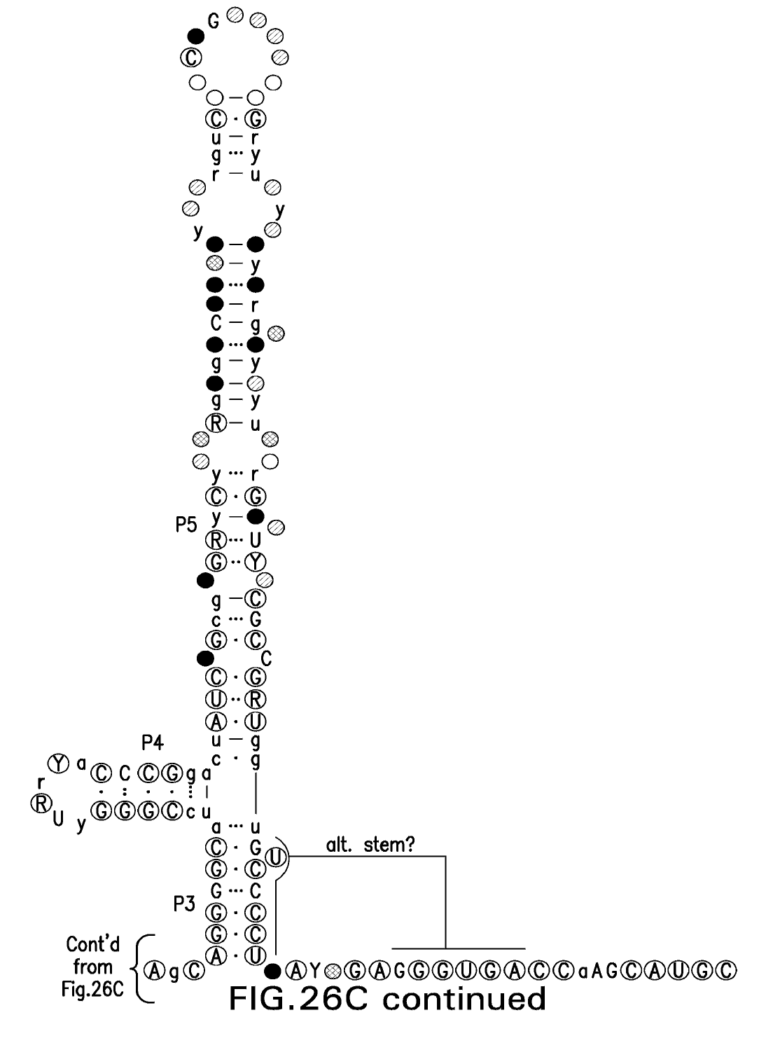

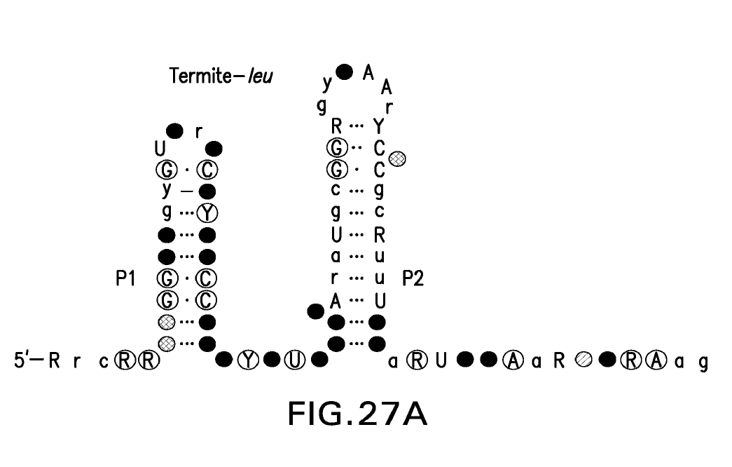

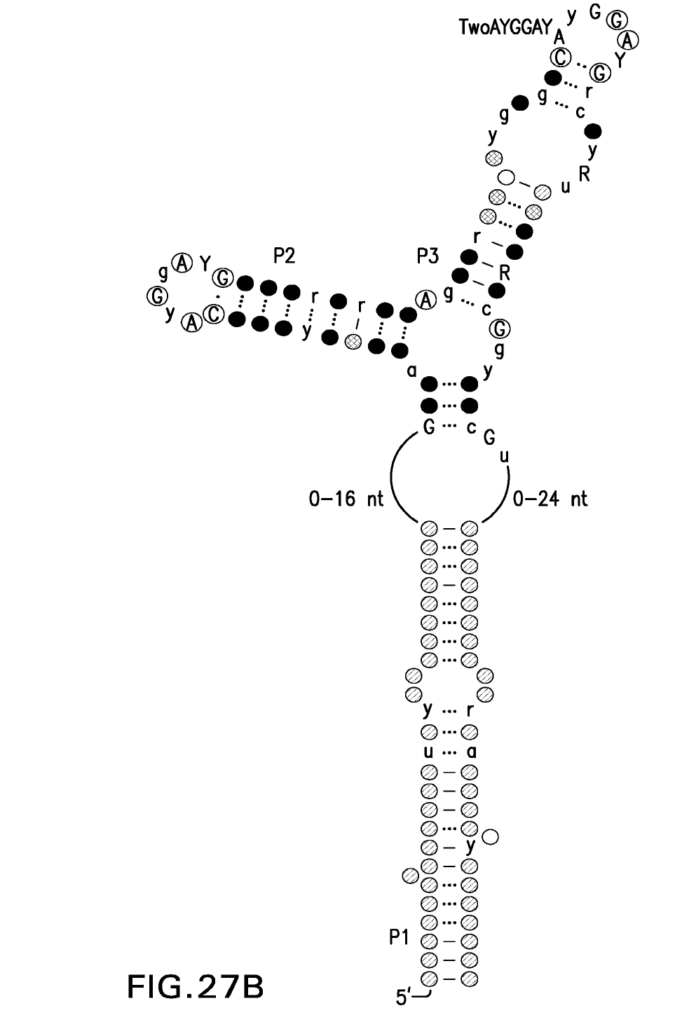

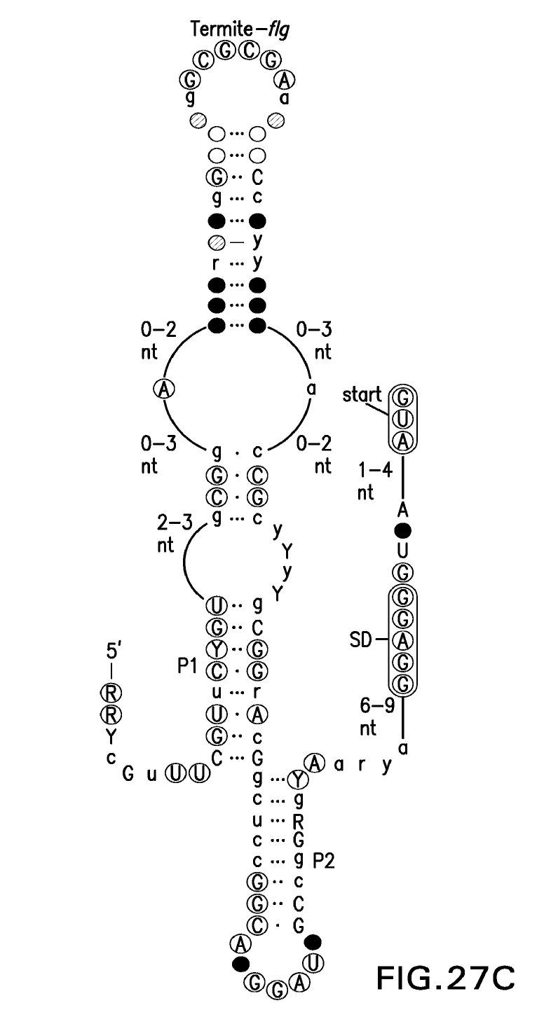

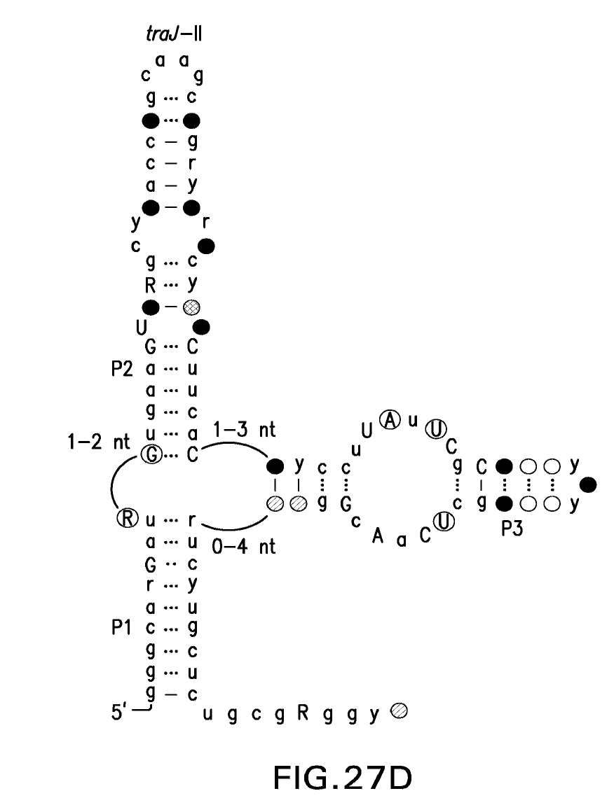

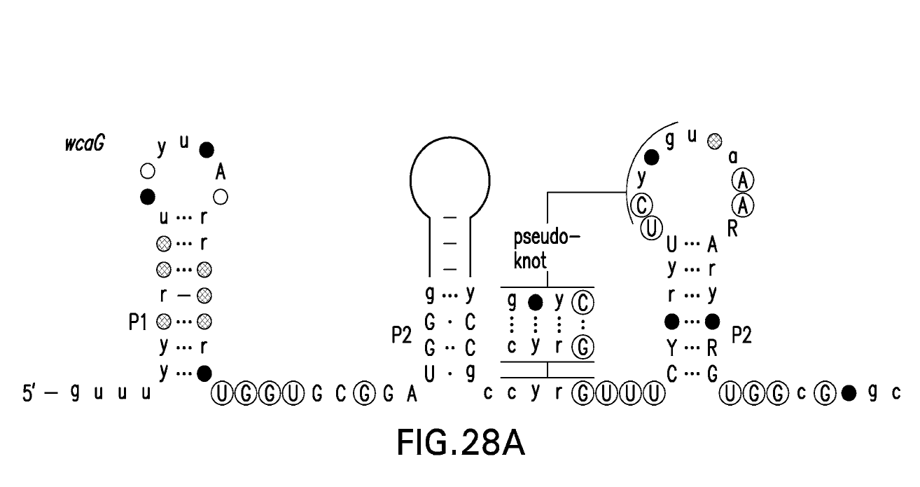

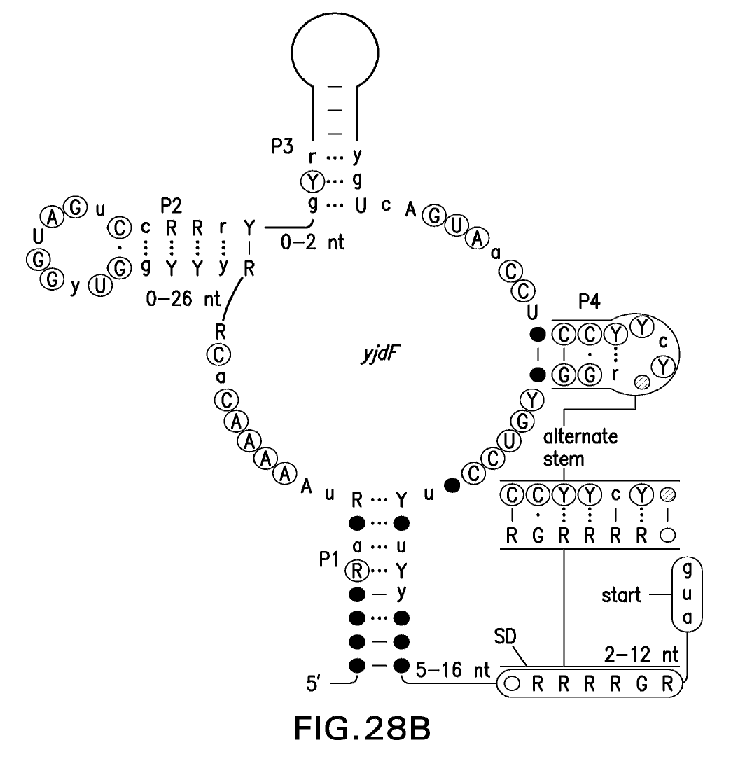

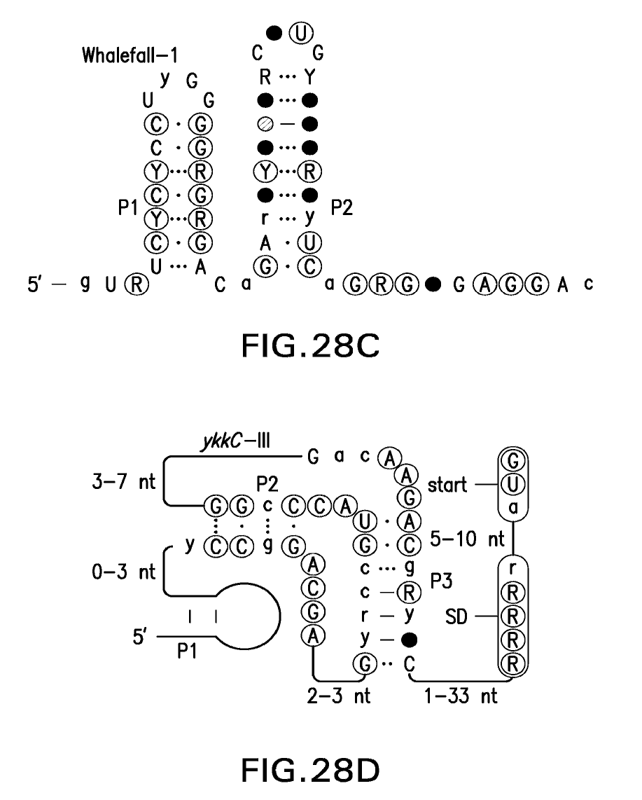

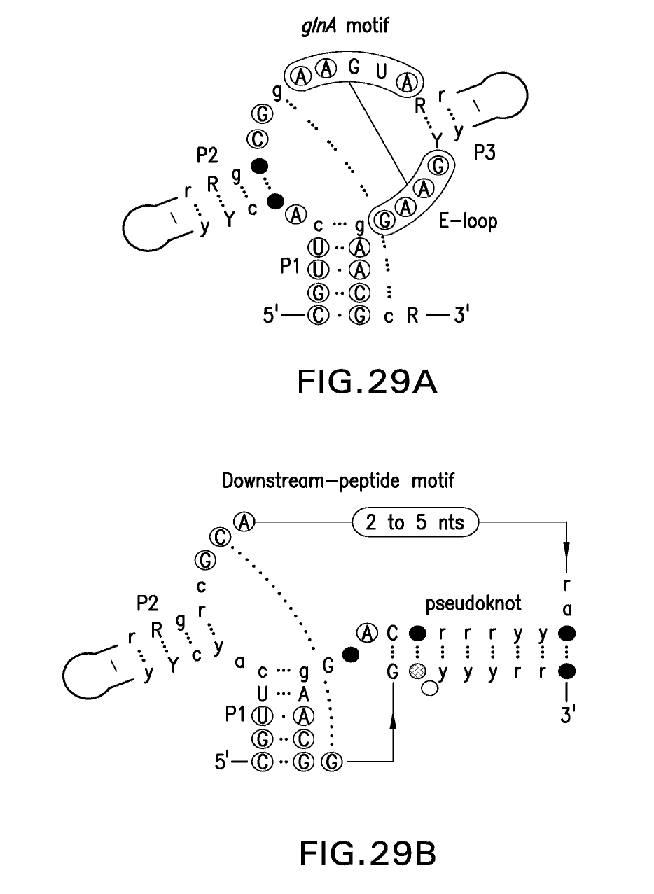

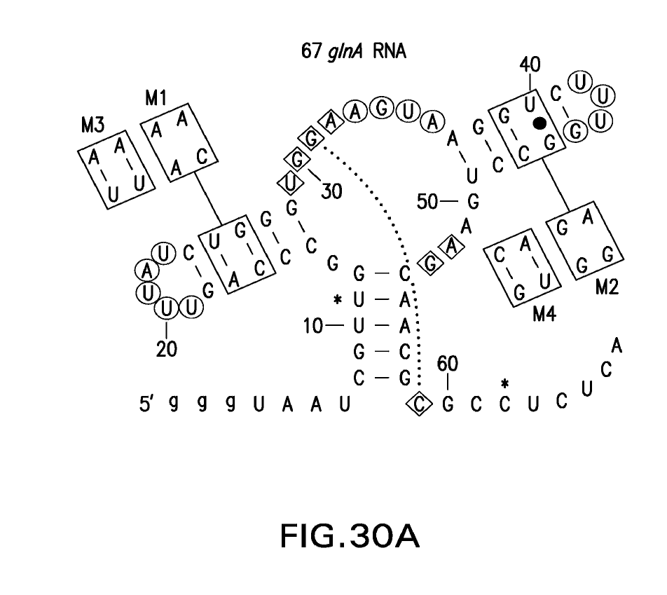

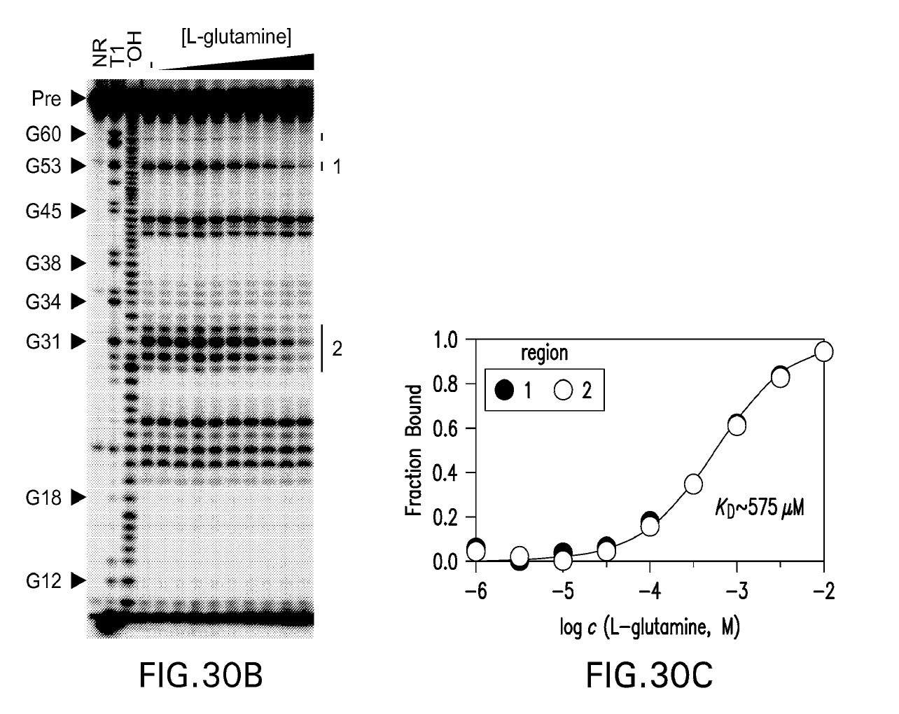

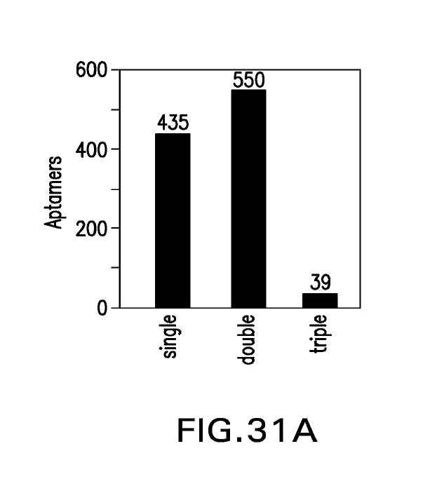

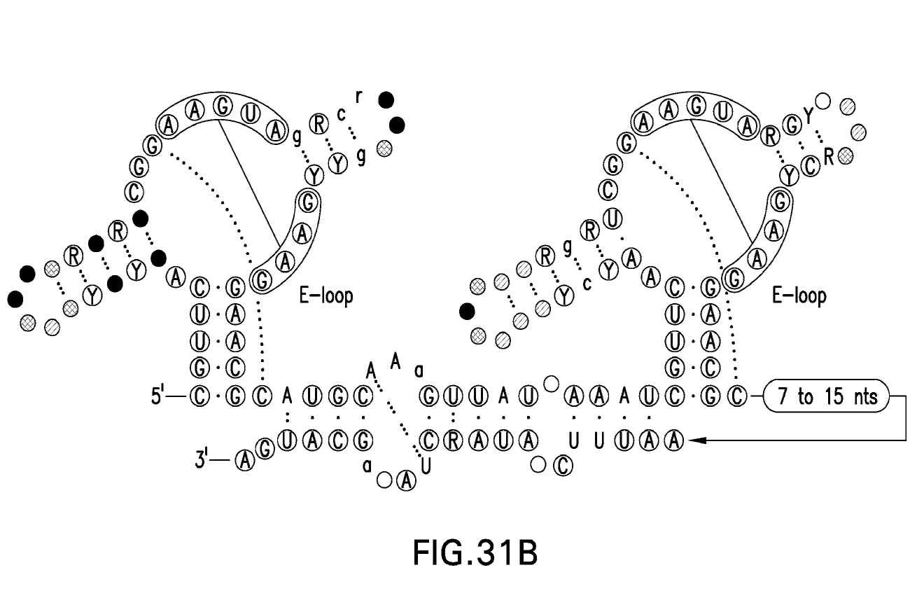

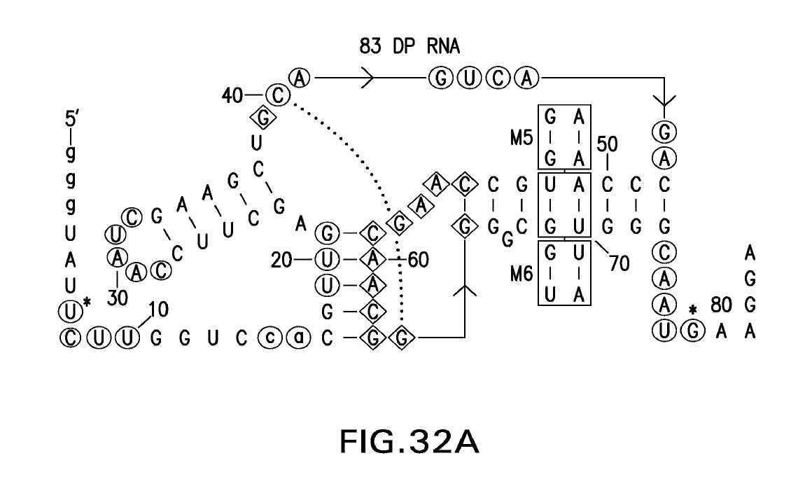

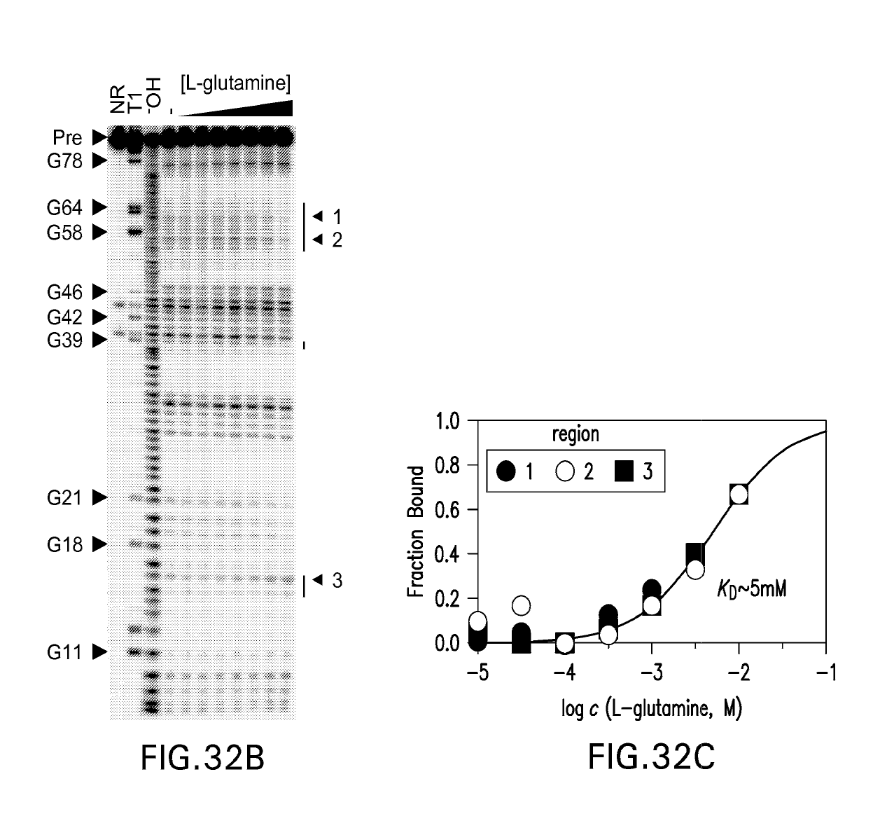

pfl RNA motifs are usually associated with genes involved in the synthesis of purines or that catalyze conversions between THF and its one-carbon adducts. On the basis of a previously published metabolism diagram (Ravcheev et al. Purine regulon of gamma-proteobacteria: a detailed description. Russian Journal of Genetics 2002, 38:1015-1025), most genes associated with pfl RNAs were found to be involved in these metabolic processes (FIG. 7).

The pfl riboswitch has been tested for ligand binding with a number of compounds (discussed below), but the most promising ligands seemed to be AICAR and PRPP. A build-up of AICAR could indicate insufficient levels of formyl-THF, without which formylation of AICAR cannot proceed. Many genes regulated by pfl RNAs could help to synthesize formyl-THF. High AICAR concentrations were a consequence of formyl-THF starvation in Salmonella typhimurium (Bochner et al. ZTP (5-amino 4-imidazole carboxamide riboside 5′-triphosphate): a proposed alarmone for 10-formyl-tetrahydrofolate deficiency. Cell 1982, 29:929-937), although this phenomenon was not observed in E. coli (Rohlman et al. Role of purine biosynthetic intermediates in response to folate stress in Escherichia coli. J Bacteriol 1990, 172:7200-7210). Since pfl RNAs are often present in an organism that is closely related to an organism lacking pfl RNAs, this RNA distribution could be consistent with a scenario in which closely related organisms differ in whether they produce high levels of AICAR in response to folate stress.

Alternately, high levels of PRPP is apparently an indicator of purine starvation, since the B. subtilis-type PurR repressor detects purine levels by sensing PRPP (Weng et al. Identification of the Bacillus subtilis pur operon repressor. Proc Natl Acad Sci USA 1995, 92:7455-7459). Note that the B. subtilis-type PurR has a distinct mechanism and is not homologous to the PurR protein found in E. coli, although their biological roles are analogous. It was hypothesized that PRPP can be a good indicator because excess adenine is phosphorylated and PRPP synthetase is inhibited by ADP, so high levels of adenine should lead to low levels of PRPP (Weng et al. Identification of the Bacillus subtilis pur operon repressor. Proc Natl Acad Sci USA 1995, 92:7455-7459). However, as noted below, the experiments with these ligands did not reveal evidence of binding.

Iiii. pheA Motif

The pheA motif is usually located upstream of pheA genes, which encode chorismate mutase. In cases where no annotated pheA gene is present, it is possible that the small ORF corresponding to pheA genes was missed.

Iiv. PhotoRC-I and PhotoRC-II Motifs

The genes associated with the PhotoRC-I motif in Synechococcus species are typically annotated as psbA genes. The psbA genes associated with these PhotoRC RNA motifs have not been studied, but psbA genes in related species have been studied. For example, multiple psbA paralogs are found S. elongatus PCC 7942 and are regulated transcriptionally and post-transcriptionally (Espinosa-Urgel et al. Expression of a Pscudomonas putida aminotransfcrasc involved in lysinc catabolism is induced in the rhizosphere. Appl Environ Microbiol 2001, 67:5219-5224). Just as psbA genes are observed in cyanophages (Platt et al. Proteomic, microarray, and signature-tagged mutagenesis analyses of anaerobic Pseudomonas aeruginosa at pH 6.5, likely representing chronic, late-stage cystic fibrosis airway conditions. J Bacteriol 2008, 190:2739-2758), a PhotoRC-II RNA is found upstream of a psbA gene in a cyanophage. Presumably the phage gene is regulated in the same way as for host-encoded psbA genes in this case. Indeed, since PhotoRC-II RNAs were found only in metagenome or phage sequences, it is possible that all PhotoRC-II RNAs detected were derived from phages or prophages.

Iv. Polynucleobacter-1 Motif

The Polynucleobacter-1 motif is found in marine environmental samples, but is also detected in Polynucleobacter sp. QLW-P1DMWA-1. The 3′ half of the motif is not always detected, but the 5′ part is well conserved among the examples found. Most Polynucleobacter-1 RNA motifs are downstream of genes classified into the family GOS11034 (Yooseph et al. The Sorcerer II Global Ocean Sampling expedition: expanding the universe of protein families. PLoS Biol 2007, 5:e16), and with possible homology to locus PSSM2_{—}218 in cyanophage P-SSM2. However, no Polynucleobacter-1 representatives were detected in any sequenced purified phage.

Ivi. potC Motif

The potC motif is located in the potential 5′ UTRs of genes predicted to encode transporters or peroxiredoxins. The motif is detected in marine metagenome sequences only.

Ivii. psaA Motif

Most highly conserved nucleotides in this structure are involved in base pairing. In contrast, most conserved positions in riboswitches do not reside in extended Watson-Crick base-paired structures. DNA corresponding to the motif can be bound by NtcA, a protein involved in nitrogen regulation that can also play a role in photosynthesis (Su et al. Computational inference and experimental validation of the nitrogen assimilation regulatory network in cyanobacterium Synechococcus sp. WH 8102. Nucleic Acids Res 2006, 34:1050-1065).

pshNH Motif

This motif is consistently found between psbN and psbH genes. Since the motif and its reverse complement are equally plausible, it is unclear which of these genes is regulated if the motif is a cis-acting regulatory RNA.

Iix. Pseudomon-1 Motif

The Pseudomon-1 motif is present in most species of Pseudomonas. It is consistently downstream of DNA polymerase I genes, and conceivably in their 3′ UTRs. It is usually, but not always, upstream of genes predicted to encode GTPases. However, these genes are in the opposite orientation to the Pseudomon-1 RNAs.

Ix. Pseudomon-2 Motif

The Pseudomon-2 motif has no apparent gene associations, so it can correspond to a trans-acting non-coding RNA. Although the alignment is supported by some covariation, the structure is not overall strongly conserved and therefore may not represent a structured RNA.

Ixi. Pseudomon-GGDEF Motif

The Pseudomon-GGDEF motif is confined to Pseudomonas syringae, where it resides 5′ of genes predicted to encode cyclic di-GMP synthases. The previously identified cyclic di-GMP riboswitch is sometimes present upstream of cyclic di-GMP synthases. However, the sequences exhibiting the Pseudomon-GGDEF motif are closely related, and so it is difficult to evaluate the conservation of structure, or sequence identities. One stem is supported by covariation, but there are also a few instances of non-canonical base pairs.

Ixii. Pseudomon-groES Motif

The groES and groEL operon is involved in the heat shock response in many bacteria. In Pseudomonas aeruginosa, experiments showed that transcription of this operon starts at one of two sites, termed P1 and P2 (Fujita et al. Transcription of the groESL operon in Pseudomonas aeruginosa PAO1. FEMS Microbiol Lett 1998, 163:237-242). P1 is located at the 5′ end of the Pseudomon-groES RNA, while P2 is located inside the RNA motif (FIG. 22). Transcripts starting at P1 and P2 are both increased at roughly the same levels during heat shock (Fujita et al. Transcription of the groESL operon in Pseudomonas aeruginosa PAO1. FEMS Microbiol Lett 1998, 163:237-242). Therefore, the RNA likely does not participate in this regulation. However, P1-initiated transcripts, which contain full-length RNA, can undergo additional regulation that is mediated by the RNA.

Ixiii. Pseudomon-Rho Motif

The Pseudomon-Rho motif consists of two hairpins with some sequence conservation that are consistently upstream of the gene encoding the Rho protein. The Rho protein interacts with RNA, indicating that the RNA motif can be part of an autoregulatory circuit to maintain appropriate levels of the Rho protein.

Ixiv. Pyrobac-1 Motif

The Pyrobac-1 motif is found in archaea in the genus Pyrobaculum. Given its lack of a gene association, it can correspond to a trans-acting RNA. Although many small nucleolar RNAs (snoRNAs) have been identified in archaea, Pyrobac-1 RNAs do not share typical features of either C/D box or H/ACA box snoRNAs.

Ixv. Pyrobac-HINT Motif The Pyrobac-HINT motif has only four known representatives, one for each of the four sequenced species in the genus Pyrobaculum. All four representatives are immediately upstream of a HINT protein (domain “cd01277”), which contains a histidine triad motif (Seraphin B: The HIT protein family: a new family of proteins present in prokaryotes, yeast and mammals. DNA Seq 1992, 3:177-179).

Ixvi. radC Motif

The radC motif is consistently in the potential 5′ UTRs of genes encoding proteins that operate on DNA, such as radC DNA repair proteins, integrases, methyltransferases that can operate on DNA and an anti-restriction protein. The most common gene is annotated as radC. The radC gene was initially thought to be involved in DNA repair, but the key mutation was later shown to be located in a different gene (Lombardo et al. radC 102 of Escherichia coli is an allele of recG. J Bacteriol 2000, 182:6287-6291; Finn et al. Pfam: clans, web tools and services. Nucleic Acids Res 2006, 34:D247-251). However, while the function of radC is currently unknown, DNA repair is broadly related to the other functions associated with radC RNAs. Although the RNAs are associated with integrases, no radC RNA was detected in any sequenced purified phage.

Ixvii. Rhizobiales-1 Motif

The Rhizobiales-1 motif is present in many species of α-proteobacteria, especially those in the order Rhizobiales. It is commonly present in many copies per genome, as many as 92 in Nitrobacter hamburgensis X14, but 40 copies is a typical number. The motif consists of a hairpin with some conserved sequence features.

Ixviii. Rhodopirellula-1 Motif

The Rhodopirellula-1 motif is a hairpin with characteristic bulges, and sequence conservation surrounding its base. The terminal loop varies widely in size, and some long variants exist that do not appear to have a stable structure. The stem itself exhibits significant covariation, but has some non-canonical base pairs. Since many of these seem to be A-C pairs, it is possible that the true RNA may be the reverse complement of the motif, although that orientation also has several A-C pairs. Rhodopirellula-1 RNAs are generally in 5′ regulatory configurations to genes that arc often short and hypothetical. In many cases where the motif appears not to be located 5′ of a coding region, it is possible that an undetected short hypothetical gene is actually present. The motif occurs in a few phyla, but is overwhelmingly the most dominant in Planctomycetes. Its name derives from the fact that it has 36 predicted instances in Rhodopirellula baltica SH 1. These occurrences tend to cluster together in the genome, although they are located at least ˜500 nucleotides apart. Rhodopirellula-1 RNAs are also present in other species of Planctomycetes, some Proteobacteria and other assorted bacteria.

Ixix. rmf Motif

NCBI GEO queries (Barrett et al. NCBI GEO: archive for high-throughput functional genomic data. Nucleic Acids Res 2009, 37:D885-890) revealed that the rmf gene in Pseudomonas aeruginosa (locus PA3049) is differentially regulated by azithromycin exposure (Nalca et al. Quorum-sensing antagonistic activities of azithromycin in Pseudomonas aeruginosa PAO1: a global approach. Antimicrob Agents Chemother 2006, 50:1680-1688) and by co-culturing with human airway epithelial cells (Chugani et al. The influence of human respiratory epithelia on Pseudomonas aeruginosa gene expression. Microb Pathog 2007, 42:29-35). The rmf RNA motif can play a role in this regulation.

Ixx. rne-II Motif

The rne-II motif is consistently in the potential 5′ UTRs of RNase E genes. It is present in species of the family Pseudomonadaceae. A cis-regulatory RNA is known that is in the 5′ UTRs of RNase E genes in enterobacteria (e.g., E. coli) (Diwa et al. An evolutionarily conserved RNA stem-loop functions as a sensor that directs feedback regulation of RNase E gene expression. Genes Dev 2000, 14:1249-1260). The enterobacterial motif is a complex structure that is a substrate for RNase E. Cleavage of the RNA by RNase E leads to reduced gene expression. The rne-II motif can perform a similar function. No similarity in sequence or structure to the previously identified element was detected, other than the general observation that both structures have many stems.

Ixxi. SAM-Chlorobi Motif Sequences conforming to strong promoters are found upstream of all SAM-Chlorobi RNA motifs, indicating that the RNAs are transcribed. These promoter sequences were validated in Bacteroidetes (Bayley et al. Analysis of cepA and other Bacteroides fragilis genes reveals a unique promoter structure. FEMS Microbiol Lett 2000, 193:149-154; Chen et al. Characterization of strong promoters from an environmental Flavobacterium hibcrnum strain by using a green fluorescent protein-based reporter system. Appl Environ Microbiol 2007, 73:1089-1100), which is a phylum that is related to the phylum Chlorobi (Gupta R S: The phylogeny and signature sequences characteristics of Fibrobacteres, Chlorobi, and Bacteroidetes. Crit. Rev Microbiol 2004, 30:123-143), in which SAM-Chlorobi RNAs are found. Therefore, these conserved promoter sequences can, in fact, facilitate transcription of SAM-Chlorobi RNAs. These putative promoter sequences are marked in the SAM-Chlorobi motif sequence alignment (data not shown).

Ixxii. SAM-I/SAM-IV Variant Riboswitch Motif

SAM-I and SAM-TV riboswitches share features in their ligand-binding core, although they have several distinctions in their overall architecture (Weinberg et al. The aptamer core of SAM-IV riboswitches mimics the ligand-binding site of SAM-I riboswitches. Rna 2008, 14:822-828). One commonality is a pseudoknot formed by the tip of P2 binding to the junction 3′ to P3. SAM-1 riboswitches have a kink turn that facilitates formation of this pseudoknot (Montange et al. Structure of the S-adenosylmethionine riboswitch regulatory mRNA element. Nature 2006, 441:1172-1175). SAM-IV riboswitches have an internal loop with a distinct sequence that can also create a turn in P2. However, most of the new-found SAM-I/SAM-IV variant RNAs entirely lack an internal loop in their P2 stem. Moreover, a pseudoknot involving the tip of P2 may not exist, as a significant base-pairing potential is not observed.

Ixxiii. SAM/SAH Riboswitch

A pseudoknot pairing is possible between the tip of the hairpin (CUUC) and the Shine-Dalgarno sequence. However, there is only one mutation observed in these sequences, and this mutation disrupts Watson-Crick pairing. Nucleotides on both sides of the putative pseudoknot do show modest reduction in cleavage in in-line probing experiments on SK209-52 RNA. When the 3′-most 5 nucleotides of this RNA are removed, ligand-mediated structure modulation is not observed. This result is consistent with a pseudoknot interaction that stabilizes the nucleotides involved.

Ixxiv. Sanguinis-Hairpin Motif

The sanguinis-hairpin motif is a hairpin that is found in Streptococcus sanguinis and S. thermophilus. In S. sanguinis, there are four repeats in one part of the genome, and three repeats in a nearby region. These repeat regions include short spacers whose sequences are not conserved.

Ixxv. sbcD Motif

The sbcD motif is in the potential 5′ UTRs of apparent operons that can include sbcD genes, and other DNA repair genes. Since the sbcD genes are not the immediately downstream gene, and since all sbcD RNA motifs are located in apparently syntenic regions, it is difficult to ascertain whether the sbcD motif is truly associated with sbcD genes. If it is, SbcD is thought to be involved in removal of palindromic DNA sequences, which can be problematic during replication (Connelly et al. The sbcC and sbcD genes of Escherichia coli encode a nuclease involved in palindrome inviability and genetic recombination. Genes Cells 1996, 1:285-291). Therefore, sbcD RNAs can operate as DNA mimics, perhaps as a feedback system of regulation for SbcD, or they can operate as ssDNA structures. sbcD RNAs are usually, but not always, located in plasmids.

Ixxvi. ScRE (Streptococcus Regulatory Element) Motif

The ScRE motif has only modest covariation, and some non-canonical nucleotides. Therefore its assignment as a structured RNA is tenuous. However, it is consistently located upstream of several non-homologous classes of protein-coding genes, which indicates it to be a functional cis-regulatory element.

Ixxvii. Soil-1 Motif

The Soil-1 motif is found only in metagenomic DNA isolated from soil samples. The motif consists of two hairpins. Although the first often ends in a GNRA tetraloop, no covariation is evident. The second stem exhibits a moderate amount of covariation, but also carries non-canonical base pairs.

Ixxviii. sucA-II Motif

The sucA-II motif is found in the potential 5′ UTRs of sucA genes in species of the genus Pseudomonas. SucA is part of an enzyme in the citric acid cycle that it responsible for creating succinate. A distinct RNA motif was previously identified upstream of sucA genes in certain β-proteobacteria (Weinberg et al., 2007).

Ixxix. sucC Motif

The sucC motif is a hairpin structure that is in the potential 5′ UTRs of an apparent sucCD operon in Pseudomonas. A potential sucC RNA in Marinobacter sp. ELB17 was also predicted, which is in a different order of γ-proteobacteria, but it is not clear if this sequence is a true homolog. In this species, sucC RNA is in the potential 5′ UTRs of a predicted polyphosphate kinase. However, while the sucC motif appears to correspond to a cis-regulatory RNA, it is not clear why polyphosphate kinases should be co-regulated with sucCD genes, indicating that the predicted homology can be a false positive. The sucC motif is one of multiple motifs that can be involved in regulating the citric acid cycle in Pseudomonas.

Ixxx. Solibacter-1 Motif

The Solibacter-1 motif is found in many copies in the species Solibacter usitatus. The motif includes a three-stem junction, but it is supported by only modest covariation, and has some nucleotide pairs that are not normally energetically favorable. In view of this observation, and the fact that it is present in many copies in one organism, the motif can correspond to a repetitive element.

Ixxxi. Termite-fig Motif

The Termite fig motif is found only in environmental sequences from a termite hindgut metagenome, and is not detected in any genome from a known species. It is in the potential 5′ UTRs of flagellar genes.

Ixxxii. Termite-flg Motif

The Termite-leu motif is found only in metagenome samples from termite hindguts, and consists of two hairpins. It is sometimes in the potential 5′ UTRs of a variety of leucine-related genes: leuA, leuB and ilvC, but often the downstream gene is in the opposite orientation. While some of these genes are likely misannotations due to the challenges inherent in annotating metagenome fragments, some are homologous to known gene families.

Some cis-regulatory RNAs are peptide leaders (Vitreschak et al. Attenuation regulation of amino acid biosynthetic operons in proteobacteria: comparative genomics analysis. FEMS Microbiol Lett 2004, 234:357-370), which contain a short ORF that encodes a peptide rich in some amino acid. When levels of this amino acid are low, ribosomal stalling leads to increased expression of the downstream gene. The product of this downstream gene is typically required to synthesize the given amino acid. Termite-leu RNAs upstream of leuA and ilvC genes contain a short ORF immediately 5′ to their first hairpin that is rich in codons for branched-chain amino acids (BCAAs) (i.e., leucine, isoleucine and valine). The leuA and ilvC gene products are involved in the synthesis of these related amino acids. Moreover, these short ORFs exhibit some mutations that change a codon for one BCAA to a codon for a different BCAA, a phenomenon that indicates that the BCAA-rich ORF can be functionally important. However, a Termite-leu RNA that is upstream of a leuB gene does not have a similar ORF.

Ixxxiii. traJ-II Motif

The traJ-II motif is typically found in the apparent 5′ UTRs of traJ genes. A previously identified motif, which is called traJ-I (Rfam accession RF00243), was identified in E. coli, and the closely related genus Salmonella (Arthur et al. FinO is an RNA chaperone that facilitates sense-antisense RNA interactions. Embo J 2003, 22:6346-6355). The traJ-II motif has no apparent similarities in sequence or structure to the earlier motif. It is present in α-, β- and γ-Proteobacteria, although in each bacterial class it is only present in a few species. This distribution can be the result of horizontal transfer via conjugation, the process in which traJ functions. Since traf-I RNAs are targets for FinP antisense RNAs, it is natural to speculate that traJ-II RNAs are also targets of an antisense RNA. The traJ-II motif has no obvious expression platform, though annotated start codons are often 20-30 nucleotides 3′ to the traJ-II RNA. The RNA can be expressed as the reverse complement, as there are fewer A-C mismatches in the reverse complementary sequences of traJ-II RNAs, and the P2 stem would have a structurally stable CUUG terminal loop.

Ixxxiv. Transposase-Resistance Motif

The Transposase-resistance motif is typically found in the potential 5′ UTRs of genes, and these genes or surrounding genes often confer antibiotics resistance. Although there are few transposons and integrases, there are more than would be expected by chance. Resistance genes are: emrE (include drug exporters), nucleotidyltransferases (includes kanamycin resistance), pfam03595 transporters (include tellurite exporters), dihydropteroate synthase (target of sulfonamide drugs), the pfam00903 domain (includes bleomycin resistance enzymes), beta-lactamase (penicillin resistance), aminoglycoside phosphotransferase and aminoglycoside acetyltransferase. In Xanthomonas campestris, a putative Transposase-resistance RNA is adjacent to a gene involved in synthesizing the pigment xanthomonadin. The motif is often present in plasmids. The motif's association with a wide variety of resistance genes can be a result of these genes being carried by repetitive elements, plasmids or phages. The hairpin structure of the motif can represent the inverted repeats that are often associated with transposases. The Transposase-resistance motif is present in a wide variety of bacteria, but is predominantly in Enterobacteria such as E. coli.

Ixxxv. TwoAYGGAY Motif

The TwoAYGGAY motif is named after its two terminal loops that have an AYGGAY subsequence. The motif is present in some Clostridia and γ-protcobactcria, but is more common in a human gut metagenome. The P1 stem that normally closes the structure is often very large (e.g., 24 base pairs with only 2 non-canonical/mismatching pairs). However, some representatives have very small P1 stems.

Ixxxvi. wcaG Motif

In two places wcaG RNAs carry a conserved UGGYG motif. Such duplicate short sequences are sometimes binding sites for a dimeric protein.

Ixxxvii. Whalefall-1 Motif

The Whalefall-1 motif is found only in metagenome sequences from whale fall (a whale carcass that has settled on the ocean floor). It consists of two hairpins, followed by a purine-rich sequence. Although this purine-rich sequence resembles a Shine-Dalgarno sequence, there is no strong evidence of a conserved gene immediately downstream of the motif. The terminal loop of the second loop often has a CUUG tetraloop.

Ixxxviii. yjdF Motif

Most predicted yjdF genes contain a yjdF RNA in their apparent 5′ UTR. In Streptococcus thermophilus, however, no yjdF gene is predicted. In Bacillus anthracis, RNA-seq experiments (Passalacqua et al. Structure and complexity of a bacterial transcriptome. J Bacteriol 2009, 191:3203-3211) indicate that transcription of yjdF RNA and the yjdF gene arises from readthrough of the upstream gene. Although expression levels of the yjdF gene appear to be modestly modulated in the conditions tested, this differential expression seems to correlate with the expression of the upstream gene. Similarly, when B. subtilis cells are grown in complex medium, tiling array experiments indicate that the upstream manPA genes are transcribed at similar levels to the yjdF RNA/gene, and form a single transcriptionally active region (TAR) (Rasmussen et al. The Transcriptionally Active Regions in the Genome of Bacillus subtilis. Mol Microbiol 2009). However, during growth in minimal medium, the manPA genes are transcribed at much lower levels, while the yjdF gene mRNA is almost as abundant as during growth in complex medium (Rasmussen et al. The Transcriptionally Active Regions in the Genome of Bacillus subtilis. Mol Microbiol 2009). Under minimal medium conditions, the TAR containing the yjdF gene (Rasmussen et al. The Transcriptionally Active Regions in the Genome of Bacillus subtilis. Mol Microbiol 2009) is predicted to begin five nucleotides upstream of the predicted start of the yjdF motif RNA.

Ixxxix. ykkC-III Motif

The ykkC-III motif exhibits somewhat more A-C mismatches in the given orientation than does its reverse complement. However, the orientation depicted herein is biological given the apparent cis-regulatory locations of the motif in the given orientation, and the fact that it is generally very close to predicted Shine-Dalgarno sequences. ykkC-III RNAs carry ACGA (SEQ ID NO:36) sequences that resemble conserved sequences in the mini-ykkC motif (FIG. 8). This could indicate a structural relationship between the motifs. In addition to a contiguous ACGA (SEQ ID NO:36) sequence, the ykkC-III motif has a possible split occurrence of ACGA (FIG. 8) that can fold into a similar conformation. However, some observations indicate that the common ACGA (SEQ ID NO:36) sequences might not be related. First, the structural contexts of the two ACGA (SEQ ID NO:36) occurrences within ykkC-III differ from the structure contexts within the mini-ykkC motif. Moreover the repetitive hairpin structure of mini-ykkC RNAs provides fewer opportunities for intricate binding sites than the pseudoknotted structure of ykkC-III. Thus, the ACGA (SEQ ID NO:36) sequences in mini-ykkC have a diminished ability to form complex tertiary interactions, as the ykkC-III ACGA (SEQ ID NO:36) sequences can. Second, it was observed that representatives of both the ykkC-III and the mini-ykkC motifs are found in a similarly wide range of phyla. Given that their opportunity to diverge is presumably comparable, it is noteworthy that the ACGA (SEQ ID NO:36) sequences are perfectly conserved in ykkC-III representatives, whereas their conservation in mini-ykkC RNAs is much looser. If the ACGA (SEQ ID NO:36) sequences serve similar structural roles, it is unclear why so much more variability is permitted in mini-ykkC RNAs.

4. Additions to Previously Characterized RNA Classes

i. 6S RNA

6S RNA is known to be present in almost all bacteria and regulates genes by binding to RNA polymerase (Barrick et al. 6S RNA is a widespread regulator of eubacterial RNA polymerase that resembles an open promoter. Rna 2005, 11:774-784). Two new motifs can represent diverged 6S RNAs. 6S-Flavo is found in Flavobacteria, which lack previously predicted 6S RNAs. Homology searches with 6S-Flavo detect a few known 6S RNAs, which is additional evidence that it represents 6S RNA. The alignment can be partial, as some pairing potential is observed that would extend the hairpin further, to make it more similar to 6S RNA lengths.

The Lacto-usp motif is found in five instances in the order Lactobacillales. It is consistently in the potential 5′ UTRs of operons containing a hypothetical gene and usp (Universal Stress Protein). Although these data indicate that Lacto-usp is a cis-regulatory RNA, three observations imply that it can correspond to 6S RNA. First, the four Lactobacillus species with Lacto-usp entirely lack a predicted 6S RNA. Second, the Lacto-usp motif conforms to the general structure of 6S RNA, a hairpin with large internal loops. Finally, 6S RNAs are already known that appear to be in the potential 5′ UTRs of operons containing usp genes in other Lactobacillales species. However, the Lacto-usp motif is noticeably shorter than most 6S RNAs, and obvious potential to extend the alignment is not observed.

ii. AdoCbl and SAM-II Riboswitches

A motif was found that resembles a previously identified class of riboswitches for adenosylcobalamin (AdoCbl) (Nahvi et al. Genetic control by a metabolite binding mRNA. Chem Biol 2002, 9:1043). The main differences are a P6 hairpin that is even shorter than previously found (Nahvi et al. Coenzyme B12 riboswitches are widespread genetic control elements in prokaryotes. Nucleic Acids Res. 2004, 32:143-150), and a stem (which we call P13) that flanks a pseudoknot. Other variants of AdoCbl riboswitches have also been observed previously (Fox et al. Multiple posttranscriptional regulatory mechanisms partner to control ethanolamine utilization in Enterococcus faecalis. Proc Natl Acad Sci USA 2009, 106:4435-4440).

Variants of SAM-II riboswitches (Corbino et al. Evidence for a second class of S-adenosylmethionine riboswitches and other regulatory RNA motifs in alpha-proteobacteria. Genome Biol. 2005, 6:R70) reveal that long insertions are possible in this motif, although longer insertions generally fold into a stable structure.

5. Ligand-Binding Experiments Using in-Line Probing

Some riboswitches were tested for metabolite binding using in-line probing experiments (Regulski et al. In-line probing analysis of riboswitches. Methods Mol Biol 2008, 419:53-67). RNAs tested were transcribed in vitro from a DNA template using RNA polymerase T7. In the experiments described below, no modulation in gel patterns was observed that would indicate metabolite binding. Also, it is possible that ligand-induced structural modulation did not result in noticeable changes in the spontaneous cleavage rates of internucleotide linkages that are visualized in in-line probing assays.

i. In-Line Probing Experiments with a pfl RNA

Experiments were performed with the following RNA encoded by Clostridium acetobutylicum ATCC 824:

(SEQ ID NO: 12)
5′-GGUAAAAUAAGAAAAUCAUGCAACUGGCGGAAAUGGAGUUCAC
CAUAGGGAGCAUGAUUAAUAUAAGAAUCGACCGCCUGGGUAAAUUAAUA-
3′.

The following metabolites were tested at 1 mM except where noted: AICAR riboside, AICAR ribotide, SAICAR, GAR, pyruvate, guanine (100 μM), hypoxanthine (40 μM), IMP, formate, THF, dihydrofolate, 5-formyl-THF, 10-formyl-THF, methylene-THF, methenyl-THF, methyl-THF, SAICAR, glutamate, glutamine, glycine, serine, aspartate, dUMP, homocysteine, SAM, AICA, CAIR, CoA, acetyl-CoA, alanine, NAD, NADH, NADP, NADPH, dCMP, histidine, D-ribose 5′-phosphate, adenine, D-ribose, NADH, SAICAR, CAIR, glycine, PRPR, ppGpp, cAMP, HMP, ATP (600 μM), CTP (600 μM), GTP (600 μM), UTP (600 μM), AMP, ADP, GMP, GDP, UMP, UDP, uridine, and tryptophan.

Due to the instability of PRPP, it was also tested in an RNase protection assay with RNase T1 and V1. The use of an RNase permits a shorter incubation time than used for in-line probing. However, no change in degradation patterns was detected with either RNase.

ii. In-Line Probing Experiments with yjdF RNA

Experiments were performed on the following three RNAs, encoded by Bacillus subtilis, which have different 3′ ends:

```
(SEQ ID NO: 13)
        5′-GGUAAAGAAUGAAAAAACACGAUUCGGUUGGUAGUCCGGAUGC
AUGAUUGAGAAUGUCAGUAACCUUCCCCUCCUCGGGAUGUCCAUCAUUCU
UUAAUAUCUUUUAUGAGGAGGGAAUCGUU-3′;
(SEQ ID NO: 14)
        5′-GGUAAAGAAUGAAAAAACACGAUUCGGUUGGUAGUCCGGAUGC
AUGAUUGAGAAUGUCAGUAACCUUCCCCUCCUCGGGAUGUCCAUCAUUCU
UUAAUAUCU-3′;
(SEQ ID NO: 15)
        5′-GGUAAAGAAUGAAAAAACACGAUUCGGUUGGUAGUCCGGAUGC
AUGAUUGAGAAUGUCAGUAACCUUCCCCUCCUCGG-3′.
```

The following metabolites were tested with each RNA at 1 mM: NAD, NADH, NADP, NADPH, ADP-ribose, glutamine, nicotinamide, nicotinic acid, glutamate, beta-nicotinamide mononucleotide, and D-ribose 5′-phosphate.

iii. In-Line Probing Experiments with SAM-Chlorobi RNA

Experiments were performed on the following four RNAs, encoded by Chlorobium tepidum TLS, which have different 3′ ends:

```
(SEQ ID NO: 16)
        5′-ggAUUUUCCGGCAUCCCCAUUACCUAUGGACACGGUGCCAAAA
GCUCUCUUGCGGGAGUUGUCCCCGGAGCUUGCCGAAAGGUUUCCCGUGUC
CCGUUUGUCCCUCCGCGACAUUCACCUUCACGAGAAAACCGCAUCGGCAA
ACCGCCGGACACCUGCCGUUCUUGUCGUUCGAUUAACAAAAAACCGAAAG
GGAAACUA-3′;
(SEQ ID NO: 17)
        5′-ggAUUUUCCGGCAUCCCCAUUACCUAUGGACACGGUGCCAAAA
GCUCUCUUGCGGGAGUUGUCCCCGGAGCUUGCCGAAAGGUUUCCCGUGUC
CCGUUUGUCCC-3′;
(SEQ ID NO: 18)
        5′-ggAUUUUCCGGCAUCCCCAUUACCUAUGGACACGGUGCCAAAA
GCUCUCUUGCGGGAGUUGUCCCCGGAGCUUGCCGAAAGGUUUCC-3′;
(SEQ ID NO: 19)
        5′-ggAUUUUCCGGCAUCCCCAUUACCUAUGGACACGGUGCCAAAA
GCUCUCUUGCGGGAGUU-3′.
```

Lowercase letters represent G nucleotides that were added to improve transcription yield. The following metabolites were tested at 1 mM: SAM, SAH, methionine, and homocysteine.

iv. In-Line Probing Experiments with pan RNA

Experiments were performed on the following RNA encoded by Geobacter metallireducens GS-15:

(SEQ ID NO: 20)
5′-ggCAAAUUGAUACUGCCUGGAUUCGUACGAACCGGGACGGAUG
GCAAUAGCCGCAACGACAAGGAAAUAGCUUUUUCUCUUGGUCUUGGUACA
UGCGCCUCCGGAA-3′

Lowercase letters represent G nucleotides that were added to improve transcription yield. The following metabolites were tested at 1 mM: pantothenate, CoA, and beta-alanine.

v. In-Line Probing Experiments with msiK RNA

Experiments were performed with the following RNA encoded by Streptomyces coelicolor A3(2):

(SEQ ID NO: 21)
5′-GGACUACACCACCACCUUCCUACAACGGAUCGUCCGGCACGUU
CCUGCCGGUAGAAGGGGGCCCUUUCAC-3′.

The following metabolites were tested at 1 mM: fructose, galactose, glucose, mannose, xylose, cellobiose, lactose, maltose, sucrose, trehalose, glucose-1-phosphate, glucose-6-phosphate, fructose-6-phosphate, and fructose-1,6-bisphophate.

vI. In-Line Probing Experiments with gabT RNA

Experiments were performed with the following two RNAs, encoded by Pseudomonas syringae pv. tomato str. DC3000, which have different 3′ ends (lowercase letters represent G nucleotides that were added to improve transcription yield):

```
(SEQ ID NO: 22)
        5′-ggUCUUGGCGGCCUGAAGGCUGCAGCAGUCGAUCAUCGUAUGC
UGUUGCAGUUGAUCCAGCCCGCUUGAUCC-3′
(SEQ ID NO: 23)
        5′-ggUCUUGGCGGCCUGAAGGCUGCAGCAGUCGAUCAUCGUAUGC
UGUUGCAGUUGAUCCAGCCCGCUUGAUCCUUGAACCACGCCGACCGAUGA
GCGGCGAAUGAGGAAUACA-3′
```

The following metabolites were tested at 1 mM except where noted: cAMP, cGMP, cyclic di-GMP, agmatine, putrescine, GABA, L-glutamine, L-glutamate, L-lysine, 2-oxoglutarate (200 μM), glutaric acid (200 μM), succinate (200 μM), and succinic semialdehyde (200 μM).

vii. In-Line Probing Experiments with rmf RNA

Experiments were performed with the following four RNAs, encoded by Pseudomonas syringae pv. tomato str. DC3000, which have different 5′ and/or 3′ ends:

```
(SEQ ID NO: 24)
        5′- gGCGCUUUGGUUAGAAAUCAACUCAGGUCAUUUCCGCAAUGG
UUAUGGCAUCAAGGCCCGCCACGCCGGCAGCGGGCCCCAACGGCAGAAGA
CUCUGCCCGACCCCACCACGGGGUCUCAGGGAUAUUACAGUCAACAGA-
3′;
(SEQ ID NO: 25)
        5′-ggAUCAUUCACAUCACCCUGCGCUUUGGUUAGAAAUCAACUC
AGGUCAUUUCCGCAAUGGUUAUGGCAUCAAGGCCCGCCACGCCGGCAGCG
GGCCCCAACGGCAGAAGACUCUGCCCGACCCCACCACGGGGUCUCAGGGA
UAUUACAGUCAACAGA- 3′;
(SEQ ID NO: 26)
        5′- gGCGCUUUGGUUAGAAAUCAACUCAGGUCAUUUCCGCAAUGG
UUAUGGCAUCAAGGCCCGCCACGCCGGCAGCGGGCCCCAACGGCAGAAGA
CUCUGCCCGACCCCACCACGGGGUCUCAGGGAUAUUACAGUCAACAGACG
AGGGCAUUACCCUAUGAGAAGA-3′;
(SEQ ID NO: 27)
        5′-ggAUCAUUCACAUCACCCUGCGCUUUGGUUAGAAAUCAACUCA
GGUCAUUUCCGCAAUGGUUAUGGCAUCAAGGCCCGCCACGCCGGCAGCGG
GCCCCAACGGCAGAAGACUCUGCCCGACCCCACCACGGGGUCUCAGGGAU
AUUACAGUCAACAGACGAGGGCAUUACCCUAUGAGAAGA -3′.
```

Lowercase letters represent G nucleotides that were added to improve transcription yield. The metabolite ppGpp was tested at 1 mM.

vIii. In-Line Probing Experiments with Downstream Peptide RNA

Experiments were performed with the following two RNAs, encoded by Synechococcus sp. CC9605, which have different 5′ and 3′ ends (lowercase letters represent G nucleotides that were added to improve transcription yield):

```
(SEQ ID NO: 28)
        5′- gGCGACCACGUUCACCUCGUCUUCGGCGAGGCGCAGUUCGAC
UCAGGCCAUGGAACGGGGACCUGAGCUUG-3′;
(SEQ ID NO: 29)
        5′- gGCUACGCGACCACGUUCACCUCGUCUUCGGCGAGGCGCAGU
UCGACUCAGGCCAUGGAACGGGGACCUGAGCUUCCUUCGAGGAACU -
3′.
```

The following metabolites were tested at 1 mM except where noted: cAMP, cGMP, cyclic di-GMP, agmatine, putrescine, GABA, L-glutamine, L-glutamate, L-lysine, 2-oxoglutarate (200 glutaric acid (200 μM), succinate (200 μM), and succinic semialdehyde (200 μM).

It is understood that the disclosed method and compositions are not limited to the particular methodology, protocols, and reagents described as these may vary. It is also to be understood that the terminology used herein is for the purpose of describing particular embodiments only, and is not intended to limit the scope of the present invention which will be limited only by the appended claims.

It must be noted that as used herein and in the appended claims, the singular forms “a”, “an”, and “the” include plural reference unless the context clearly dictates otherwise.

Thus, for example, reference to “a riboswitch” includes a plurality of such riboswitches, reference to “the riboswitch” is a reference to one or more riboswitches and equivalents thereof known to those skilled in the art, and so forth.

“Optional” or “optionally” means that the subsequently described event, circumstance, or material may or may not occur or be present, and that the description includes instances where the event, circumstance, or material occurs or is present and instances where it does not occur or is not present.

Ranges may be expressed herein as from “about” one particular value, and/or to “about” another particular value. When such a range is expressed, also specifically contemplated and considered disclosed is the range from the one particular value and/or to the other particular value unless the context specifically indicates otherwise. Similarly, when values are expressed as approximations, by use of the antecedent “about,” it will be understood that the particular value forms another, specifically contemplated embodiment that should be considered disclosed unless the context specifically indicates otherwise. It will be further understood that the endpoints of each of the ranges are significant both in relation to the other endpoint, and independently of the other endpoint unless the context specifically indicates otherwise. Finally, it should be understood that all of the individual values and sub-ranges of values contained within an explicitly disclosed range are also specifically contemplated and should be considered disclosed unless the context specifically indicates otherwise. The foregoing applies regardless of whether in particular cases some or all of these embodiments are explicitly disclosed.

Unless defined otherwise, all technical and scientific terms used herein have the same meanings as commonly understood by one of skill in the art to which the disclosed method and compositions belong. Although any methods and materials similar or equivalent to those described herein can be used in the practice or testing of the present method and compositions, the particularly useful methods, devices, and materials are as described. Publications cited herein and the material for which they are cited are hereby specifically incorporated by reference. Nothing herein is to be construed as an admission that the present invention is not entitled to antedate such disclosure by virtue of prior invention. No admission is made that any reference constitutes prior art. The discussion of references states what their authors assert, and applicants reserve the right to challenge the accuracy and pertinency of the cited documents. It will be clearly understood that, although a number of publications are referred to herein, such reference does not constitute an admission that any of these documents forms part of the common general knowledge in the art.

Throughout the description and claims of this specification, the word “comprise” and variations of the word, such as “comprising” and “comprises,” means “including but not limited to,” and is not intended to exclude, for example, other additives, components, integers or steps.

Those skilled in the art will recognize, or be able to ascertain using no more than routine experimentation, many equivalents to the specific embodiments of the method and compositions described herein. Such equivalents are intended to be encompassed by the following claims.

### REFERENCES

Abreu-Goodger C, Merino E: RibEx: a web server for locating riboswitches and other conserved bacterial regulatory elements. Nucleic Acids Res 2005, 33:W690-692.
Altschul S F, Madden T L, Schaffer A A, Zhang J, Zhang Z, Miller W, Lipman D J: Gapped BLAST and PSI-BLAST: a new generation of protein database search programs. Nucleic Acids Research 1997, 25:3389-3402.
Antao V P, Tinoco I, Jr.: Thermodynamic parameters for loop formation in RNA and DNA hairpin tetraloops. Nucleic Acids Res 1992, 20:819-824.
Amvig K B, Young D B: Identification of small RNAs in Mycobacterium tuberculosis. Mol Microbiol 2009, 73:397-408.
Arthur D C, Ghetu A F, Gubbins M J, Edwards R A, Frost L S, Glover J N: FinO is an RNA chaperone that facilitates sense-antisense RNA interactions. Embo J2003, 22:6346-6355.
Axmann I M, Kensche P, Vogel J, Kohl S, Herzel H, Hess W R: Identification of cyanobacterial non-coding RNAs by comparative genome analysis. Genome Biology 2005, 6:R73.
Axmann I M, Kensche P, Vogel J, Kohl S, Herzel H, Hess W R: Identification of cyanobacterial non-coding RNAs by comparative genome analysis. Genome Biology 2005, 6:R73.
Barrett T, Troup D B, Wilhite S E, Ledoux P, Rudnev D, Evangelista C, Kim I F, Soboleva A, Tomashevsky M, Marshall K A, Phillippy K H, Sherman P M, Muertter R N, Edgar R: NCBI GEO: archive for high-throughput functional genomic data. Nucleic Acids Res 2009, 37:D885-890.
Barrick J E, Breaker R R. The distributions, mechanisms, and structures of metabolite-binding riboswitches. Genome Biol 2007; 8:R239.
Barrick J E, Breaker R R: The distributions, mechanisms, and structures of metabolite-binding riboswitches. Genome Biol. 2007, 8:R239.
Barrick J E, Corbino K A, Winkler W C, Nahvi A, Mandal M, Collins J, Lee M, Roth A, Sudarsan N, Jona I, et al. New RNA motifs suggest an expanded scope for riboswitches in bacterial genetic control. Proc Natl Acad Sci USA 2004; 101:6421-6426.
Barrick J E, Corbino K A, Winkler W C, Nahvi A, Mandal M, Collins J, Lee M, Roth A, Sudarsan N, Jona I, Wickiser J K, Breaker R R: New RNA motifs suggest an expanded scope for riboswitches in bacterial genetic control. Proc Natl Acad Sci USA 2004, 101:6421-6426.
Barrick J E, Sudarsan N, Weinberg Z, Ruzzo W L, Breaker R R: 6S RNA is a widespread regulator of eubacterial RNA polymerase that resembles an open promoter. Rna 2005, 11:774-784.
Bayley D P, Rocha E R, Smith C J: Analysis of cepA and other Bacteroides fragilis genes reveals a unique promoter structure. FEMS Microbiol Lett 2000, 193:149-154.
Bayley D P, Rocha E R, Smith C J: Analysis of cepA and other Bacteroides fragilis genes reveals a unique promoter structure. FEMS Microbiol Lett 2000, 193:149-154.
Bennett B D, Kimball E H, Gao M, Osterhout R, Van Dien S J, Rabinowitz J D. Absolute metabolite concentrations and implied enzyme active site occupancy in Escherichia coli. Nat Chem Biol 2009; 5:593-599.
Bertram R, Schlicht M, Mahr K, Nothaft H, Saier M H, Jr., Titgemeyer F: In silico and transcriptional analysis of carbohydrate uptake systems of Streptomyces coelicolor A3(2). J Bacteriol 2004, 186:1362-1373.
Bochner B R, Ames B N: ZTP (5-amino4-imidazole carboxamide riboside 5′-triphosphate): a proposed alarmone for 10-formyl-tetrahydrofolate deficiency. Cell 1982, 29:929-937.
Bonomo R A, Szabo D: Mechanisms of multidrug resistance in Acinetobacter species and Pseudomonas aeruginosa. Clin Infect Dis 2006, 43 Suppl 2:S49-56.
Breaker R R. Riboswitches: from ancient gene-control systems to modern drug targets. Future Microbiol 2009; 4:771-773.
Cardineau G A, Curtiss R, 3rd: Nucleotide sequence of the asd gene of Streptococcus mutans. Identification of the promoter region and evidence for attenuator-like sequences preceding the structural gene. J Biol Chem 1987, 262:3344-3353.
Cheah M T, Wachter A, Sudarsan N, Breaker R R. Control of alternative RNA splicing and gene expression by eukaryotic riboswitches. Nature 2007; 447:497-500.
Chen S, Bagdasarian M, Kaufman M G, Walker E D: Characterization of strong promoters from an environmental Flavobacterium hibernum strain by using a green fluorescent protein-based reporter system. Appl Environ Microbiol 2007, 73:1089-1100.
Chen S, Bagdasarian M, Kaufman M G, Walker E D: Characterization of strong promoters from an environmental Flavobacterium hibernum strain by using a green fluorescent protein-based reporter system. Appl Environ Microbiol 2007, 73:1089-1100.
Chou H T, Kwon D H, Hegazy M, Lu C D: Transcriptome analysis of agmatine and putrescine catabolism in Pseudomonas aeruginosa PAO1. J Bacteriol 2008, 190:1966-1975.
Chugani S, Greenberg E P: The influence of human respiratory epithelia on Pseudomonas aeruginosa gene expression. Microb Pathog 2007, 42:29-35.
Citron M, Schuster H: The c4 repressors of bacteriophages P1 and P7 arc antisense RNAs. Cell 1990, 62:591-598.
Citron M, Schuster H: The c4 repressors of bacteriophages P1 and P7 are antisense RNAs. Cell 1990, 62:591-598.
Cochrane J C, Lipchock S V, Smith K D, Strobel S A. Structural and chemical basis for glucosamine 6-phosphate binding and activation of the glmS ribozyme. Biochemistry 2009; 48:3239-3246.
Connelly J C, Leach D R: The sbcC and sbcD genes of Escherichia coli encode a nuclease involved in palindrome inviability and genetic recombination. Genes Cells 1996, 1:285-291.
Corbino K A, Barrick J E, Lim J, Welz R, Tucker B J, Puskarz I, Mandal M, Rudnick N D, Breaker R R: Evidence for a second class of S-adenosylmethionine riboswitches and other regulatory RNA motifs in alpha-proteobacteria. Genome Biol. 2005, 6:R70.
Corbino K A, Barrick J E, Lim J, Welz R, Tucker B J, Puskarz I, Mandal M, Rudnick N D, Breaker R R: Evidence for a second class of S-adenosylmethionine riboswitches and other regulatory RNA motifs in alpha-proteobacteria. Genome Biol. 2005, 6:R70.
Croft M T, Moulin M, Webb M E, Smith A G. Thiamine biosynthesis in algae is regulated by riboswitches. Proc Natl Acad Sci USA 2007; 104:20770-20775.
Dambach M D, Winkler W C. Expanding roles for metabolite-sensing regulatory RNAs. Curr Opin Microbiol 2009; 12:161-169.
Das R, Laederach A, Pearlman S M, Herschlag D, Altman R B. SAFA: Semi-automated footprinting analysis software for high-throughput quantification of nucleic acid footprinting experiments. RNA 2005; 11:344-354. del Val C, Rivas E, Torres-Quesada O, Toro N, Jimenez-Zurdo JI: Identification of differentially expressed small non-coding RNAs in the legume endosymbiont Sinorhizobium meliloti by comparative genomics. Mol Microbiol 2007, 66:1080-1091.
DeLong E F, Preston C M, Mincer T, Rich V, Hallam S J, Frigaard N U, Martinez A, Sullivan M B, Edwards R, Brito B R, Chisholm S W, Karl D M: Community genomics among stratified microbial assemblages in the ocean's interior. Science 2006, 311:496-503.
Derzelle S, Bolotin A, Mistou M Y, Rul F: Proteome analysis of Streptococcus thermophilus grown in milk reveals pyruvatc formatc-lyasc as the major upregulated protein. Appl Environ Microbiol 2005, 71:8597-8605.
Diwa A, Bricker A L, Jain C, Belasco J G: An evolutionarily conserved RNA stem-loop functions as a sensor that directs feedback regulation of RNase E gene expression. Genes Dev 2000, 14:1249-1260.
Duan K, Liu C Q, Supple S, Dunn N W: Involvement of antisense RNA in replication control of the lactococcal plasmid pND324. FEMS Microbiol Lett 1998, 164:419-426.
Eddy S R, Durbin R: RNA Sequence Analysis Using Covariance Models. Nucleic Acids Research 1994, 22:2079-2088.
Eddy S R: A memory-efficient dynamic programming algorithm for optimal alignment of a sequence to an RNA secondary structure. BMC Bioinformatics 2002, 3:18.
Espinosa-Urgel M, Ramos J L: Expression of a Pseudomonas putida aminotransferase involved in lysine catabolism is induced in the rhizosphere. Appl Environ Microbiol 2001, 67:5219-5224.
Finn R D, Mistry J, Schuster-Bockler B, Griffiths-Jones S, Hollich V, Lassmann T, Moxon S, Marshall M, Khanna A, Durbin R, Eddy S R, Sonnhammer E L, Bateman A: Pfam: clans, web tools and services. Nucleic Acids Res 2006, 34:D247-251.
Forchhammer K. Global carbon/nitrogen control by PII signal transduction in cyanobacteria: from signals to targets. FEMS Microbiol Rev 2004; 28:319-333.
Forchhammer K. Glutamine signalling in bacteria. Front Biosci 2007; 12:358-370.
Forchhammer K: Global carbon/nitrogen control by PII signal transduction in cyanobacteria: from signals to targets. FEMS Microbiol Rev 2004, 28:319-333.
Fox K A, Ramesh A, Stearns J E, Bourgogne A, Reyes-Jara A, Winkler W C, Garsin D A: Multiple posttranscriptional regulatory mechanisms partner to control ethanolamine utilization in Enterococcus faecalis. Proc Natl Acad Sci USA 2009, 106:4435-4440.
Frias-Lopez J, Shi Y, Tyson G W, Coleman M L, Schuster S C, Chisholm S W, Delong E F: Microbial community gene expression in ocean surface waters. Proc Natl Acad Sci USA 2008, 105:3805-3810.
Fuchs R T, Grundy F J, Henkin T M: The S(MK) box is a new SAM-binding RNA for translational regulation of SAM synthetase. Nat. Struct. Mol. Biol. 2006, 13:226-233.
Fujita M, Amemura A, Aramaki H: Transcription of the groESL operon in Pseudomonas aeruginosa PAO1. FEMS Microbiol Lett 1998, 163:237-242.
Garcia Martin H, Ivanova N, Kunin V, Warnecke F, Barry K W, McHardy A C, Yeates C, He S, Salamov A A, Szeto E, Dalin E, Putnam N H, Shapiro H J, Pangilinan J L, Rigoutsos I, Kyrpides N C, Blackall L L, McMahon K D, Hugenholtz P: Metagenomic analysis of two enhanced biological phosphorus removal (EBPR) sludge communities. Nat Biotechnol 2006, 24:1263-1269.
Gardner P P, Daub J, Tate J G, Nawrocki E P, Kolbe D L, Lindgreen S, Wilkinson A C, Finn R D, Griffiths-Jones S, Eddy S R, Bateman A: Rfam: updates to the RNA families database. Nucleic Acids Res 2009, 37:D136-140.
Gardner P P, Daub J, Tate J G, Nawrocki E P, Kolbe D L, Lindgreen S, Wilkinson A C, Finn R D, Griffiths-Jones S, Eddy S R, Bateman A: Rfam: updates to the RNA families database. Nucleic Acids Res 2009, 37:D136-140.
Geissmann T, Chevalier C, Cros M J, Boisset S, Fechter P, Noirot C, Schrenzel J, Francois P, Vandenesch F, Gaspin C, Romby P: A search for small noncoding RNAs in Staphylococcus aureus reveals a conserved sequence motif for regulation. Nucleic Acids Res 2009.
Georg J, Voss B, Scholz I, Mitschke J, Wilde A, Hess W R: Evidence for a major role of antisense RNAs in cyanobacterial gene regulation. Mol Syst Biol 2009, 5:305.
Gilbert S D, Stoddard C D, Wise S J, Batey R T. Thermodynamic and kinetic characterization of ligand binding to the purine riboswitch aptamer domain. J Mol Biol 2006; 359:754-768.
Gill S R, Pop M, Deboy R T, Eckburg P B, Tumbaugh P J, Samuel B S, Gordon J I, Relman D A, Fraser-Liggett C M, Nelson K E: Metagenomic analysis of the human distal gut microbiome. Science 2006, 312:1355-1359.
Goldman J C. Identification of nitrogen as a growth-limiting nutrient in wastewaters and coastal marine waters through continuous culture algal assays. Water Res 1976; 10:97-104.
Gonzalez N, Heeb S, Valverde C, Kay E, Reimmann C, Junier T, Haas D: Genome-wide search reveals a novel GacA-regulated small RNA in Pseudomonas species. BMC Genomics 2008, 9:167.
Guarneros G, Montanez C, Hernandez T, Court D: Posttranscriptional control of bacteriophage lambda gene expression from a site distal to the gene. Proc Natl Acad Sci USA 1982, 79:238-242.
Gupta R S: The phylogeny and signature sequences characteristics of Fibrobacteres, Chlorobi, and Bacteroidetes. Crit. Rev Microbiol 2004, 30:123-143.
Hendriksen W T, Bootsma H J, Estevao S, Hoogenboezem T, de Jong A, de Groot R, Kuipers O P, Hermans P W: CodY of Streptococcus pneumoniae: link between nutritional gene regulation and colonization. J Bacteriol 2008, 190:590-601.
Hobbs M, Reeves P R: The JUMPstart sequence: a 39 bp element common to several polysaccharide gene clusters. Mol Microbiol 1994, 12:855-856.
Hobbs M, Reeves P R: The JUMPstart sequence: a 39 bp element common to several polysaccharide gene clusters. Mol Microbiol 1994, 12:855-856.
Hurtubise Y, Shareck F, Kluepfel D, Morosoli R: A cellulase/xylanase-negative mutant of Streptomyces lividans 1326 defective in cellobiose and xylobiose uptake is mutated in a gene encoding a protein homologous to ATP-binding proteins. Mol Microbiol 1995, 17:367-377.
Hurtubise Y, Shareck F, Kluepfel D, Morosoli R: A cellulase/xylanase-negative mutant of Streptomyces lividans 1326 defective in cellobiose and xylobiose uptake is mutated in a gene encoding a protein homologous to ATP-binding proteins. Mol Microbiol 1995, 17:367-377.
Jarrige A C, Mathy N, Portier C: PNPase autocontrols its expression by degrading a double-stranded structure in the pnp mRNA leader. Embo J2001, 20:6845-6855.
Jiang P, Peliska J A, Ninfa A J. Enzymological characterization of the signal-transducing uridylyltransferase/uridylyl-removing enzyme (EC 2.7.7.59) of Escherichia coli and its interaction with the PII protein. Biochemistry 1998; 37:12782-12794.
Johansen L E, Nygaard P, Lassen C, Agerso Y, Saxild H H: Definition of a second Bacillus subtilis pur regulon comprising the pur and xpt-pbuX operons plus pbuG, nupG (yxjA), and pbuE (ydhL). J Bacteriol 2003, 185:5200-5209.
Kim K, Meyer R J: Copy-number of broad host-range plasmid R1162 is regulated by a small RNA. Nucleic Acids Res 1986, 14:8027-8046.
Klein R J, Misulovin Z, Eddy S R: Noncoding RNA genes identified in AT-rich hyperthermophiles. Proceedings of the National Academy of Sciences of the Unites States of America 2002, 99:7542-7547.
Knudsen B, Hein J: Pfold: RNA secondary structure prediction using stochastic context-free grammars. Nucleic Acids Res 2003, 31:3423-3428.
Kok J: Inducible gene expression and environmentally regulated genes in lactic acid bacteria. Antonie Van Leeuwenhoek 1996, 70:129-145.
Kurokawa K, Itoh T, Kuwahara T, Oshima K, Toh H, Toyoda A, Takami H, Morita H, Sharma V K, Srivastava T P, Taylor T D, Noguchi H, Mori H, Ogura Y, Ehrlich D S, Itoh K, Takagi T, Sakaki Y, Hayashi T, Hattori M: Comparative metagenomics revealed commonly enriched gene sets in human gut microbiomes. DNA Res. 2007, 14:169-181.
Kwon M, Strobel S A. Chemical basis of glycine riboswitch cooperativity. RNA 2008; 14:25-34.
Leaphart A B, Thompson D K, Huang K, Alm E, Wan X F, Arkin A, Brown S D, Wu L, Yan T, Liu X, Wickham G S, Zhou J: Transcriptome profiling of Shewanella oneidensis gene expression following exposure to acidic and alkaline pH. J Bacteriol 2006, 188:1633-1642.
Lee J C: Structural studies of ribosomal RNA based on cross-analysis of comparative models and three-dimensional crystal structures. Austin, Tex.: University of Texas; 2003. Dissertation.
Leeds J A, Welch R A: Enhancing transcription through the Escherichia coli hemolysin operon, hlyCABD: RfaH and upstream JUMPStart DNA sequences function together via a postinitiation mechanism. J Bacteriol 1997, 179:3519-3527.
Leeds J A, Welch R A: Enhancing transcription through the Escherichia coli hemolysin operon, hlyCABD: RfaH and upstream JUMPStart DNA sequences function together via a postinitiation mechanism. J Bacteriol 1997, 179:3519-3527.
Lemon K P, Earl A M, Vlamakis H C, Aguilar C, Kolter R: Biofilm development with an emphasis on Bacillus subtilis. Curr Top Microbiol Immunol 2008, 322:1-16.
Leoff C, Saile E, Sue D, Wilkins P, Quinn C P, Carlson R W, Kannenberg E L: Cell wall carbohydrate compositions of strains from the Bacillus cereus group of species correlate with phylogenetic relatedness. J Bacteriol 2008, 190:112-121.
Liang W, Silva A J, Benitez J A: The cyclic AMP receptor protein modulates colonial morphology in Vibrio cholerae. Appl Environ Microbiol 2007, 73:7482-7487.
Lindell D, Jaffe J D, Coleman M L, Futschik M E, Axmann I M, Rector T, Kettler G, Sullivan M B, Steen R, Hess W R, Church G M, Chisholm S W: Genome-wide expression dynamics of a marine virus and host reveal features of co-evolution. Nature 2007, 449:83-86.
Liu J M, Livny J, Lawrence M S, Kimball M D, Waldor M K, Camilli A: Experimental discovery of sRNAs in Vibrio cholerae by direct cloning, 5S/tRNA depletion and parallel sequencing. Nucleic Acids Res 2009, 37:e46.
Livny J, Brencic A, Lory S, Waldor M K: Identification of 17 Pseudomonas aeruginosa sRNAs and prediction of sRNA-encoding genes in 10 diverse pathogens using the bioinformatic tool sRNAPredict2. Nucleic Acids Res 2006, 34:3484-3493.
Livny J, Teonadi H, Livny M, Waldor M K: High-throughput, kingdom-wide prediction and annotation of bacterial non-coding RNAs. PLoS One 2008, 3:e3197.
Loh E, Dussurget O, Gripenland J, Vaitkevicius K, Tiensuu T, Mandin P, Repoila F, Buchrieser C, Cossart P, Johansson J. A trans-acting riboswitch controls expression of the virulence regulator PrfA in listeria monocytogenes. Cell 2009; 139:770-779.
Lombardo M J, Rosenberg S M: radC 102 of Escherichia coli is an allele of recG. J Bacteriol 2000, 182:6287-6291.
Lowe T M, Eddy S R: tRNAscan-SE: a program for improved detection of transfer RNA genes in genomic sequence. Nucleic Acids Research 1997, 25:955-964.
Mandal M, Lee M, Barrick J E, Weinberg Z, Emilsson G M, Ruzzo W L, Breaker R R. A glycine-dependent riboswitch that uses cooperative binding to control gene expression. Science 2004; 306:275-279.
Mandal M, Lee M, Barrick J E, Weinberg Z, Emilsson G M, Ruzzo W L, Breaker R R: A glycine-dependent riboswitch that uses cooperative binding to control gene expression. Science 2004, 306:275-279.
Marchais A, Naville M, Bohn C, Bouloc P, Gautheret D: Single-pass classification of all noncoding sequences in a bacterial genome using phylogenetic profiles. Genome Res 2009, 19:1084-1092.
Marchler-Bauer A, Anderson J B, Cherukuri P F, DeWeese-Scott C, Geer L Y, Gwadz M, He S, Hurwitz D I, Jackson J D, Ke Z, Lanczycki C J, Liebert C A, Liu C, Lu F, Marchler G H, Mullokandov M, Shoemaker B A, Simonyan V, Song J S, Thiessen P A, Yamashita R A, Yin J J, Zhang D, Bryant S H: CDD: a Conserved Domain Database for protein classification. Nucleic Acids Research 2005, 33:192-196.
Markowitz V M, lvanova N N, Szeto E, Palaniappan K, Chu K, Dalevi D, Chen I M, Grechkin Y, Dubchak I, Anderson I, Lykidis A, Mavromatis K, Hugenholtz P, Kyrpides N C: IMG/M: a data management and analysis system for metagenomes. Nucleic Acids Res 2008, 36:D534-538.
Marolda C L, Valvano M A: Promoter region of the Escherichia coli O7-specific lipopolysaccharide gene cluster: structural and functional characterization of an upstream untranslated mRNA sequence. J Bacteriol 1998, 180:3070-3079.
Marolda C L, Valvano M A: Promoter region of the Escherichia coli O7-specific lipopolysaccharide gene cluster: structural and functional characterization of an upstream untranslated mRNA sequence. J Bacteriol 1998, 180:3070-3079.
Mattheakis L, Vu L, Sor F, Nomura M: Retroregulation of the synthesis of ribosomal proteins L14 and L24 by feedback repressor S8 in Escherichia coli. Proc Natl Acad Sci USA 1989, 86:448-452.
McGowan C C, Necheva A S, Forsyth M H, Cover T L, Blaser M J: Promoter analysis of Helicobacter pylori genes with enhanced expression at low pH. Mol Microbiol 2003, 48:1225-1239.
McGowan C C, Necheva A S, Forsyth M H, Cover T L, Blaser M J: Promoter analysis of Helicobacter pylori genes with enhanced expression at low pH. Mol Microbiol 2003, 48:1225-1239.
Meyer I M: A practical guide to the art of RNA gene prediction. Brief Bioinform 2007, 8:396-414.
Meyer M M, Ames T D, Smith D P, Weinberg Z, Schwalbach M S, Giovannoni S J, Breaker R R: Identification of candidate structured RNAs in the marine organism ‘Candidatus Pelagibacter ubique’. BMC Genomics 2009, 10:268.
Montange R K, Batey R T: Riboswitches: emerging themes in RNA structure and function. Annu Rev Biophys 2008, 37:117-133.
Montange R K, Batey R T: Structure of the S-adenosylmethionine riboswitch regulatory mRNA element. Nature 2006, 441:1172-1175.
Muramatsu M, Hihara Y: Characterization of high-light-responsive promoters of the psaAB genes in Synechocystis sp. PCC 6803. Plant Cell Physiol 2006, 47:878-890.
Muramatsu M, Hihara Y: Coordinated high-light response of genes encoding subunits of photosystem 1 is achieved by AT-rich upstream sequences in the cyanobacterium Synechocystis sp. strain PCC 6803. J Bacteriol 2007, 189:2750-2758.
Muro-Pastor M I, Reyes J C, Florencio F J. Cyanobacteria perceive nitrogen status by sensing intracellular 2-oxoglutarate levels. J Biol Chem 2001; 276:38320-38328.
Nahvi A, Barrick J E, Breaker R R: Coenzyme B12 riboswitchcs arc widespread genetic control elements in prokaryotes. Nucleic Acids Res. 2004, 32:143-150.
Nahvi A, Sudarsan N, Ebert M S, Zou X, Brown K L, Breaker R R: Genetic control by a metabolite binding mRNA. Chem Biol 2002, 9:1043.
Nakhamchik A, Wilde C, Rowe-Magnus D A: Cyclic-di-GMP regulates extracellular polysaccharide production, biofilm formation, and rugose colony development by Vibrio vulnificus. Appl Environ Microbiol 2008, 74:4199-4209.
Nalca Y, Jansch L, Bredenbruch F, Geffers R, Buer J, Haussler S: Quorum-sensing antagonistic activities of azithromycin in Pseudomonas aeruginosa PAO1: a global approach. Antinzicrob Agents Chenzother 2006, 50:1680-1688.
Narberhaus F, Vogel J: Regulatory RNAs in prokaryotes: here, there and everywhere. Mol Microbiol 2009, 74:261-269.
Nawrocki E P, Kolbe D L, Eddy S R: Infernal 1.0: inference of RNA alignments. Bioinformatics 2009, 25:1335-1337.
Nieto J M, Bailey M J, Hughes C, Koronakis V: Suppression of transcription polarity in the Escherichia coli haemolysin operon by a short upstream element shared by polysaccharide and DNA transfer determinants. Mol Microbiol 1996, 19:705-713.
Nieto J M, Bailey M J, Hughes C, Koronakis V: Suppression of transcription polarity in the Escherichia coli haemolysin operon by a short upstream element shared by polysaccharide and DNA transfer determinants. Mol Microbiol 1996, 19:705-713.
Niven G W, El-Sharoud W M: Ribosome modulation factor. In: Bacterial physiology: a molecular approach Edited by El-Sharoud W M. Berlin: Springer-Verlag; 2008.
Noguchi H, Park J, Takagi T: MetaGene: prokaryotic gene finding from environmental genome shotgun sequences. Nucleic Acids Res 2006, 34:5623-5630.
Ochsner U A, Wilderman P J, Vasil A I, Vasil M L: GeneChip expression analysis of the iron starvation response in Pseudomonas aeruginosa: identification of novel pyoverdine biosynthesis genes. Mol Microbiol 2002, 45:1277-1287.
Odenbreit S, Faller G, Haas R: Role of the alpAB proteins and lipopolysaccharide in adhesion of Helicobacter pylori to human gastric tissue. Int J Med Microbiol 2002, 292:247-256.
Pace N R, Thomas B C, Woese C R: Probing RNA structure, function, and history by comparative analysis. In: The RNA World, 2nd edition Edited by Gesteland R F, Cech T R, Atkins J F. Cold Spring Harbor, N.Y.: Cold Spring Harbor Laboratory Press; 1999.
Padalon-Brauch G, Hcrshbcrg R, Elgrably-Wciss M, Baruch K, Rosenshine I, Margalit H, Altuvia S: Small RNAs encoded within genetic islands of Salmonella typhimurium show host-induced expression and role in virulence. Nucleic Acids Res 2008, 36:1913-1927.
Panagiotidis C H, Boos W, Shuman H A: The ATP-binding cassette subunit of the maltose transporter MalK antagonizes MalT, the activator of the Escherichia coli mal regulon. Mol Microbiol 1998, 30:535-546.
Parche S, Amon J, Jankovic I, Rezzonico E, Beleut M, Barutcu H, Schendel I, Eddy M P, Burkovski A, Arigoni F, Titgemeyer F: Sugar transport systems of Bifidobacterium longum NCC2705. J Mol Microbiol Biotechnol 2007, 12:9-19.
Passalacqua K D, Varadarajan A, Ondov B D, Okou D T, Zwick M E, Bergman N H: Structure and complexity of a bacterial transcriptome. J Bacteriol 2009, 191:3203-3211.
Perkins T T, Kingsley R A, Fookes M C, Gardner P P, James K D, Yu L, Assefa S A, He M, Croucher N J, Pickard D J, Maskell D J, Parkhill J, Choudhary J, Thomson N R, Dougan G: A strand-specific RNA-Seq analysis of the transcriptome of the typhoid bacillus Salmonella typhi. PLoS Genet. 2009, 5:e1000569.
Pichon C, Felden B: Small RNA genes expressed from Staphylococcus aureus genomic and pathogenicity islands with specific expression among pathogenic strains. Proc Natl Acad Sci USA 2005, 102:14249-14254.
Platt M D, Schurr M J, Sauer K, Vazquez G, Kukavica-Ibrulj I, Potvin E, Levesque R C, Fedynak A, Brinkman F S, Schurr J, Hwang S H, Lau G W, Limbach P A, Rowe J J, Lieberman M A, Barraud N, Webb J, Kjelleberg S, Hunt D F, Hassett D J: Proteomic, microarray, and signature-tagged mutagenesis analyses of anaerobic Pseudomonas aeruginosa at pH 6.5, likely representing chronic, late-stage cystic fibrosis airway conditions. J Bacteriol 2008, 190:2739-2758.
Poiata E, Meyer M M, Ames T D, Breaker R R: A variant riboswitch aptamer class for S-adenosylmethionine common in marine bacteria. Rna 2009, 15:2046-2056.
Pruitt K, Tatusova T, Maglott D: NCBI Reference Sequence (RefSeq): a curated non-redundant sequence database of genomes, transcripts and proteins. Nucleic Acids Res. 2005, 33:501-504.
Rasmussen S, Nielsen H B, Jarmer H: The Transcriptionally Active Regions in the Genome of Bacillus subtilis. Mol Microbiol 2009.
Rasmussen S, Nielsen H B, Jarmer H: The Transcriptionally Active Regions in the Genome of Bacillus subtilis. Mol Microbiol 2009.
Ravcheev D A, Gelfand M S, Mironov A A, Rakhmaminova A B: Purine regulon of gamma-proteobacteria: a detailed description. Russian Journal of Genetics 2002, 38:1015-1025.
Regulski E E, Breaker R R. In-line probing analysis of riboswitches. Methods Mol Biol 2008; 419:53-67.
Regulski E E, Breaker R R: In-line probing analysis of riboswitches. Methods Mol Biol 2008, 419:53-67.
Regulski E E, Moy R H, Weinberg Z, Barrick J E, Yao Z, Ruzzo W L, Breaker R R: A widespread riboswitch candidate that controls bacterial genes involved in molybdenum cofactor and tungsten cofactor metabolism. Mol Microbiol 2008, 68:918-932.
Rivas E, Eddy S R: Noncoding RNA gene detection using comparative sequence analysis. BMC Bioinformatics 2001, 2:8.
Rodionov D A, Vitreschak A G, Mironov A A, Gelfand M S. Regulation of lysine biosynthesis and transport genes in bacteria: yet another RNA riboswitch? Nucleic Acids Res 2003; 31:6748-6757.
Rohlman C E, Matthews R G: Role of purine biosynthetic intermediates in response to folate stress in Escherichia coli. J Bacteriol 1990, 172:7200-7210.
Rohwer F, Thurber R V: Viruses manipulate the marine environment. Nature 2009, 459:207-212.
Roth A, Breaker R R. The structural and functional diversity of metabolite-binding riboswitches. Annu Rev Biochem 2009; 78:305-334.
Roth A, Breaker R R: The Structural and Functional Diversity of Metabolite-Binding Riboswitches. Annu Rev Biochem 2009.
Rusch D B, Halpern A L, Sutton G, Heidelberg K B, Williamson S, Yooseph S, Wu D, Eisen J A, Hoffman J M, Remington K, Beeson K, Tran B, Smith H, Baden-Tillson H, Stewart C, Thorpe J, Freeman J, Andrews-Pfannkoch C, Venter J E, Li K, Kravitz S, Heidelberg J F, Utterback T, Rogers Y H, Falcon L I, Souza V, Bonilla-Rosso G, Eguiarte L E, Karl DM, Sathyendranath S, Platt T, Bermingham E, Gallardo V, Tamayo-Castillo G, Ferrari M R, Strausberg R L, Nealson K, Friedman R, Frazier M, Venter J C: The Sorcerer II Global Ocean Sampling expedition: northwest Atlantic through eastern tropical Pacific. PLoS Biol 2007, 5:c77.
Saito S, Kakeshita H, Nakamura K: Novel small RNA-encoding genes in the intergenic regions of Bacillus subtilis. Gene 2009, 428:2-8.
Schattner P: Searching for RNA genes using base-composition statistics. Nucleic Acids Research 2002, 30:2076-2082.
Schlösser A, Kampers T, Schrempf H: The Streptomyces ATP-binding component MsiK assists in cellobiose and maltose transport. J Bacteriol 1997, 179:2092-2095.
Schlösser A, Kampers T, Schrempf H: The Streptomyces ATP-binding component MsiK assists in cellobiose and maltose transport. J Bacteriol 1997, 179:2092-2095.
Seraphin B: The HIT protein family: a new family of proteins present in prokaryotes, yeast and mammals. DNA Seq 1992, 3:177-179.
Shi Y, Tyson G W, DeLong E F: Metatranscriptomics reveals unique microbial small RNAs in the ocean's water column. Nature 2009, 459:266-269.
Smith K D, Lipchock S V, Ames T D, Wang J, Breaker R R, Strobel S A. Structural basis of ligand binding by a c-di-GMP riboswitch. Nat Struct Mol Biol 2009; 16:1218-1223.
Sonnleitner E, Sorger-Domenigg T, Madej M J, Findeiss S, Hackermuller J, Huttenhofer A, Stadler P F, Blasi U, Moll I: Detection of small RNAs in Pseudomonas aeruginosa by RNomics and structure-based bioinformatic tools. Microbiology 2008, 154:3175-3187.
Soukup G A, Breaker R R. Relationship between internucleotide linkage geometry and the stability of RNA. RNA 1999; 5:1308-1325.
Soukup G A, Breaker R R: Relationship between internucleotide linkage geometry and the stability of RNA. RNA 1999, 5:1308-1325.
Sriramulu D D, Nimtz M, Romling U: Proteome analysis reveals adaptation of Pseudomonas aeruginosa to the cystic fibrosis lung environment. Proteomics 2005, 5:3712-3721.
Steglich C, Futschik M E, Lindell D, Voss B, Chisholm S W, Hess W R: The challenge of regulation in a minimal photoautotroph: non-coding RNAs in Prochlorococcus. PLoS Genet. 2008, 4:e1000173.
Steglich C, Futschik M E, Lindell D, Voss B, Chisholm S W, Hess W R: The challenge of regulation in a minimal photoautotroph: non-coding RNAs in Prochlorococcus. PLoS Genet. 2008, 4:e1000173.
Storz G, Zhcng M: Oxidative stress. In: Bacterial stress responses Edited by Storz G, Hengge-Aronis R. Washington, D.C.: ASM Press; 2000.
Su Z, Mao F, Dam P, Wu H, Olman V, Paulsen I T, Palenik B, Xu Y: Computational inference and experimental validation of the nitrogen assimilation regulatory network in cyanobacterium Synechococcus sp. WH 8102. Nucleic Acids Res 2006, 34:1050-1065.
Sudarsan N, Barrick J E, Breaker R R: Metabolite-binding RNA domains are present in the genes of eukaryotes. RNA 2003, 9:644-647.
Sudarsan N, Hammond M C, Block K F, Welz R, Barrick J E, Roth A, Breaker R R. Tandem riboswitch architectures exhibit complex gene control functions. Science 2006; 314:300-304.
Sudarsan N, Lee E R, Weinberg Z, Moy R H, Kim J N, Link K H, Breaker R R: Riboswitches in eubacteria sense the second messenger cyclic di-GMP. Science 2008, 321:411-413.
Sudarsan N, Wickiser J K, Nakamura S, Ebert M S, Breaker R R. An mRNA structure in bacteria that controls gene expression by binding lysine. Genes Dev 2003; 17:2688-2697.
Sullivan M B, Coleman M L, Weigele P, Rohwer F, Chisholm S W: Three Prochlorococcus cyanophage genomes: signature features and ecological interpretations. PLoS Biol 2005, 3:e144.
Swiercz J P, Hindra, Bobek J, Haiser H J, Di Berardo C, Tjaden B, Elliot M A: Small non-coding RNAs in Streptomyces coelicolor. Nucleic Acids Res 2008, 36:7240-7251.
Tezuka T, Hara H, Ohnishi Y, Horinouchi S: Identification and gene disruption of small noncoding RNAs in Streptomyces griseus. J Bacteriol 2009, 191:4896-4904.
Toledo-Arana A, Dussurget O, Nikitas G, Sesto N, Guet-Revillet H, Balestrino D, Loh E, Gripenland J, Tiensuu T, Vaitkevicius K, Barthelemy M, Vergassola M, Nahori M A, Soubigou G, Regnault B, Coppee J Y, Lecuit M, Johansson J, Cossart P: The Listeria transcriptional landscape from saprophytism to virulence. Nature 2009, 459:950-956.
Tolonen A C, Aach J, Lindell D, Johnson Z I, Rector T, Steen R, Church G M, Chisholm S W: Global gene expression of Prochlorococcus ecotypes in response to changes in nitrogen availability. Mol Syst Biol 2006, 2:53.
Torres-Cabassa A, Gottesman S, Frederick R D, Dolph P J, Coplin D L: Control of extracellular polysaccharide synthesis in Erwinia stewartii and Escherichia coli K-12: a common regulatory function. J Bacteriol 1987, 169:4525-4531.
Tringe S G, von Mering C, Kobayashi A, Salamov A A, Chen K, Chang H W, Podar M, Short J M, Mathur E J, Detter J C, Bork P, Hugenholtz P, Rubin E M: Comparative metagenomics of microbial communities. Science 2005, 308:554-557.
Tseng H, Weinberg Z, Gore J, Breaker R R, Ruzzo W L. Finding non-coding RNAs through genome-scale clustering. J Bioinform Comput Biol 2009; 7:373-388.
Tseng H H, Weinberg Z, Gore J, Breaker R R, Ruzzo W L: Finding non-coding RNAs through genome-scale clustering. J Bioinform Comput Biol 2009, 7:373-388.
Turnbaugh P J, Ley R E, Mahowald M A, Magrini V, Mardis E R, Gordon J I: An obesity-associated gut microbiome with increased capacity for energy harvest. Nature 2006, 444:1027-1031.
Tyson G W, Chapman J, Hugenholtz P, Allen E E, Ram R J, Richardson P M, Solovyev V V, Rubin E M, Rokhsar D S, Banfield J F: Community structure and metabolism through reconstruction of microbial genomes from the environment. Nature 2004, 428:37-43.
Ueland P M: Pharmacological and biochemical aspects of S-adenosylhomocysteine and S-adenosylhomocysteine hydrolase. Pharmacol Rev 1982, 34:223-253.
Ulve V M, Sevin E W, Cheron A, Barloy-Hubler F: Identification of chromosomal alpha-proteobacterial small RNAs by comparative genome analysis and detection in Sinorhizobium meliloti strain 1021. BMC Genomics 2007, 8:467.
Valverde C, Livny J, Schluter J P, Reinkensmeier J, Becker A, Parisi G: Prediction of Sinorhizobium meliloti sRNA genes and experimental detection in strain 2011. BMC Genomics 2008, 9:416.
Vázquez-Bermúdez MF, Herrero A, Flores E. Carbon supply and 2-oxoglutarate effects on expression of nitrate reductase and nitrogen-regulated genes in Synechococcus sp. strain PCC 7942. FEMS Microbiol Lett 2003; 221:155-159.
Vencato M, Tian F, Alfano J R, Buell C R, Cartinhour S, DeClerck G A, Guttman D S, Stavrinides J, Joardar V, Lindeberg M, Bronstein P A, Mansfield J W, Myers C R, Collmer A, Schneider D J: Bioinformatics-enabled identification of the HrpL regulon and type 111 secretion system effector proteins of Pseudomonas syringae pv. phaseolicola 1448A. Mol Plant Microbe Interact 2006, 19:1193-1206.
Venter J C, Remington K, Heidelberg J F, Halpern A L, Rusch D, Eisen J A, Wu D, Paulsen I, Nelson K E, Nelson W, Fouts D E, Levy S, Knap A H, Lomas M W, Nealson K, White O, Peterson J, Hoffman J, Parsons R, Baden-Tillson H, Pfannkoch C, Rogers Y H, Smith H O: Environmental genome shotgun sequencing of the Sargasso Sea. Science 2004, 304:66-74.
Vitreschak A G, Lyubetskaya E V, Shirshin M A, Gelfand M S, Lyubetsky V A: Attenuation regulation of amino acid biosynthetic operons in proteobacteria: comparative genomics analysis. FEMS Microbiol Lett 2004, 234:357-370.
Wachter A, Tunc-Ozdemir M, Grove B C, Green P J, Shintani D K, Breaker R R. Riboswitch control of gene expression in plants by splicing and alternative 3′ end processing of mRNAs. Plant Cell 2007; 19:3437-3450.
Wachter A. Riboswitch-mediated control of gene expression in eukaryotes. RNA Biol 2010; 7:67-76.
Walt A, Kahn M L: The fixA and fixB genes are necessary for anaerobic carnitine reduction in Escherichia coli. J Bacteriol 2002, 184:4044-4047.
Wang J X, Breaker R R: Riboswitches that sense S-adenosylmethionine and S-adenosylhomocysteine. Biochem Cell Biol 2008, 86:157-168.
Wang J X, Lee E R, Morales D R, Lim J, Breaker R R: Riboswitches that sense S-adenosylhomocysteine and activate genes involved in coenzyme recycling. Mol Cell 2008, 29:691-702.
Wang J X, Lee E R, Morales D R, Lim J, Breaker R R: Riboswitches that sense S-adenosylhomocysteine and activate genes involved in coenzyme recycling. Mol Cell 2008, 29:691-702.
Wang L, Jensen S, Hallman R, Reeves P R: Expression of the O antigen gene cluster is regulated by RfaH through the JUMPstart sequence. FEMS Microbiol Lett 1998, 165:201-206.
Wang L, Jensen S, Hallman R, Reeves P R: Expression of the O antigen gene cluster is regulated by RfaH through the JUMPstart sequence. FEMS Microbiol Lett 1998, 165:201-206.
Warnecke F, Luginbuhl P, Ivanova N, Ghassemian M, Richardson T H, Stege J T, Cayouette M, McHardy A C, Djordjevic G, Aboushadi N, Sorek R, Tringe S G, Podar M, Martin H G, Kunin V, Dalevi D, Madejska J, Kirton E, Platt D, Szeto E, Salamov A, Barry K, Mikhailova N, Kyrpides N C, Matson E G, Ottesen E A, Zhang X, Hernandez M, Murillo C, Acosta L G, Rigoutsos I, Tamayo G, Green B D, Chang C, Rubin E M, Mathur E J, Robertson D E, Hugenholtz P, Leadbetter J R: Mctagcnomic and functional analysis of hindgut microbiota of a wood-feeding higher termite. Nature 2007, 450:560-565.
Waters L S, Storz G: Regulatory RNAs in bacteria. Cell 2009, 136:615-628.
Weinberg Z, Barrick J E, Yao Z, Roth A, Kim J N, Gore J, Wang J X, Lee E R, Block K F, Sudarsan N, et al. Identification of 22 candidate structured RNAs in bacteria using the CMfinder comparative genomics pipeline. Nucleic Acids Res 2007; 35:4809-4819.
Weinberg Z, Barrick J E, Yao Z, Roth A, Kim J N, Gore J, Wang J X, Lee E R, Block K F, Sudarsan N, Neph S, Tompa M, Ruzzo W L, Breaker R R: Identification of 22 candidate structured RNAs in bacteria using the CMfinder comparative genomics pipeline. Nucleic Acids Res. 2007, 35:4809-4819.
Weinberg Z, Barrick J E, Yao Z, Roth A, Kim J N, Gore J, Wang J X, Lee E R, Block K F, Sudarsan N, Neph S, Tompa M, Ruzzo W L, Breaker R R: Identification of 22 candidate structured RNAs in bacteria using the CMfinder comparative genomics pipeline. Nucleic Acids Res. 2007, 35:4809-4819.
Weinberg Z, Perreault J, Meyer M M, Breaker R R: Extraordinary structured noncoding RNAs revealed by bacterial metagenome analysis. Nature 2009: accepted.
Weinberg Z, Regulski E E, Hammond M C, Barrick J E, Yao Z, Ruzzo W L, Breaker R R: The aptamer core of SAM-IV riboswitches mimics the ligand-binding site of SAM-I riboswitches. Rna 2008, 14:822-828.
Weinberg Z, Regulski E E, Hammond M C, Barrick J E, Yao Z, Ruzzo W L, Breaker R R: The aptamer core of SAM-IV riboswitches mimics the ligand-binding site of SAM-I riboswitches. Rna 2008, 14:822-828.
Weinberg Z, Ruzzo W L: Sequence-based heuristics for faster annotation of non-coding RNA families. Bioinformatics 2006, 22:35-39.
Weinberg Z, Wang J, Bogue J, Yang J, Corbino K, Moy R, Breaker R R. Comparative genomics reveals 104 candidate structured RNAs from bacteria, archaea, and their metagenomes. Genome Biol 2010; 11:R31.
Welz R, Breaker R R. Ligand binding and gene control characteristics of tandem riboswitches in Bacillus anthracis. RNA 2007; 13:573-582.
Welz R, Breaker R R: Ligand binding and gene control characteristics of tandem riboswitches in Bacillus anthracis. Rna 2007, 13:573-582.
Weng M, Nagy P L, Zalkin H: Identification of the Bacillus subtilis pur operon repressor. Proc Natl Acad Sci USA 1995, 92:7455-7459.
Wickiser J K, Cheah M T, Breaker R R, Crothers D M. The kinetics of ligand binding by an adenine-sensing riboswitch. Biochemistry 2005; 44:13404-13414.
Wickiser J K, Winkler W C, Breaker R R, Crothers D M. The speed of RNA transcription and metabolite binding kinetics operate an FMN riboswitch. Mol Cell 2005; 18:49-60.
Wijayarathna C D, Wachi M, Nagai K: Isolation of ftsI and murE genes involved in peptidoglycan synthesis from Corynebacterium glutamicum. Appl Microbiol Biotechnol 2001, 55:466-470.
Winkler W C, Nahvi A, Roth A, Collins J A, Breaker R R. Control of gene expression by a natural metabolite-responsive ribozyme. Nature 2004; 428:281-286.
Winkler W C, Nahvi A, Sudarsan N, Barrick J E, Breaker R R: An mRNA structure that controls gene expression by binding S-adenosylmethionine Nat. Struct. Biol. 2003, 10:701-707.
Woyke T, Teeling H, Ivanova N N, Huntemann M, Richter M, Gloeckner F O, Boffelli D, Anderson I J, Barry K W, Shapiro H J, Szeto E, Kyrpides N C, Mussmann M, Amann R, Bergin C, Ruehland C, Rubin E M, Dubilier N: Symbiosis insights through metagenomic analysis of a microbial consortium. Nature 2006, 443:950-955.
Yamanishi Y, Mihara H, Osaki M, Muramatsu H, Esaki N, Sato T, Hizukuri Y, Goto S, Kanehisa M: Prediction of missing enzyme genes in a bacterial metabolic network. Reconstruction of the lysine-degradation pathway of Pseudomonas aeruginosa. Febs J2007, 274:2262-2273.
Yao Z, Barrick J, Weinberg Z, Neph S, Breaker R, Tompa M, Ruzzo W L: A computational pipeline for high-throughput discovery of cis-regulatory noncoding RNA in prokaryotes. PLoS Comput. Biol. 2007, 3:e126.
Yao Z, Barrick J, Weinberg Z, Neph S, Breaker R, Tompa M, Ruzzo W L: A computational pipeline for high-throughput discovery of cis-regulatory noncoding RNA in prokaryotes. PLoS Comput. Biol. 2007, 3:e126.
Yao Z, Barrick J, Weinberg Z, Neph S, Breaker R R, Tompa M, Ruzzo W L. A computational pipeline for high-throughput discovery of cis-regulatory noncoding RNA in prokaryotes. PLoS Comput Biol 2007; 3:e126.
Yao Z, Weinberg Z, Ruzzo W L: CMfinder—a covariance model based RNA motif finding algorithm. Bioinformatics 2006, 22:445-452.
Yao Z: Genome scale search of noncoding RNAs: bacteria to vertebrates. Seattle, Wash.: University of Washington; 2008. Dissertation.
Yoder-Himes D R, Chain P S, Zhu Y, Wurtzel O, Rubin E M, Tiedje J M, Sorek R: Mapping the Burkholderia cenocepacia niche response via high-throughput sequencing. Proc Natl Acad Sci USA 2009, 106:3976-3981.
Yooseph S, Sutton G, Rusch D B, Halpern A L, Williamson S J, Remington K, Eisen J A, Heidelberg K B, Manning G, Li W, Jaroszewski L, Cieplak P, Miller C S, Li H, Mashiyama S T, Joachimiak M P, van Belle C, Chandonia J M, Soergel D A, Zhai Y, Natarajan K, Lee S, Raphael B J, Bafna V, Friedman R, Brenner S E, Godzik A, Eisenberg D, Dixon J E, Taylor S S, Strausberg R L, Frazier M, Venter J C: The Sorcerer TI Global Ocean Sampling expedition: expanding the universe of protein families. PLoS Biol 2007, 5:e16.
Yooseph S, Sutton G, Rusch D B, Halpern A L, Williamson S J, Remington K, Eisen J A, Heidelberg K B, Manning G, Li W, Jaroszewski L, Cieplak P, Miller C S, Li H, Mashiyama S T, Joachimiak M P, van Belle C, Chandonia J M, Soergel D A, Zhai Y, Natarajan K, Lee S, Raphael B J, Bafna V, Friedman R, Brenner S E, Godzik A, Eisenberg D, Dixon J E, Taylor S S, Strausberg R L, Frazier M, Venter J C: The Sorcerer II Global Ocean Sampling expedition: expanding the universe of protein families. PLoS Biol 2007, 5:e16.
Zengel J M, Lindahl L: Diverse mechanisms for regulating ribosomal protein synthesis in \taxonEscherichia coli. Prog Nucleic Acid Res Mol Biol 1994, 47:331-370.

1. A regulatable gene expression construct comprising
a nucleic acid molecule encoding an RNA comprising a riboswitch operably linked to a coding region, wherein the riboswitch regulates expression of the RNA, wherein the riboswitch and coding region are heterologous, wherein the riboswitch is an S-adenosylhomocysteine-repsonsive riboswitch, a crcB riboswitch, a ykkC-III riboswitch, an S-adenosylmethionine-repsonsive riboswitch, a SAM/SAH riboswitch, a glutamine-responsive riboswitch, a glutamine riboswitch, a glnA riboswitch, a Downstream-peptide riboswitch, a pfl riboswitch, a yjdF riboswitch, a manA riboswitch, a wcaG riboswitch, an epsC riboswitch, a psaA riboswitch, a psbA riboswitch, a PhotoRC-I riboswitch, a PhotoRC-II riboswitch, or a psbNH riboswitch.

2. The construct of claim 1 wherein the riboswitch comprises an aptamer domain and an expression platform domain, wherein the aptamer domain and the expression platform domain are heterologous.

3. The construct of claim 1, wherein the riboswitch comprises two or more aptamer domains and an expression platform domain, wherein at least one of the aptamer domains and the expression platform domain are heterologous.

4. The construct of claim 3, wherein at least two of the aptamer domains exhibit cooperative binding.

5. A riboswitch, wherein the riboswitch is a non-natural derivative of a naturally-occurring riboswitch, wherein the naturally-occurring riboswitch is an S-adenosylhomocysteine-repsonsive riboswitch, a crcB riboswitch, a ykkC-III riboswitch, an S-adenosylmethionine-repsonsive riboswitch, a SAM/SAH riboswitch, a glutamine-responsive riboswitch, a glutamine riboswitch, a glnA riboswitch, a Downstream-peptide riboswitch, a pfl riboswitch, a yjdF riboswitch, a manA riboswitch, a wcaG riboswitch, an epsC riboswitch, a psaA riboswitch, a psbA riboswitch, a PhotoRC-I riboswitch, a PhotoRC-II riboswitch, or a psbNH riboswitch.

6. The riboswitch of claim 5, wherein the riboswitch comprises an aptamer domain and an expression platform domain, wherein the aptamer domain and the expression platform domain are heterologous.

7. The riboswitch of claim 6, wherein the riboswitch comprises a crcB motif, a ykkC-III motif, a SAM/SAH motif, a glnA motif, a Downstream-peptide motif, a a pfl motif, a yjdF motif, a manA motif, a wcaG motif, a epsC motif, a psaA motif, a psbA motif, a PhotoRC-I motif, a PhotoRC-II motif, or a psbNH motif.

8. The riboswitch of claim 5, wherein the riboswitch is activated by a trigger molecule, wherein the riboswitch produces a signal when activated by the trigger molecule.

9. The construct of claim 1, wherein the riboswitch has one of the consensus structures of FIG. 1, FIG. 2, FIG. 3, FIG. 4, or FIG. 5.

10. The construct of claim 1, wherein the riboswitch comprises an aptamer domain and an expression platform domain wherein the aptamer domain is derived from a naturally-occurring S-adenosylhomocysteine-repsonsive riboswitch, crcB riboswitch, a ykkC-III riboswitch, S-adenosylmethionine-repsonsive riboswitch, SAM/SAH riboswitch, glutamine-responsive riboswitch, glutamine riboswitch, glnA riboswitch, Downstream-peptide riboswitch, pfl riboswitch, yjdF riboswitch, manA riboswitch, wcaG riboswitch, epsC riboswitch, psaA riboswitch, psbA riboswitch, PhotoRC-I riboswitch, PhotoRC-II riboswitch, or psbNH riboswitch.

11. The construct of claim 10, wherein the aptamer domain is the aptamer domain of a naturally-occurring S adenosylhomocysteine-repsonsive riboswitch, crcB riboswitch, a ykkC-III riboswitch, S-adenosylmethionine-repsonsive riboswitch, SAM/SAH riboswitch, glutamine-responsive riboswitch, glutamine riboswitch, glnA riboswitch, Downstream-peptide riboswitch, pfl riboswitch, yjdF riboswitch, manA riboswitch, wcaG riboswitch, epsC riboswitch, psaA riboswitch, psbA riboswitch, PhotoRC-I riboswitch, PhotoRC-II riboswitch, or psbNH riboswitch.

12. The construct of claim 10, wherein the aptamer domain has the consensus structure of an aptamer domain of the naturally-occurring riboswitch.

13. The construct of claim 10, wherein the aptamer domain consists of only base pair conservative changes of the naturally-occurring riboswitch.

14. A method of detecting a compound of interest, the method comprising
bringing into contact a sample and a riboswitch, wherein the riboswitch is activated by the compound of interest, wherein the riboswitch produces a signal when activated by the compound of interest, wherein the riboswitch produces a signal when the sample contains the compound of interest, wherein the riboswitch is an S-adenosylhomocysteine-repsonsive riboswitch, a crcB riboswitch, a ykkC-III riboswitch, an S-adenosylmethionine-repsonsive riboswitch, a SAM/SAH riboswitch, a glutamine-responsive riboswitch, a glutamine riboswitch, a glnA riboswitch, a Downstream-peptide riboswitch, a pfl riboswitch, a yjdF riboswitch, a manA riboswitch, a wcaG riboswitch, an epsC riboswitch, a psaA riboswitch, a psbA riboswitch, a PhotoRC-I riboswitch, a PhotoRC-II riboswitch, or a psbNH riboswitch.

15. The method of claim 14, wherein the riboswitch changes conformation when activated by the compound of interest, wherein the change in conformation produces a signal via a conformation dependent label.

16. The method of claim 14, wherein the riboswitch changes conformation when activated by the compound of interest, wherein the change in conformation causes a change in expression of an RNA linked to the riboswitch, wherein the change in expression produces a signal.

17. The method of claim 16, wherein the signal is produced by a reporter protein expressed from the RNA linked to the riboswitch.

18. A method comprising
(a) testing a compound for altering gene expression of a gene encoding an RNA comprising a riboswitch, wherein the alteration is via the riboswitch, wherein the riboswitch is an S-adenosylhomocysteine-repsonsive riboswitch, a crcB riboswitch, a ykkC-III riboswitch, an S-adenosylmethionine-repsonsive riboswitch, a SAM/SAH riboswitch, a glutamine-responsive riboswitch, a glutamine riboswitch, a glnA riboswitch, a Downstream-peptide riboswitch, a pfl riboswitch, a yjdF riboswitch, a manA riboswitch, a wcaG riboswitch, an epsC riboswitch, a psaA riboswitch, a psbA riboswitch, a PhotoRC-I riboswitch, a PhotoRC-II riboswitch, or a psbNH riboswitch,
(b) altering gene expression by bringing into contact a cell and a compound that altered gene expression in step (a),
wherein the cell comprises a gene encoding an RNA comprising a riboswitch, wherein the compound inhibits expression of the gene by binding to the riboswitch.

19. A method of identifying riboswitches, the method comprising
assessing in-line spontaneous cleavage of an RNA molecule in the presence and absence of a compound, wherein the RNA molecule is encoded by a gene regulated by the compound,
wherein a change in the pattern of in-line spontaneous cleavage of the RNA molecule indicates a riboswitch, wherein (a) the RNA comprises an S-adenosylhomocysteine-repsonsive riboswitch or a derivative of an S-adenosylhomocysteine-repsonsive riboswitch and the compound is S-adenosylhomocysteine, (b) the RNA comprises an S-adenosylmethionine-repsonsive riboswitch or a derivative of an S-adenosylmethionine-repsonsive riboswitch and the compound is S-adenosylmethionine, or (c) a glutamine-responsive riboswitch or a derivative of a glutamine-responsive riboswitch and the compound is glutamine.

20. A method of altering gene expression, the method comprising
bringing into contact a compound and a cell, wherein the cell comprises a gene encoding an RNA comprising an S-adenosylhomocysteine-repsonsive riboswitch, a crcB riboswitch, a ykkC-III riboswitch, an S-adenosylmethionine-repsonsive riboswitch, a SAM/SAH riboswitch, a glutamine-responsive riboswitch, a glutamine riboswitch, a glnA riboswitch, a Downstream-peptide riboswitch, a pfl riboswitch, a yjdF riboswitch, a manA riboswitch, a wcaG riboswitch, an epsC riboswitch, a psaA riboswitch, a psbA riboswitch, a PhotoRC-I riboswitch, a PhotoRC-II riboswitch, or a psbNH riboswitch.

21. The method of any of claim 20, wherein the cell has been identified as being in need of altered gene expression.

22. The method of claim 20, wherein the cell is a bacterial cell.

23. The method of claim 22, wherein the compound kills or inhibits the growth of the bacterial cell.

24. The method of claim 20, wherein the compound and the cell are brought into contact by administering the compound to a subject.

25. The method of claim 24, wherein the cell is a bacterial cell in the subject, wherein the compound kills or inhibits the growth of the bacterial cell.

26. The method of claim 25, wherein the subject has a bacterial infection.

27. The method of claim 20, wherein the compound is administered in combination with another antimicrobial compound.

28. The method of claim 20, wherein the compound inhibits bacterial growth in a biofilm.


SEQUENCE LISTING (301 sequences)

SEQ ID NO: 1 - DNA, 50 bp, Artificial Sequence
  chemically synthesized; primer
taatacgactcactatagggtaatcgttggcccagtttatctgggtggaa

SEQ ID NO: 2 - DNA, 49 bp, Artificial Sequence
  chemically synthesized; primer
tgagaggcgcgttgcttcaggccaaagaccttacttccacccagataaa

SEQ ID NO: 3 - DNA, 50 bp, Artificial Sequence
  chemically synthesized; primer
taatacgactcactatagggtaatcgttggcccagtttatcaaggtggaa

SEQ ID NO: 4 - DNA, 50 bp, Artificial Sequence
  chemically synthesized; primer
taatacgactcactatagggtaatcgttggccttgtttatcaaggtggaa

SEQ ID NO: 5 - DNA, 49 bp, Artificial Sequence
  chemically synthesized; primer
tgagaggcgcgttgcttcaggccaaagaccttacttccaccttgataaa

SEQ ID NO: 6 - DNA, 49 bp, Artificial Sequence
  chemically synthesized; primer
tgagaggcgcgttgcttcagcccaaagtccttacttccacccagataaa

SEQ ID NO: 7 - DNA, 49 bp, Artificial Sequence
  chemically synthesized; primer
tgagaggcgcgttgcttcagctcaaagtgcttacttccacccagataaa

SEQ ID NO: 8 - DNA, 58 bp, Artificial Sequence
  chemically synthesized; primer
taatacgactcactatagggtattcttggtccacgttgagcttccaatcgaagctgca

SEQ ID NO: 9 - DNA, 58 bp, Artificial Sequence
  chemically synthesized; primer
tccttcattgcccacgcccccgttgcttggcatgggtctgactgcagcttcgattgga

SEQ ID NO: 10 - DNA, 62 bp, Artificial Sequence
  chemically synthesized; primer
tccttcattgccctcgcccccgttgcttggcctgggtctgactgcagcttcgattggaagct

SEQ ID NO: 11 - DNA, 62 bp, Artificial Sequence
  chemically synthesized; primer
tccttcattgccctagcccccgttgcttggccagggtctgactgcagcttcgattggaagct

SEQ ID NO: 12 - RNA, 92 nt, Clostridium acetobutylicum
  misc_feature (1)..(92)
gguaaaauaagaaaaucaugcaacuggcggaaauggaguucaccauagggagcaugauuaauauaagaaucgaccgccuggguaaauuaaua

SEQ ID NO: 13 - RNA, 122 nt, Bacillus subtilis
  misc_feature (1)..(122)
gguaaagaaugaaaaaacacgauucgguugguaguccggaugcaugauugagaaugucaguaaccuuccccuccucgggauguccaucauucuuuaauaucuuuuaugaggagggaaucguu

SEQ ID NO: 14 - RNA, 102 nt, Bacillus subtilis
  misc_feature (1)..(102)
gguaaagaaugaaaaaacacgauucgguugguaguccggaugcaugauugagaaugucaguaaccuuccccuccucgggauguccaucauucuuuaauaucu

SEQ ID NO: 15 - RNA, 78 nt, Bacillus subtilis
  misc_feature (1)..(78)
gguaaagaaugaaaaaacacgauucgguugguaguccggaugcaugauugagaaugucaguaaccuuccccuccucgg

SEQ ID NO: 16 - RNA, 201 nt, Chlorobium tepidum
  misc_feature (1)..(201)
ggauuuuccggcauccccauuaccuauggacacggugccaaaagcucucuugcgggaguuguccccggagcuugccgaaagguuucccgugucccguuugucccuccgcgacauucaccuucacgagaaaaccgcaucggcaaaccgccggacaccugccguucuugucguucgauuaacaaaaaaccgaaagggaaacua

SEQ ID NO: 17 - RNA, 104 nt, Chlorobium tepidum
  misc_feature (1)..(104)
ggauuuuccggcauccccauuaccuauggacacggugccaaaagcucucuugcgggaguuguccccggagcuugccgaaagguuucccgugucccguuuguccc

SEQ ID NO: 18 - RNA, 87 nt, Chlorobium tepidum
  misc_feature (1)..(87)
ggauuuuccggcauccccauuaccuauggacacggugccaaaagcucucuugcgggaguuguccccggagcuugccgaaagguuucc

SEQ ID NO: 19 - RNA, 60 nt, Chlorobium tepidum
  misc_feature (1)..(60)
ggauuuuccggcauccccauuaccuauggacacggugccaaaagcucucuugcgggaguu

SEQ ID NO: 20 - RNA, 106 nt, Geobacter metallireducens
  misc_feature (1)..(106)
ggcaaauugauacugccuggauucguacgaaccgggacggauggcaauagccgcaacgacaaggaaauagcuuuuucucuuggucuugguacaugcgccuccggaa

SEQ ID NO: 21 - RNA, 70 nt, Streptomyces coelicolor
  misc_feature (1)..(70)
ggacuacaccaccaccuuccuacaacggaucguccggcacguuccugccgguagaagggggcccuuucac

SEQ ID NO: 22 - RNA, 72 nt, Pseudomonas syringae
  misc_feature (1)..(72)
ggucuuggcggccugaaggcugcagcagucgaucaucguaugcuguugcaguugauccagcccgcuugaucc

SEQ ID NO: 23 - RNA, 112 nt, Pseudomonas syringae
  misc_feature (1)..(112)
ggucuuggcggccugaaggcugcagcagucgaucaucguaugcuguugcaguugauccagcccgcuugauccuugaaccacgccgaccgaugagcggcgaaugaggaauaca

SEQ ID NO: 24 - RNA, 140 nt, Pseudomonas syringae
  misc_feature (1)..(140)
ggcgcuuugguuagaaaucaacucaggucauuuccgcaaugguuauggcaucaaggcccgccacgccggcagcgggccccaacggcagaagacucugcccgaccccaccacggggucucagggauauuacagucaacaga

SEQ ID NO: 25 - RNA, 158 nt, Pseudomonas syringae
  misc_feature (1)..(158)
ggaucauucacaucacccugcgcuuugguuagaaaucaacucaggucauuuccgcaaugguuauggcaucaaggcccgccacgccggcagcgggccccaacggcagaagacucugcccgaccccaccacggggucucagggauauuacagucaacaga

SEQ ID NO: 26 - RNA, 164 nt, Pseudomonas syringae
  misc_feature (1)..(164)
ggcgcuuugguuagaaaucaacucaggucauuuccgcaaugguuauggcaucaaggcccgccacgccggcagcgggccccaacggcagaagacucugcccgaccccaccacggggucucagggauauuacagucaacagacgagggcauuacccuaugagaaga

SEQ ID NO: 27 - RNA, 182 nt, Pseudomonas syringae
  misc_feature (1)..(182)
ggaucauucacaucacccugcgcuuugguuagaaaucaacucaggucauuuccgcaaugguuauggcaucaaggcccgccacgccggcagcgggccccaacggcagaagacucugcccgaccccaccacggggucucagggauauuacagucaacagacgagggcauuacccuaugagaaga

SEQ ID NO: 28 - RNA, 71 nt, Synechococcus CC9605
  misc_feature (1)..(71)
ggcgaccacguucaccucgucuucggcgaggcgcaguucgacucaggccauggaacggggaccugagcuug

SEQ ID NO: 29 - RNA, 88 nt, Synechococcus CC9605
  misc_feature (1)..(88)
ggcuacgcgaccacguucaccucgucuucggcgaggcgcaguucgacucaggccauggaacggggaccugagcuugcuucgaggaacu

SEQ ID NO: 30 - protein, 6 aa, Cyanobacteria
  MISC_FEATURE (4)..(4): X can be any amino acid
YRGXXY

SEQ ID NO: 31 - RNA, 25 nt, Artificial Sequence
  chemically synthesized; RNA motif
ycacaacggcuuccugrcgugryrr

SEQ ID NO: 32 - RNA, 52 nt, Artificial Sequence
  chemically synthesized;  RNA motif
gguaccugucacaacggcuuccuggcgugacgaggugaccucaguggagcaa

SEQ ID NO: 33 - RNA, 10 nt, Artificial Sequence
  chemically synthesized;  RNA motif
rgcugaugac

SEQ ID NO: 34 - RNA, 12 nt, Artificial Sequence
  chemically synthesized;  RNA motif
yryracugrcgr

SEQ ID NO: 35 - RNA, 11 nt, Artificial Sequence
  chemically synthesized;  RNA motif
ycgycugggcr

SEQ ID NO: 36 - RNA, 12 nt, Artificial Sequence
  chemically synthesized;  RNA motif
gugacugaauaa

SEQ ID NO: 37 - RNA, 12 nt, Artificial Sequence
  chemically synthesized;  RNA motif
uaaggurayrrg

SEQ ID NO: 38 - RNA, 10 nt, Artificial Sequence
  chemically synthesized;  RNA motif
agguggugcu

SEQ ID NO: 39 - RNA, 13 nt, Artificial Sequence
  chemically synthesized;  RNA motif
gyrccguacccgg

SEQ ID NO: 40 - RNA, 11 nt, Artificial Sequence
  chemically synthesized;  RNA motif
gacaagacgry

SEQ ID NO: 41 - RNA, 11 nt, Artificial Sequence
  chemically synthesized;  RNA motif
ruaaaaacacr

SEQ ID NO: 42 - RNA, 20 nt, Artificial Sequence
  chemically synthesized;  RNA motif
ryyygguygguaguccrrry

SEQ ID NO: 43 - RNA, 12 nt, Artificial Sequence
  chemically synthesized;  RNA motif
ygucaguaaccu

SEQ ID NO: 44 - RNA, 13 nt, Artificial Sequence
  chemically synthesized;  RNA motif
gguraugucuccu

SEQ ID NO: 45 - RNA, 32 nt, Artificial Sequence
  chemically synthesized;  RNA motif
uuaucaayggaggcacucggyyrugcuguggg

SEQ ID NO: 46 - RNA, 10 nt, Artificial Sequence
  chemically synthesized;  RNA motif
ccuuagacgc

SEQ ID NO: 47 - RNA, 10 nt, Artificial Sequence
  chemically synthesized;  RNA motif
grygugaggr

SEQ ID NO: 48 - RNA, 13 nt, Artificial Sequence
  chemically synthesized;  RNA motif
ggguurraugccc

SEQ ID NO: 49 - RNA, 13 nt, Artificial Sequence
  chemically synthesized;  RNA motif
uggugcggauggg

SEQ ID NO: 50 - RNA, 14 nt, Artificial Sequence
  chemically synthesized;  RNA motif
yccgccyrguuucy

SEQ ID NO: 51 - RNA, 10 nt, Artificial Sequence
  chemically synthesized;  RNA motif
cggaaguarr

SEQ ID NO: 52 - RNA, 13 nt, Artificial Sequence
  chemically synthesized;  RNA motif
yygaaggaacgcr

SEQ ID NO: 53 - RNA, 10 nt, Artificial Sequence
  chemically synthesized;  RNA motif
cguucaycyy

SEQ ID NO: 54 - RNA, 24 nt, Artificial Sequence
  chemically synthesized;  RNA motif
ycagcgguccccucuuuggggccc

SEQ ID NO: 55 - RNA, 10 nt, Artificial Sequence
  chemically synthesized;  RNA motif
agcuycagug

SEQ ID NO: 56 - RNA, 12 nt, Artificial Sequence
  chemically synthesized;  RNA motif
yrcugrcgcccg

SEQ ID NO: 57 - RNA, 12 nt, Artificial Sequence
  chemically synthesized;  RNA motif
cggggccccgry

SEQ ID NO: 58 - RNA, 15 nt, Artificial Sequence
  chemically synthesized;  RNA motif
ycggagggguggccc

SEQ ID NO: 59 - RNA, 36 nt, Artificial Sequence
  chemically synthesized;  RNA motif
ccaccucgaycccgucccucgaggaacgacucgaug

SEQ ID NO: 60 - RNA, 37 nt, Artificial Sequence
  chemically synthesized;  RNA motif
aaccguyycyucggrrrcayuuyuuuccguucucaug

SEQ ID NO: 61 - RNA, 20 nt, Artificial Sequence
  chemically synthesized;  RNA motif
gauacuguaggggycrryyc

SEQ ID NO: 62 - RNA, 27 nt, Artificial Sequence
  chemically synthesized;  RNA motif
gggyygruccugucgagagaugugaug

SEQ ID NO: 63 - RNA, 11 nt, Artificial Sequence
  chemically synthesized;  RNA motif
aaccragaccu

SEQ ID NO: 64 - RNA, 12 nt, Artificial Sequence
  chemically synthesized;  RNA motif
gucuyucauayc

SEQ ID NO: 65 - RNA, 15 nt, Artificial Sequence
  chemically synthesized;  RNA motif
ycaauggcgggccrc

SEQ ID NO: 66 - RNA, 17 nt, Artificial Sequence
  chemically synthesized;  RNA motif
ycggyggauuacaaagr

SEQ ID NO: 67 - RNA, 14 nt, Artificial Sequence
  chemically synthesized;  RNA motif
augcgaugagucga

SEQ ID NO: 68 - RNA, 21 nt, Artificial Sequence
  chemically synthesized;  RNA motif
racgcaggagcggayyuugay

SEQ ID NO: 69 - RNA, 14 nt, Artificial Sequence
  chemically synthesized;  RNA motif
aruugugugaaccu

SEQ ID NO: 70 - RNA, 13 nt, Artificial Sequence
  chemically synthesized;  RNA motif
ggccgcuuuuuuu

SEQ ID NO: 71 - RNA, 26 nt, Artificial Sequence
  chemically synthesized;  RNA motif
gurucycyccuyggggrgrgacagar

SEQ ID NO: 72 - RNA, 10 nt, Artificial Sequence
  chemically synthesized;  RNA motif
ruguraggag

SEQ ID NO: 73 - RNA, 13 nt, Artificial Sequence
  chemically synthesized;  RNA motif
ycacruccugygr

SEQ ID NO: 74 - RNA, 12 nt, Artificial Sequence
  chemically synthesized;  RNA motif
ayyyrcruccug

SEQ ID NO: 75 - RNA, 15 nt, Artificial Sequence
  chemically synthesized;  RNA motif
ugrcggygggagrgr

SEQ ID NO: 76 - RNA, 9 nt, Artificial Sequence
  chemically synthesized;  RNA motif
cycrccgyy

SEQ ID NO: 77 - RNA, 10 nt, Artificial Sequence
  chemically synthesized;  RNA motif
rarugaauyy

SEQ ID NO: 78 - RNA, 13 nt, Artificial Sequence
  chemically synthesized;  RNA motif
caagguuuugcyy

SEQ ID NO: 79 - RNA, 14 nt, Artificial Sequence
  chemically synthesized;  RNA motif
yggcaaaacgcuug

SEQ ID NO: 80 - RNA, 30 nt, Artificial Sequence
  chemically synthesized;  RNA motif
rgyyruruggcgcgagcgcccauccacccc

SEQ ID NO: 81 - RNA, 13 nt, Artificial Sequence
  chemically synthesized;  RNA motif
gcgccruayggcu

SEQ ID NO: 82 - RNA, 30 nt, Artificial Sequence
  chemically synthesized;  RNA motif
gacccgyccggyuguccccccggrcggguc

SEQ ID NO: 83 - RNA, 10 nt, Artificial Sequence
  chemically synthesized;  RNA motif
gcrcugugar

SEQ ID NO: 84 - RNA, 11 nt, Artificial Sequence
  chemically synthesized;  RNA motif
ccaaugyyyyr

SEQ ID NO: 85 - RNA, 22 nt, Artificial Sequence
  chemically synthesized;  RNA motif
gagrrrcgccuucgggugayyr

SEQ ID NO: 86 - RNA, 16 nt, Artificial Sequence
  chemically synthesized;  RNA motif
rrcgcyuuygrgygry

SEQ ID NO: 87 - RNA, 10 nt, Artificial Sequence
  chemically synthesized;  RNA motif
rggacgaccy

SEQ ID NO: 88 - RNA, 10 nt, Artificial Sequence
  chemically synthesized;  RNA motif
gggacgrccc

SEQ ID NO: 89 - RNA, 15 nt, Artificial Sequence
  chemically synthesized;  RNA motif
ugrcggygggagrgr

SEQ ID NO: 90 - RNA, 12 nt, Artificial Sequence
  chemically synthesized;  RNA motif
rarrggugaguy

SEQ ID NO: 91 - RNA, 10 nt, Artificial Sequence
  chemically synthesized;  RNA motif
rgayyyuuga

SEQ ID NO: 92 - RNA, 19 nt, Artificial Sequence
  chemically synthesized;  RNA motif
yggucyucggurguggyyy

SEQ ID NO: 93 - RNA, 13 nt, Artificial Sequence
  chemically synthesized;  RNA motif
rrrcuucgaucga

SEQ ID NO: 94 - RNA, 15 nt, Artificial Sequence
  chemically synthesized;  RNA motif
uguaguaaaacuaca

SEQ ID NO: 95 - RNA, 11 nt, Artificial Sequence
  chemically synthesized;  RNA motif
auccaagaauu

SEQ ID NO: 96 - RNA, 19 nt, Artificial Sequence
  chemically synthesized;  RNA motif
cygcccayaaggccrgyyg

SEQ ID NO: 97 - RNA, 11 nt, Artificial Sequence
  chemically synthesized;  RNA motif
gccuuuccgcc

SEQ ID NO: 98 - RNA, 14 nt, Artificial Sequence
  chemically synthesized;  RNA motif
uggagcaagccaug

SEQ ID NO: 99 - RNA, 11 nt, Artificial Sequence
  chemically synthesized;  RNA motif
augcgaugagu

SEQ ID NO: 100 - RNA, 21 nt, Artificial Sequence
  chemically synthesized;  RNA motif
racgcaggagcggayyuugay

SEQ ID NO: 101 - RNA, 14 nt, Artificial Sequence
  chemically synthesized;  RNA motif
aruugugugaaccu

SEQ ID NO: 102 - RNA, 10 nt, Artificial Sequence
  chemically synthesized;  RNA motif
uggugggraa

SEQ ID NO: 103 - RNA, 22 nt, Artificial Sequence
  chemically synthesized;  RNA motif
yarugugaaauucauurgcugu

SEQ ID NO: 104 - RNA, 15 nt, Artificial Sequence
  chemically synthesized;  RNA motif
gagygccacccarya

SEQ ID NO: 105 - RNA, 17 nt, Artificial Sequence
  chemically synthesized;  RNA motif
agyccryuguygaayga

SEQ ID NO: 106 - RNA, 26 nt, Artificial Sequence
  chemically synthesized;  RNA motif
ggccaggaaaagucuaruucurcaau

SEQ ID NO: 107 - RNA, 13 nt, Artificial Sequence
  chemically synthesized;  RNA motif
ugcccurgurrcy

SEQ ID NO: 108 - RNA, 10 nt, Artificial Sequence
  chemically synthesized;  RNA motif
cccuccurgu

SEQ ID NO: 109 - RNA, 10 nt, Artificial Sequence
  chemically synthesized;  RNA motif
gucaacggua

SEQ ID NO: 110 - RNA, 12 nt, Artificial Sequence
  chemically synthesized; RNA motif
uaaggaguugac

SEQ ID NO: 111 - RNA, 13 nt, Artificial Sequence
  chemically synthesized; RNA motif
ycacruccugygr

SEQ ID NO: 112 - RNA, 12 nt, Artificial Sequence
  chemically synthesized; RNA motif
ayyyrcruccug

SEQ ID NO: 113 - RNA, 10 nt, Artificial Sequence
  chemically synthesized; RNA motif
uuyaaguccg

SEQ ID NO: 114 - RNA, 10 nt, Artificial Sequence
  chemically synthesized; RNA motif
rarugaauyy

SEQ ID NO: 115 - RNA, 13 nt, Artificial Sequence
  chemically synthesized; RNA motif
caagguuuugcyy

SEQ ID NO: 116 - RNA, 14 nt, Artificial Sequence
  chemically synthesized; RNA motif
yggcaaaacgcuug

SEQ ID NO: 117 - RNA, 12 nt, Artificial Sequence
  chemically synthesized; RNA motif
ruaugygaauau

SEQ ID NO: 118 - RNA, 17 nt, Artificial Sequence
  chemically synthesized; RNA motif
uaugccuccccggcaua

SEQ ID NO: 119 - RNA, 21 nt, Artificial Sequence
  chemically synthesized; RNA motif
ryurgauygggaracucauca

SEQ ID NO: 120 - RNA, 15 nt, Artificial Sequence
  chemically synthesized; RNA motif
ycaauggcgggccrc

SEQ ID NO: 121 - RNA, 17 nt, Artificial Sequence
  chemically synthesized; RNA motif
ycggyggauuacaaagr

SEQ ID NO: 122 - RNA, 13 nt, Artificial Sequence
  chemically synthesized; RNA motif
cucaaaucyuguy

SEQ ID NO: 123 - RNA, 10 nt, Artificial Sequence
  chemically synthesized; RNA motif
yrgugcggcu

SEQ ID NO: 124 - RNA, 12 nt, Artificial Sequence
  chemically synthesized; RNA motif
ugcgrcgggcag

SEQ ID NO: 125 - RNA, 14 nt, Artificial Sequence
  chemically synthesized; RNA motif
grcauuggccggcc

SEQ ID NO: 126 - RNA, 13 nt, Artificial Sequence
  chemically synthesized; RNA motif
yugcccgcyuucg

SEQ ID NO: 127 - RNA, 13 nt, Artificial Sequence
  chemically synthesized; RNA motif
rrraaargcggcy

SEQ ID NO: 128 - RNA, 13 nt, Artificial Sequence
  chemically synthesized; RNA motif
ggccgcuuuuuuu

SEQ ID NO: 129 - RNA, 19 nt, Artificial Sequence
  chemically synthesized; RNA motif
uyagycaaygguugrcuga

SEQ ID NO: 130 - RNA, 26 nt, Artificial Sequence
  chemically synthesized; RNA motif
crrrurauuugugcuuugguuayuug

SEQ ID NO: 131 - RNA, 15 nt, Artificial Sequence
  chemically synthesized; RNA motif
rgyruyucuyuyuau

SEQ ID NO: 132 - RNA, 17 nt, Artificial Sequence
  chemically synthesized; RNA motif
arrauugygrgagyryr

SEQ ID NO: 133 - RNA, 24 nt, Artificial Sequence
  chemically synthesized; RNA motif
cgguggrrcggcugcaaaagagcc

SEQ ID NO: 134 - RNA, 23 nt, Artificial Sequence
  chemically synthesized; RNA motif
ycycacyggguaaaaucarrrur

SEQ ID NO: 135 - RNA, 13 nt, Artificial Sequence
  chemically synthesized; RNA motif
gccgggcraygca

SEQ ID NO: 136 - RNA, 10 nt, Artificial Sequence
  chemically synthesized; RNA motif
ggcugcaacc

SEQ ID NO: 137 - RNA, 10 nt, Artificial Sequence
  chemically synthesized; RNA motif
ruguraggag

SEQ ID NO: 138 - RNA, 11 nt, Artificial Sequence
  chemically synthesized; RNA motif
ggycgyucyyu

SEQ ID NO: 139 - RNA, 10 nt, Artificial Sequence
  chemically synthesized; RNA motif
rcauccrugy

SEQ ID NO: 140 - RNA, 15 nt, Artificial Sequence
  chemically synthesized; RNA motif
aycrcgrcauuygyr

SEQ ID NO: 141 - RNA, 10 nt, Artificial Sequence
  chemically synthesized; RNA motif
rgcugaugac

SEQ ID NO: 142 - RNA, 10 nt, Artificial Sequence
  chemically synthesized; RNA motif
cguucaycyy

SEQ ID NO: 143 - RNA, 15 nt, Artificial Sequence
  chemically synthesized; RNA motif
cggragagcgaycyg

SEQ ID NO: 144 - RNA, 12 nt, Artificial Sequence
  chemically synthesized; RNA motif
yrygagcgauyr

SEQ ID NO: 145 - RNA, 12 nt, Artificial Sequence
  chemically synthesized; RNA motif
uyrgugcruyyg

SEQ ID NO: 146 - RNA, 27 nt, Artificial Sequence
  chemically synthesized; RNA motif
rcrgaaccgcaucgcucgggragrrcr

SEQ ID NO: 147 - RNA, 24 nt, Artificial Sequence
  chemically synthesized; RNA motif
uucuycucgcggyucygurgyccy

SEQ ID NO: 148 - RNA, 45 nt, Artificial Sequence
  chemically synthesized; RNA motif
ugccacaggcrucaagggguccgaaaggcuccugaugyrcyugrc

SEQ ID NO: 149 - RNA, 10 nt, Artificial Sequence
  chemically synthesized; RNA motif
acuraguaar

SEQ ID NO: 150 - RNA, 27 nt, Artificial Sequence
  chemically synthesized; RNA motif
grggcyarggaaagguaaggrgyaara

SEQ ID NO: 151 - RNA, 20 nt, Artificial Sequence
  chemically synthesized; RNA motif
yccuuacuaaguacuurrrr

SEQ ID NO: 152 - RNA, 23 nt, Artificial Sequence
  chemically synthesized; RNA motif
yyagarrguaryagggguruugr

SEQ ID NO: 153 - RNA, 27 nt, Artificial Sequence
  chemically synthesized; RNA motif
raagcycgguacyycurugaaaucugg

SEQ ID NO: 154 - RNA, 38 nt, Artificial Sequence
  chemically synthesized; RNA motif
ggurcaagggagcyraguagggucyguugagccuggca

SEQ ID NO: 155 - RNA, 39 nt, Artificial Sequence
  chemically synthesized; RNA motif
cggucaagauacgrgcgguuuurcgyucgccguagcuga

SEQ ID NO: 156 - RNA, 13 nt, Artificial Sequence
  chemically synthesized; RNA motif
gguraugucuccu

SEQ ID NO: 157 - RNA, 32 nt, Artificial Sequence; identical to SEQ ID NO: 45
  chemically synthesized; RNA motif

SEQ ID NO: 158 - RNA, 10 nt, Artificial Sequence
  chemically synthesized; RNA motif
ccuuagacgc

SEQ ID NO: 159 - RNA, 10 nt, Artificial Sequence
  chemically synthesized; RNA motif
grygugaggr

SEQ ID NO: 160 - RNA, 13 nt, Artificial Sequence
  chemically synthesized; RNA motif
ggguurraugccc

SEQ ID NO: 161 - RNA, 32 nt, Artificial Sequence
  chemically synthesized; RNA motif
yygyrrcagucgaucaucgyaugcugycrcrr

SEQ ID NO: 162 - RNA, 15 nt, Artificial Sequence
  chemically synthesized; RNA motif
ccggrcuugayccgg

SEQ ID NO: 163 - RNA, 10 nt, Artificial Sequence
  chemically synthesized; RNA motif
cggaaguarr

SEQ ID NO: 164 - RNA, 13 nt, Artificial Sequence
  chemically synthesized; RNA motif
yygaaggaacgcr

SEQ ID NO: 165 - RNA, 20 nt, Artificial Sequence
  chemically synthesized; RNA motif
yyayaagcycugauguaacu

SEQ ID NO: 166 - RNA, 37 nt, Artificial Sequence
  chemically synthesized; RNA motif
aayyucaaucaaggagcaucccauugayaaggaaaay

SEQ ID NO: 167 - RNA, 16 nt, Artificial Sequence
  chemically synthesized; RNA motif
ccargggcgguagcru

SEQ ID NO: 168 - RNA, 14 nt, Artificial Sequence
  chemically synthesized; RNA motif
gryyyacayuagyu

SEQ ID NO: 169 - RNA, 16 nt, Artificial Sequence
  chemically synthesized; RNA motif
rgcuarugugrguccu

SEQ ID NO: 170 - RNA, 13 nt, Artificial Sequence
  chemically synthesized; RNA motif
aagagacguccuc

SEQ ID NO: 171 - RNA, 13 nt, Artificial Sequence
  chemically synthesized; RNA motif
ggrcgucuuuuuu

SEQ ID NO: 172 - RNA, 14 nt, Artificial Sequence
  chemically synthesized; RNA motif
rryyyruraruaac

SEQ ID NO: 173 - RNA, 16 nt, Artificial Sequence
  chemically synthesized; RNA motif
cygaaucggaauacur

SEQ ID NO: 174 - RNA, 24 nt, Artificial Sequence
  chemically synthesized; RNA motif
yagrauccauauucrcugcgayay

SEQ ID NO: 175 - RNA, 41 nt, Artificial Sequence
  chemically synthesized; RNA motif
uuccuguuaaruaacagcuugraayaaauuuaaagaauaaa

SEQ ID NO: 176 - RNA, 12 nt, Artificial Sequence
  chemically synthesized; RNA motif
caaaaauaaaaa

SEQ ID NO: 177 - RNA, 14 nt, Artificial Sequence
  chemically synthesized; RNA motif
ucgucugaaacgar

SEQ ID NO: 178 - RNA, 14 nt, Artificial Sequence
  chemically synthesized; RNA motif
ygagugraagauga

SEQ ID NO: 179 - RNA, 10 nt, Artificial Sequence
  chemically synthesized; RNA motif
acggaucguc

SEQ ID NO: 180 - RNA, 14 nt, Artificial Sequence
  chemically synthesized; RNA motif
ggcacguaccugcc

SEQ ID NO: 181 - RNA, 16 nt, Artificial Sequence
  chemically synthesized; RNA motif
cuuccccrrygycagg

SEQ ID NO: 182 - RNA, 12 nt, Artificial Sequence
  chemically synthesized; RNA motif
cggryrgggryc

SEQ ID NO: 183 - RNA, 20 nt, Artificial Sequence
  chemically synthesized; RNA motif
gcgcyyayuucgyccucygy

SEQ ID NO: 184 - RNA, 11 nt, Artificial Sequence
  chemically synthesized; RNA motif
rcgaaacccgc

SEQ ID NO: 185 - RNA, 15 nt, Artificial Sequence
  chemically synthesized; RNA motif
caggayrrrggarra

SEQ ID NO: 186 - RNA, 14 nt, Artificial Sequence
  chemically synthesized; RNA motif
uygauuaacgcyyg

SEQ ID NO: 187 - RNA, 11 nt, Artificial Sequence
  chemically synthesized; RNA motif
yuggaagcaug

SEQ ID NO: 188 - RNA, 12 nt, Artificial Sequence
  chemically synthesized; RNA motif
gugacugaauaa

SEQ ID NO: 189 - RNA, 12 nt, Artificial Sequence
  chemically synthesized; RNA motif
uaaggurayrrg

SEQ ID NO: 190 - RNA, 10 nt, Artificial Sequence
  chemically synthesized; RNA motif
agguggugcu

SEQ ID NO: 191 - RNA, 10 nt, Artificial Sequence
  chemically synthesized; RNA motif
arcaagcaaa

SEQ ID NO: 192 - RNA, 23 nt, Artificial Sequence
  chemically synthesized; RNA motif
ayggrrraygcccgauuucugua

SEQ ID NO: 193 - RNA, 23 nt, Artificial Sequence
  chemically synthesized; RNA motif
cagaaauaugggcguyyuccgur

SEQ ID NO: 194 - RNA, 15 nt, Artificial Sequence
  chemically synthesized; RNA motif
gcuugyurgrgaryu

SEQ ID NO: 195 - RNA, 10 nt, Artificial Sequence
  chemically synthesized; RNA motif
aggrgguryy

SEQ ID NO: 196 - RNA, 28 nt, Artificial Sequence
  chemically synthesized; RNA motif
ugcrugcgggrrgcgaccauauuuyuug

SEQ ID NO: 197 - RNA, 15 nt, Artificial Sequence
  chemically synthesized; RNA motif
agraucauugcuuua

SEQ ID NO: 198 - RNA, 25 nt, Artificial Sequence
  chemically synthesized; RNA motif
ugryuucauucaruugaaryccuca

SEQ ID NO: 199 - RNA, 18 nt, Artificial Sequence
  chemically synthesized; RNA motif
gyraguacgycguaagay

SEQ ID NO: 200 - RNA, 11 nt, Artificial Sequence
  chemically synthesized; RNA motif
grcggaaarcu

SEQ ID NO: 201 - RNA, 10 nt, Artificial Sequence
  chemically synthesized; RNA motif
ggryrryayc

SEQ ID NO: 202 - RNA, 10 nt, Artificial Sequence
  chemically synthesized; RNA motif
grryyryacy

SEQ ID NO: 203 - RNA, 15 nt, Artificial Sequence
  chemically synthesized; RNA motif
gacygrgacrgrrrr

SEQ ID NO: 204 - RNA, 11 nt, Artificial Sequence
  chemically synthesized; RNA motif
yucgcgaaaaa

SEQ ID NO: 205 - RNA, 11 nt, Artificial Sequence
  chemically synthesized; RNA motif
acuauyrraaa

SEQ ID NO: 206 - RNA, 13 nt, Artificial Sequence
  chemically synthesized; RNA motif
gaarucgcaagay

SEQ ID NO: 207 - RNA, 43 nt, Artificial Sequence
  chemically synthesized; RNA motif
gaaaaucccgragggucgcaagaucaaucgggauuuuyrcuuu

SEQ ID NO: 208 - RNA, 24 nt, Artificial Sequence
  chemically synthesized; RNA motif
ycagcgguccccucuuuggggccc

SEQ ID NO: 209 - RNA, 10 nt, Artificial Sequence
  chemically synthesized; RNA motif
agcuycagug

SEQ ID NO: 210 - RNA, 12 nt, Artificial Sequence
  chemically synthesized; RNA motif
yrcugrcgcccg

SEQ ID NO: 211 - RNA, 12 nt, Artificial Sequence
  chemically synthesized; RNA motif
cggggccccgry

SEQ ID NO: 212 - RNA, 15 nt, Artificial Sequence
  chemically synthesized; RNA motif
ycggagggguggccc

SEQ ID NO: 213 - RNA, 36 nt, Artificial Sequence; identical to SEQ ID NO: 59
  chemically synthesized; RNA motif

SEQ ID NO: 214 - RNA, 12 nt, Artificial Sequence
  chemically synthesized; RNA motif
yryracugrcgr

SEQ ID NO: 215 - RNA, 11 nt, Artificial Sequence
  chemically synthesized; RNA motif
ycgycugggcr

SEQ ID NO: 216 - RNA, 15 nt, Artificial Sequence
  chemically synthesized; RNA motif
yuyuyyyrygggyrc

SEQ ID NO: 217 - RNA, 10 nt, Artificial Sequence
  chemically synthesized; RNA motif
gygcccryrr

SEQ ID NO: 218 - RNA, 37 nt, Artificial Sequence; identical to SEQ ID NO: 60
  chemically synthesized; RNA motif

SEQ ID NO: 219 - RNA, 11 nt, Artificial Sequence
  chemically synthesized; RNA motif
aaccragaccu

SEQ ID NO: 220 - RNA, 12 nt, Artificial Sequence
  chemically synthesized; RNA motif
gucuyucauayc

SEQ ID NO: 221 - RNA, 11 nt, Artificial Sequence
  chemically synthesized; RNA motif
aaccragaccu

SEQ ID NO: 222 - RNA, 12 nt, Artificial Sequence
  chemically synthesized; RNA motif
gucuyucauayc

SEQ ID NO: 223 - RNA, 21 nt, Artificial Sequence
  chemically synthesized; RNA motif
uaggauyuuuacccuuuccua

SEQ ID NO: 224 - RNA, 49 nt, Artificial Sequence
  chemically synthesized; RNA motif
guuguguuaaguguuggcagayaucyggaccccgcccuagcgguycrga

SEQ ID NO: 225 - RNA, 54 nt, Artificial Sequence
  chemically synthesized; RNA motif
cuurraccccgaacuucycccuccccauaygaagyycgggguuuuuuuugccyg

SEQ ID NO: 226 - RNA, 20 nt, Artificial Sequence
  chemically synthesized; RNA motif
gauacuguaggggycrryyc

SEQ ID NO: 227 - RNA, 27 nt, Artificial Sequence
  chemically synthesized; RNA motif
gggyygruccugucgagagaugugaug

SEQ ID NO: 228 - RNA, 12 nt, Artificial Sequence
  chemically synthesized; RNA motif
uucggccycgcr

SEQ ID NO: 229 - RNA, 19 nt, Artificial Sequence
  chemically synthesized; RNA motif
yyuyyycryygcccycugc

SEQ ID NO: 230 - RNA, 15 nt, Artificial Sequence
  chemically synthesized; RNA motif
gccgucgccgaygca

SEQ ID NO: 231 - RNA, 14 nt, Artificial Sequence
  chemically synthesized; RNA motif
gcgauccugucgcc

SEQ ID NO: 232 - RNA, 12 nt, Artificial Sequence
  chemically synthesized; RNA motif
yygcggcgcggc

SEQ ID NO: 233 - RNA, 20 nt, Artificial Sequence
  chemically synthesized; RNA motif
raccccgcggcaggggrcyy

SEQ ID NO: 234 - RNA, 20 nt, Artificial Sequence
  chemically synthesized; RNA motif
cycuyuaagycccccacccr

SEQ ID NO: 235 - RNA, 27 nt, Artificial Sequence
  chemically synthesized; RNA motif
uaccaagcugaaagucaauucyggycr

SEQ ID NO: 236 - RNA, 11 nt, Artificial Sequence
  chemically synthesized; RNA motif
yyucgaugruu

SEQ ID NO: 237 - RNA, 10 nt, Artificial Sequence
  chemically synthesized; RNA motif
gcrcugugar

SEQ ID NO: 238 - RNA, 15 nt, Artificial Sequence
  chemically synthesized; RNA motif
accgcaagguyycug

SEQ ID NO: 239 - RNA, 10 nt, Artificial Sequence
  chemically synthesized; RNA motif
ygcggyucgc

SEQ ID NO: 240 - RNA, 11 nt, Artificial Sequence
  chemically synthesized; RNA motif
aacccgccgry

SEQ ID NO: 241 - RNA, 17 nt, Artificial Sequence
  chemically synthesized; RNA motif
rgrrgagaucgacaaug

SEQ ID NO: 242 - RNA, 16 nt, Artificial Sequence
  chemically synthesized; RNA motif
yygycrgcrcrugccc

SEQ ID NO: 243 - RNA, 15 nt, Artificial Sequence
  chemically synthesized; RNA motif
grycuucraggcurc

SEQ ID NO: 244 - RNA, 30 nt, Artificial Sequence; identical to SEQ ID NO: 80
  chemically synthesized; RNA motif

SEQ ID NO: 245 - RNA, 13 nt, Artificial Sequence
  chemically synthesized; RNA motif
gcgccruayggcu

SEQ ID NO: 246 - RNA, 11 nt, Artificial Sequence
  chemically synthesized; RNA motif
gacccgyccgg

SEQ ID NO: 247 - RNA, 19 nt, Artificial Sequence
  chemically synthesized; RNA motif
yuguccccccggrcggguc

SEQ ID NO: 248 - RNA, 30 nt, Artificial Sequence; identical to SEQ ID NO: 80
  chemically synthesized; RNA motif

SEQ ID NO: 249 - RNA, 13 nt, Artificial Sequence
  chemically synthesized; RNA motif
gcgccruayggcu

SEQ ID NO: 250 - RNA, 30 nt, Artificial Sequence; identical to SEQ ID NO: 82
  chemically synthesized; RNA motif

SEQ ID NO: 251 - RNA, 10 nt, Artificial Sequence
  chemically synthesized; RNA motif
graaagugrr

SEQ ID NO: 252 - RNA, 10 nt, Artificial Sequence
  chemically synthesized; RNA motif
aycgguuuyc

SEQ ID NO: 253 - RNA, 11 nt, Artificial Sequence
  chemically synthesized; RNA motif
ycgcaaygryg

SEQ ID NO: 254 - RNA, 15 nt, Artificial Sequence
  chemically synthesized; RNA motif
acgccggcagcgrrc

SEQ ID NO: 255 - RNA, 18 nt, Artificial Sequence
  chemically synthesized; RNA motif
caggucaacarrugaggg

SEQ ID NO: 256 - RNA, 11 nt, Artificial Sequence
  chemically synthesized; RNA motif
aayacccuaug

SEQ ID NO: 257 - RNA, 27 nt, Artificial Sequence
  chemically synthesized; RNA motif
auacgugyaggguggagaugyacarcy

SEQ ID NO: 258 - RNA, 10 nt, Artificial Sequence
  chemically synthesized; RNA motif
ucggacygyu

SEQ ID NO: 259 - RNA, 22 nt, Artificial Sequence
  chemically synthesized; RNA motif
yrguugauuccuccuccugacy

SEQ ID NO: 260 - RNA, 13 nt, Artificial Sequence
  chemically synthesized; RNA motif
rcagyaagcagga

SEQ ID NO: 261 - RNA, 63 nt, Artificial Sequence
  chemically synthesized; RNA motif
grcaccgaccrugagagucgugugugccgaacgccguuuccggyagcccggaaaccaygguac

SEQ ID NO: 262 - RNA, 12 nt, Artificial Sequence
  chemically synthesized; RNA motif
rgcacygcgucc

SEQ ID NO: 263 - RNA, 12 nt, Artificial Sequence
  chemically synthesized; RNA motif
ggayaagguccu

SEQ ID NO: 264 - RNA, 32 nt, Artificial Sequence
  chemically synthesized; RNA motif
yggcauccccauuaccuauggacacggugccr

SEQ ID NO: 265 - RNA, 11 nt, Artificial Sequence
  chemically synthesized; RNA motif
arrcycyggrr

SEQ ID NO: 266 - RNA, 14 nt, Artificial Sequence
  chemically synthesized; RNA motif
ruuuuccgugucca

SEQ ID NO: 267 - RNA, 10 nt, Artificial Sequence
  chemically synthesized; RNA motif
rcauyargag

SEQ ID NO: 268 - RNA, 13 nt, Artificial Sequence
  chemically synthesized; RNA motif
cacugraaggugg

SEQ ID NO: 269 - RNA, 15 nt, Artificial Sequence
  chemically synthesized; RNA motif
rcgrguugaccycaa

SEQ ID NO: 270 - RNA, 80 nt, Artificial Sequence
  chemically synthesized; RNA motif
aacyaaucyayagugrcuuauuccaaguauaccacuugggcuuuggcaguagcuaacugcrcuaaauauaauauaaggag

SEQ ID NO: 271 - RNA, 50 nt, Artificial Sequence
  chemically synthesized; RNA motif
gccggucuccuccacguaggggaaccaucgugcagccguuaacggcuuac

SEQ ID NO: 272 - RNA, 31 nt, Artificial Sequence
  chemically synthesized; RNA motif
ggaagucagcaccaccucaggucaacgcuau

SEQ ID NO: 273 - RNA, 21 nt, Artificial Sequence
  chemically synthesized; RNA motif
auaaaaacuguugguauugcg

SEQ ID NO: 274 - RNA, 25 nt, Artificial Sequence
  chemically synthesized; RNA motif
ycacaacggcuuccugrcgugryrr

SEQ ID NO: 275 - RNA, 33 nt, Artificial Sequence
  chemically synthesized; RNA motif
gygcrgcgugaccaugcugyrcgaggacgacyu

SEQ ID NO: 276 - RNA, 29 nt, Artificial Sequence
  chemically synthesized; RNA motif
agyrccacgyccccggaagggrcguggyr

SEQ ID NO: 277 - RNA, 11 nt, Artificial Sequence
  chemically synthesized; RNA motif
ccaaugyyyyr

SEQ ID NO: 278 - RNA, 22 nt, Artificial Sequence
  chemically synthesized; RNA motif
gagrrrcgccuucgggugayyr

SEQ ID NO: 279 - RNA, 16 nt, Artificial Sequence
  chemically synthesized; RNA motif
rrcgcyuuygrgygry

SEQ ID NO: 280 - RNA, 19 nt, Artificial Sequence
  chemically synthesized; RNA motif
gucacagguggygcggyry

SEQ ID NO: 281 - RNA, 12 nt, Artificial Sequence
  chemically synthesized; RNA motif
gcgcaruaccua

SEQ ID NO: 282 - RNA, 13 nt, Artificial Sequence
  chemically synthesized; RNA motif
acgaaracggura

SEQ ID NO: 283 - RNA, 12 nt, Artificial Sequence
  chemically synthesized; RNA motif
agcycayaaagc

SEQ ID NO: 284 - RNA, 37 nt, Artificial Sequence
  chemically synthesized; RNA motif
gacyagcaggggcauccgggyurryacccggacuauc

SEQ ID NO: 285 - RNA, 18 nt, Artificial Sequence
  chemically synthesized; RNA motif
gagggugaccaagcaugc

SEQ ID NO: 286 - RNA, 11 nt, Artificial Sequence
  chemically synthesized; RNA motif
araugcggrgy

SEQ ID NO: 287 - RNA, 12 nt, Artificial Sequence
  chemically synthesized; RNA motif
aaryccgcruuu

SEQ ID NO: 288 - RNA, 10 nt, Artificial Sequence
  chemically synthesized; RNA motif
cayggaygrc

SEQ ID NO: 289 - RNA, 16 nt, Artificial Sequence
  chemically synthesized; RNA motif
rrycguuucguucygu

SEQ ID NO: 290 - RNA, 25 nt, Artificial Sequence
  chemically synthesized; RNA motif
ccgcyyyygcggracggcuccggca

SEQ ID NO: 291 - RNA, 13 nt, Artificial Sequence
  chemically synthesized; RNA motif
gccggrgyaarya

SEQ ID NO: 292 - RNA, 10 nt, Artificial Sequence
  chemically synthesized; RNA motif
gggcargaur

SEQ ID NO: 293 - RNA, 11 nt, Artificial Sequence
  chemically synthesized; RNA motif
yccuuauucgc

SEQ ID NO: 294 - RNA, 17 nt, Artificial Sequence
  chemically synthesized; RNA motif
rucyugcucugcgrggy

SEQ ID NO: 295 - RNA, 13 nt, Artificial Sequence
  chemically synthesized; RNA motif
uggugcggauggg

SEQ ID NO: 296 - RNA, 14 nt, Artificial Sequence
  chemically synthesized; RNA motif
yccgccyrguuucy

SEQ ID NO: 297 - RNA, 26 nt, Artificial Sequence
  chemically synthesized; RNA motif
gurucycyccuyggggrgrgacagar

SEQ ID NO: 298 - RNA, 11 nt, Artificial Sequence
  chemically synthesized; RNA motif
ruaaaaacacr

SEQ ID NO: 299 - RNA, 20 nt, Artificial Sequence
  chemically synthesized; RNA motif
ryyygguygguaguccrrry

SEQ ID NO: 300 - RNA, 13 nt, Artificial Sequence
  chemically synthesized; RNA motif
gyrccguacccgg

SEQ ID NO: 301 - RNA, 11 nt, Artificial Sequence
  chemically synthesized; RNA motif
gacaagacgry